SEQUENCE LISTING (75 sequences)

SEQ ID NO: 1 - protein, 748 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNDGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQRGNLADQDYTKTARQAATADVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSINVDFAVNTEGVYSEPRPIGTRYLTRNL

SEQ ID NO: 2 - protein, 748 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESEPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNDGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQRGNLADQDYTKTARQAATADVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSINVDFAVNTEGVYSEPRPIGTRYLTRNL

SEQ ID NO: 3 - protein, 748 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNDGSQAVGRSSFYCLEYFPSQMLRTGNSFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQRGNLADQDYTKTARQAATADVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSINVDFAVNTEGVYSEPRPIGTRYLTRNL

SEQ ID NO: 4 - protein, 748 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNDGSQAVGRSSFYCLEYFPSQMLRTGNNFQFTYTFEDVPFHSSYAHSQSLDRLMNPLIDQYLYFLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQRGNLADQDYTKTARQAATADVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSINVDFAVNTEGVYSEPRPIGTRYLTRNL

SEQ ID NO: 5 - protein, 748 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNDGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQQVSTTTGQNNNSNFAWTAGTKYHLNGRNSLANPGIAMATHKDDEERFFPSNGILIFGKQNAARDNADYSDVMLTSEEEIKTTNPVATEEYGIVADNLQRGNLADQDYTKTARQAATADVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSINVDFAVNTEGVYSEPRPIGTRYLTRNL

SEQ ID NO: 6 - protein, 748 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRILEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNDGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQRGNLADQDYTKTARQAATADVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSINVDFAVNTEGVYSEPRPIGTRYLTRNL

SEQ ID NO: 7 - protein, 748 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNDGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERYFPSNGILIFGKQNAARDNADYSDVMLTSEEEIKTTNPVATEEYGIVADNLQRGNLADQDYTKTARQAATADVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSINVDFAVNTEGVYSEPRPIGTRYLTRNL

SEQ ID NO: 8 - protein, 748 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNDGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQRGNLADQDYTKTARQAATSDVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSINVDFAVNTEGVYSEPRPIGTRYLTRNL

SEQ ID NO: 9 - protein, 748 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNDGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTAGQNNNSNFAWTAGTKYHLNGRNSLANPGIAMATHKDDEERFFPSNGILIFGKQNAARDNADYSDVMLTSEEEIKTTNPVATEEYGIVADNLQRGNLADQDYTKTARQAATADVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSINVDFAVNTEGVYSEPRPIGTRYLTRNL

SEQ ID NO: 10 - protein, 748 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRILEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNDGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERYFPSNGILIFGKQNAARDNADYSDVMLTSEEEIKTTNPVATEEYGIVADNLQRGNLADQDYTKTARQAATADVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSINVDFAVNTEGVYSEPRPIGTRYLTRNL

SEQ ID NO: 11 - protein, 748 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRILEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNDGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQRGNLADQDYTKTARQAATSDVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSINVDFAVNTEGVYSEPRPIGTRYLTRNL

SEQ ID NO: 12 - protein, 748 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRILEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNDGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTAGQNNNSNFAWTAGTKYHLNGRNSLANPGIAMATHKDDEERFFPSNGILIFGKQNAARDNADYSDVMLTSEEEIKTTNPVATEEYGIVADNLQRGNLADQDYTKTARQAATADVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSINVDFAVNTEGVYSEPRPIGTRYLTRNL

SEQ ID NO: 13 - protein, 747 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESEPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNDGSQAVGRSSFYCLEYFPSQMLRTGNSFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQRGNLADQDYTKTARQAATADVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSINVDFAVNTEGVYSEPRPIGTRYLTRN

SEQ ID NO: 14 - protein, 748 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRILEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNDGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTAGQNNNSNFAWTAGTKYHLNGRNSLANPGIAMATHKDDEERYFPSNGILIFGKQNAARDNADYSDVMLTSEEEIKTTNPVATEEYGIVADNLQRGNLADQDYTKTARQAATADVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSINVDFAVNTEGVYSEPRPIGTRYLTRNL

SEQ ID NO: 15 - protein, 748 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRILEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNDGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTAGQNNNSNFAWTAGTKYHLNGRNSLANPGIAMATHKDDEERFFPSNGILIFGKQNAARDNADYSDVMLTSEEEIKTTNPVATEEYGIVADNLQRGNLADQDYTKTARQAATSDVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSINVDFAVNTEGVYSEPRPIGTRYLTRNL

SEQ ID NO: 16-19 - skipped (4 entries)

SEQ ID NO: 20 - protein, 735 aa, synthetic construct [sequence omitted]

SEQ ID NO: 21 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNDGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLADQDYTKTARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 22-29 - skipped (8 entries)

SEQ ID NO: 30 - protein, 747 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQGQRGNEARVREAQAAQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 31 - protein, 20 aa, synthetic construct
RGNLADQDYTKTARQAATAD

SEQ ID NO: 32 - protein, 20 aa, synthetic construct
RGNLADQDYTKTARQAATSD

SEQ ID NO: 33 - protein, 14 aa, synthetic construct
LQQQNTAPQIGTVN

SEQ ID NO: 34 - DNA, 36 bp, synthetic construct
  source 1..36: mol_type = other DNA
tggcgactcagagtcagagccagaccctcaacctct

SEQ ID NO: 35 - DNA, 39 bp, synthetic construct
  source 1..39: mol_type = other DNA
tgctgagaaccggcaacagcttccagtttacttacacct

SEQ ID NO: 36 - DNA, 21 bp, synthetic construct
  source 1..21: mol_type = other DNA
catcgcaaggctacatgatgg

SEQ ID NO: 37 - DNA, 21 bp, synthetic construct
  source 1..21: mol_type = other DNA
ctccacacttctcaaccggtc

SEQ ID NO: 38 - protein, 417 aa, synthetic construct
MTLHSNSTTSPLFPNISSSWVHSPSEAGLPLGTVTQLGSYNISQETGNFSSNDTSSDPLGGHTIWQVVFIAFLTGFLALVTIIGNILVIVAFKVNKQLKTVNNYFLLSLACADLIIGVISMNLFTTYIIMNRWALGNLACDLWLSIDCVASNASVMNLLVISFDRYFSITSPFRYQSLMTRARAGVMIGLAWVISFVLWAPAILFWQYFVGKRTVPPGECFIQFLSEPTITFGTAIAGFYMPVTIMTILYWRVYREAKEQIRKIDRCEGRFYGSQEQPQPPPLPQHQPILGNGRASKRKTSRVMAMREHKALQTLSAILLAFIITWTPYNIMVLVNTFCDSCIPKTYWNLGYWLCYINSTVNPVCYALCNKTFRTTFKTLLLCQCDKRKRRKQQYQQRQSVIFHKRVPEQALADPPV

SEQ ID NO: 39 - DNA, 1000 bp, Homo sapiens
  source 1..1000: mol_type = genomic DNA
gggttcaccacatcagccaccactcctgcttctgagcacagggtgctctcctcttgagctcagcttctgcttttgcagccaagcattcttgctgctgctgcctgcctgcccacccgcctgggcttgcagcccgccactttactttctccagccctgataccagctgagaagtctccctgcagctgctagttcctgcccaggaccatgtgtgtggatgctgcttgggagaagcgggcacttgctcctggcactgatcccagctgagtttctcctgttgatttctggaccactgatgctgttgctgaggaggtatttcccggcatccctccccctgagacaccggctaaggaccagcctaaacgcaaggtaaccttcacctctttcccatgggattttccccagctgggatatctctgctttctcctggaactaggtcctccagctggcaacatatctctctctctctctctctctctctctctctctctctctctgtgtgtgtgtgtgtgtgttgggggagaagaggagactgttttaggtattgctctgtcctagggcttccccgatacctgctgaatccaatgcaaagtggcctttcccctggctctggagacatttccgaggcaggcacctgtcatctgccacccccgtcctcctcctcgagctccctttctccccctcccccagcccattattctgcttcagccttttgtgtcagtggcagagggctgaagggatgtctttgcccttctggcaggcaggacagtgtcaggatggaccgcgctgccagaagccgacgctagcgagggaggtgtgaagagttggccagaatgaccaactcctcctccacatccacctcctccaccaccggtggctcgctgctgctgctctgcgaggaagaggagtcgtgggcgggccggcgcatcccggtgtcactcctgtattcgggcctggccatcgggggcacgctggccaacggcatggtcatctatctcgtgtc

SEQ ID NO: 40 - DNA, 900 bp, Homo sapiens
  source 1..900: mol_type = genomic DNA
gggttcaccacatcagccaccactcctgcttctgagcacagggtgctctcctcttgagctcagcttctgcttttgcagccaagcattcttgctgctgctgcctgcctgcccacccgcctgggcttgcagcccgccactttactttctccagccctgataccagctgagaagtctccctgcagctgctagttcctgcccaggaccatgtgtgtggatgctgcttgggagaagcgggcacttgctcctggcactgatcccagctgagtttctcctgttgatttctggaccactgatgctgttgctgaggaggtatttcccggcatccctccccctgagacaccggctaaggaccagcctaaacgcaaggtaaccttcacctctttcccatgggattttccccagctgggatatctctgctttctcctggaactaggtcctccagctggcaacatatctctctctctctctctctctctctctctctctctctctctgtgtgtgtgtgtgtgtgttgggggagaagaggagactgttttaggtattgctctgtcctagggcttccccgatacctgctgaatccaatgcaaagtggcctttcccctggctctggagacatttccgaggcaggcacctgtcatctgccacccccgtcctcctcctcgagctccctttctccccctcccccagcccattattctgcttcagccttttgtgtcagtggcagagggctgaagggatgtctttgcccttctggcaggcaggacagtgtcaggatggaccgcgctgccagaagccgacgctagcgagggaggtgtgaagagttggccagaatgaccaactcctcctccacatccacctcctccaccaccggtggctcgctgctgctgctctgcgaggaag

SEQ ID NO: 41 - DNA, 100 bp, Homo sapiens
  source 1..100: mol_type = genomic DNA
tccttttacaagttggaagctattgaataattcatgccaagcgtggaaaggctgtcttttccctggatgatgtataactaagcaggagagagcttcgagt

SEQ ID NO: 42 - DNA, 600 bp, Homo sapiens
  source 1..600: mol_type = genomic DNA
actgaataatattccattgtctagatgtaccactgacatctgctttaaatcttttgcccatttcatttttaccctctaaccatttcattactaaaacacaaaacaaactccggaaaggagtttgttgatgagaaaacactgaaagtacgatttaaattgaggaatggagattttctgtggataattgtctatgttccactttaccaaagaaaaatgaatttcaagtagtgcataaagtaattctcgttaaaaaatgtgggaataaagagatcagtcctgggtactgggaatatatttatctcacagaaaaatcttgcccacaaaatgcatcagactgagatgaaatactctagggtatattaaattttattctaatattgaagggagggaaaaaaaggacggaaagaaaaatgaaacaggatgaaaaataaggtgattgggagagagaaaagacagagaaacttgttaacgacaggactttctgagcaagatatttttcttccttttacaagttggaagctattgaataattcatgccaagcgtggaaaggctgtcttttccctggatgatgtataactaagcaggagagagcttcgagt

SEQ ID NO: 43 - DNA, 1500 bp, Homo sapiens
  source 1..1500: mol_type = genomic DNA
cagaaagttttaacagaaagggcctttgaagtgactgactgtgaacttatttgacaagatgaaaataatccatcgcaaggctacatgatggatgctcggcttagtatctgggtgtgctgcaaccccacctaacatttttttctattatgccatgttaccatattcccatattgagttacattttcaaatacatgacttttccagtgaataattgagagaactccttttctgaaaaaattcccacatggaatggataaatgaacacttgcaccaaagaagatgccaacatcagactgcaaatctccaagacagacaagacaaaaagttctgaagtgaaaagtcatctttgttcacactgtggattattcaggtttcttgctttccccctgacacacatttttttaaaattgactttatttttttagagcaattttagattcatggcaaatttgagtgaaagatacagagatttcccatgtactccctgcccctcgcatacatagcctctcccattatcaaaatcccccaccagagtggttcgtctgttacaattgaatgtacgttgacacatcattccccaaagtccatggcttacactgatgtttcacattctgccactttgggcaaatttataagtacacagatccactattatggtatcatacagagtagttccactgtcctaagaatcttccgtgatcatctattcattcctcctttccccctaactcgggcaaccactggtctttttaactgtctccatagttttgccttctcaagaatgtcatagttggtagcatacagtatgtagtcctttccagttggcttctgtcacttagtgatacacacttaaggttccttcatgtcttttcatggctggacagctcatttctttttagcactgaataatattccattgtctagatgtaccactgacatctgctttaaatcttttgcccatttcatttttaccctctaaccatttcattactaaaacacaaaacaaactccggaaaggagtttgttgatgagaaaacactgaaagtacgatttaaattgaggaatggagattttctgtggataattgtctatgttccactttaccaaagaaaaatgaatttcaagtagtgcataaagtaattctcgttaaaaaatgtgggaataaagagatcagtcctgggtactgggaatatatttatctcacagaaaaatcttgcccacaaaatgcatcagactgagatgaaatactctagggtatattaaattttattctaatattgaagggagggaaaaaaaggacggaaagaaaaatgaaacaggatgaaaaataaggtgattgggagagagaaaagacagagaaacttgttaacgacaggactttctgagcaagatatttttcttccttttacaagttggaagctattgaataattcatgccaagcgtggaaaggctgtcttttccctggatgatgtataactaagcaggagagagcttcgagt

SEQ ID NO: 44 - DNA, 2259 bp, Homo sapiens
  source 1..2259: mol_type = genomic DNA
catcgcaaggctacatgatggatgctcggcttagtatctgggtgtgctgcaaccccacctaacatttttttctattatgccatgttaccatattcccatattgagttacattttcaaatacatgacttttccagtgaataattgagagaactccttttctgaaaaaattcccacatggaatggataaatgaacacttgcaccaaagaagatgccaacatcagactgcaaatctccaagacagacaagacaaaaagttctgaagtgaaaagtcatctttgttcacactgtggattattcaggtttcttgctttccccctgacacacatttttttaaaattgactttatttttttagagcaattttagattcatggcaaatttgagtgaaagatacagagatttcccatgtactccctgcccctcgcatacatagcctctcccattatcaaaatcccccaccagagtggttcgtctgttacaattgaatgtacgttgacacatcattccccaaagtccatggcttacactgatgtttcacattctgccactttgggcaaatttataagtacacagatccactattatggtatcatacagagtagttccactgtcctaagaatcttccgtgatcatctattcattcctcctttccccctaactcgggcaaccactggtctttttaactgtctccatagttttgccttctcaagaatgtcatagttggtagcatacagtatgtagtcctttccagttggcttctgtcacttagtgatacacacttaaggttccttcatgtcttttcatggctggacagctcatttctttttagcactgaataatattccattgtctagatgtaccactgacatctgctttaaatcttttgcccatttcatttttaccctctaaccatttcattactaaaacacaaaacaaactccggaaaggagtttgttgatgagaaaacactgaaagtacgatttaaattgaggaatggagattttctgtggataattgtctatgttccactttaccaaagaaaaatgaatttcaagtagtgcataaagtaattctcgttaaaaaatgtgggaataaagagatcagtcctgggtactgggaatatatttatctcacagaaaaatcttgcccacaaaatgcatcagactgagatgaaatactctagggtatattaaattttattctaatattgaagggagggaaaaaaaggacggaaagaaaaatgaaacaggatgaaaaataaggtgattgggagagagaaaagacagagaaacttgttaacgacaggactttctgagcaagatatttttcttccttttacaagttggaagctattgaataattcatgccaagcgtggaaaggctgtcttttccctggatgatgtataactaagcaggagagagcttcgagtgggttcaccacatcagccaccactcctgcttctgagcacagggtgctctcctcttgagctcagcttctgcttttgcagccaagcattcttgctgctgctgcctgcctgcccacccgcctgggcttgcagcccgccactttactttctccagccctgataccagctgagaagtctccctgcagctgctagttcctgcccaggaccatgtgtgtggatgctgcttgggagaagcgggcacttgctcctggcactgatcccagctgagtttctcctgttgatttctggaccactgatgctgttgctgaggaggtatttcccggcatccctccccctgagacaccggctaaggaccagcctaaacgcaaggtaaccttcacctctttcccatgggattttccccagctgggatatctctgctttctcctggaactaggtcctccagctggcaacatatctctctctctctctctctctctctctctctctctctctctgtgtgtgtgtgtgtgtgttgggggagaagaggagactgttttaggtattgctctgtcctagggcttccccgatacctgctgaatccaatgcaaagtggcctttcccctggctctggagacatttccgaggcaggcacctgtcatctgccacccccgtcctcctcctcgagctccctttctccccctcccccagcccattattctgcttcagccttttgtgtcagtggcagagggctgaagggatgtctttgcccttctggcaggcaggacagtgtcaggatggaccgcgctgccagaagccgacgctagcgagggaggtgtgaagagttggccag

SEQ ID NO: 45 - DNA, 1395 bp, Homo sapiens
  source 1..1395: mol_type = genomic DNA
tctagatgtaccactgacatctgctttaaatcttttgcccatttcatttttaccctctaaccatttcattactaaaacacaaaacaaactccggaaaggagtttgttgatgagaaaacactgaaagtacgatttaaattgaggaatggagattttctgtggataattgtctatgttccactttaccaaagaaaaatgaatttcaagtagtgcataaagtaattctcgttaaaaaatgtgggaataaagagatcagtcctgggtactgggaatatatttatctcacagaaaaatcttgcccacaaaatgcatcagactgagatgaaatactctagggtatattaaattttattctaatattgaagggagggaaaaaaaggacggaaagaaaaatgaaacaggatgaaaaataaggtgattgggagagagaaaagacagagaaacttgttaacgacaggactttctgagcaagatatttttcttccttttacaagttggaagctattgaataattcatgccaagcgtggaaaggctgtcttttccctggatgatgtataactaagcaggagagagcttcgagtgggttcaccacatcagccaccactcctgcttctgagcacagggtgctctcctcttgagctcagcttctgcttttgcagccaagcattcttgctgctgctgcctgcctgcccacccgcctgggcttgcagcccgccactttactttctccagccctgataccagctgagaagtctccctgcagctgctagttcctgcccaggaccatgtgtgtggatgctgcttgggagaagcgggcacttgctcctggcactgatcccagctgagtttctcctgttgatttctggaccactgatgctgttgctgaggaggtatttcccggcatccctccccctgagacaccggctaaggaccagcctaaacgcaaggtaaccttcacctctttcccatgggattttccccagctgggatatctctgctttctcctggaactaggtcctccagctggcaacatatctctctctctctctctctctctctctctctctctctgtgtgtgtgtgtgtgtgttgggggagaagaggagactgttttaggtattgctctgtcctagggcttccccgatacctgctgaatccaatgcaaagtggcctttcccctggctctggagacatttccgaggcaggcacctgtcatttgccacccccgtcctcctcctcgagctccctttctccccctcccccagcccattattctgcttcagccttttgtgtcagtggcagagggctgaagggatgtctttgcccttctggcaggcaggacagtgtcaggatggaccgcgctgccagaagccgacgctagcgagggaggtgtgaa

SEQ ID NO: 46 - DNA, 896 bp, Homo sapiens
  source 1..896: mol_type = genomic DNA
atgccaagcgtggaaaggctgtcttttccctggatgatgtataactaagcaggagagagcttcgagtgggttcaccacatcagccaccactcctgcttctgagcacagggtgctctcctcttgagctcagcttctgcttttgcagccaagcattcttgctgctgctgcctgcctgcccacccgcctgggcttgcagcccgccactttactttctccagccctgataccagctgagaagtctccctgcagctgctagttcctgcccaggaccatgtgtgtggatgctgcttgggagaagcgggcacttgctcctggcactgatcccagctgagtttctcctgttgatttctggaccactgatgctgttgctgaggaggtatttcccggcatccctccccctgagacaccggctaaggaccagcctaaacgcaaggtaaccttcacctctttcccatgggattttccccagctgggatatctctgctttctcctggaactaggtcctccagctggcaacatatctctctctctctctctctctctctctctctctctctctctgtgtgtgtgtgtgtgtgttgggggagaagaggagactgttttaggtattgctctgtcctagggcttccccgatacctgctgaatccaatgcaaagtggcctttcccctggctctggagacatttccgaggcaggcacctgtcatctgccacccccgtcctcctcctcgagctccctttctccccctcccccagcccattattctgcttcagccttttgtgtcagtggcagagggctgaagggatgtctttgcccttctggcaggcaggacagtgtcaggatggaccgcgctgccagaagccgacgctagcgagggaggtgtgaagagttggccag

SEQ ID NO: 47 - DNA, 2152 bp, Homo sapiens
  source 1..2152: mol_type = genomic DNA
ccaagcactgtgttacacatctcctcattaaaatggtatagcaaccttttctggtcgatattatgatcccatttttcaatttttacttttgagtgaaggacattgattcttagagattgtaaggaacttgcccaaggctgccggctggttagtggcagagctgggatgggaaagtccgtattttccaccgcgctgtattagtgagggctctttgtgtcacttctgtgcataactccgccccaagttaaaaggctcgctgctctcgacaggtcctcctcctcctcccgctaggtcctcctcctcctcccgccaggtcctcctcctccttctcctcccggggccctgccctgcgggtctcacctgctgctccgcggggtcccggcgggggccggatagcgccccctggcggcaggtgggcgcccgcgcagcgcatgctaatgagcccgcggcgccgctcgcggattggctggcgcggattccagctgctttccaagtcagcggcgcctagtgagagtcaggggggcccggcccgcgccctccccgcccagccgcctccccgtcgacgcccagggctggggcgagccaggctgcctttcgaacttggggggcttctcctcttgtctcccactggtgctctggctgtgaatccatccaggggccaggatgacaatccgacaccaaggccagcagtacaggccgaggatggcatttctccaaaaggtaaccctggcccctcacctggaccctgggaggagggcgggggaccgcatctgcgaagcgtccgaggacaagaggggcaggagtggagagcacccctttgggtcgatttgtgctgctcgattttattaagtgacttctttgctgtgtgtgtcgatattgatggctataataattaggttttcgtgtgccctgcctgtagtggagaatctgaagccagcgctgatggtctgcgggaggtgaaaacaacctgttactcgggagaggcaggttcagttcaggctgtggcagatttgagacagcctgctgatattgttgtaagacgaccagatgtgacattgccatttttgccttttttttctgatcccaaactgcactattttaaagtgtgtgtgtgtgtacacatagaccatgaaaacggggtgatttgtaggataaagggccaccccatttacaatgccctgagagccctcatttgagtgtgtctatctgtgcatctcctgctgtctgcgctggcggcagaagtcccaaccttttcgcttcccttgcgaatgctttcccagacaaagtgaccgggcacagggtgtgcacacaggatggacatgtgggaggtggaatgcacctgtggggtgatgtgtttggggacaaagtggttcatttagttctgagcttctggtataatcactgtccgatgagatctacagaggattaggtcccgggtcacatctttgtccttcctcccacccgtagccacttttcacagcgtagtgtccatcttcttaggcttatcttgttgagtagaattagttgaatgttttgtgacaagctgagctctccctgagcccctggagctcagcaaatttgtatagtcagtgaccgctgtcctgatttgcccaggactttcaatgctaaaacctggacagtcctggggaccccgagacggtcgcatttggtccacctgtttcaagatgctcgtgctcatcttccgggtagcagaggtgtgggtatcatctgtaaagcagacaggctgaatgttgtctcactcagaaagaaccctgagagcctgtattgcagataccagttactaaaagcagaagaaactgcctaagtcggctatctcaaagccacacatcatcttccaggatttatcctaggactgtctcctcccaggcaggacagctctggcttgggggctcagagggtaattgtcaagagataaattcagtttgtttattgaatctctggcattcttttccatcatgactcttctctggtagattctcacccctgtgtccacctttgcattttgaggtccctctcagtgcagtggaaattgccctgcacaactttcaaaattgttctcgtcagtcggctttttcctgttctgtcttgcagattgaagcgctcgtgaaggacatgcagaacccagagacag

SEQ ID NO: 48 - DNA, 1665 bp, Homo sapiens
  source 1..1665: mol_type = genomic DNA
tacagagtagttccactgtcctaagaatcttccgtgatcatctattcattcctcctttccccctaactcgggcaaccactggtctttttaactgtctccatagttttgccttctcaagaatgtcatagttggtagcatacagtatgtagtcctttccagttggcttctgtcacttagtgatacacacttaaggttccttcatgtcttttcatggctggacagctcatttctttttagcactgaataatattccattgtctagatgtaccactgacatctgctttaaatcttttgcccatttcatttttaccctctaaccatttcattactaaaacacaaaacaaactccggaaaggagtttgttgatgagaaaacactgaaagtacgatttaaattgaggaatggagattttctgtggataattgtctatgttccactttaccaaagaaaaatgaatttcaagtagtgcataaagtaattctcgttaaaaaatgtgggaataaagagatcagtcctgggtactgggaatatatttatctcacagaaaaatcttgcccacaaaatgcatcagactgagatgaaatactctagggtatattaaattttattctaatattgaagggagggaaaaaaaggacggaaagaaaaatgaaacaggatgaaaaataaggtgattgggagagagaaaagacagagaaacttgttaacgacaggactttctgagcaagatatttttcttccttttacaagttggaagctattgaataattcatgccaagcgtggaaaggctgtcttttccctgggtgatgtataactaagcaggagagagcttcgagtgggttcaccacatcagccaccactcctgcttctgagcacagggtgctctcctcttgagctcagcttctgcttttgcagccaagcattcttgctgctgctgcctgcctgcccacccgcctgggcttgcagcccgccactttactttctccagccctgataccagctgagaagtctccctgcagctgctagttcctgcccaggaccaggtgtgtggacgctgcttgggagaagcgggcacttgctcctggcactgatcccagctgagtttctcctgttgatttctggaccactgacgctgttgctgaggaggtatttcctggcatccctccccctgagacaccggctaaggaccagcctaaacgcaaggtaaccttcacctctttcccatgggattttccccagctgggatatctctgctttctcctggaactaggtcctccagctggcaacatctctctctctctctctctctctctctctctctgtgtgtgtgtgtgtgtgtgtgtgtgttgggggagaagaggagactgttttaggtattgctctgtcctagggcttccccgatacctgctgaatccaatgcaaagtggcctttcccctggctctggagacatttccgaggcaggcacctgtcatctgccacccccgtcctcctcctcgagctccctttctccccctcccccagcccattattctgcttcagccttttgtgtcagtggcagagggctgaagggatgtctttgcccttctggcaggcaggacagtgtcaggatggaccgcgctgccagaagccgacgctagcgagggaggtgtgaagagttggccag

SEQ ID NO: 49 - protein, 414 aa, Homo sapiens
MGTLHNNSTTSPLFPNISSSWIHSPSDAGLPPGTVTHFGSYNVSRAAGNFSSPDGTTDDPLGGHTVWQVVFIAFLTGILALVTIIGNILVIVSFKVNKQLKTVNNYFLLSLACADLIIGVISMNLFTTYIIMNRWALGNLACDLWLAIDCVASNASVMNLLVISFDRYFSITSPFRYQSLMTRARAGVMIGLAWVISFVLWAPAILFWQYFVGKRTVPPGECFIQFLSEPTITFGTAIAGFYMPVTIMTILYWRVYREAKEQIRKIDRCEGRFYGSQEQPQPPPLPQHQPILGNGRASKRKTSRVMAMREHKALQTLSAILLAFIITWTPYNIMVLVNTFCDSCIPKTFWNLGYWLCYINSTVNPVCYALCNKTFRTTFKMLLLCQCDKKKRRKQQYQQRQSVIFHKRAPEQAL

SEQ ID NO: 50 - protein, 414 aa, Homo sapiens
MGTLHNNSTTSPLFPNISSSWIHSPSDAGLPPGTVTHFGSYNVSRAAGNFSSPDGTTDDPLGGHTVWQVVFIAFLTGILALVTIIGNILVIVSFKVNKQLKTVNNYFLLSLACADLIIGVISMNLFTTYIIMNRWALGNLACDLWLSIDCVASNASVMNLLVISFDRYFSITSPFRYQSLMTRARAGVMIGLAWVISFVLWAPAILFWQYFVGKRTVPPGECFIQFLSEPTITFGTAIAGFYMPVTIMTILYWRVYREAKEQIRKIDRCEGRFYGSQEQPQPPPLPQHQPILGNGRASKRKTSRVMAMREHKALQTLSAILLAFIITWTPYNIMVLVNTFCDSCIPKTYWNLGYWLCYINSTVNPVCYALCNKTFRTTFKMLLLCQCDKKKRRKQQYQQRQSVIFHKRAPEQAL

SEQ ID NO: 51 - DNA, 1667 bp, Homo sapiens
  source 1..1667: mol_type = genomic DNA
tacagagtagttccactgtcctaagaatcttccgtgatcatctattcattcctcctttccccctaactcgggcaaccactggtctttttaactgtctccatagttttgccttctcaagaatgtcatagttggtagcatacagtatgtagtcctttccagttggcttctgtcacttagtgatacacacttaaggttccttcatgtcttttcatggctggacagctcatttctttttagcactgaataatattccattgtctagatgtaccactgacatctgctttaaatcttttgcccatttcatttttaccctctaaccatttcattactaaaacacaaaacaaactccggaaaggagtttgttgatgagaaaacactgaaagtacgatttaaattgaggaatggagattttctgtggataattgtctatgttccactttaccaaagaaaaatgaatttcaagtagtgcataaagtaattctcgttaaaaaatgtgggaataaagagatcagtcctgggtactgggaatatatttatctcacagaaaaatcttgcccacaaaatgcatcagactgagatgaaatactctagggtatattaaattttattctaatattgaagggagggaaaaaaaggacggaaagaaaaatgaaacaggatgaaaaataaggtgattgggagagagaaaagacagagaaacttgttaacgacaggactttctgagcaagatatttttcttccttttacaagttggaagctattgaataattcatgccaagcgtggaaaggctgtcttttccctggatgatgtataactaagcaggagagagcttcgagtgggttcaccacatcagccaccactcctgcttctgagcacagggtgctctcctcttgagctcagcttctgcttttgcagccaagcattcttgctgctgctgcctgcctgcccacccgcctgggcttgcagcccgccactttactttctccagccctgataccagctgagaagtctccctgcagctgctagttcctgcccaggaccatgtgtgtggatgctgcttgggagaagcgggcacttgctcctggcactgatcccagctgagtttctcctgttgatttctggaccactgatgctgttgctgaggaggtatttcccggcatccctccccctgagacaccggctaaggaccagcctaaacgcaaggtaaccttcacctctttcccatgggattttccccagctgggatatctctgctttctcctggaactaggtcctccagctggcaacatatctctctctctctctctctctctctctctctctctctctctgtgtgtgtgtgtgtgtgttgggggagaagaggagactgttttaggtattgctctgtcctagggcttccccgatacctgctgaatccaatgcaaagtggcctttcccctggctctggagacatttccgaggcaggcacctgtcatctgccacccccgtcctcctcctcgagctccctttctccccctcccccagcccattattctgcttcagccttttgtgtcagtggcagagggctgaagggatgtctttgcccttctggcaggcaggacagtgtcaggatggaccgcgctgccagaagccgacgctagcgagggaggtgtgaagagttggccag

SEQ ID NO: 52 - DNA, 21 bp, synthetic construct
  source 1..21: mol_type = other DNA
gaggtgtgaagagttggccag

SEQ ID NO: 53 - protein, 6 aa, synthetic construct
SSSTDP

SEQ ID NO: 54 - protein, 16 aa, synthetic construct
RGNLADQDYTKTARQA

SEQ ID NO: 55 - protein, 8 aa, synthetic construct
NQSSTTAP

SEQ ID NO: 56 - protein, 18 aa, synthetic construct
LQRGNLADQDYTKTARQA

SEQ ID NO: 57 - protein, 4 aa, synthetic construct
KSIN

SEQ ID NO: 58 - DNA, 36 bp, synthetic construct
  source 1..36: mol_type = other DNA
agaggttgagggtctggctctgactctgagtcgcca

SEQ ID NO: 59 - protein, 748 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESEPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNDGSQAVGRSSFYCLEYFPSQMLRTGNSFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQRGNLADQDYTKTARQAATADVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSINVDFAVNTEGVYSEPRPIGTRYLTRNL

SEQ ID NO: 60 - DNA, 21 bp, synthetic construct
  source 1..21: mol_type = other DNA
ctggccaactcttcacacctc

SEQ ID NO: 61 - protein, 24 aa, synthetic construct
LQRGNLADQDYTKTARQAATADVN

SEQ ID NO: 62 - protein, 8 aa, synthetic construct
SSSTDPAT

SEQ ID NO: 63 - protein, 20 aa, synthetic construct
FQRGNLADQDYTKTARQAAT

SEQ ID NO: 64 - protein, 8 aa, synthetic construct
SSTTAPAT

SEQ ID NO: 65 - protein, 4 aa, synthetic construct
DPQF

SEQ ID NO: 66 - protein, 20 aa, synthetic construct
LQRGNLADQDYTKTARQAAT

SEQ ID NO: 67 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNHGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 68 - protein, 748 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNHGTKTIANNLTSTIQVFTDSEYQLPYVLGSAHQGCLPPFPADVFMIPQYGYLTLNDGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQRGNLADQDYTKTARQAATADVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSINVDFAVNTEGVYSEPRPIGTRYLTRNL

SEQ ID NO: 69 - protein, 748 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESEPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNHGTKTIANNLTSTIQVFTDSEYQLPYVLGSAHQGCLPPFPADVFMIPQYGYLTLNDGSQAVGRSSFYCLEYFPSQMLRTGNSFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQRGNLADQDYTKTARQAATADVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSINVDFAVNTEGVYSEPRPIGTRYLTRNL

SEQ ID NO: 70 - protein, 52 aa, synthetic construct
LACADLIIGVISMNLFTTYIIMNRWALGNLACDLWLAIDCVASNASVMNLLV

SEQ ID NO: 71 - protein, 52 aa, synthetic construct
LACADLIIGVISMNLFTTYIIMNRWALGNLACDLWLSIDCVASNASVMNLLV

SEQ ID NO: 72 - protein, 40 aa, synthetic construct
MVLVNTFCDSCIPKTFWNLGYWLCYINSTVNPVCYALCNK

SEQ ID NO: 73 - protein, 40 aa, synthetic construct
MVLVNTFCDSCIPKTYWNLGYWLCYINSTVNPVCYALCNK

SEQ ID NO: 74 - protein, 8 aa, synthetic construct
FQSSSTDP

SEQ ID NO: 75 - protein, 8 aa, synthetic construct
LQSSSTDP

(19) United States
(12) Patent Application Publication
(10) Pub. No.: US 2025/0381307 A1
(43) Pub. Date: Dec. 18, 2025

(54) RETRO-AAV AND USE IN TREATING NEURODEGENERATIVE DISEASES

(71) Applicant: Emugen Therapeutics LLC, Woburn, MA (US)

(72) Inventors: Zhonghua LU, Shenzhen (CN); Yefei CHEN, Cambridge, MA (US); Ji DAI, Shenzhen (CN); Jianbang LIN, Shenzhen (CN); Taian LIU, Shenzhen (CN); Jianqing ZHANG, Shenzhen (CN); Xian GAO, Belmont, MA (US); Jonathan WILDE, Waltham, MA (US)

(21) Appl. No.: 19/255,609

(22) Filed: Jun. 30, 2025

Related U.S. Application Data
(63) Continuation of application No. PCT/US2024/010340, filed on Jan. 4, 2024.
(60) Provisional application No. 63/437,213, filed on Jan. 5, 2023.
(60) Provisional application No. 63/589,867, filed on Oct. 12, 2023.

(51) Int. Cl. A61K 48/00; C12N 15/86

(52) U.S. Cl. CPC A61K 48/0058; C12N 15/86; C12N 2750/14143; C12N 2830/008

(57) ABSTRACT

The present invention provides for retro adeno-associated virus (retro-AAV) virions comprising a variant capsid polypeptide, wherein the variant capsid polypeptide comprises one or more alterations that promote retrograde transport of the retro-AAV by a neuron, and a nucleic acid comprising a heterologous gene of interest operatively coupled to a GPR88 and/or a R9P1 regulatory region.

### CROSS-REFERENCE

This application is a continuation of International Application No. PCT/US2024/010340, filed Jan. 4, 2024, which claims the benefit of U.S. Provisional Application No. 63/437,213, filed Jan. 5, 2023, and U.S. Provisional Application No. 63/589,867, filed Oct. 12, 2023, which applications are incorporated herein by reference.

### INCORPORATION BY REFERENCE OF SEQUENCE LISTING

The instant application contains a Sequence Listing which has been submitted electronically in XML format and is hereby incorporated by reference in its entirety. Said XML copy, created on Dec. 20, 2023, is named 062692-503C01US_SL.xml and is 89,597 bytes in size.

### BACKGROUND

Adeno-associated viruses (AAV) are small (25 nm) viruses belonging to the Parvovirus family which infect humans and other primate species. AAV are used as delivery vectors for gene therapy as they are capable of establishing a latent infection whereby the AAV genome is incorporated into the host chromosome without provoking a destructive T cell immune response. Approximately 13 serotypes of AAV have been isolated from the wild.

### SUMMARY

Parkinson's disease (PD) is a debilitating neurodegenerative disorder. Its symptoms are typically treated with levodopa or dopamine receptor agonists, but their action lacks specificity due to the wide distribution of dopamine receptors in the central nervous system and the periphery. This disclosure includes development of a gene therapy strategy to selectively manipulate PD-affected circuitry. Targeting striatal D1 medium spiny neurons (MSNs) whose activity may be chronically suppressed in PD, a therapeutic strategy was engineered that may include a highly efficient novel retrograde AAV, promoter elements with strong D1-MSN activity, and a chemogenetic effector to enable precise D1-MSN activation after systemic ligand administration. Application of this therapeutic approach can rescue locomotion, tremor, and motor skill defects in PD, supporting the usefulness of targeted circuit modulation tools for the treatment of PD in humans.

The present disclosure provides chimeric AAV viruses which have variant capsid polypeptides that promote retrograde transport of the AAV by a neuron. The present disclosure also provides chimeric AAV viruses which comprise a heterologous gene of interest operatively coupled to regulatory elements which have increased gene expression in neurons. The present disclosure provides AAVs for use in methods for treating Parkinson's disease. The present disclosure provides designer receptors exclusively activated by designer drugs (DREADD) for use in methods for treating Parkinson's disease.

The present disclosure provides AAV variant capsid proteins and virions that provide said variant capsid proteins with higher infectivity especially in neuronal cells. The variants allow for improved delivery of gene therapies, therapeutic proteins, and/or designer receptors, including to neuronal tissue (e.g., dopaminergic medium spiny neurons). Such improved delivery can be used in the treatment of Parkinson's disease.

The present disclosure provides promoters and promoter sequences with increased gene expression, especially in neuronal cells. The promoters allow for improved expression of gene therapies, therapeutic proteins, and/or designer receptors, in neuronal tissue (e.g., dopaminergic medium spiny neurons). Such improved expression can be used in the treatment of Parkinson's disease.

In some embodiments, the present disclosure provides a retro adeno-associated virus (retro-AAV), wherein the retro-AAV comprises an adeno-associated virus capsid polypeptide comprising one or more alterations that promote retrograde transport of the retro-AAV by a neuron, and a nucleic acid comprising a heterologous gene of interest operatively coupled to a GPR88 regulatory region and/or a R9P1 regulatory region.

In some embodiments, the retro-AAV is a serotype selected from AAV2, AAV8 or a combination thereof. In some embodiments, the one or more alterations that promote retrograde transport of the retro-AAV by a neuron is selected from the list consisting of an insertion of SEQ ID NO: 31, an aspartic acid substitution at an amino acid residue corresponding to position 385 of SEQ ID NO: 1, an isoleucine and asparagine (IN) substitution at an amino acid residue corresponding to positions 721 and 722 of SEQ ID NO: 1, and combinations thereof.

In some embodiments, the retro-AAV comprises an alteration at one or more amino acid residues corresponding to V125, V183, N411, Y447, R490, T495, or F536 of SEQ ID NO: 1. In some embodiments, the capsid polypeptide comprises an amino acid sequence that possesses at least 90%, 95%, 97%, 98%, 99% sequence identity or that is identical to the amino acid sequence set forth in SEQ ID NO: 1, wherein the variant capsid polypeptide comprises an alteration to SEQ ID NO: 1 at an amino acid selected from the list consisting of any one or more of V125, V183, N411, Y447, R490, T495, F536, and A606. In some embodiments, the capsid polypeptide comprises an alteration to SEQ ID NO: 1 corresponding to an amino acid selected from the list consisting of any two or more of V125, V183, N411, Y447, R490, T495, F536, and A606. In some embodiments, the capsid polypeptide comprises an alteration to SEQ ID NO: 1 corresponding to an amino acid selected from the list consisting of any three or more of V125, V183, N411, Y447, R490, T495, F536, and A606. In some embodiments, the capsid polypeptide comprises a substitution to SEQ ID NO: 1 corresponding to an amino acid selected from the list consisting of any one or more of V125I, V183E, N411S, Y447F, R490Q, T495A, F536Y, and A606S. In some embodiments, the capsid polypeptide comprises a substitution to SEQ ID NO: 1 corresponding to an amino acid selected from the list consisting of any two or more of V125I, V183E, N411 S, Y447F, R490Q, T495A, F536Y, and A606S. In some embodiments, the capsid polypeptide comprises a substitution to SEQ ID NO: 1 corresponding to an amino acid selected from the list consisting of any three or more of V125I, V183E, N411S, Y447F, R490Q, T495A, F536Y, and A606S.

In some embodiments, the capsid polypeptide comprises a substitution corresponding to V125I and N411S of SEQ ID NO: 1. In some embodiments, the capsid polypeptide comprises a substitution corresponding to V125I, F536Y, T495A of SEQ ID NO: 1. In some embodiments, the capsid polypeptide comprises a substitution corresponding to V125I, A606S, and a T495A of SEQ ID NO: 1. In some embodiments, the capsid polypeptide comprises the amino acid sequence set forth in any one of SEQ ID NOs 1 to 15. In some embodiments, the capsid polypeptide comprises the amino acid sequence set forth in any one of SEQ ID NOs 2 to 15.

In some embodiments, the nucleic acid comprising a heterologous gene of interest is operatively coupled to a regulatory element, wherein the regulatory element comprises a nucleotide sequence corresponding to a genomic sequence positioned 3′ to a translational start site of an endogenous GPR88 gene. In some embodiments, the genomic sequence positioned 3′ to a translational start site of the endogenous GPR88 gene is partially positioned in an intron. In some embodiments, the genomic sequence positioned 3′ to a translational start site of the endogenous GPR88 gene is positioned in an intron.

In some embodiments, the genomic sequence positioned 3′ to a translational start site of the endogenous GPR88 gene is positioned less than about 1,000 nucleotides 3′ to the translational start site of the endogenous GPR88 gene. In some embodiments, the regulatory element comprises a nucleotide sequence that is at least 80%, 85%, 90%, 95%, 97%, 98%, 99% homologous to the nucleotide sequence set forth in SEQ ID NO: 39. In some embodiments, the regulatory element comprises a nucleotide sequence that is identical to the nucleotide sequence set forth in SEQ ID NO: 39.

In some embodiments, the genomic sequence positioned 3′ to a translational start site of the endogenous GPR88 gene is positioned less than about 900 nucleotides 3′ to the translational start site of the endogenous GPR88 gene. In some embodiments, the regulatory element comprises a nucleotide sequence that is at least 80%, 85%, 90%, 95%, 97%, 98%, 99% homologous to the nucleotide sequence set forth in SEQ ID NO: 40. In some embodiments, the regulatory element comprises a nucleotide sequence that is identical to the nucleotide sequence set forth in SEQ ID NO: 40.

In some embodiments, the regulatory element comprises a nucleotide sequence corresponding to a genomic sequence positioned 5′ to the translational start site of the endogenous GPR88 gene. In some embodiments, the genomic sequence positioned 5′ to a translational start site of the endogenous GPR88 gene is positioned less than about 100 nucleotides 5′ to the translational start site of the endogenous GPR88 gene. In some embodiments, the regulatory element comprises a nucleotide sequence that is at least 80%, 85%, 90%, 95%, 97%, 98%, 99% homologous to the nucleotide sequence set forth in SEQ ID NO: 41. In some embodiments, the regulatory element comprises a nucleotide sequence that is identical to the nucleotide sequence set forth in SEQ ID NO: 41.

In some embodiments, the genomic sequence positioned 5′ to a translational start site of the endogenous GPR88 gene is positioned less than about 600 nucleotides 5′ to the translational start site of the endogenous GPR88 gene. In some embodiments, the regulatory element comprises a nucleotide sequence that is at least 80%, 85%, 90%, 95%, 97%, 98%, 99% homologous to the nucleotide sequence set forth in SEQ ID NO: 42. In some embodiments, the regulatory element comprises a nucleotide sequence that is identical to the nucleotide sequence set forth in SEQ ID NO: 42.

In some embodiments, the genomic sequence positioned 5′ to a translational start site of the endogenous GPR88 gene is positioned less than about 1,500 nucleotides 5′ to the translational start site of the endogenous GPR88 gene. In some embodiments, the regulatory element comprises a nucleotide sequence that is at least 80%, 85%, 90%, 95%, 97%, 98%, 99% homologous to the nucleotide sequence set forth in SEQ ID NO: 43. In some embodiments, the regulatory element comprises a nucleotide sequence that is identical to the nucleotide sequence set forth in SEQ ID NO: 43.

In some embodiments, the regulatory element comprises a nucleotide sequence that is at least 80%, 85%, 90%, 95%, 97%, 98%, 99% homologous to the nucleotide sequence set forth in any one of SEQ ID NOs: 44, 45, 46, or 48. In some embodiments, the regulatory element comprises a nucleotide sequence that is at least 80%, 85%, 90%, 95%, 97%, 98%, 99% homologous to the nucleotide sequence set forth in any one of SEQ ID NOs: 44, 45, 46, or 48. In some embodiments, the nucleic acid comprising a heterologous gene of interest is operatively coupled to a regulatory element, wherein the regulatory comprises a nucleotide sequence that is at least 80%, 85%, 90%, 95%, 97%, 98%, 99% homologous to the nucleotide sequence set forth in SEQ ID NO: 47.

In some embodiments, the heterologous gene of interest is 3′ to the regulatory element. In some embodiments, the heterologous gene of interest possesses therapeutic utility. In some embodiments, the heterologous gene of interest comprises a neurotrophic factor, an RNA guided nuclease, an enzyme, or a DREADD. In some embodiments, the heterologous gene of interest comprises a DREADD. In some embodiments, the gene of interest comprises a DREADD. In some embodiments, the DREADD is selected from the list consisting of one or more of rM3Ds, hM3Ds, or hM3Ds(A147S-F349Y).

In some embodiments, the DREADD is rM3Ds. In some embodiments, the DREADD comprises an amino acid sequence exhibiting at least about 90%, 95%, 97%, 98%, 99% identity to or is identical to SEQ ID NO: 38. In some embodiments, the heterologous gene of interest comprises one or more of hM3Dq, hM1Dq, hMD5q, hM4Di, hM2Di, or BDNF. In some embodiments, the DREADD is hM3Ds. In some embodiments, the DREADD comprises an amino acid sequence exhibiting at least about 90%, 95%, 97%, 98%, 99% identity to or is identical to SEQ ID NO: 49. In some embodiments, the DREADD is hM3Ds(A147S-F349Y). In some embodiments, the DREADD comprises an amino acid sequence exhibiting at least about 90%, 95%, 97%, 98%, 99% identity to or is identical to SEQ ID NO: 50.

In some embodiments, the heterologous gene of interest exhibits increased expression of the heterologous gene of interest compared to the promoter of the hSYN1 gene in a neuron of the striatum. In some embodiments, the heterologous gene of interest exhibits at least a 2-fold increase in expression of the heterologous gene of interest compared to the promoter of the hSYN1 gene in a neuron of the striatum. In some embodiments, the heterologous gene of interest exhibits at least a 5-fold increase in expression of the heterologous gene of interest compared to the promoter of the hSYN1 gene in a neuron of the striatum. In some embodiments, the heterologous gene of interest exhibits at least a 25-fold increase in expression of the heterologous gene of interest compared to the promoter of the hSYN1 gene in a neuron of the striatum.

In some embodiments, the present disclosure provides a pharmaceutical composition comprising a pharmaceutically acceptable, carrier, excipient, or diluent and the retro-AAV. In some embodiments, the retro-AAV and/or the pharmaceutical composition are used in a method to express a polypeptide in a neuron of the striatum. In some embodiments, the neuron of the striatum is a D1 dopaminergic medium spiny neuron. In some embodiments, the retro-AAV and/or the pharmaceutical composition are used in a method to genetically engineer a neuron of the striatum. In some embodiments, the neuron of the striatum is a D1 dopaminergic medium spiny neuron.

In some embodiments, the retro-AAV and/or the pharmaceutical composition are used in a method to treat a neurodegenerative disease in an individual. In some embodiments, the neurodegenerative disease comprises Parkinson's disease.

In some embodiments, the present disclosure provides a method to express a polypeptide in a neuron of the striatum of an individual comprising administering the retro-AAV or the pharmaceutical composition to the individual thereby expressing the polypeptide the neuron of the striatum. In some embodiments, the neuron of the striatum is a DJ dopaminergic medium spiny neuron. In some embodiments, the present disclosure provides a method to genetically engineer a neuron of the striatum of an individual comprising administering the retro-AAV or the pharmaceutical composition to the individual thereby genetically engineering the neuron of the striatum. In some embodiments, the neuron of the striatum is a D1 dopaminergic medium spiny neuron.

In some embodiments, the present disclosure provides a method to treat an individual afflicted with a neurodegenerative disease comprising administering the retro-AAV or the pharmaceutical composition to the individual afflicted with a neurodegenerative disease thereby treating the neurodegenerative disease. In some embodiments, the neurodegenerative disease comprises Parkinson s disease. In some embodiments, the individual is a mammal. In some embodiments, the individual is a human.

In some embodiments, the present disclosure provides a method to express and activate a DREADD in the central nervous system of an individual comprising administering to the individual the retro-AAV or the pharmaceutical composition and a ligand that activates the DREADD, thereby activating the DREADD in the central nervous system of the individual. In some embodiments, the DREADD is expressed and activated int in a neuron of the striatum. In some embodiments, the neuron of the striatum is a D1 dopaminergic medium spiny neuron. In some embodiments, the individual is a mammal. In some embodiments, the individual is a human. In some embodiments, activating the DREADD in the central nervous system of the individual treats a neurodegenerative disorder. In some embodiments, the neurodegenerative disorder comprises Parkinson's disease. In some embodiments, the ligand that activates the DREADD comprises quetiapine or clozapine. In some embodiments, the ligand that activates the DREADD comprises quetiapine. In some embodiments, the ligand that activates the DREADD comprises clozapine. In some embodiments, the retro-AAV and the ligand that activates the DREADD are administered separately.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1A illustrates the mutations that were introduced at three sites in AAV8 capsid protein to make AAV8R, including N385D; at position 588, an insertion of the amino acid sequence RGNLADQDYTKTARQAATAD (SEQ ID NO: 31) from rAAV2 retro where the bolded portion represents an additional 10-aa insertion; and TS720-721IN. Two additional mutations of V183E and N411 S were incorporated in to AAV8R12 capsid protein. The figure discloses SEQ ID NOS 33, 61 and 61, respectively, in order of appearance. FIG. 1B illustrates the mutations made to generate AAV1R, AAV5R, and AAV6R. To generate AAV1R, the amino acid sequence of SSSTDP (SEQ ID NO: 53) beginning at position 586 of AAV1 was replaced with RGNLADQDYTKTARQA (SEQ ID NO: 54) and two point mutations, N383D and A709I, were made. To generate AAV5R, the amino acid sequence NQSSTTAP (SEQ ID NO: 55) beginning at position 575 of AAV5 was replaced with LQRGNLADQDYTKTARQA (SEQ ID NO: 56) and 695-698DPQF was mutated to KSIN (SEQ ID NO: 57). To generate AAV6R, the amino acid sequence SSSTDP (SEQ ID NO: 53) beginning at position 586 of AAV6 was replaced with RGNLADQDYTKTARQA (SEQ ID NO: 54) and two point mutations, N383D and A709I, were made. The figure discloses SEQ ID NOS 62-66, 57, 62 and 66, respectively, in order of appearance. FIG. 1C illustrates that AAV1R, AAV5R, and AAV6R labeling striating projection neurons is inefficient as barely any neurons are labeled. FIG. 1D illustrates images and percentages of retrogradely labeled neurons in the SNr and its upstream brain regions by nigral injection of AAV8R12 showing brain regions with EYFP+ cells. SNr: substantia nigra pars reticulata. STN: subthalamic nucleus. SC: superior colliculus. OFC: orbital frontal cortex. ACAv: anterior cingulate cortex, ventral. ILA: infralimbic cortex. PrL: prelimbic cortex. FrA: frontal association cortex. Scale bar, 1 mm. FIG. 1E illustrates images and percentages of retrogradely labeled neurons in the SNr and its upstream brain regions by nigral injection of AAV8R12 showing Quantitation of EYFP+ cells. n=3 mice per group, data are represented as mean±SEM.

FIG. 2A illustrates how viruses were injected into the SNr of wild type mice. FIG. 2B-2C illustrate how AAV8R and AAV8R12 show markedly increased efficiency in labeling striatonigral projection neurons over rAAV2-retro. Scale bar (FIG. 2B), 100 μm. n=3 mice per group (FIG. 2C), error bars indicate SEM, one-way ANOVA with post-hoc Tukey's test (F(2, 6)=48.66; rAAV2-retro vs. AAV8R: 2078±262.4 vs. 10092±463.9, P=0.0032; rAAV2-retro vs. AAV8R12: 2078±262.4 vs. 16026±1654, P=0.0002; AAV8R vs. AAV8R12: 10092±463.9 vs. 16026±1654, P=0.0137). FIG. 2D-2E illustrate robustly improved retrograde labeling efficiency of accumbal MSNs compared to rAAV2-retro. FIG. 2D shows representative images of mouse accumbal projection neurons labeled by rAAV2-retro, AAV8R, and AAV8R12 after delivery into dorsolateral ventral pallidum (scale bar, 100 μm. aca, anterior commissure; AcbC, nucleus accumbens core). FIG. 2E shows representative images of mouse accumbal projection neurons labeled by rAAV2-retro, AAV8R, and AAV8R12 after delivery into lateral hypothalamus (LH) (scale bar, 100 μm. AcbSh, nucleus accumbens shell).

FIG. 3A-3C illustrate the strategy for identification of highly active MSN promoters. A list of striatum-enriched genes were identified based on in situ hybridization data (FIG. 3A). Putative promoter sequences were determined by H3K4me1 and H3K27ac epigenetic marks (FIG. 3B) and cloned into an AAV vector expressing EYFP (FIG. 3C). WPRE is woodchuck hepatitis post-transcriptional regulatory element; pA is polyadenylation signal; ITR is inverted terminal repeats.

FIG. 4A-4B illustrate howG88P2, 2G88P3, and G88P7 promoters showed increased efficiency, as evidenced by the proliferation of labeled neurons, in driving reporter expression in MSNs as compared to several commonly used promoters, including human Synapsin-1 (hSyn), CMV early enhancer/chicken R actin (CAG), and Elongation Factor 1 alpha (EF1α). Scale bar (FIG. 4A), 100 μm. n=3 mice per group (FIG. 4B), error bars indicate mean±SEM, one-way ANOVA with post-hoc Dunnett's test, (F(5, 12)=13.49; hSyn vs. EF1α: 16052±1630 vs. 6954±698.7 P=0.4749; hSyn vs. CAG: 16052±1630 vs. 4610±707.7, P=0.2824; hSyn vs. G88P2: 16052±1630 vs. 36290±3705, P=0.0258; hSyn vs. G88P3: 16052±1630 vs. 37026±6646, P=0.0209; hSyn vs. G88P7: 16052±1630 vs. 39460±7281, P=0.0104).

FIG. 5A illustrates Striatal neurons co-labelled (arrowheads) for AAV8R12-G88P3-EYFP and Drd1, but not Drd2, after viral injection into the SNr. Scale bar, 5 μm. FIG. 5B is a plot showing quantitation of Drd1+ and Drd2+ cells amongst EYFP+ cells; n=3 mice per group, and data are represented as mean SEM.

FIG. 6A illustrates after nigral delivery of AAV8R12-G88P3-HA-hM3Dq, intraperitoneal (i.p.) delivery of CNO induced ipsiversive rotations whereas intracranial (i.e.) CNO infusion into the dorsomedial striatum induced contraversive rotations. n=8 mice per group, error bars indicate mean±SEM, unpaired t-test, (Saline i.p. vs. CNO i.p.: 45.25±4.128 vs. 87.84±4.666, t=6.836, P<0.0001; Saline i.e. vs. CNO i.e.: 49.63±5.879 vs. 15.08±3.435, t=5.074, P=0.0002). FIG. 6B illustrates nigral neurons co-labelled for AAV8R12-G88P3-HA-hM3Dq and c-Fos after intraperitoneal, but not intracranial, infusion of CNO. Scale bar, 20 μm. FIG. 6C illustrates after nigral delivery of AAV8R12-G88P3-EYFP, both intraperitoneal (i.p.) delivery of CNO and intracranial (i.e.) CNO infusion into the dorsomedial striatum showed no effects on rotation behavior of mice. n=5 mice per group, error bars indicate mean±SEM, unpaired t-test, (Saline i.p. vs. CNO i.p.: 47.08±10.5 vs. 44.72±10.89, t=0.1561, df=8, P=0.8798; Saline i.e. vs. CNO i.e.: 54.22±8.766 vs. 61.39±5.774, t=0.6838, df=8, P=0.5134). FIG. 6D includes representative images of retrogradely labeled neurons in the SNr and upstream brain regions by nigral injection of AAV8R12-G88P3-HA-hM3Dq, where the scale bar represents 1 mm. FIG. 6E illustrates retrograde labeling of mouse striatal neurons with Drd1 ISH (left panel, arrowheads) and Drd2 ISH (right panel, arrowheads) after AAV8R12-G88P3-HA-hM3Dq injection into the SNr, where the scale bars represent 10 μm. FIG. 6F is a plot showing quantitation of Drd1+ and Drd2+ cells amongst all HA+ cells, where n=3 mice per group and data are represented as mean±SEM. FIG. 6G illustrates electrophysiological responses to CNO in retrogradely labeled D1-MSNs after AAV8R12-G88P3-HA-hM3Dq-2A-EYFP injection into the SNr. Whole-cell patch clamp recordings were conducted from EYFP+ cells in ex vivo slices. FIG. 6G includes representative traces (left panel) and quantitation (right panel) of action potentials induced by somatic current injection at baseline and after CNO administration, where n=7 cells from 4 mice, data are represented as mean±SEM, two-tailed paired t-test, and ***p<0.001. FIG. 6H-6I depict chemogenetic BG direct pathway manipulation in mice 3 weeks after nigral delivery of AAV8R12-G88P3-HA-hM3Dq and delivery of CNO via either intraperitoneal (i.p.) injection (FIG. 6H) or intracranial (i.e.) infusion targeting the dorsomedial striatum (FIG. 6I). Percentage of contraversive rotations was quantified (left panels), n=8 mice per group, data are represented as mean±SEM, two-tailed unpaired t-test, ***p<0.001. HA and c-Fos antibody staining in mouse SNr after CNO delivery (right panels). Scale bars, 20 μm.

FIG. 7A illustrates after nigral delivery of AAV8R12-G88P7-rM3Ds-2A-EYFP, both intraperitoneal and intracranial delivery of CNO induced contraversive rotations. n=8 mice per group, error bars indicate mean±SEM, unpaired t-test, (Saline i.p. vs. CNO i.p.: 45.95±4.972 vs. 80.05±5.168, t=4.754, P=0.0003; Saline i.e. vs. CNO i.e.: 58.31±4.742 vs. 80.77±4.187, t=3.551, P=0.0032). FIG. 7B illustrates no nigral neurons co-labelled for AAV8R12-G88P7-rM3Ds-2A-EYFP and c-Fos after intraperitoneal or intracranial infusion of CNO. Scale bar, 20 μm. FIG. 7C illustrates after nigral delivery of AAV8R12-G88P7-EYFP, both intraperitoneal and intracranial delivery of CNO showed no effects on rotation behavior of mice. n=5 mice per group, error bars indicate mean±SEM, unpaired t-test, (Saline i.p. vs. CNO i.p.: 48.68±7.203 vs. 46.88±7.497, t=0.1733, df=8, P=0.8667; Saline i.e. vs. CNO i.e.: 41.11±12.73 vs. 34.55±7.536, t=0.4435, df=8, P=0.6691). FIG. 7D includes representative images of retrogradely labeled neurons in the SNr and upstream brain regions by nigral injection of AAV8R12-G88P7-rM3Ds-2A-EYFP, where the scale bar represents 1 mm. FIG. 7E includes images of retrograde labeling of mouse striatal neurons with Drd1 ISH (left, arrowheads) and Drd2 ISH (right, arrowheads) after AAV8R12-G88P7-rM3Ds-2A-EYFP injection into the SNr, where scale bars represent 10 μm. FIG. 7F is a plot showing quantitation of Drd1+ and Drd2+ cells amongst EYFP+ cells, where n=3 mice per group, and data are represented as mean SEM. FIG. 7G illustrates electrophysiological responses to CNO in retrogradely labeled D1-MSNs after AAV8R12-G88P7-rM3Ds-2A-EYFP injection into the SNr. Whole-cell patch clamp recordings were conducted from EYFP+ cells in ex vivo slices. FIG. 7G includes representative traces (left panel) and quantitation (right panel) of action potentials induced by somatic current injection at baseline and after CNO administration. n=10 cells from 6 mice, data are represented as mean±SEM, two-tailed paired t-test, ****p<0.0001. FIG. 7H-7I depict chemogenetic manipulation of a BG direct pathway in mice 3 weeks after nigral delivery of AAV8R12-G88P7-rM3Ds-2A-EYFP and delivery of CNO via either i.p. injection (FIG. 7H) or i.e. infusion targeting the dorsomedial striatum (FIG. 7I). Percentage of contraversive rotations was quantified (left panels), n=8 mice per group, data are represented as mean±SEM, two-tailed unpaired t-test, **p<0.01, ***p<0.001. EYFP and c-Fos antibody staining in mouse SNr after CNO delivery (right panels). Scale bars, 20 μm.

FIG. 8A-8B illustrate representative heatmaps of macaques received nigral injections of AAV8R12-G88P7-HA-hM3Dq (FIG. 8A) or AAV8R12-G88P7-rM3Ds-2A-EYFP (FIG. 8B) following saline or CNO infusions. Animals administered with CNO spent less time in the higher portion of the observing cages. FIG. 8C-8H illustrate quantitation of velocity of ipsiversive rotations (FIG. 8C, FIG. 8F), total distance traveled (FIG. 8D, FIG. 8G), and immobile time (FIG. 8E, FIG. 8H) of macaques that received nigral injections of AAV8R12-G88P3-HA-hM3Dq (FIG. 8C-8E) or AAV8R12-G88P7-rM3Ds-2A-EYFP (FIG. 8F-8H) following saline or CNO infusion. n=3 monkeys per group (FIG. 8C-8E), n=6 monkeys per group (FIG. 8F-8H), data are represented as mean±SEM, two-tailed paired t-test, n.s., not significant.

FIG. 9A illustrates AAV8R12-G88P3-mCherry injected into the SNr of a cynomolgus macaque; labeled neurons were found throughout the caudate and putamen nuclei. Positions of coronal sections along the anterior-posterior axis are indicated as distance from EBZ (ear bar zero). Scale bar, 5 mm. FIG. 9B illustrates labeled neurons in the caudate and putamen nuclei. Scale bar, 100 μm. FIG. 9C illustrates retrograde labelling of striatal neurons with DRD1 ISH (top panel, arrowheads) and DRD2 ISH (bottom panel, arrowheads) after AAV8R12-G88P3-mCherry injection into the macaque SNr. Co-labeling was only observed for DRD1. Scale bar, 20 μm. FIG. 9D-9E illustrates activation of BG direct pathway in mice 12 months after nigral delivery of AAV8R12-G88P7-rM3Ds-2A-EYFP and delivery of CNO via i.p. injection (FIG. 9D) and the percentage of rotational behavior (ipsiversive rotations and contraversive rotations) was quantified (FIG. 9E), n=6 mice per group, data error bars indicate mean±SEM, two-tailed unpaired t-test, **p<0.01. FIG. 9F illustrates robust labeling of D1-MSN after nigral injection of AAV8R12 in a cynomolgus macaque with somas of retrogradely labeled neurons throughout the caudate and putamen which were extracted from fluorescent images (FIG. 9A). Positions of coronal sections along the anterior-posterior axis are indicated as the distance from EBZ. Scale bar, 2 mm. FIG. 9G is a plot showing quantitation of DRD1+ and DRD2+ cells amongst mCherry+ cells, where n=6 sections from 1 macaque, and data are represented as mean±SEM.

FIG. 10A illustrates a representative top view movement tracing plot of an observation cage housing a macaque that received nigral injections of AAV8R12-G88P3-HA-hM3Dq in observation cage after i.e. CNO infusion into the dorsomedial caudate of macaque. FIG. 10B illustrates a representative top view tracing plot of macaques received nigral injections of AAV8R12-NP3-rM3Ds-2A-EYFP in observation cage after intramuscular (i.m.) CNO injection in macaques. Macaques that received nigral injections of AAV8R12-G88P3-HA-hM3Dq (monkey ID: CM045, CM049) or AAV8R12-G88P7-rM3Ds-2A-EYFP (Monkey ID: CM048, CM051) showed markedly increased contraversive rotations (FIG. 10C) as shown is the quantitation of time on the top compartment of the observation cage after intracranial or systemic CNO infusions, respectively. n=4 monkeys per group, error bars indicate the mean±SEM, two-tailed paired t-test (t=3.276, P=0.0469, Saline vs. CNO: 1.004±0.2667 vs. 6.649±1.662). Macaques received nigral injections of AAV8R12-G88P3-HA-hM3Dq or AAV8R12-G88P7-rM3Ds-2A-EYFP spent less time in the top portion of the observing cage (FIG. 10D), shown is the quantitation of velocity of contraversive rotations after CNO infusion in macaques n=4 monkeys per group, error bars indicate the mean±SEM, two-tailed paired t-test *p<0.05 (t=3.605, P=0.0366, Saline vs. CNO: 68.42±10.7 vs. 33.74±2.641) and showed increased speed during contraversive rotations (FIG. 10G) (paired t-test, t=4.06, P=0.0269, Saline vs. CNO: 38.42±5.465 vs. 46.98±6.669) after intracranial or systemic CNO infusions, respectively. No significant differences were found for the immobile time (FIG. 10E) (paired t-test, t=2.211, P=0.1140, Saline vs. CNO: 107.1±2.322 vs. 89.13±8.379), total distance (FIG. 10F) (paired t-test, t=2.272, P=0.1077, Saline vs. CNO: 71.53±15.4 vs. 97.37±19.41), velocity of contraversive rotations (FIG. 10G), or velocity of ipsiversive rotations (FIG. 10H) (paired t-test, t=1.001, P=0.3905, Saline vs. CNO: 37.43±3.264 vs. 41.37±6.566). n=4, error bars indicate mean±SEM, paired t-test. FIG. 10I includes representative images of retrograde labeling throughout the basal ganglia after nigral injection of AAV8R12-G88P3-HA-hM3Dq. Positions of coronal sections along the anterior-posterior axis are indicated as the distance from EBZ. Scale bar, 5 mm. FIG. 10J includes high-magnification images of labeled hM3Dq+ neurons in the caudate and putamen nuclei in the macaque. Scale bars, 20 μm. FIG. 10K includes retrograde labeling of hM3Dq+ striatal neurons (green) with DRD1 ISH (left panel, magenta, arrowheads) and DRD2 ISH (right panel, magenta, arrowheads). Scale bars, 20 μm. FIG. 10L illustrates quantitation of DRD1+ and DRD2+ cells amongst all HA+ cells. n=6 sections from 1 macaque per group, data are represented as mean±SEM. FIG. 10M includes representative images of retrograde labeling throughout the basal ganglia after nigral injection of AAV8R12-G88P7-rM3Ds-2A-EYFP. Positions of coronal sections along the anterior-posterior axis are indicated as the distance from EBZ. Scale bar, 5 mm. FIG. 10N includes high-magnification images of labeled rM3Ds+ neurons in the caudate and putamen nuclei in the macaque. Scale bars, 20 μm. FIG. 10O illustrates retrograde labeling of rM3Ds+ striatal neurons (green) with DRD1 ISH (left panel, magenta, arrowheads) and DRD2 ISH (right panel, magenta, arrowheads). Scale bars, 20 μm. FIG. 10P illustrates quantitation of DRD1+ and DRD2+ cells amongst all EYFP+ cells. n=6 sections from 1 macaque per group, data are represented as mean±SEM. FIG. 10Q depicts a representative top view movement tracing plot of an observation cage housing a macaque that received i.e. CNO infusion into the dorsomedial caudate 8 weeks after nigral injections of AAV8R12-G88P3-HA-hM3Dq. FIG. 10R is a plot showing quantitation of a ratio of contraversive rotations to ipsiversive rotations after CNO infusion in relation to FIG. 10Q. n=3 monkeys per group, data are represented as mean±SEM, two-tailed paired t-test, *p<0.05. FIG. 10S is a plot showing quantitation of a ratio of contraversive rotations to ipsiversive rotations after CNO infusion in relation to FIG. 10B. n=6 monkeys per group, data are represented as mean±SEM, two-tailed paired t-test, *p<0.05. FIG. 10T-10U depict quantitation of time on the top compartment of the observation cage (FIG. 10T) and velocity of contraversive rotations (FIG. 10U) after i.e. CNO infusion in macaques received nigral injections of AAV8R12-G88P3-HA-hM3Dq. n=3 monkeys per group, data are represented as mean±SEM, two-tailed paired t-test, *p<0.05. FIG. 10V-10W depict quantitation of time on the top compartment of the observation cage (FIG. 10V) and velocity of contraversive rotations (FIG. 10W) after i.m. CNO injection in macaques received nigral injections of AAV8R12-G88P7-rM3Ds-2A-EYFP. n=6 monkeys per group, data are represented as mean±SEM, two-tailed paired t-test, **p<0.01, ***p<0.001.

FIG. 11A-11B illustrate representative images of naive macaques following saline or CNO infusions from top (FIG. 11A) and side (FIG. 11B) view of the observation cage. Animals showed no differences in rotation behavior and in time spending in the higher portion of the observing cages. No significant differences were found for the percentage of contraversive and ipsiversive rotation (FIG. 11C) (paired t-test, t=1.709, df=2, P=0.2296, Saline vs. CNO: 0.767±0.4296 vs. 0.8384±0.4152), time spending in the higher portion of the observing cages (FIG. 11D) (paired t-test, t=1.141, df=2, P=0.3720, Saline vs. CNO: 55.73±2.64 vs. 60.43±4.542), immobile time (FIG. 11E) (paired t-test, t=0.02596, df=3, P=0.9816, Saline vs. CNO: 149.9±21.61 vs. 149.8±22.65), total distance (FIG. 11F) (paired t-test, t=1.505, df=2, P=0.2713, Saline vs. CNO: 53.12±15.3 vs. 59.48±18.65), speed of contraversive rotations (FIG. 11G) (paired t-test, t=0.134, df=2, P=0.9057, Saline vs. CNO: 39.07±5.889 vs. 39.63±7.092) or speed of ipsiversive rotations (FIG. 11H) (paired t-test, t=1.129, df=2, P=0.3762, Saline vs. CNO: 41.49±3.96 vs. 42.79±4.705) after intracranial or systemic CNO infusions, respectively. n=3 monkeys per group, error bars indicate mean±SEM, paired t-test.

FIG. 12A-12F illustrate the electrophysiological analyses of macaques after chemogenetic activation of the basal ganglia direct pathway. FIG. 12A illustrates the schematics of AAV8R12-G88P3-HA-hM3Dq injection in SNr and electrophysiological recording in caudate following CNO infusion into the dorsomedial caudate (monkey ID: CM045). FIG. 12B illustrates response time course of a typical neuron in caudate following CNO infusion into the dorsomedial caudate. The x-axis indicates the period (in minute) for counting spikes, and the y-axis indicates the normalized spike count. Each inset shows the raw spike trace (top portion of inset) and waveforms (bottom portion of inset) at a specific time point indicated by the arrow. FIG. 12C illustrates a total number of 34 cells in caudate was recorded from monkey CM045, among which 70.6% of the cells (n=24 cells) were activated, 8.8% (n=3 cells) were inactivated, while 20.6% (n=7 cells) remained unchanged. FIG. 12D illustrates the schematics of AAV8R12-G88P7-rM3Ds-2A-EYFP injection in SNr and electrophysiological recording in caudate following intramuscular CNO injection (monkey ID: CM048). FIG. 12E illustrates the response time course of a typical neuron in caudate following intramuscular CNO injection. The x-axis indicates the period (in minute) for counting spikes, and the y-axis indicates the normalized spike count. Each inset shows the raw spike trace (top portion of inset) and waveforms (bottom portion of inset) at a specific time point indicated by the arrow. FIG. 12F illustrates a total number of 38 cells in caudate was recorded from monkey CM048, among which 65.8% of the cells (n=25 cells) were activated, 7.9% (n=3 cells) were inactivated, while 26.3% (n=10 cells) remained unchanged. FIG. 12G includes a chematic of AAV8R12-G88P7-HA-rM3Ds-2A-Cre injection into SNr, AAV9-EF1α-DIO-ChR2-EYFP injection into the caudate/putamen, and electrophysiological recording combined with optical stimulation in the caudate/putamen following intramuscular CNO injection in anesthetized macaques (left panel). The raw spike trace and waveform of a typical neuron in the caudate upon blue light (473 nm) illumination (right panel). FIG. 12H illustrates spike counts over repeated optogenetic stimulations of the typical neuron shown in FIG. 12G. FIG. 12I illustrates a raw spike trace and waveform at baseline and after intramuscular CNO administration of the typical neuron shown in FIG. 12G. FIG. 12J plots a response time-course of retrogradely labeled caudate/putamen neurons (n=5) identified by opto-tagging after intramuscular CNO injection. The x-axis indicates the period (in minutes) for counting spikes, and the y-axis indicates the normalized population response. FIG. 12K includes a schematic of AAV8R12-G88P3-HA-hM3Dq injection into SNr and electrophysiological recording in the caudate following CNO infusion into the dorsomedial caudate (left panel) in anesthetized macaques. Right panel shows the response time-course of activated caudate neurons (n=23) following CNO infusion into the dorsomedial caudate. The x-axis indicates the period (in minutes) for counting spikes, and the y-axis indicates the normalized population response. The inset shows the raw spike trace and waveform of a typical neuron in the caudate at baseline and 50-60 min after CNO infusion. FIG. 12L illustrates: for CNO infusions, 34 total cells were recorded in the caudate, among which 67.6% of the cells (n=23 cells) were activated, 2.9% (n=1 cells) were inactivated, and 29.4% (n=10 cells) remained unchanged; for saline infusions, 32 total cells were recorded in the caudate, among which 18.8% of the cells (n=6 cells) were activated, 40.6% (n=13 cells) were inactivated, and 40.6% (n=13 cells) remained unchanged. FIG. 12M includes a schematic of AAV8R12-G88P7-rM3Ds-2A-EYFP injection into SNr and electrophysiological recording in the caudate/putamen following intramuscular CNO injection (left panel) in anesthetized macaques. Right panel shows the response time-course of activated caudate/putamen neurons (n=19) following CNO injection. The x-axis indicates the period (in minutes) for counting spikes, and the y-axis indicates the normalized population response. The inset shows the raw spike trace and waveform of atypical neuron in the caudate at baseline and 50-60 min after CNO infusion. FIG. 12N illustrates: for CNO administrations, 38 total cells were recorded in the caudate/putamen, among which 50% of the cells (n=19 cells) were activated, 13.2% (n=5 cells) were inactivated, and 36.8% (n=14 cells) remained unchanged; for saline administrations, 30 total cells were recorded in the caudate/putamen, among which 20% of the cells (n=6 cells) were activated, 53.3% (n=16 cells) were inactivated, and 26.7% (n=8 cells) remained unchanged.

FIG. 13A-13F illustrate that chemogenetic activation of D1 MSNs reversed PD symptoms in mice. FIG. 13A illustrates a scheme of stereotaxic injections and behavioral analyses in PD mice. FIG. 13B illustrates representative images of tyrosine hydroxylase (TH) staining in control and PD animals. Dopaminergic neuron in SNc (down) and their terminals in Cpu (up) degenerated robustly. Scale bar, 1000 μm (up), 500 um (down). FIG. 13C illustrates representative tracing images of mice in open field test. FIG. 13D-13E illustrates that motor behavior was significantly reduced after 6-OHDA lesion as a quantitation of total distance (FIG. 13D) and immobile time (FIG. 13E). Chemogenetic activation of D1 MSNs significantly rescued motor deficient in PD model mice n=8, error bars indicate mean±SEM, paired t-test, **p<0.01. FIG. 13F illustrates that motor skill was significantly reduced after 6-OHDA lesion. Chemogenetic activation of D1 MSNs partially rescued motor deficient in PD model mice n=8, error bars indicate mean±SEM, paired t-test, *p<0.05, **p<0.01. FIG. 13G includes a plot illustrating a quantitation of a number of SNc TH+ cells in control and 6-OHDA-treated mice (n=4 mice per group, data represented as mean±SEM, two-tailed unpaired t-test, ****p<0.0001). FIG. 13H includes additional representative traces of mice in an open field test, before and after 6-OHDA lesion, and after saline or CNO treatment in lesioned animals. FIG. 13I shows CNO delivery, which selectively activates D1 MSNs, drastically reversed the dyskinesia-like phenotype of PD mice. FIG. 13J shows that CNO partially rescued the motor skill deficit of PD mice in a rotor-rod test. FIG. 13K shows that CNO partially rescued the motor skill deficit of 6-OHDA treated mice in a rotarod test. FIG. 13L illustrates stereotaxic injections of AAV8R12-G88P7-EYFP into SNr in a parkinsonian mouse model by 6-OHDA-mediated dopaminergic cell death, followed by administration of CNO. FIG. 13M includes a plot quantifying total distance traveled in an open field test in mice received nigral AAV8R12-G88P7-EYFP injections. n=8 mice per group, data are represented as mean±SEM, one-way ANOVA with post-hoc Tukey's test, ***p<0.001, ****p<0.0001, n.s., not significant. FIG. 13N includes a plot quantifying immobile time in an open field test in mice received nigral AAV8R12-G88P7-EYFP injections. n=8 mice per group, data are represented as mean±SEM, one-way ANOVA with post-hoc Tukey's test, ***p<0.001, ****p<0.0001, n.s., not significant.FIG. 13O is a plot quantifying latency to fall in a rotarod test in mice received nigral AAV8R12-G88P7-EYFP injections. n=8 mice per group, data are represented as mean±SEM, one-way ANOVA with post-hoc Dunnett's test, **p<0.01, ***p<0.001, n.s., not significant. FIG. 13P illustrates electrophysiological responses to CNO in retrogradely labeled D1-MSNs after AAV8R12-G88P7-rM3Ds-2A-EYFP injection into the SNr. Whole-cell patch clamp recordings were conducted from EYFP+ cells in ex vivo slices. Representative traces (left panels) and quantitation (right panels) of action potentials induced by somatic current injection at baseline and after CNO administration. n=11 cells from 7 mice (L), data are represented as mean±SEM, two-tailed paired t-test, **p<0.01, n.s., not significant. FIG. 13Q illustrates electrophysiological responses to CNO in retrogradely labeled D1-MSNs after AAV8R12-G88P7-EYFP injection into the SNr. Whole-cell patch clamp recordings were conducted from EYFP+ cells in ex vivo slices. Representative traces (left panels) and quantitation (right panels) of action potentials induced by somatic current injection at baseline and after CNO administration. n=8 cells from 3 mice (M), data are represented as mean±SEM, two-tailed paired t-test, **p<0.01, n.s., not significant.

FIG. 14 illustrates a scheme of stereotaxic injections and behavioral analyses in PD monkeys.

FIG. 15A illustrates representative images of Tyrosine hydroxylase (TH) staining in control and MPP+-injected animals. Dopaminergic neurons showed robust degeneration in SNc and their terminals in CPu. Scale bar, 5000 μm (Cd and Put), 50 um (SNc). FIG. 15B is a plot showing quantitation of number of SNc TH+ cells in control and MPP+-treated macaques. n=6 sections from 1 macaque per group, data are represented as mean±SEM, two-tailed unpaired t-test, ***p<0.001. FIG. 15C-15F illustrate: raw spike trace and waveform of atypical neuron in the caudate at baseline and 50-60 min after CNO (FIG. 15C) or DCZ (FIG. 15E) administration. The response time-course of activated caudate/putamen neurons following CNO (FIG. 15D, n=19 cells) or DCZ (FIG. 15F, n=13 cells) administration in anesthetized macaques. The x-axis indicates the period (in minutes) for counting spikes, and the y-axis indicates the normalized population response. FIG. 15G illustrates: for CNO administrations, 36 total cells were recorded in the caudate/putamen, among which 52.8% of the cells (n=19 cells) were activated, 13.9% (n=5 cells) were inactivated, and 33.3% (n=12 cells) remained unchanged. For DCZ administrations, 31 total cells were recorded in the caudate/putamen, among which 41.9% of the cells (n=13 cells) were activated, 9.7% (n=3 cells) were inactivated, and 48.4% (n=15 cells) remained unchanged. For saline administrations, 39 total cells were recorded in the caudate/putamen, among which 17.9% of the cells (n=7 cells) were activated, 33.4% (n=13 cells) were inactivated, and 48.7% (n=19 cells) remained unchanged. FIG. 15H includes a plot showing quantitation of total PD scores of macaques before and after DCZ treatment. n=4 monkeys per group, data are represented as mean±SEM, one-way ANOVA with post-hoc Dunnett's test, *p<0.05, **p<0.01, n.s., not significant. FIG. 15I plots total activity of macaques in the observation cage was divided into low, mid, and high mobility. Quantitation of the percentage of high mobility fraction indicated the activity changes in macaques after MPP+ lesion and after DCZ treatment compared with pre-lesion state. n=4 monkeys per group, data are represented as mean±SEM, one-way ANOVA with post-hoc Dunnett's test, *p<0.05, **p<0.01.

FIG. 16A-16L illustrate chemogenetic activation of D1-MSNs reversed parkinsonian symptoms in macaques; shown are representative traces of the quantitation of travel distance (FIG. 16A and FIG. 16C) and activity plot showing quantitation of time (FIG. 16B and FIG. 16D) of macaques in an observation cage. Macaques that received MPP+ showed markedly reduced total activity (FIG. 16E), travel distance (FIG. 16G) and (FIG. 16H) immobile time. DCZ treatment could successfully rescue motor deficits. n=4 monkeys per group, error bars indicate means±SEM, one-way ANOVA with post-hoc Dunnett's test (FIG. 16C), two-tailed paired t-test (FIG. 16D), *p<0.05, **p<0.01, n.s., not significant. Total activity of macaques was grouped into low, mid and high mobility. Macaques received MPP+ lesion showed motor balance deficits and rarely resided in the top portion of the observation cage, DCZ treatment significantly reversed this phenotype. n=4 monkeys per group, error bars indicate mean±SEM, paired t-test, *p<0.05, **p<0.01. (FIG. 16F). Total PD scores and separated PD score of PD macaques before and after DCZ treatment. n=4 monkeys per group, error bars indicate mean±SEM, paired t-test, *p<0.05, **p<0.01 (FIG. 16I-16J). Representative EMG plot of biceps in macaques. A typical PD-related 4-6 hz tremor signal (FIG. 16K) was removed after DCZ treatment. Macaques on DCZ treatment significantly reversed dyskinesia phenotype, as shown by representative quantitation of dyskinesia score at 2 weeks and 1 month after treatment with DCZ or L-Dopa. n=3 monkeys per group, data are represented as mean±SEM, two-tailed unpaired t-test, *p<0.05, ****p<0.0001 (FIG. 16L).

FIG. 17A illustrates the quantitation of percentage of tremor episode per 10 minutes. EMG was recorded continuously for 120 minutes after intramuscular delivery of DCZ. n=3 monkeys per group, error bars indicate mean±SEM, one-way ANOVA with post-hoc Dunnett's test, **p<0.01, n.s., not significant. FIG. 17B illustrates the quantitation of success rate of hand to mouth movement. DCZ treatment partially restored this motor skill. n=3 monkeys per group, error bars indicate mean±SEM, one-way ANOVA with post-hoc Dunnett's test, **p<0.01. FIG. 17C is a plot showing quantitation of travel distance after MPP+ lesion and DCZ treatment compared with pre-lesion state. n=4 monkeys per group, data are represented as mean±SEM, one-way ANOVA with post-hoc Dunnett's test, *p<0.05, n.s., not significant. FIG. 17D is a plot showing quantitation of immobile time after MPP+ lesion and DCZ treatment compared with pre-lesion state. n=4 monkeys per group, data are represented as mean±SEM, one-way ANOVA with post-hoc Dunnett's test, *p<0.05, n.s., not significant. FIG. 17E is a plot showing quantitation of time on the top compartment of the observation cage after MPP+ lesion and DCZ treatment compared with pre-lesion state. n=4 monkeys per group, data are represented as mean±SEM, one-way ANOVA with post-hoc Dunnett's test, *p<0.05, **p<0.01, n.s., not significant.

FIG. 18A-18C illustrate the total PD score of parkinsonian macaques before and after L-Dopa treatment. n=3 monkeys per group, error bars indicate mean±SEM, paired t-test, *p<0.05, **p<0.01 (FIG. 18A). Comparison of efficiency of DCZ and L-Dopa. DCZ achieved comparable efficiency to L-Dopa (FIG. 18B). DCZ reached stable efficacy faster than L-Dopa and its effect lasted over 24 hr (FIG. 18C). n=3 monkeys per group, error bars indicate mean±SEM, paired t-test, *p<0.05.

FIG. 19A depicts the alignment of AAV2 (SEQ ID NO: 20), rAAV2-retro (SEQ ID NO: 21), rAAV8-retro (SEQ ID NO: 1), and AAV8 (SEQ ID NO: 30). Alignment was performed using ClustalOmega Multiple Sequence Alignment (www.ebi.ac.uk/Tools/msa/clustalo/). A “*” symbol indicates perfect alignment; a “:” symbol indicates a site belonging to a group exhibiting strong similarity, and a “.” symbol indicates a site belonging to a group exhibiting weak similarity. FIG. 19B depicts and alignment of Cap protein sequences of AAV2, rAAV2-retro, AAV8, AAV8R, and AAV8R12, aligned with Clustal Omega and the result shown was illustrated with MViewer 1.63. FIG. 19B discloses SEQ ID NOS 20-21 and 67-69, respectively, in order of appearance.

FIG. 20 illustrates that Seroquel (quetiapine; QTP) stimulates movement of mice with SNr expression of the DREADD rM3Ds.

FIG. 21 illustrates that Seroquel (quetiapine; QTP) does not simulate movement of mice with SNr expression of the DREADD hM3Ds.

FIG. 22 shows an alignment of rM3Ds and hM3Ds. The figure discloses SEQ ID NOS 70-73, respectively, in order of columns.

FIG. 23 illustrates that Seroquel increased luciferase levels of hM3Ds-A147S-F349Y at the same levels observed for rM3Ds but did not increase luciferase levels for wild-type hM3Ds.

FIG. 24A plots quantification of total PD score of macaques before, 3 days, 1 week, and 2 weeks after L-Dopa treatment. n=3 monkeys per group, data are represented as mean±SEM, one-way ANOVA with post-hoc Dunnett's test, *p<0.05, n.s., not significant. FIG. 24B plots quantification of change in PD score at 3 days, 1 week, and 2 weeks after initial, sustained administration of DCZ or L-Dopa. n=3 monkeys per group, data are represented as mean±SEM, two-tailed paired t-test, *p<0.05, n.s., not significant. FIG. 24C plots quantification of total PD score by a single dose of DCZ or L-Dopa after drugs had reached steady-state efficacy. n=3 monkeys per group, data are represented as mean±SEM, two-tailed paired t-test, *p<0.05, n.s., not significant. FIG. 24D plots corticospinal fluid (CSF) concentration of DCZ measured at 6, 12, and 24 hours after i.m. delivery (0.3 mg/kg) by LC-MS. n=3 monkeys per group, data are represented as mean±SEM. FIG. 24E plots quantification of dyskinesia score at 2 weeks, 1 month, and 4 months after treatment with DCZ or L-Dopa. n=3 monkeys per group, data are represented as mean±SEM, two-tailed unpaired t-test, *p<0.05, ****p<0.0001. FIG. 24F illustrates: for extended L-Dopa treatment, animals were administered with L-Dopa once daily for 4 months. One month of washout was allowed before administration of DCZ. FIG. 24G plots quantification of total PD score of macaques before, 1, 2, and 4 months after L-Dopa treatment. n=3 monkeys per group, data are represented as mean±SEM, one-way ANOVA with post-hoc Dunnett's test, *p<0.05. FIG. 24H plots quantification of total PD score of macaques before, 1, and 2 months after DCZ treatment following extended L-Dopa administration and washout (shown in FIG. 24F). n=3 monkeys per group, data are represented as mean±SEM, one-way ANOVA with post-hoc Dunnett's test, *p<0.05. FIG. 24I plots quantification of travel distance before, 1, and 2 months after DCZ treatment following extended L-Dopa administration and washout (shown in FIG. 24F). n=3 monkeys per group, data are represented as mean SEM, one-way ANOVA with post-hoc Dunnett's test, *p<0.05. FIG. 24J plots quantification of immobile time before, 1, and 2 months after DCZ treatment following extended L-Dopa administration and washout (shown in FIG. 24F). n=3 monkeys per group, data are represented as mean±SEM, one-way ANOVA with post-hoc Dunnett's test, *p<0.05. FIG. 24K plots quantification Quantitation of time on the top compartment of the observation cage before, 1, and 2 months after DCZ treatment following extended L-Dopa administration and washout (shown in FIG. 24F). n=3 monkeys per group, data are represented as mean SEM, one-way ANOVA with post-hoc Dunnett's test, *p<0.05. FIG. 24L plots quantification of dyskinesia score at 1 and 2 months after DCZ treatment following extended L-Dopa administration and washout (shown in FIG. 24F). n=3 monkeys per group, data are represented as mean SEM.

FIG. 25 is a schematic showing locations of mutations introduced at two or three sites in the AAV1/5/6 capsid proteins to make AAV1R, AAV5R, and AAV6R. The schematic is relevant to retrograde AAV tracers for D1-MSNs, and FIG. 1C. FIG. 25 discloses SEQ ID NOS 74, 63, 55, 65-66, 57, 75, and 66, respectively, in order of appearance.

FIG. 26A-26E depict characterization of labeling specificity after intravenous delivery of AAV-PHP.eB-G88P7-EYFP. FIG. 26A includes co-staining of transduced neurons (EYFP) with parvalbumin (PV) after intravenous delivery of AAV-PHP.eB-G88P7-EYFP or AAVPHP.eB-hSyn-EYFP. Arrowheads indicate double+ cells. Scale bars, 50 μm. FIG. 26B includes co-staining of transduced neurons (EYFP) with somatostatin (SST) after intravenous delivery of AAV-PHP.eB-G88P7-EYFP or AAVPHP.eB-hSyn-EYFP. Arrowheads indicate double+ cells. Scale bars, 50 μm. FIG. 26C includes co-staining of transduced neurons (EYFP) with ChAT after intravenous delivery of AAV-PHP.eB-G88P7-EYFP or AAVPHP.eB-hSyn-EYFP. FIG. 26D includes quantitation of PV+, SST+, and ChAT+ cells amongst EYFP+ cells in the striatum after intravenous delivery of AAV-PHP.eB-G88P7-EYFP or AAV-PHP.eB-hSyn-EYFP. n=6 mice per group, data are represented as mean±SEM. FIG. 26E includes co-staining of transduced neurons (EYFP) with Drd1 and Drd2 after intravenous delivery of AAV-PHP.eB-G88P7-EYFP. Arrowheads indicate double+ cells. Scale bar, 20 μm.

FIG. 27A-27D depict characterization of striatonigral projection neurons after nigral delivery of AAV8R12-G88P7-EYFP. FIG. 27A illustrates retrograde labeling by stereotaxic injections of AAV8R12-G88P7-EYFP into the SNr and AAV9-G88P7-DIO-tdTomato into the striatum in Drd1-Cre or Drd2-Cre mice. FIG. 27B illustrates retrograde labeling of striatal neurons (EYFP, green, arrowheads) and Cre-driven tdTomato expression (tdT, magenta) in Drd1-Cre (top panels) or Drd2-Cre (bottom panels) mice. Scale bars, 50 μm (low magnification, left), 10 μm (high magnification, right). FIG. 27C illustrates quantitation of tdT+ and tdT− cells among all EYFP+ cells from striatal regions with dense labeling after nigral injection of AAV8R12-G88P7-EYFP and striatal injection of AAV9-G88P7-DIO-tdTomato in Drd1-Cre mice. n=3 mice per group. FIG. 27D illustrates quantitation of tdT+ and tdT− cells among all EYFP+ cells from striatal regions with dense labeling after nigral injection of AAV8R12-G88P7-EYFP and striatal injection of AAV9-G88P7-DIO-tdTomato in Drd2-Cre mice. n=3 mice per group.

FIG. 28A-28B depict percentages of retrogradely labeled neurons in the SNr and upstream brain regions by nigral injection of AAV8R12-G88P3-HA-hM3Dq and AAV8R12-G88P7-rM3Ds-2A-EYFP. FIG. 28A illustrates 1uantitation of EYFP+ cells after nigral injection of AAV8R12-G88P3-HA-hM3Dq. n=3 mice per group, data are represented as mean±SEM. FIG. 28B illustrates 1uantitation of EYFP+ cells after nigral injection of AAV8R12-G88P7-rM3Ds-2A-EYFP. n=3 mice per group, data are represented as mean±SEM.

FIG. 29A-29F depict striatal slice electrophysiological recordings after nigral injection of AAV8R12 expressing DREADDs. FIG. 29A illustrates latency of a first AP after current injection before and after CNO incubation. n=5 cells from 3 mice, data are represented as mean±SEM, two-tailed paired t-test, ***p<0.001. FIG. 29B illustrates basal activity without current injection recorded in slices prepared from mice received nigral AAV8R12-G88P3-HA-hM3Dq-2A-EYFP injections before and after CNO incubation. n=6 cells from 4 mice, data are represented as mean±SEM, two-tailed paired t-test, n.s., not significant. FIG. 29C illustrates resting membrane potential recorded in slices prepared from mice received nigral AAV8R12-G88P3-HA-hM3Dq-2A-EYFP injections before and after CNO incubation. n=6 cells from 4 mice, data are represented as mean±SEM, two-tailed paired t-test, n.s., not significant. FIG. 29D illustrates latency of a first AP after current injection before and after CNO incubation. n=7 cells from 5 mice, data are represented as mean±SEM, two-tailed paired t-test, ***p<0.001. FIG. 29E illustrates basal activity without current injection recorded in slices prepared from mice received nigral AAV8R12-G88P7-rM3Ds-2A-EYFP injections before and after CNO incubation. n=8 cells from 7 mice, data are represented as mean SEM, two-tailed paired t-test, n.s., not significant. FIG. 29F illustrates resting membrane potential recorded in slices prepared from mice received nigral AAV8R12-G88P7-rM3Ds-2A-EYFP injections before and after CNO incubation. n=8 cells from 7 mice, data are represented as mean±SEM, two-tailed paired t-test, n.s., not significant.

FIG. 30A-30D depict chemogenetic manipulation of mice injected with AAV8R12-G88P3/G88P7-EYFP. FIG. 30A illustrates effects of CNO on rotational behavior of mice after nigral injection of AAV8R12-G88P3-EYFP followed by intraperitoneal (i.p.) delivery. n=5 mice per group, data are represented as mean±SEM, two-tailed unpaired t-test, n.s., not significant. FIG. 30B illustrates effects of CNO on rotational behavior of mice after nigral injection of AAV8R12-G88P3-EYFP followed by intracranial (i.e.) infusion into the dorsomedial striatum. n=5 mice per group, data are represented as mean SEM, two-tailed unpaired t-test, n.s., not significant. FIG. 30C illustrates effects of CNO on rotational behavior of mice after nigral injection of AAV8R12-G88P7-EYFP followed by i.p. delivery. n=5 mice per group, data are represented as mean±SEM, two-tailed unpaired t-test, n.s., not significant. FIG. 30D illustrates effects of CNO on rotational behavior of mice after nigral injection of AAV8R12-G88P7-EYFP followed by infusion into the dorsomedial striatum. n=5 mice per group, data are represented as mean±SEM, two-tailed unpaired t-test, n.s., not significant.

FIG. 31A-31E depict testing of opto-tagging in striatonigral projection neurons in mice. FIG. 31A illustrates retrograde labeling by stereotaxic injections of AAV8R12-G88P7-HA-rM3Ds-2A-Cre into the SNr and AAV9-EF1α-DIO-ChR2-EYFP into the striatum in C57BL/6J mice. FIG. 31B illustrates HA and EYFP staining in striatal sections. Arrowheads indicate cells that were positive for both HA and EYFP. Scale bar, 10 μm. FIG. 31C illustrates whole-cell patch clamp recordings were conducted from EYFP+ cells in ex vivo slices. The raw spike trace of a typical neuron in the striatum upon blue light (473 nm) illumination. FIG. 31D illustrates electrophysiological response to CNO in optically identified striatonigral projection neurons. Representative traces (left panel) and quantitation (right panel) of action potentials induced by somatic current injection at baseline and after CNO administration. n=6 cells from 3 mice, data are represented as mean±SEM, two-tailed paired t-test, *p<0.05. FIG. 31E is a plot of induced action potentials comparing baseline and CNO.

FIG. 32A-32F depict striatal slice electrophysiological recordings after nigral injection of AAV8R12 in parkinsonian mice. FIG. 32A illustrates latency of the first AP after current injection before and after CNO incubation. n=8 cells from 7 mice, data are represented as mean±SEM, two-tailed paired t-test, ****p<0.0001. FIG. 32B-32C illustrate basal activity without current injection (FIG. 32B) and resting membrane potential (FIG. 32C) were recorded in slice prepared from parkinsonian mice received nigral AAV8R12-G88P7-rM3Ds-2A-EYFP injections before and after CNO incubation. n=12 cells from 8 mice, data are represented as mean±SEM, two-tailed paired t-test, n.s., not significant. FIG. 32D illustrates latency of the first AP after current injection before and after CNO incubation. n=6 cells from 3 mice, data are represented as mean±SEM, two-tailed paired t-test, n.s., not significant. FIG. 32E-32F illustrate basal activity without current injection (FIG. 32E) and resting membrane potential (FIG. 32F) were recorded in slice prepared from parkinsonian mice received nigral AAV8R12-G88P7-EYFP injections before and after CNO incubation. n=7 cells from 3 mice, data are represented as mean±SEM, two-tailed paired t-test, n.s., not significant.

FIG. 33A illustrates BG direct pathway manipulation in a parkinsonian macaque model by stereotaxic injections of AAV8R12-G88P7-rM3Ds-2A-EYFP into SNr, followed by MPP+-mediated depletion of SNc dopaminergic neurons, and DCZ-mediated activation of rM3Ds. FIG. 33B is a schematic of injection sites in a SNr of macaques. A guiding grid installed above SN was used to obtain the coordinates from MRI images and to guide the targeting during the injection. Nine distributed sites (green spot) were selected for injection to cover as much of the SNr as possible.

FIG. 34A-34D depict effects of saline administration on parkinsonian symptoms in macaques. FIG. 34A illustrates 1uantification of total PD score of macaques before, 3 days, 1 week, and 2 weeks after saline administration. n=4 monkeys per group, data are represented as mean±SEM, one-way ANOVA with post-hoc Dunnett's test, n.s., not significant. FIG. 34B-34D illustrate travel distance (FIG. 34B), immobile time (FIG. 34C), and time spent on the top compartment of the observation cage (FIG. 34D) in macaques after MPP+ lesion and after saline administration compared with pre-lesion state. n=4 monkeys per group, data are represented as mean±SEM, one-way ANOVA with post-hoc Dunnett's test, *p<0.05, n.s., not significant.

FIG. 35A-35G depict: DCZ alone doesn't alter motor-related behaviors in naïve macaques. FIG. 35A includes representative top-view movement tracing plots of an observation cage housing naïve macaques following intramuscular (i.m.) saline or DCZ (0.3 mg/kg) infusions. FIG. 35B includes representative side-view heatmaps of macaques following i.m. saline or DCZ (0.3 mg/kg) infusion. FIG. 35C illustrates quantitation of the ratio of contraversive rotations to ipsiversive rotations after saline or DCZ (0.3 mg/kg) infusion in macaques. n=4 monkeys per group, data are represented as mean±SEM, two-tailed paired t-test, n.s., not significant. FIG. 35D illustrates uantitation of time on the top compartment of the observation cage after saline or DCZ (0.3 mg/kg) infusion in macaques. n=4 monkeys per group, data are represented as mean SEM, two-tailed paired t-test, n.s., not significant. FIG. 35E-35F illustrate quantitation of total distance traveled (FIG. 35E) and immobile time (FIG. 35F) after saline or DCZ (0.3 mg/kg) infusion in macaques. n=4 monkeys per group, data are represented as mean±SEM, two-tailed paired t-test, n.s., not significant. FIG. 35G illustrates total activity of macaques in the observation cage after saline or DCZ (0.3 mg/kg) infusion was divided into low, mid, and high mobility. n=4 monkeys per group, data are represented as mean±SEM, two-tailed paired t-test, n.s., not significant.

FIG. 36A-36F depict effects of D1-MSN activation on dyskinesia-like behavior and health state-related blood factors in parkinsonian macaques. FIG. 36A illustrates quantitation of dyskinesia score at 2 months, 4 months, and 8 months after treatment with DCZ. n=3 monkeys per group. FIG. 36B-36F illustrate blood test results on alanine aminotransferase (ALT, FIG. 36B), aspartate aminotransferase (AST, FIG. 36C), gamma-glutamyl transferase (GGT, FIG. 36D), creatinine (CREA, FIG. 36E), blood urea nitrogen (BUN, FIG. 36F) in macaques that received nigral injections of AAV8R12-G88P7-rM3Ds-2A-EYFP before and after DCZ treatment. n=3 monkeys per group, data are represented as mean±SEM.

FIG. 37A-37F depict effects of L-Dopa treatment on parkinsonian symptoms in macaques. FIG. 37A illustrates separated PD scores of MPP+-treated macaques before and after L-Dopa treatment. n=3 monkeys per group, data are represented as mean±SEM, two-tailed paired t-test, *p<0.05, **p<0.01, n.s., not significant. FIG. 37B includes representative top view movement traces of macaques in an observation cage. FIG. 37C-37E illustrate travel distance (FIG. 37C), time spent on the top compartment of the observation cage (FIG. 37D), and immobile time (FIG. 37E) in macaques after MPP+ lesion and after treatment with L-Dopa compared with pre-lesion state. n=3 monkeys per group, data are represented as mean±SEM, one-way ANOVA with post-hoc Dunnett's test, *p<0.05, n.s., not significant. FIG. 37F illustrates total activity of macaques in the observation cage was divided into low, mid, and high mobility. Quantitation of the percentage of high mobility fraction indicated the activity changes in macaques after MPP+ lesion and after L-Dopa treatment compared with pre-lesion state. n=3 monkeys per group, data are represented as mean SEM, one-way ANOVA with post-hoc Dunnett's test, *p<0.05, n.s., not significant.

### DETAILED DESCRIPTION

Gene therapy using viral vectors works by introducing genetic material (e.g., a transgene or a nuclease) into the nucleus of a cell using a vector. Viral vectors, vectors built to resemble viruses without causing viral infections, are used to deliver gene therapies to cells (e.g., mammalian cells) as the viral vectors are able to pass through a cell's membrane and deliver their cargo genetic material into the nucleus of the host cell. The host cell can then utilize the newly-introduced genetic material to provide the desired treatment effect. Described herein are promoters that can be used with viral and non-viral vectors to achieve neuronal specific expression.

Adeno-associated viruses (AAV) can be used as delivery vectors for gene therapies as they are capable of establishing a latent infection whereby the AAV genome is incorporated into the host chromosome without provoking a destructive T cell immune response. Different types of AAVs allow for targeting of different cells for more nuanced delivery of gene therapies inside the body.

AAVs, although they can target many types of cells, do not allow for complete control over the target cell population. The present disclosure provides recombinant AAVs (rAAV) which can target specific cell types for use in methods of disease treatment. The present disclosure also provides rAAVs which have increased infectivity of medium spiny neurons. In one embodiment, rAAVs which target medium spiny neurons are used to treat Parkinson's disease.

AAV receptors (AAVR) are the receptors essential for the entry of AAVs into cells. Engineered AAVRs can be used to create designer receptors exclusively activated by designer drugs (DREADD), especially in order to target neuronal tissues. In one embodiment, DREADDs for medium spiny neurons are used to guide AAV gene therapy treatments for the treatment of Parkinson's disease.

Gene therapies rely on the ability to express heterologous genes of interest in a way to provide the desired treatment effect. The present disclosure provides promoters which can enhance translation of genes of interest for use in methods of disease treatment. The present disclosure also provides recombinant AAVs (rAAVs) coupled to regulatory elements (e.g., promoters) which have increased gene expression of the genes of interest in medium spiny neurons. In one embodiment, the rAAVs coupled to the regulatory elements (e.g., promoters) target medium spiny neurons and are used to treat Parkinson's disease.

Parkinson's disease (PD) is a common neurodegenerative disorder that affects more than 6 million people worldwide. A pathophysiological signature of PD may include loss of dopaminergic neurons in the midbrain, but its cause may be unclear. PD symptoms may be treated with dopamine precursor levodopa (L-Dopa) or dopamine receptor agonists to restore the activity of basal ganglia (BG) movement control pathways. However, the action of these drugs sometimes lacks specificity due to widespread distribution of dopamine receptors in the brain and peripheral organs, which may contribute to non-BG consumption of the drugs or disturbance of other central and peripheral dopamine systems. Therefore, development of precision therapeutic solutions for PD that enable selective modulation of the specific neuronal populations and circuits affected in PD without interference of other dopaminergic pathways is in demand.

An effective and precise way to manipulate unique cell types may include using genetically-encoded recombinases that are specifically expressed in a cell types of interest, but this approach is often not feasible for clinical interventions. An alternative approach may employ promoters or enhancers of genes expressed by unique cell types to drive cell type-specific expression, but it may be that only a handful of identified neuronal promoters maintain endogenous gene expression specificity across rodent and primate models. Retrograde AAV tracers have been developed that may differ from traditional AAV vectors by their ability to infect neurons through axonal terminals, and it may be that a recombinase-free system for targeting and modulating specialized projection neuron types can be useful or constructed with any of the following components: (1) a retrograde AAV that can effectively infect axons of selected projection neurons; (2) a promoter or enhancer that drives high levels of gene expression in target projection neurons; and (3) a chemogenetic effector that can control neuronal excitation of the specifically labeled projection neurons. This strategy may not need genetically modified animals and thus may be more useful for clinical applications in humans. In parkinsonian rodents, dopamine loss may induce repression of direct pathway activity and targeted activation of striatal D1 dopamine receptor-expressing medium spiny neurons (D1-MSNs) and effectively rescue core motor symptoms. Since D1-MSNs are, in some instances, the only major cell type in the striatum projecting to the substantia nigra pars reticulata (SNr), they may represent an ideal target for implementing a circuit-specific modulatory approach for PD. As such, this disclosure includes development of a recombinase-free, retrograde AAV-based strategy to precisely isolate and modulate D1-MSNs, and investigations into its efficacy in reversing PD symptoms.

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

### Definitions

As used herein the term “about” refers to an amount that is near the stated amount by 10% or less.

As used herein the term “individual,” “patient,” or “subject” refers to individuals diagnosed with, suspected of being afflicted with, or at-risk of developing at least one disease for which the described compositions and method are useful for treating. In certain embodiments the individual is a mammal. In certain embodiments, the mammal is a mouse, rat, rabbit, dog, cat, horse, cow, sheep, pig, goat, llama, alpaca, or yak. In certain embodiments, the individual is a human.

The terms “polypeptide” and “protein” are used interchangeably to refer to a polymer of amino acid residues and are not limited to a minimum length. Polypeptides, including the provided antibodies and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The polypeptides described herein can be encoded by a nucleic acid. A nucleic acid is a type of polynucleotide comprising two or more nucleotide bases. The terms “nucleic acid” and “nucleic acid molecule” can be used interchangeably. The terms refer to nucleic acids of any composition form, such as deoxyribonucleic acid (DNA, e.g., complementary DNA (cDNA), genomic DNA (gDNA) and the like), ribonucleic acid (RNA, e.g., message RNA (mRNA), short inhibitory RNA (siRNA), ribosomal RNA (rRNA), transfer RNA (tRNA), microRNA, RNA highly expressed by the fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single stranded, double-stranded and the like). A nucleic acid may be, or may be from, a plasmid, phage, autonomously replicating sequence (ARS), centromere, artificial chromosome, chromosome, or other nucleic acid able to replicate or be replicated in vitro or in a host cell, a cell, a cell nucleus or cytoplasm of a cell in certain embodiments. A nucleic acid in some embodiments can be from a single chromosome (e.g., a nucleic acid sample may be from one chromosome of a sample obtained from a diploid organism). Nucleic acids also include derivatives, variants and analogs of RNA or DNA synthesized, replicated or amplified from single-stranded (“sense” or “antisense”, “plus” strand or “minus” strand, “forward” reading frame or “reverse” reading frame) and double stranded polynucleotides. Deoxyribonucleotides include deoxyadenosine, deoxycytidine, deoxyguanosine and deoxythymidine. For RNA, the base cytosine is replaced with uracil and the sugar 2′ position includes a hydroxyl moiety. A nucleic acid may be prepared using a nucleic acid obtained from a subject as a template. In some embodiments, the nucleic acid is a component of a vector that can be used to transfer the polypeptide encoding polynucleotide into a cell. A heterologous nucleic acid is a nucleic acid that is exogenous to a cell or cell population being modified. A heterologous nucleic acid may comprise a gene or nucleotide sequence that is a modified from an endogenous gene or may comprise a recombinant gene or nucleic acid sequence. Heterologous nucleic acids may comprise regulatory sequences, encode fusions to endogenous genes or other modifications that increase the therapeutic or diagnostic potential of a gene or nucleotide sequence.

As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes encoded by the vectors are referred to herein as “expression vectors.” Expression vectors can suitably initiate expression of a gene of interest operatively coupled to promoter, such promoters can be “universal,” that is, active in all or many different cell types (e.g., CMV promoter), or tissue or cell specific, that is, active in a certain subset of cells or tissues. Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

“Heterologous” as used herein in reference to a nucleic acid, gene, polypeptide or protein is a nucleic acid, gene, polypeptide or protein that is not a natural component of the adeno-associated viruses (AAVs) described herein or naturally regulated in cis by any of the promoters described herein. Heterologous nucleic acids may encode a gene or RNA (e.g., antisense or siRNA) not normally expressed by the AAVs described herein including synthetic, mammalian, or human genes or RNAs.

As described herein “operatively coupled” refers to the arrangement of a promoter or regulatory region to an open reading frame (e.g., gene of interest or target gene) on a nucleic acid molecule that results in transcription of the open reading frame. Generally, a regulatory region will be 5′ to the open reading frame such and may comprise one or more intervening nucleotides that do not significantly inhibit transcription of the open reading frame.

A designer receptor exclusively activated by designer drugs (DREADD) is a class of artificially engineered protein receptors used in the field of chemogenetics which are selectively activated by certain ligands. They can be used in biomedical research such as neuroscience to manipulate the activity of neurons. Non-limiting examples of DREADDs can be found in Urban DJ and Roth BL, 2015, DREADDs (Designer Receptors Exclusively Activated by Designer Drugs): Chemogenetic Tools with Therapeutic Utility, Annu. Rev. Pharmacol. Toxicol. 55:15.1-15.19 and Roth, 2016, DREADDs for Neuroscientists, Neuron. 89:683-694.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application.

As used herein, the term “serotype” refers to a distinguishable strain of a microorganism. A serotype can be defined as a group of organisms that have the same type and number of surface antigens. Serotypes may or may not differ from strains, which are isolates of a single culture. Serotypes may or may not differ from genotypes which have different sets of genes.

Disclosed herein, in some embodiments, are nucleic acid or protein sequences. Any inconsistency between a sequence in the sequence listing and written description should normally be resolved in favor of the written description.

### Adeno-Associated Viruses (AAV)

AAVs are viruses composed of non-enveloped icosahedral capsid protein shells that contain a linear single-stranded DAN genome. The genomes of AAV vectors retain their packaging signals (also known as inverted terminal repeats, or ITRs) but replace other viral sequences with exogenous DNA of choice. The DNA of interest flanked by the AAV ITRs can be referred to as a transgene expression cassette.

The transgene expression cassette is packaged in an AAV capsid for the infection and transduction of target cells. After entering the body, viral capsids interact with receptors on the surface of a target cell. The viral capsids are then internalized into the target cell through endocytosis. Intracellular trafficking through the endocytic and/or proteasomal compartment is followed by endosomal escape, nuclear import, virion uncoating, and viral DNA double-strand conversion that leads to the transcription and expression of the transgene. AAV vectors can be produced as in Kimura et al. production of adeno-associated virus vectors for in vitro and in vivo applications, Sci Rep 9, 12601 (2019).

There are several AAV serotypes which can include, but are not limited to AAV1, AAV2, AAV3, AAV4, AAV5, AAV6, AAV7, AAV8, AAV9, AAV10, AAV 11, AAV12, AAV13, Rh10, PHP.B, PHP.eB, and PHP.S. AAV vectors can include elements from any one serotype, a mixture of serotypes, hybrids or chimeras of different serotypes, or a combination thereof.

Recombinant AAVs (rAAV) are built of single-stranded DNA (ssDNA). These ssDNA viral vectors have high transduction rates and have the property of stimulating endogenous homologous recombination, a DNA repair mechanism, without causing double strand DNA breaks in the genome. In various embodiments, a recombinant AAV vector includes a sequence derived from an AAV1, AAV2, AAV3, AAV4, AAV5, AAV6, AAV7, AAV8, AAV9, AAV10, AAV 11, AAV12, AAV13, Rh10, PHP.B, PHP.eB, or PHP.S serotype, or a mixture, hybrid, or chimera of any of the foregoing AAV serotypes. In one embodiment, a recombinant AAV vector includes a sequence derived from AAV2. In one embodiment, a recombinant AAV vector includes a sequence derived from AAV8. In one embodiment, a recombinant AAV vector includes a sequence derived from AAV9. In one embodiment, a recombinant AAV vector includes a sequence derived from AAV2 and AAV8. In one embodiment, a recombinant AAV vector includes a sequence derived from AAV2 and AAV9. In one embodiment, a recombinant AAV vector includes a sequence derived from AAV8 and AAV9. In one embodiment, a recombinant AAV vector includes a sequence derived from AAV2, AAV8, and AAV9.

In a further embodiment, AAV vectors include capsids derived from AAV1, AAV2, AAV3, AAV4, AAV5, AAV6, AAV7, AAV8, AAV9, AAV10, AAV 11, AAV12, AAV13, Rh10, PHP.B, PHP.eB, or PHP.S as well as variants (e.g., capsid variants, such as amino acid insertions, additions and substitutions) thereof. AAV capsids can include a VP1 protein, a VP2 protein, and/or a VP3 protein wherein VP2 and VP3, can be amino-terminal truncations of VP1.

Retrograde infections spread a virus from axon terminals to the parent neurons, the opposite direction of a nerve impulse. Retrograde AAVs (AAV-retro) can be used to study specific neuronal populations.

### AAV Capsid Polypeptides

The AAV vector has a protective protein shell called a capsid. AAV capsid polypeptides are the primary interface between the host and the vector genome. Specificity and efficiency of transduction of AAV particles are dependent on vector capsids (e.g., the AAV capsid polypeptide).

In one embodiment, a rAAV virion can comprise a variant capsid polypeptide, wherein the variant capsid polypeptide comprises a modification to an amino acid. A modification to an amino acid can comprise an amino acid addition, an amino acid deletion, an amino acid substitution, an amino acid alteration or a combination thereof. The variant capsid polypeptide can comprise an amino acid alteration to SEQ ID NO: 1. The alteration can be one that allows for retrograde transport of the AAV along the axon of the neuron, thus allowing the AAV to reach the body of the neuron where it may allow for transcription of a heterologous nucleic acid included therein. In sequence IDs, bolded and underlined portions are retro insertions. In the sequence IDs, bolded portions are capsid mutations.

The variant capsid polypeptide can comprise an amino acid alteration at any one or more, any two or more, any three or more, any four or more, any five or more, any six or more, any seven or more, or all eight alterations of V125, V183, N411, Y447, R490, T495, F536, or A606 of SEQ ID NO: 1. Alternatively, or in addition to, the variant capsid polypeptide can comprise an amino acid substitution at any one or more, any two or more, any three or more, any four or more, any five or more, any six or more, any seven or more, or all eight alternations of V125, V183, N411, Y447, R490, T495, F536, or A606 of SEQ ID NO: 1.

In one embodiment, the V125 substitution can be V125I. In another embodiment, the V183 substitution can be V183E. In another embodiment, the N411 substitution can be N411 S. In another embodiment, the Y447 substitution can be Y447F. In another embodiment, the R490 substitution can be R490Q. In another embodiment, the T495 substitution can be T495A. In another embodiment, the F536 substitution can be F536Y. In another embodiment, the A606 substitution can be A606S. In another embodiment, the variant capsid polypeptide comprises at least one or more, at least two or more, at least three or more, at least four or more, at least five or more, at least six or more, at least seven or more, or all eight of the substitutions from the list comprising V125I, V183E, N411 S, Y447F, R490Q, T495A, F536Y, and A606S.

In one embodiment, the variant capsid polypeptide comprises a V125I substitution and a V183E substitution. Alternatively, the variant capsid polypeptide comprises a V125I substitution and a N411 S substitution. Alternatively, the variant capsid polypeptide comprises a V125I substitution and a Y447F substitution. Alternatively, the variant capsid polypeptide comprises a V125I substitution and a R490Q substitution. Alternatively, the variant capsid polypeptide comprises a V125I substitution and a T495A substitution. Alternatively, the variant capsid polypeptide comprises a V125I substitution and a F536Y substitution. Alternatively, the variant capsid polypeptide comprises a V125I substitution and a A606S substitution. Alternatively, the variant capsid polypeptide comprises a V183E substitution and a N411S substitution. Alternatively, the variant capsid polypeptide comprises a V183E substitution and a Y447F substitution. Alternatively, the variant capsid polypeptide comprises a V183E substitution and a R490Q substitution. Alternatively, the variant capsid polypeptide comprises a V183E substitution and a T495A substitution. Alternatively, the variant capsid polypeptide comprises a V183E substitution and a F536Y substitution. Alternatively, the variant capsid polypeptide comprises a V183E substitution and a A606S substitution. Alternatively, the variant capsid polypeptide comprises a N411S substitution and a Y447F substitution. Alternatively, the variant capsid polypeptide comprises a N411S substitution and a R490Q substitution. Alternatively, the variant capsid polypeptide comprises a N411S substitution and a T495A substitution. Alternatively, the variant capsid polypeptide comprises a N411S substitution and a F536Y substitution. Alternatively, the variant capsid polypeptide comprises a N411S substitution and a A606S substitution. Alternatively, the variant capsid polypeptide comprises a Y447F substitution and a R490Q substitution. Alternatively, the variant capsid polypeptide comprises a Y447F substitution and a T495A substitution. Alternatively, the variant capsid polypeptide comprises a Y447F substitution and a F536Y substitution. Alternatively, the variant capsid polypeptide comprises a Y447F substitution and a A606S substitution. Alternatively, the variant capsid polypeptide comprises a R490Q substitution and a T495A substitution. Alternatively, the variant capsid polypeptide comprises a F536Y substitution and a T495A substitution. Alternatively, the variant capsid polypeptide comprises a R490Q substitution and a A606S substitution. Alternatively, the variant capsid polypeptide comprises a T495A substitution and a F536Y substitution. Alternatively, the variant capsid polypeptide comprises a T495A substitution and a A606S substitution. Alternatively, the variant capsid polypeptide comprises a F536Y substitution and a A606S substitution.

In another embodiment, the variant capsid polypeptide comprises a V125I substitution, a V183E substitution, and a N411S substitution. In another embodiment, the variant capsid polypeptide comprises a V125I substitution, a V183E substitution, and a Y447F substitution. In another embodiment, the variant capsid polypeptide comprises a V125I substitution, a V183E substitution, and a R490Q substitution. In another embodiment, the variant capsid polypeptide comprises a V125I substitution, a V183E substitution, and a T495A substitution. In another embodiment, the variant capsid polypeptide comprises a V125I substitution, a V183E substitution, and a F536Y substitution. In another embodiment, the variant capsid polypeptide comprises a V125I substitution, a V183E substitution, and a A606S substitution. In another embodiment, the variant capsid polypeptide comprises a V125I substitution, a N411 S substitution, and a Y447F substitution. In another embodiment, the variant capsid polypeptide comprises a V125I substitution, a N411 S substitution, and a R490Q substitution. In another embodiment, the variant capsid polypeptide comprises a V125I substitution, a N411 S substitution, and a T495A substitution. In another embodiment, the variant capsid polypeptide comprises a V125I substitution, a N411 S substitution, and a F536Y substitution. In another embodiment, the variant capsid polypeptide comprises a V125I substitution, a N411 S substitution, and a A606S substitution. In another embodiment, the variant capsid polypeptide comprises a V125I substitution, a Y447F substitution, and a R490Q substitution. In another embodiment, the variant capsid polypeptide comprises a V125I substitution, a Y447F substitution, and a T495A substitution. In another embodiment, the variant capsid polypeptide comprises a V125I substitution, a Y447F substitution, and a F536Y substitution. In another embodiment, the variant capsid polypeptide comprises a V125I substitution, a Y447F substitution, and a A606S substitution. In another embodiment, the variant capsid polypeptide comprises a V125I substitution, a R490Q substitution, and a T495A substitution. In another embodiment, the variant capsid polypeptide comprises a V125I substitution, a R490Q substitution, and a F536Y substitution. In another embodiment, the variant capsid polypeptide comprises a V125I substitution, a R490Q substitution, and a A606S substitution. In another embodiment, the variant capsid polypeptide comprises a V125I substitution, a T495A substitution, and a F536Y substitution. In another embodiment, the variant capsid polypeptide comprises a V125I substitution, a T495A substitution, and a A606S substitution. In another embodiment, the variant capsid polypeptide comprises a V125I substitution, a F536Y substitution, and a A606S substitution. In another embodiment, the variant capsid polypeptide comprises a V183E substitution, a N411S substitution, and a Y447F substitution. In another embodiment, the variant capsid polypeptide comprises a V183E substitution, a N411 S substitution, and a R490Q substitution. In another embodiment, the variant capsid polypeptide comprises a V183E substitution, a N411 S substitution, and a T495A substitution. In another embodiment, the variant capsid polypeptide comprises a V183E substitution, a N411 S substitution, and a F536Y substitution. In another embodiment, the variant capsid polypeptide comprises a V183E substitution, a N411S substitution, and a A606S substitution. In another embodiment, the variant capsid polypeptide comprises a V183E substitution, a Y447F substitution, and a R490Q substitution. In another embodiment, the variant capsid polypeptide comprises a V183E substitution, a Y447F substitution, and a T495A substitution. In another embodiment, the variant capsid polypeptide comprises a V183E substitution, a Y447F substitution, and a F536Y substitution. In another embodiment, the variant capsid polypeptide comprises a V183E substitution, a Y447F substitution, and a A606S substitution. In another embodiment, the variant capsid polypeptide comprises a V183E substitution, a R490Q substitution, and a T495A substitution. In another embodiment, the variant capsid polypeptide comprises a V183E substitution, a R490Q substitution, and a F536Y substitution. In another embodiment, the variant capsid polypeptide comprises a V183E substitution, a R490Q substitution, and a A606S substitution. In another embodiment, the variant capsid polypeptide comprises a V183E substitution, a T495A substitution, and a F536Y substitution. In another embodiment, the variant capsid polypeptide comprises a V183E substitution, a T495A substitution, and a A606S substitution. In another embodiment, the variant capsid polypeptide comprises a V183E substitution, a F536Y substitution, and a A606S substitution. In another embodiment, the variant capsid polypeptide comprises a N411S substitution, a Y447F substitution, and a R490Q substitution. In another embodiment, the variant capsid polypeptide comprises a N411S substitution, a Y447F substitution, and a T495A substitution. In another embodiment, the variant capsid polypeptide comprises a N411S substitution, a Y447F substitution, and a F536Y substitution. In another embodiment, the variant capsid polypeptide comprises a N411 S substitution, a Y447F substitution, and a A606S substitution. In another embodiment, the variant capsid polypeptide comprises a N411S substitution, a R490Q substitution, and a T495A substitution. In another embodiment, the variant capsid polypeptide comprises a N411S substitution, a R490Q substitution, and a F536Y substitution. In another embodiment, the variant capsid polypeptide comprises a N411S substitution, a R490Q substitution, and a A606S substitution. In another embodiment, the variant capsid polypeptide comprises a N411S substitution, a T495A substitution, and a A606S substitution. In another embodiment, the variant capsid polypeptide comprises a N411 S substitution, a T495A substitution, and a F536Y substitution. In another embodiment, the variant capsid polypeptide comprises a N411S substitution, a T495A substitution, and a A606S substitution. In another embodiment, the variant capsid polypeptide comprises a N411S substitution, a F536Y substitution, and a A606S substitution. In another embodiment, the variant capsid polypeptide comprises a Y447F substitution, a R490Q substitution, and a T495A substitution. In another embodiment, the variant capsid polypeptide comprises a Y447F substitution, a R490Q substitution, and a F536Y substitution. In another embodiment, the variant capsid polypeptide comprises a Y447F substitution, a R490Q substitution, and a A606S substitution. In another embodiment, the variant capsid polypeptide comprises a Y447F substitution, a T495A substitution, and a F536Y substitution. In another embodiment, the variant capsid polypeptide comprises a Y447F substitution, a T495A substitution, and a A606S substitution. In another embodiment, the variant capsid polypeptide comprises a Y447F substitution, a F536Y substitution, and a A606S substitution. In another embodiment, the variant capsid polypeptide comprises a R490Q substitution, a T495A substitution, and a F536Y substitution. In another embodiment, the variant capsid polypeptide comprises a R490Q substitution, a T495A substitution, and a A606S substitution. In another embodiment, the variant capsid polypeptide comprises a R490Q substitution, a F536Y substitution, and a A606S substitution. In another embodiment, the variant capsid polypeptide comprises a T495A substitution, a F536Y substitution, and a A606S substitution.

The variant capsid polypeptide can comprise the amino acid sequence set forth in SEQ ID NO: 1. The variant capsid polypeptide can have at least about 80% sequence identity to SEQ ID NO: 1, at least about 82% sequence identity to SEQ ID NO: 1, at least about 84% sequence identity to SEQ ID NO: 1, at least about 86% sequence identity to SEQ ID NO: 1, at least about 88% sequence identity to SEQ ID NO: 1, at least about 90% sequence identity to SEQ ID NO: 1, at least about 91% sequence identity to SEQ ID NO: 1, at least about 92% sequence identity to SEQ ID NO: 1, at least about 93% sequence identity to SEQ ID NO: 1, at least about 94% sequence identity to SEQ ID NO: 1, at least about 95% sequence identity to SEQ ID NO: 1, at least about 96% sequence identity to SEQ ID NO: 1, at least about 97% sequence identity to SEQ ID NO: 1, at least about 98% sequence identity to SEQ ID NO: 1, or at least about 99% sequence identity to SEQ ID NO: 1.

The variant capsid polypeptide can comprise the amino acid sequence set forth in SEQ ID NO: 2. The variant capsid polypeptide can have at least about 80% sequence identity to SEQ ID NO: 2, at least about 82% sequence identity to SEQ ID NO: 2, at least about 84% sequence identity to SEQ ID NO: 2, at least about 86% sequence identity to SEQ ID NO: 2, at least about 88% sequence identity to SEQ ID NO: 2, at least about 90% sequence identity to SEQ ID NO: 2, at least about 91% sequence identity to SEQ ID NO: 2, at least about 92% sequence identity to SEQ ID NO: 2, at least about 93% sequence identity to SEQ ID NO: 2, at least about 94% sequence identity to SEQ ID NO: 2, at least about 95% sequence identity to SEQ ID NO: 2, at least about 96% sequence identity to SEQ ID NO: 2, at least about 97% sequence identity to SEQ ID NO: 2, at least about 98% sequence identity to SEQ ID NO: 2, or at least about 99% sequence identity to SEQ ID NO: 2.

The variant capsid polypeptide can comprise the amino acid sequence set forth in SEQ ID NO: 3. The variant capsid polypeptide can have at least about 80% sequence identity to SEQ ID NO: 3, at least about 82% sequence identity to SEQ ID NO: 3, at least about 84% sequence identity to SEQ ID NO: 3, at least about 86% sequence identity to SEQ ID NO: 3, at least about 88% sequence identity to SEQ ID NO: 3, at least about 90% sequence identity to SEQ ID NO: 3, at least about 91% sequence identity to SEQ ID NO: 3, at least about 92% sequence identity to SEQ ID NO: 3, at least about 93% sequence identity to SEQ ID NO: 3, at least about 94% sequence identity to SEQ ID NO: 3, at least about 95% sequence identity to SEQ ID NO: 3, at least about 96% sequence identity to SEQ ID NO: 3, at least about 97% sequence identity to SEQ ID NO: 3, at least about 98% sequence identity to SEQ ID NO: 3, or at least about 99% sequence identity to SEQ ID NO: 3.

The variant capsid polypeptide can comprise the amino acid sequence set forth in SEQ ID NO: 4. The variant capsid polypeptide can have at least about 80% sequence identity to SEQ ID NO: 4, at least about 82% sequence identity to SEQ ID NO: 4, at least about 84% sequence identity to SEQ ID NO: 4, at least about 86% sequence identity to SEQ ID NO: 4, at least about 88% sequence identity to SEQ ID NO: 4, at least about 90% sequence identity to SEQ ID NO: 4, at least about 91% sequence identity to SEQ ID NO: 4, at least about 92% sequence identity to SEQ ID NO: 4, at least about 93% sequence identity to SEQ ID NO: 4, at least about 94% sequence identity to SEQ ID NO: 4, at least about 95% sequence identity to SEQ ID NO: 4, at least about 96% sequence identity to SEQ ID NO: 4, at least about 97% sequence identity to SEQ ID NO: 4, at least about 98% sequence identity to SEQ ID NO: 4, or at least about 99% sequence identity to SEQ ID NO: 4.

The variant capsid polypeptide can comprise the amino acid sequence set forth in SEQ ID NO: 5. The variant capsid polypeptide can have at least about 80% sequence identity to SEQ ID NO: 5, at least about 82% sequence identity to SEQ ID NO: 5, at least about 84% sequence identity to SEQ ID NO: 5, at least about 86% sequence identity to SEQ ID NO: 5, at least about 88% sequence identity to SEQ ID NO: 5, at least about 90% sequence identity to SEQ ID NO: 5, at least about 91% sequence identity to SEQ ID NO: 5, at least about 92% sequence identity to SEQ ID NO: 5, at least about 93% sequence identity to SEQ ID NO: 5, at least about 94% sequence identity to SEQ ID NO: 5, at least about 95% sequence identity to SEQ ID NO: 5, at least about 96% sequence identity to SEQ ID NO: 5, at least about 97% sequence identity to SEQ ID NO: 5, at least about 98% sequence identity to SEQ ID NO: 5, or at least about 99% sequence identity to SEQ ID NO: 5.

The variant capsid polypeptide can comprise the amino acid sequence set forth in SEQ ID NO: 6. The variant capsid polypeptide can have at least about 80% sequence identity to SEQ ID NO: 6, at least about 82% sequence identity to SEQ ID NO: 6, at least about 84% sequence identity to SEQ ID NO: 6, at least about 86% sequence identity to SEQ ID NO: 6, at least about 88% sequence identity to SEQ ID NO: 6, at least about 90% sequence identity to SEQ ID NO: 6, at least about 91% sequence identity to SEQ ID NO: 6, at least about 92% sequence identity to SEQ ID NO: 6, at least about 93% sequence identity to SEQ ID NO: 6, at least about 94% sequence identity to SEQ ID NO: 6, at least about 95% sequence identity to SEQ ID NO: 6, at least about 96% sequence identity to SEQ ID NO: 6, at least about 97% sequence identity to SEQ ID NO: 6, at least about 98% sequence identity to SEQ ID NO: 6, or at least about 99% sequence identity to SEQ ID NO: 6.

The variant capsid polypeptide can comprise the amino acid sequence set forth in SEQ ID NO: 7. The variant capsid polypeptide can have at least about 80% sequence identity to SEQ ID NO: 7, at least about 82% sequence identity to SEQ ID NO: 7, at least about 84% sequence identity to SEQ ID NO: 7, at least about 86% sequence identity to SEQ ID NO: 7, at least about 88% sequence identity to SEQ ID NO: 7, at least about 90% sequence identity to SEQ ID NO: 7, at least about 91% sequence identity to SEQ ID NO: 7, at least about 92% sequence identity to SEQ ID NO: 7, at least about 93% sequence identity to SEQ ID NO: 7, at least about 94% sequence identity to SEQ ID NO: 7, at least about 95% sequence identity to SEQ ID NO: 7, at least about 96% sequence identity to SEQ ID NO: 7, at least about 97% sequence identity to SEQ ID NO: 7, at least about 98% sequence identity to SEQ ID NO: 7, or at least about 99% sequence identity to SEQ ID NO: 7.

The variant capsid polypeptide can comprise the amino acid sequence set forth in SEQ ID NO: 8. The variant capsid polypeptide can have at least about 80% sequence identity to SEQ ID NO: 8, at least about 82% sequence identity to SEQ ID NO: 8, at least about 84% sequence identity to SEQ ID NO: 8, at least about 86% sequence identity to SEQ ID NO: 8, at least about 88% sequence identity to SEQ ID NO: 8, at least about 90% sequence identity to SEQ ID NO: 8, at least about 91% sequence identity to SEQ ID NO: 8, at least about 92% sequence identity to SEQ ID NO: 8, at least about 93% sequence identity to SEQ ID NO: 8, at least about 94% sequence identity to SEQ ID NO: 8, at least about 95% sequence identity to SEQ ID NO: 8, at least about 96% sequence identity to SEQ ID NO: 8, at least about 97% sequence identity to SEQ ID NO: 8, at least about 98% sequence identity to SEQ ID NO: 8, or at least about 99% sequence identity to SEQ ID NO: 8.

The variant capsid polypeptide can comprise the amino acid sequence set forth in SEQ ID NO: 9. The variant capsid polypeptide can have at least about 80% sequence identity to SEQ ID NO: 9, at least about 82% sequence identity to SEQ ID NO: 9, at least about 84% sequence identity to SEQ ID NO: 9, at least about 86% sequence identity to SEQ ID NO: 9, at least about 88% sequence identity to SEQ ID NO: 9, at least about 90% sequence identity to SEQ ID NO: 9, at least about 91% sequence identity to SEQ ID NO: 9, at least about 92% sequence identity to SEQ ID NO: 9, at least about 93% sequence identity to SEQ ID NO: 9, at least about 94% sequence identity to SEQ ID NO: 9, at least about 95% sequence identity to SEQ ID NO: 9, at least about 96% sequence identity to SEQ ID NO: 9, at least about 97% sequence identity to SEQ ID NO: 9, at least about 98% sequence identity to SEQ ID NO: 9, or at least about 99% sequence identity to SEQ ID NO: 9.

The variant capsid polypeptide can comprise the amino acid sequence set forth in SEQ ID NO: 10. The variant capsid polypeptide can have at least about 80% sequence identity to SEQ ID NO: 10, at least about 82% sequence identity to SEQ ID NO: 10, at least about 84% sequence identity to SEQ ID NO: 10, at least about 86% sequence identity to SEQ ID NO: 10, at least about 88% sequence identity to SEQ ID NO: 10, at least about 90% sequence identity to SEQ ID NO: 10, at least about 91% sequence identity to SEQ ID NO: 10, at least about 92% sequence identity to SEQ ID NO: 10, at least about 93% sequence identity to SEQ ID NO: 10, at least about 94% sequence identity to SEQ ID NO: 10, at least about 95% sequence identity to SEQ ID NO: 10, at least about 96% sequence identity to SEQ ID NO: 10, at least about 97% sequence identity to SEQ ID NO: 10, at least about 98% sequence identity to SEQ ID NO: 10, or at least about 99% sequence identity to SEQ ID NO: 10.

The variant capsid polypeptide can comprise the amino acid sequence set forth in SEQ ID NO: 11. The variant capsid polypeptide can have at least about 80% sequence identity to SEQ ID NO: 11, at least about 82% sequence identity to SEQ ID NO: 11, at least about 84% sequence identity to SEQ ID NO: 11, at least about 86% sequence identity to SEQ ID NO: 11, at least about 88% sequence identity to SEQ ID NO: 11, at least about 90% sequence identity to SEQ ID NO: 11, at least about 91% sequence identity to SEQ ID NO: 11, at least about 92% sequence identity to SEQ ID NO: 11, at least about 93% sequence identity to SEQ ID NO: 11, at least about 94% sequence identity to SEQ ID NO: 11, at least about 95% sequence identity to SEQ ID NO: 11, at least about 96% sequence identity to SEQ ID NO: 11, at least about 97% sequence identity to SEQ ID NO: 11, at least about 98% sequence identity to SEQ ID NO: 11, or at least about 99% sequence identity to SEQ ID NO: 11.

The variant capsid polypeptide can comprise the amino acid sequence set forth in SEQ ID NO: 12. The variant capsid polypeptide can have at least about 80% sequence identity to SEQ ID NO: 12, at least about 82% sequence identity to SEQ ID NO: 12, at least about 84% sequence identity to SEQ ID NO: 12, at least about 86% sequence identity to SEQ ID NO: 12, at least about 88% sequence identity to SEQ ID NO: 12, at least about 90% sequence identity to SEQ ID NO: 12, at least about 91% sequence identity to SEQ ID NO: 12, at least about 92% sequence identity to SEQ ID NO: 12, at least about 93% sequence identity to SEQ ID NO: 12, at least about 94% sequence identity to SEQ ID NO: 12, at least about 95% sequence identity to SEQ ID NO: 12, at least about 96% sequence identity to SEQ ID NO: 12, at least about 97% sequence identity to SEQ ID NO: 12, at least about 98% sequence identity to SEQ ID NO: 12, or at least about 99% sequence identity to SEQ ID NO: 12.

The variant capsid polypeptide can comprise the amino acid sequence set forth in SEQ ID NO: 13. The variant capsid polypeptide can have at least about 80% sequence identity to SEQ ID NO: 13, at least about 82% sequence identity to SEQ ID NO: 13, at least about 84% sequence identity to SEQ ID NO: 13, at least about 86% sequence identity to SEQ ID NO: 13, at least about 88% sequence identity to SEQ ID NO: 13, at least about 90% sequence identity to SEQ ID NO: 13, at least about 91% sequence identity to SEQ ID NO: 13, at least about 92% sequence identity to SEQ ID NO: 13, at least about 93% sequence identity to SEQ ID NO: 13, at least about 94% sequence identity to SEQ ID NO: 13, at least about 95% sequence identity to SEQ ID NO: 13, at least about 96% sequence identity to SEQ ID NO: 13, at least about 97% sequence identity to SEQ ID NO: 13, at least about 98% sequence identity to SEQ ID NO: 13, or at least about 99% sequence identity to SEQ ID NO: 13.

The variant capsid polypeptide can comprise the amino acid sequence set forth in SEQ ID NO: 14. The variant capsid polypeptide can have at least about 80% sequence identity to SEQ ID NO: 14, at least about 82% sequence identity to SEQ ID NO: 14, at least about 84% sequence identity to SEQ ID NO: 14, at least about 86% sequence identity to SEQ ID NO: 14, at least about 88% sequence identity to SEQ ID NO: 14, at least about 90% sequence identity to SEQ ID NO: 14, at least about 91% sequence identity to SEQ ID NO: 14, at least about 92% sequence identity to SEQ ID NO: 14, at least about 93% sequence identity to SEQ ID NO: 14, at least about 94% sequence identity to SEQ ID NO: 14, at least about 95% sequence identity to SEQ ID NO: 14, at least about 96% sequence identity to SEQ ID NO: 14, at least about 97% sequence identity to SEQ ID NO: 14, at least about 98% sequence identity to SEQ ID NO: 14, or at least about 99% sequence identity to SEQ ID NO: 14.

The variant capsid polypeptide can comprise the amino acid sequence set forth in SEQ ID NO: 15. The variant capsid polypeptide can have at least about 80% sequence identity to SEQ ID NO: 15, at least about 82% sequence identity to SEQ ID NO: 15, at least about 84% sequence identity to SEQ ID NO: 15, at least about 86% sequence identity to SEQ ID NO: 15, at least about 88% sequence identity to SEQ ID NO: 15, at least about 90% sequence identity to SEQ ID NO: 15, at least about 91% sequence identity to SEQ ID NO: 15, at least about 92% sequence identity to SEQ ID NO: 15, at least about 93% sequence identity to SEQ ID NO: 15, at least about 94% sequence identity to SEQ ID NO: 15, at least about 95% sequence identity to SEQ ID NO: 15, at least about 96% sequence identity to SEQ ID NO: 15, at least about 97% sequence identity to SEQ ID NO: 15, at least about 98% sequence identity to SEQ ID NO: 15, or at least about 99% sequence identity to SEQ ID NO: 15.

In another embodiment, the variant capsid polypeptide comprises an alteration which comprises an insertion of SEQ ID NO:31, an aspartic acid substitution at an amino acid residue corresponding to position 385 of SEQ ID NO: 1, an isoleucine and asparagine (IN) substitution at an amino acid residue corresponding to positions 721 and 722 of SEQ ID NO: 1, or combinations thereof.

In certain embodiments, the variant capsid polypeptide comprises an amino acid alteration to increase retrograde transport of the rAAV virion. In some embodiments, the alteration to increase retrograde transport of the rAAV can comprise SEQ ID NO 31. The amino acid alteration to increase retrograde transport of the rAAV virion can have at least about 80% sequence identity to SEQ ID NO: 31, at least about 82% sequence identity to SEQ ID NO: 31, at least about 84% sequence identity to SEQ ID NO: 31, at least about 86% sequence identity to SEQ ID NO: 31, at least about 88% sequence identity to SEQ ID NO: 31, at least about 90% sequence identity to SEQ ID NO: 31, at least about 91% sequence identity to SEQ ID NO: 31, at least about 92% sequence identity to SEQ ID NO: 31, at least about 93% sequence identity to SEQ ID NO: 31, at least about 94% sequence identity to SEQ ID NO: 31, at least about 95% sequence identity to SEQ ID NO: 31, at least about 96% sequence identity to SEQ ID NO: 31, at least about 97% sequence identity to SEQ ID NO: 31, at least about 98% sequence identity to SEQ ID NO: 31, or at least about 99% sequence identity to SEQ ID NO: 31. The sequence identity can preserve the infectivity of the retrograde AAV of SEQ ID NO: 31.

The alteration to increase retrograde transport of the rAAV can comprise SEQ ID NO 32. The amino acid alteration to increase retrograde transport of the rAAV virion can have at least about 80% sequence identity to SEQ ID NO: 32, at least about 82% sequence identity to SEQ ID NO: 32, at least about 84% sequence identity to SEQ ID NO: 32, at least about 86% sequence identity to SEQ ID NO: 32, at least about 88% sequence identity to SEQ ID NO: 32, at least about 90% sequence identity to SEQ ID NO: 32, at least about 91% sequence identity to SEQ ID NO: 32, at least about 92% sequence identity to SEQ ID NO: 32, at least about 93% sequence identity to SEQ ID NO: 32, at least about 94% sequence identity to SEQ ID NO: 32, at least about 95% sequence identity to SEQ ID NO: 32, at least about 96% sequence identity to SEQ ID NO: 32, at least about 97% sequence identity to SEQ ID NO: 32, at least about 98% sequence identity to SEQ ID NO: 32, or at least about 99% sequence identity to SEQ ID NO: 32. The sequence identity can preserve the infectivity of the retrograde AAV of SEQ ID NO: 32.

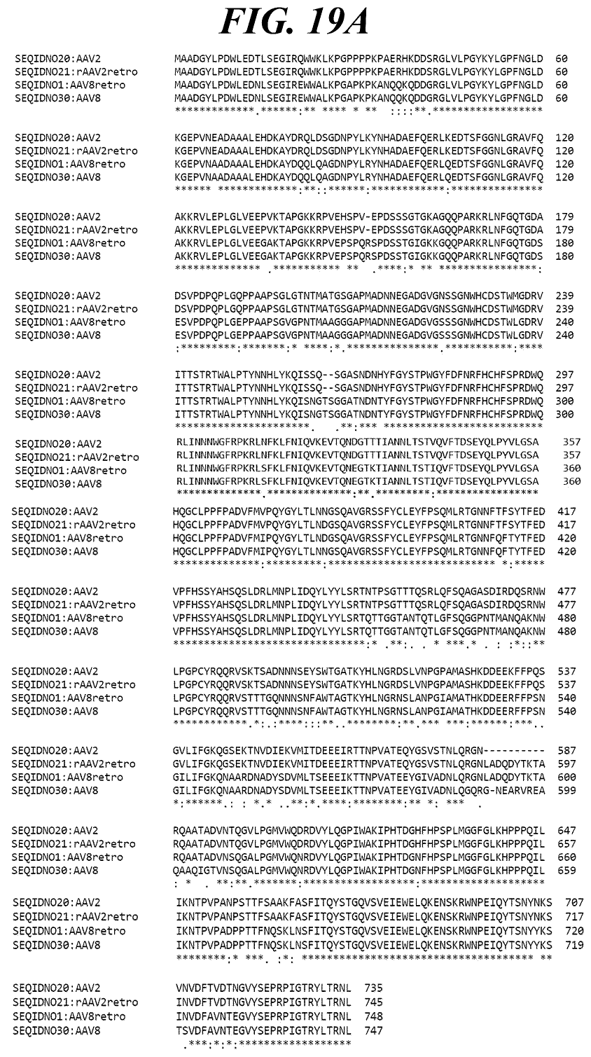

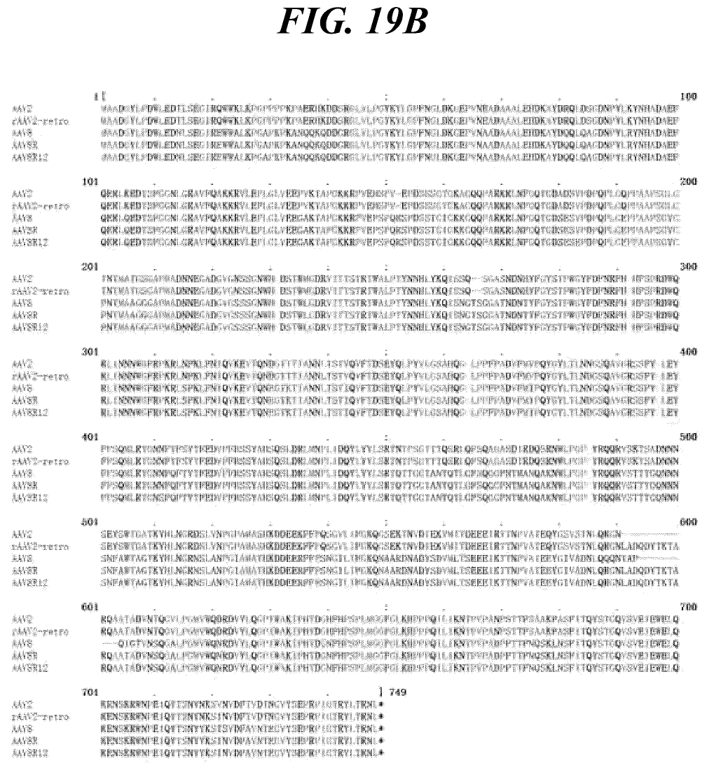

In certain embodiments, a sequence can correspond to the sequence identity of one or more of sequences 1, 2, 3, 4, 5, 6, 7, 8, 9, 10, 11, 12, 13, 14, 15, 31, or 32. A corresponding sequence is a sequence that, while it possesses a high degree of identity, also has certain deletions, insertions, etc. that make it not exactly align with the sequence to which it is compared. For example, in FIG. 19A, an alignment comparison is made between AAV2 and AAV8. The alignment shows that although there is a one amino acid deletion in AAV2 at the corresponding position 152 in AAV8, amino acid 154 in AAV8 still corresponds to amino acid 153 in AAV2. Allowing for this corresponding but not exact identity comparison shows more accurately that AAV2 has 82% identity with AAV8. FIG. 19B includes a similar alignment comparison.

In certain embodiments, a sequence can have a mutation that corresponds to one or more of V125I, V183E, N411 S, Y447F, R490Q, T495A, F536Y, and A606S. For example, as seen in FIG. 19A, mutation V125I can occur at position 125; alternatively, it can occur at a position that corresponds in an alignment with position 125 due to a deletion, insertion, etc. For example, mutation V183E can occur at position 183; alternatively, it can occur at a position that corresponds in an alignment with position 183 due to a deletion, insertion, etc. For example, mutation N411S can occur at position 411, as it does in AAV8; alternatively, mutation N411S can occur at corresponding position 408 (N408S), as is seen in AAV2 in FIG. 19A. For example, mutation Y447F can occur at position 447, as it does in AAV8; alternatively, mutation Y447F can occur at corresponding position 444 (Y444F), as is seen in AAV2 in FIG. 19A. For example, mutation R490Q can occur at position 490, as it does in AAV8; alternatively, mutation R490Q can occur at corresponding position 487 (R487Q), as is seen in AAV2 in FIG. 19A. For example, mutation T495A can occur at position 495, as it does in AAV8; alternatively, mutation T495A can occur at corresponding position 492 (T492A), as is seen in AAV2 in FIG. 19A. For example, mutation F536Y can occur at position 536, as it does in AAV8; alternatively, mutation F536Y can occur at corresponding position 533 (F533Y), as is seen in AAV2 in FIG. 19A. For example, mutation A606S can occur at position 606, as it does in rAAV8R; alternatively, mutation A606S can occur at corresponding position 593 (A593S), as is seen in AAV2 in FIG. 19A. The sequence may include a sequence or mutation shown in FIG. 19A or FIG. 19B.

Retrograde transport can be increased by at least about 1-fold, at least about 2-fold, at least about 3-fold, at least about 4-fold, at least about 5-fold, at least about 6-fold, at least about 7-fold, at least about 8-fold, at least about 9-fold, or at least about 10-fold more than the retrograde transport of the rAAV control. In certain embodiments, the increased retrograde transport is in a medium spiny neuron. In certain embodiments, the increased retrograde transport is in a D1 medium spiny neuron.

In another embodiment, the variant capsid polypeptide comprises an alternation that decreases retrograde transport. Retrograde transport can be decreased by at least about 1-fold, at least about 2-fold, at least about 3-fold, at least about 4-fold, at least about 5-fold, at least about 6-fold, at least about 7-fold, at least about 8-fold, at least about 9-fold, or at least about 10-fold more than the retrograde transport of the rAAV control.

In another embodiment the cell can be a nerve cell. Non-limiting examples of nerve cells include neurons and glial cells. In one embodiment, a nerve cell can be a neuron. In one embodiment, the variant capsid polypeptide comprises an alternation that increases retrograde transport along the axon of the neuron, along the dendrites of the neuron, through the neuron cell body, or a combination thereof.

In some embodiments, the modified variant capsid polypeptide can increase the infectivity of a rAAV virion into a neuron. Non-limiting examples of neurons include excitatory neurons (e.g., dopaminergic neurons or acetylcholinergic neurons) and inhibitory neurons (e.g., GABAergic neurons or medium spiny neurons).

In some embodiments, the modified variant capsid polypeptide can increase the infectivity of a rAAV virion by at least about 1-fold, at least about 2-fold, at least about 3-fold, at least about 4-fold, at least about 5-fold, at least about 6-fold, at least about 7-fold, at least about 8-fold, at least about 9-fold, or at least about 10-fold as compared to the baseline activity of rAAV2-retro control. In certain embodiments the increased infectivity is in a medium spiny neuron, such as a D1 medium spiny neuron.

### Heterologous Nucleic Acids

In one embodiment, the rAAV can further comprise a heterologous nucleic acid (e.g., a DNA or an RNA). In another embodiment, the heterologous nucleic acid can comprise one or more sequences to direct integration into a genomic location of a cell. A cell can be a bacterial, archaeal, plant, fungal or animal cell. An animal cell can be an amphibian cell, reptilian cell, mammalian cell, avian cell, or fish cell. A mammalian cell can be any cell from or derived from any mammal (e.g. a human a hamster, a mouse, a monkey, a rat, a pig, a cow, or a rabbit).

A heterologous nucleic acid can comprise a sequence comprising a regulatory element (e.g., a promoter). Alternatively, or in addition to, a heterologous nucleic acid sequence can comprise an open reading frame of a gene of interest. A gene of interest can be a non-coding region (e.g., a UTR, or a promoter). Alternatively, a gene of interest can be a polypeptide. A gene of interest can be an endogenous gene. Alternatively, a gene of interest can be an exogenous gene. A gene of interest can possess therapeutic utility (e.g., can aide in treating a neurological disorder). A gene of interest can comprise a neurotrophic factor, an RNA guided nuclease, an enzyme, or a DREADD.

A gene of interest can be the G protein-coupled receptor 88 (GPR88) gene. GPR88 encodes a receptor found almost exclusively in the striatum, a brain structure that controls motor function and cognition. Defects in GPR88 have been associated with chorea, speech delay, learning difficulties, and some neuropsychiatric disorders.

A polypeptide can be an antibody, a contractile protein, an enzyme, a hormonal protein, a structural protein, a storage protein, a small molecule, or a transport protein. Non-limiting examples of enzymes include hydrolase, isomerases, nucleases, ligases, transferases, and oxidoreductases. In one embodiment, a gene of interest is a nuclease or a neurotrophic factor (e.g., brain derived neurotrophic factor (BDNF)). A nuclease can be an RNA guided nuclease. An RNA guided nuclease can be a programmable endonuclease (e.g., Cas or Cas9), which can be used to perform targeted genome editing. A programmable endonuclease can interact with a guide RNA to form a CRISPR/Cas or CRISPR/Cas9 complex. In certain embodiments, a gene of interest can be a DREADD (e.g., hM3Dq, hM1Dq, hMD5q, hM4Di, or hM2Di).

### Expression Vectors

The promoters described herein can be a component of different types of expression vectors and used to initiate expression of different heterologous genes. In certain embodiments, the expression vector is a viral vector. In certain embodiments, the expression vector is a naked DNA vector, such as a plasmid, bacterial artificial chromosome, or yeast artificial chromosome. In certain embodiments, the viral vector is an adenovirus, a lentivirus, or an adeno-associated virus.

### Regulatory Elements

In one embodiment, the rAAV virion comprises a regulatory element which comprises a nucleotide sequence corresponding to a genomic sequence. A regulatory element can be a nucleic acid or small molecule which is needed to turn a gene on or off Examples of a regulatory element includes, but is not limited to a promoter, a repressor, an activator, a silencer, or an enhancer. A regulatory element can be a promoter. A promoter can be necessary for binding enzymes or other factors that initiate transcription of DNA to mRNA. Alternatively, a promoter can be sufficient for binding enzymes or other factors that initiate transcription of DNA to mRNA.

A regulatory element can have proximity to a gene along a chromosome. Alternatively, a regulatory element can have proximity to a gene through the spatial folding of DNA inside the nucleus. A regulatory element (e.g., a promoter) can be located near the transcriptional start site (TSS) of a gene. A regulatory element can be found 5′ to the start site a gene. Alternatively, a regulatory element can be found 3′ to the start site of a gene. A regulatory element can be found partly or completely within the intronic regions of a gene. In certain embodiments, the gene is GPR88. The TSS for GPR88 is the guanine which corresponds to position 1431 of SEQ ID NO: 44, position 582 of SEQ ID NO: 45, and position 68 of SEQ ID NO: 46.

A regulatory element can be positioned less than about 5,000 nucleotides, less than about 4,000 nucleotides, less than about 3,000 nucleotides, less than about 2,000 nucleotides, less than about 1,000 nucleotides, less than about 900 nucleotides, less than about 800 nucleotides, less than about 700 nucleotides, less than about 600 nucleotides, less than about 500 nucleotides, less than about 400 nucleotides, less than about 300 nucleotides, less than about 200 nucleotides, or less than about 100 nucleotides 3′ to the translational start site of a gene. In certain embodiments, the gene is GPR88.

A regulatory element can be positioned more than about 100 nucleotides, more than about 200 nucleotides, more than about 300 nucleotides, more than about 400 nucleotides, more than about 500 nucleotides, more than about 600 nucleotides, more than about 700 nucleotides, more than about 800 nucleotides, more than about 900 nucleotides, more than about 1,000 nucleotides, more than about 2,000 nucleotides, more than about 3,000 nucleotides, more than about 4,000 nucleotides, or more than about 5,000 nucleotides 3′ to the translational start site of a gene. In certain embodiments, the gene is GPR88.

A regulatory element can be positioned less than about 5,000 nucleotides, less than about 4,000 nucleotides, less than about 3,000 nucleotides, less than about 2,000 nucleotides, less than about 1,000 nucleotides, less than about 900 nucleotides, less than about 800 nucleotides, less than about 700 nucleotides, less than about 600 nucleotides, less than about 500 nucleotides, less than about 400 nucleotides, less than about 300 nucleotides, less than about 200 nucleotides, or less than about 100 nucleotides 5′ to the translational start site of a gene. In certain embodiments, the gene is GPR88.

A regulatory element can be positioned more than about 100 nucleotides, more than about 200 nucleotides, more than about 300 nucleotides, more than about 400 nucleotides, more than about 500 nucleotides, more than about 600 nucleotides, more than about 700 nucleotides, more than about 800 nucleotides, more than about 900 nucleotides, more than about 1,000 nucleotides, more than about 2,000 nucleotides, more than about 3,000 nucleotides, more than about 4,000 nucleotides, or more than about 5,000 nucleotides 5′ to the translational start site of a gene. In certain embodiments, the gene is GPR88.

The regulatory element can comprise the nucleic acid sequence set forth in SEQ ID NO: 39. The variant capsid polypeptide can have at least about 80% sequence identity to SEQ ID NO: 39, at least about 82% sequence identity to SEQ ID NO: 39, at least about 84% sequence identity to SEQ ID NO: 39, at least about 86% sequence identity to SEQ ID NO: 39, at least about 88% sequence identity to SEQ ID NO: 39, at least about 90% sequence identity to SEQ ID NO: 39, at least about 91% sequence identity to SEQ ID NO: 39, at least about 92% sequence identity to SEQ ID NO: 39, at least about 93% sequence identity to SEQ ID NO: 39, at least about 94% sequence identity to SEQ ID NO: 39, at least about 95% sequence identity to SEQ ID NO: 39, at least about 96% sequence identity to SEQ ID NO: 39, at least about 97% sequence identity to SEQ ID NO: 39, at least about 98% sequence identity to SEQ ID NO: 39, or at least about 99% sequence identity to SEQ ID NO: 39.

The regulatory element can comprise the nucleic acid sequence set forth in SEQ ID NO: 40. The variant capsid polypeptide can have at least about 80% sequence identity to SEQ ID NO: 40, at least about 82% sequence identity to SEQ ID NO: 40, at least about 84% sequence identity to SEQ ID NO: 40, at least about 86% sequence identity to SEQ ID NO: 40, at least about 88% sequence identity to SEQ ID NO: 40, at least about 90% sequence identity to SEQ ID NO: 40, at least about 91% sequence identity to SEQ ID NO: 40, at least about 92% sequence identity to SEQ ID NO: 40, at least about 93% sequence identity to SEQ ID NO: 40, at least about 94% sequence identity to SEQ ID NO: 40, at least about 95% sequence identity to SEQ ID NO: 40, at least about 96% sequence identity to SEQ ID NO: 40, at least about 97% sequence identity to SEQ ID NO: 40, at least about 98% sequence identity to SEQ ID NO: 40, or at least about 99% sequence identity to SEQ ID NO: 40.

The regulatory element can comprise the nucleic acid sequence set forth in SEQ ID NO: 41. The variant capsid polypeptide can have at least about 80% sequence identity to SEQ ID NO: 41, at least about 82% sequence identity to SEQ ID NO: 41, at least about 84% sequence identity to SEQ ID NO: 41, at least about 86% sequence identity to SEQ ID NO: 41, at least about 88% sequence identity to SEQ ID NO: 41, at least about 90% sequence identity to SEQ ID NO: 41, at least about 91% sequence identity to SEQ ID NO: 41, at least about 92% sequence identity to SEQ ID NO: 41, at least about 93% sequence identity to SEQ ID NO: 41, at least about 94% sequence identity to SEQ ID NO: 41, at least about 95% sequence identity to SEQ ID NO: 41, at least about 96% sequence identity to SEQ ID NO: 41, at least about 97% sequence identity to SEQ ID NO: 41, at least about 98% sequence identity to SEQ ID NO: 41, or at least about 99% sequence identity to SEQ ID NO: 41.

The regulatory element can comprise the nucleic acid sequence set forth in SEQ ID NO: 42. The variant capsid polypeptide can have at least about 80% sequence identity to SEQ ID NO: 42, at least about 82% sequence identity to SEQ ID NO: 42, at least about 84% sequence identity to SEQ ID NO: 42, at least about 86% sequence identity to SEQ ID NO: 42, at least about 88% sequence identity to SEQ ID NO: 42, at least about 90% sequence identity to SEQ ID NO: 42, at least about 91% sequence identity to SEQ ID NO: 42, at least about 92% sequence identity to SEQ ID NO: 42, at least about 93% sequence identity to SEQ ID NO: 42, at least about 94% sequence identity to SEQ ID NO: 42, at least about 95% sequence identity to SEQ ID NO: 42, at least about 96% sequence identity to SEQ ID NO: 42, at least about 97% sequence identity to SEQ ID NO: 42, at least about 98% sequence identity to SEQ ID NO: 42, or at least about 99% sequence identity to SEQ ID NO: 42.

The regulatory element can comprise the nucleic acid sequence set forth in SEQ ID NO: 43. The variant capsid polypeptide can have at least about 80% sequence identity to SEQ ID NO: 43, at least about 82% sequence identity to SEQ ID NO: 43, at least about 84% sequence identity to SEQ ID NO: 43, at least about 86% sequence identity to SEQ ID NO: 43, at least about 88% sequence identity to SEQ ID NO: 43, at least about 90% sequence identity to SEQ ID NO: 43, at least about 91% sequence identity to SEQ ID NO: 43, at least about 92% sequence identity to SEQ ID NO: 43, at least about 93% sequence identity to SEQ ID NO: 43, at least about 94% sequence identity to SEQ ID NO: 43, at least about 95% sequence identity to SEQ ID NO: 43, at least about 96% sequence identity to SEQ ID NO: 43, at least about 97% sequence identity to SEQ ID NO: 43, at least about 98% sequence identity to SEQ ID NO: 43, or at least about 99% sequence identity to SEQ ID NO: 43.

The regulatory element can comprise the nucleic acid sequence set forth in SEQ ID NO: 44. The variant capsid polypeptide can have at least about 80% sequence identity to SEQ ID NO: 44, at least about 82% sequence identity to SEQ ID NO: 44, at least about 84% sequence identity to SEQ ID NO: 44, at least about 86% sequence identity to SEQ ID NO: 44, at least about 88% sequence identity to SEQ ID NO: 44, at least about 90% sequence identity to SEQ ID NO: 44, at least about 91% sequence identity to SEQ ID NO: 44, at least about 92% sequence identity to SEQ ID NO: 44, at least about 93% sequence identity to SEQ ID NO: 44, at least about 94% sequence identity to SEQ ID NO: 44, at least about 95% sequence identity to SEQ ID NO: 44, at least about 96% sequence identity to SEQ ID NO: 44, at least about 97% sequence identity to SEQ ID NO: 44, at least about 98% sequence identity to SEQ ID NO: 44, or at least about 99% sequence identity to SEQ ID NO: 44.

The regulatory element can comprise the nucleic acid sequence set forth in SEQ ID NO: 45. The variant capsid polypeptide can have at least about 80% sequence identity to SEQ ID NO: 45, at least about 82% sequence identity to SEQ ID NO: 45, at least about 84% sequence identity to SEQ ID NO: 45, at least about 86% sequence identity to SEQ ID NO: 45, at least about 88% sequence identity to SEQ ID NO: 45, at least about 90% sequence identity to SEQ ID NO: 45, at least about 91% sequence identity to SEQ ID NO: 45, at least about 92% sequence identity to SEQ ID NO: 45, at least about 93% sequence identity to SEQ ID NO: 45, at least about 94% sequence identity to SEQ ID NO: 45, at least about 95% sequence identity to SEQ ID NO: 45, at least about 96% sequence identity to SEQ ID NO: 45, at least about 97% sequence identity to SEQ ID NO: 45, at least about 98% sequence identity to SEQ ID NO: 45, or at least about 99% sequence identity to SEQ ID NO: 45.

The regulatory element can comprise the nucleic acid sequence set forth in SEQ ID NO: 46. The variant capsid polypeptide can have at least about 80% sequence identity to SEQ ID NO: 46, at least about 82% sequence identity to SEQ ID NO: 46, at least about 84% sequence identity to SEQ ID NO: 46, at least about 86% sequence identity to SEQ ID NO: 46, at least about 88% sequence identity to SEQ ID NO: 46, at least about 90% sequence identity to SEQ ID NO: 46, at least about 91% sequence identity to SEQ ID NO: 46, at least about 92% sequence identity to SEQ ID NO: 46, at least about 93% sequence identity to SEQ ID NO: 46, at least about 94% sequence identity to SEQ ID NO: 46, at least about 95% sequence identity to SEQ ID NO: 46, at least about 96% sequence identity to SEQ ID NO: 46, at least about 97% sequence identity to SEQ ID NO: 46, at least about 98% sequence identity to SEQ ID NO: 46, or at least about 99% sequence identity to SEQ ID NO: 46.

The regulatory element can comprise the nucleic acid sequence set forth in SEQ ID NO: 47. The variant capsid polypeptide can have at least about 80% sequence identity to SEQ ID NO: 47, at least about 82% sequence identity to SEQ ID NO: 47, at least about 84% sequence identity to SEQ ID NO: 47, at least about 86% sequence identity to SEQ ID NO: 47, at least about 88% sequence identity to SEQ ID NO: 47, at least about 90% sequence identity to SEQ ID NO: 47, at least about 91% sequence identity to SEQ ID NO: 47, at least about 92% sequence identity to SEQ ID NO: 47, at least about 93% sequence identity to SEQ ID NO: 47, at least about 94% sequence identity to SEQ ID NO: 47, at least about 95% sequence identity to SEQ ID NO: 47, at least about 96% sequence identity to SEQ ID NO: 47, at least about 97% sequence identity to SEQ ID NO: 47, at least about 98% sequence identity to SEQ ID NO: 47, or at least about 99% sequence identity to SEQ ID NO: 47.

The regulatory element can comprise the nucleic acid sequence set forth in SEQ ID NO: 48. The variant capsid polypeptide can have at least about 80% sequence identity to SEQ ID NO: 48, at least about 82% sequence identity to SEQ ID NO: 48, at least about 84% sequence identity to SEQ ID NO: 48, at least about 86% sequence identity to SEQ ID NO: 48, at least about 88% sequence identity to SEQ ID NO: 48, at least about 90% sequence identity to SEQ ID NO: 48, at least about 91% sequence identity to SEQ ID NO: 48, at least about 92% sequence identity to SEQ ID NO: 48, at least about 93% sequence identity to SEQ ID NO: 48, at least about 94% sequence identity to SEQ ID NO: 48, at least about 95% sequence identity to SEQ ID NO: 48, at least about 96% sequence identity to SEQ ID NO: 48, at least about 97% sequence identity to SEQ ID NO: 48, at least about 98% sequence identity to SEQ ID NO: 48, or at least about 99% sequence identity to SEQ ID NO: 48.

A regulatory element can be a promoter. A promoter can be tissue specific. Alternatively, a promoter can be cell-type specific. Non-limiting examples of cell-type specific promoters include neuron specific promoters, muscle specific promoters, blood cell specific promoters, skin cell specific promoters, endothelial cell specific promoters, or epithelial cell specific promoters.

A cell-type specific promoter can be neuron specific. A neuron specific promoter is a promoter that only functions in neurons to turn on and/or off genes that are specific to neurons. A neuron specific promoter can be a synapsin I (SYN) promoter (e.g., hSYN1), a calcium/calmodulin-dependent protein kinase II (CamKII) promoter, a tubulin alpha I, a neuron-specific enolase, a platelet-derived growth factor beta chain promoter, an astrocyte-specific glial fibrillary acidic protein (GFAP) promoter, a cerebellar Purkinje cell-specific L7-6 promoter, a dopamine receptor D1 (DRD1) promoter, a dopamine receptor D2 (DRD2) promoter, a parvalbumin (Pvalb) promoter, or a distal-less homeobox (Dlx) promoter.

A regulatory element (e.g., a promoter and/or enhancer) can increase expression of the heterologous gene of interest compared to the promoter of the hSYN1 (human Synapsin I receptor) gene in the neuron of the striatum. A regulatory element can increase expression of the heterologous gene of interest compared to the promoter of the hSYN1 gene in the neuron of the striatum by at least or about 1-fold, at least or about 2-fold, at least or about 3-fold, at least or about 4-fold, at least or about 5-fold, at least or about 6-fold, at least or about 7-fold, at least or about 8-fold, at least or about 9-fold, at least or about 10-fold, at least or about 20-fold, at least or about 30-fold, at least or about 40-fold, at least or about 50-fold, at least or about 60-fold at least or about 70-fold, at least or about 80-fold, at least or about 90-fold, at least or about 100-fold, at least or about 200-fold, at least or about 300-fold, at least or about 400-fold, at least or about 500-fold, or at least or about 1,000-fold.

A regulatory element can increase expression of the heterologous gene of interest compared to the promoter of the hSYN1 gene in the neuron of the striatum by at most or about 1,000-fold, at most or about 500-fold, at most or about 400-fold, at most or about 300-fold, at most or about 200-fold, at most or about 100-fold, at most or about 90-fold, at most or about 80-fold, at most or about 70-fold, at most or about 60-fold, at most or about 50-fold, at most or about 40-fold, at most or about 30-fold, at most or about 20-fold, at most or about 10-fold at most or about 9-fold, at most or about 8-fold, at most or about 7-fold, at most or about 6-fold, at most or about 5-fold, at most or about 4-fold, at most or about 3-fold, at most or about 2-fold, or at most or about 1-fold.

A regulatory element can increase expression of the heterologous gene of interest compared to the promoter of the hSYN1 gene in the neuron of the striatum by at least or about 1%, at least or about 2%, at least or about 3%, at least or about 4%, at least or about 5%, at least or about 6%, at least or about 7%, at least or about 8%, at least or about 9%, at least or about 10%, at least or about 20%, at least or about 30%, at least or about 40%, at least or about 50%, at least or about 60% at least or about 70%, at least or about 80%, at least or about 90%, at least or about 100%, at least or about 200%, at least or about 300%, at least or about 400%, at least or about 500%, or at least or about 1,000%.

A regulatory element can increase expression of the heterologous gene of interest compared to the promoter of the hSYN1 gene in the neuron of the striatum by at most or about 1,000%, at most or about 500%, at most or about 400%, at most or about 300%, at most or about 200%, at most or about 100%, at most or about 90%, at most or about 80%, at most or about 70%, at most or about 60%, at most or about 50%, at most or about 40%, at most or about 30%, at most or about 20%, at most or about 10% at most or about 9%, at most or about 8%, at most or about 7%, at most or about 6%, at most or about 5%, at most or about 4%, at most or about 3%, at most or about 2%, or at most or about 1%.

A regulatory element (e.g., a promoter) can be delivered into a cell through methods such as, but not limited to CRISPR knock-in methods, lentiviral methods, adenoviral methods, plasmid gene transfer methods, or cellular disruption methods. A nucleic acid comprising a regulatory element can be included in a viral vector. A viral vector can be an adeno-associated viral (AAV) vector.

### Methods for Expressing Genes and Delivery to the CNS

Gene therapy is a technique that modifies a subject's genes to treat or cure disease. Gene therapies can work by several mechanisms such as, but not limited to, replacing a disease-causing gene with a healthy copy of the gene, inactivating a disease-causing gene that is not functioning properly, or introducing a new or modified gene into the body to help treat a disease. Gene therapies can introduce genetic material into a cell using plasmid DNA, viral vectors, bacterial vectors, gene editing technology, or patient-derived cellular gene therapy products.

Delivery of the gene therapy into a subject can occur through intravenous (IV) injection, oral administration, intramuscular injection, subcutaneous injection, intrathecal therapy, rectal administration, vaginal administration, or inhalation.

The nervous system is organized into two main parts, the central nervous system (CNS) and the peripheral nervous system (PNS). The CNS is the processing center of the body and consists of the brain and the spinal cord. Accessing the CNS can require a therapy to bypass the blood-brain barrier. Strategies to deliver therapeutics to the CNS include, but are not limited to, intra-arterial chemotherapy, direct injection of therapeutic substances into intracranial lesions, and use of nanoparticles for drug delivery.

In certain embodiments, the rAAV virions of the present disclosure can be injected into a subject. In certain embodiments, injections can be given directly into the brain. In some embodiments, the pharmaceutical composition is directly injected into the striatum, which is a nucleus in the subcortical basal ganglia of the forebrain.

In some embodiments, the injected gene can target a specific type of neurons or set of neurons in the brain. In some embodiments, the neuron can be a neuron of the striatum. In some embodiments, the neuron can be a medium spiny neuron. In some embodiments, the neuron can be a dopaminergic medium spiny neuron. In some embodiments the dopaminergic medium spiny neuron is a D1 dopaminergic medium spiny neuron.

Injections can be performed using stereotactic surgery or intracerebral injections. Stereotactic surgery is a minimally invasive form of surgical intervention that makes use of a three-dimensional coordinate system to locate small targets inside the body and to perform on them some action such as ablation, biopsy, lesion, injection, stimulation, implantation, radiosurgery (SRS), etc. Intracerebral injection, such as intracerebroventricular injection, is an invasive injection technique of substances directly into the cerebrospinal fluid in cerebral ventricles in order to bypass the blood-brain barrier.

In some embodiments, the genetic therapy of the disclosure is used to genetically engineer a neuron. In some embodiments the genetic engineering method results in the expression of the polypeptide by the neuron. In some embodiments, the peptide expressed by the neuron is translated to form a therapeutic protein. In some embodiments, the therapeutic protein delivered by the AAV of the present disclosure is useful for targeting and treating a neurodegenerative disease such as Parkinson's disease. In certain embodiments, described herein is a method of preparing a Parkinson's disease treatment comprising administering one or more pharmaceutically acceptable excipients, carriers, or diluents and an rAAV virion of the present disclosure.

Parkinson's disease (PD) is a progressive nervous system disorder that affects movement. Symptoms start gradually, sometimes starting with a barely noticeable tremor in just one hand. Tremors are common, but the disorder also commonly causes stiffness or slowing of movement. Symptoms of PD include tremors, bradykinesia, rigid muscles, impaired posture and balance, loss of automatic movements, or speech changes.

A tremor, also known as shaking, is an involuntary, rhythmic muscle contraction leading to shaking movements in one or more parts of the body. A PD treatment method of the present disclosure may reduce tremors in patients by at least 10%, 20%, 30%, 40%, 50%, 60%, 70%, 80%, 90%, or 100%.

Bradykinesia is an impairment of voluntary motor control and slow movements or freezing. Bradykinesia may appear as a reduction in automatic movements such as blinking or swinging of arms while walking, or it may manifest as trouble initiating intentional movements or just slowness of actions. A PD treatment method of the present disclosure may reduce bradykinesia in patients by at least 10%, 20%, 30%, 40%, 50%, 60%, 70%, 80%, 90%, or 100%.

Muscle rigidity, also known as muscle tension, rigor, or stiffness, is characterized by the inability of the muscles to relax normally. The condition can affect any of the muscles in the body, causing sharp pain that makes it difficult to move. A PD treatment method of the present disclosure may reduce muscle rigidity in patients by at least 10%, 20%, 30%, 40%, 50%, 60%, 70%, 80%, 90%, or 100%.

Postural instability, or impairment of posture and/or balance, is the inability to maintain equilibrium under dynamic and static conditions such as preparation of movements, perturbations, and quiet stance. Postural instability can appear as a tendency to be unstable when standing. Postural instability can manifest as a tendency to fall or the inability to keep oneself from falling. A PD treatment method of the present disclosure may reduce postural instability in patients by at least 10%, 20%, 30%, 40%, 50%, 60%, 70%, 80%, 90%, or 100%.

Automatic movements are movements people often make without conscious thought (e.g., blinking or arm swinging while walking). A PD treatment method of the present disclosure may reduce the loss of automatic movements in patients by at least 10%, 20%, 30%, 40%, 50%, 60%, 70%, 80%, 90%, or 100%.

Speech changes can include fluency disorders (an unusual repetition of sounds or rhythm), voice disorders (an atypical tone of voice), or articulation disorders (distortion of certain sounds). A PD treatment method of the present disclosure may reduce a change in speech patterns in patients by at least 10%, 20%, 30%, 40%, 50%, 60%, 70%, 80%, 90%, or 100%.

In some embodiments, the present disclosure provides a method to express and activate a DREADD in the central nervous system of an individual comprising administering to the individual the retro-AAV or the pharmaceutical composition and a ligand that activates the DREADD, thereby activating the DREADD in the central nervous system of the individual. In some embodiments, the DREADD is expressed and activated int in a neuron of the striatum. In some embodiments, the neuron of the striatum is a D1 dopaminergic medium spiny neuron. In some embodiments, the individual is a mammal. In some embodiments, the individual is a human. In some embodiments, activating the DREADD in the central nervous system of the individual treats a neurodegenerative disorder. In some embodiments, the neurodegenerative disorder comprises Parkinson's disease. In some embodiments, the ligand that activates the DREADD comprises quetiapine or clozapine. In some embodiments, the ligand that activates the DREADD comprises quetiapine. In some embodiments, the ligand that activates the DREADD comprises clozapine. In some embodiments, the retro-AAV and the ligand that activates the DREADD are administered separately.

### Designer Receptor Exclusively Activated by Designer Drugs (DREADDs)

An rAAV virion can comprise a designer receptor exclusively activated by designer drugs (DREADD) as the gene of interest. A DREADD, also known at a receptor activated solely by a synthetic ligand, can be a class of artificially engineered protein receptors which can be selectively activated by certain ligands. A DREADD can be rM3Ds.

The DREADD can comprise the amino acid sequence set forth in SEQ ID NO: 38. The variant capsid polypeptide can have at least about 80% sequence identity to SEQ ID NO: 38, at least about 82% sequence identity to SEQ ID NO: 38, at least about 84% sequence identity to SEQ ID NO: 38, at least about 86% sequence identity to SEQ ID NO: 38, at least about 88% sequence identity to SEQ ID NO: 38, at least about 90% sequence identity to SEQ ID NO: 38, at least about 91% sequence identity to SEQ ID NO: 38, at least about 92% sequence identity to SEQ ID NO: 38, at least about 93% sequence identity to SEQ ID NO: 38, at least about 94% sequence identity to SEQ ID NO: 38, at least about 95% sequence identity to SEQ ID NO: 38, at least about 96% sequence identity to SEQ ID NO: 38, at least about 97% sequence identity to SEQ ID NO: 38, at least about 98% sequence identity to SEQ ID NO: 38, or at least about 99% sequence identity to SEQ ID NO: 38.

A DREADD can be HM3Ds. The DREADD can comprise the amino acid sequence set forth in. The variant capsid polypeptide can have at least about 80% sequence identity to SEQ ID NO: 49, at least about 82% sequence identity to SEQ ID NO: 49, at least about 84% sequence identity to SEQ ID NO: 49, at least about 86% sequence identity to SEQ ID NO: 49, at least about 88% sequence identity to SEQ ID NO: 49, at least about 90% sequence identity to SEQ ID NO: 49, at least about 91% sequence identity to SEQ ID NO: 49, at least about 92% sequence identity to SEQ ID NO: 49, at least about 93% sequence identity to SEQ ID NO: 49, at least about 94% sequence identity to SEQ ID NO: 49, at least about 95% sequence identity to SEQ ID NO: 49, at least about 96% sequence identity to SEQ ID NO: 49, at least about 97% sequence identity to SEQ ID NO: 49, at least about 98% sequence identity to SEQ ID NO: 49, or at least about 99% sequence identity to SEQ ID NO: 49.

A DREADD for uses with the methods and systems described herein can be HM3Ds (A147S-F349Y). The DREADD can comprise the amino acid sequence set forth in SEQ ID NO: 50. The variant capsid polypeptide can have at least about 80% sequence identity to SEQ ID NO: 50, at least about 82% sequence identity to SEQ ID NO: 50, at least about 84% sequence identity to SEQ ID NO: 50, at least about 86% sequence identity to SEQ ID NO: 50, at least about 88% sequence identity to SEQ ID NO: 50, at least about 90% sequence identity to SEQ ID NO: 50, at least about 91% sequence identity to SEQ ID NO: 50, at least about 92% sequence identity to SEQ ID NO: 50, at least about 93% sequence identity to SEQ ID NO: 50, at least about 94% sequence identity to SEQ ID NO: 50, at least about 95% sequence identity to SEQ ID NO: 50, at least about 96% sequence identity to SEQ ID NO: 50, at least about 97% sequence identity to SEQ ID NO: 50, at least about 98% sequence identity to SEQ ID NO: 50, or at least about 99% sequence identity to SEQ ID NO: 50.

The DREADDs can be used with certain ligands that lead to activation of the DREADDs and a desired physiological effect. The ligand can be clozapine or quetiapine. In certain embodiments, the DREADD ligand is clozapine. In certain embodiments, the DREADD ligand is quetiapine. The DREADD ligand can be administered separately from the Retro-AAV encoding the DREADD. The DREADD ligand in the case of an FDA or EMA approved drug can be administered at or below a dose that is the approved dosage. The DREADD ligand in the case of an FDA or EMA approved drug can be administered on schedule that is the same or different than an approved schedule.

### Pharmaceutical Compositions

In certain embodiments the rAAV virions of the present disclosure are included in a pharmaceutical composition comprising one or more pharmaceutically acceptable excipients, carriers, stabilizers, dispersing agents, suspending agents, thickening agents, and/or diluents. The pharmaceutical composition facilitates administration of the compound to an organism. Pharmaceutical compositions can be administered in therapeutically-effective amounts as pharmaceutical compositions by various forms and routes including, for example, intravenous, subcutaneous, intramuscular, inhalation, oral, parenteral, ophthalmic, otic, subcutaneous, transdermal, nasal, intravitreal, intratracheal, intrapulmonary, transmucosal, vaginal, and topical administration.

Formulations can be modified depending upon the route of administration chosen. Pharmaceutical compositions comprising a compound described herein can be manufactured, for example, by mixing, dissolving, emulsifying, encapsulating, entrapping, or compression processes.

Non-limiting examples of dosage forms suitable for use in a method disclosed herein include feed, food, pellet, lozenge, liquid, elixir, aerosol, inhalant, spray, powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, nutraceuticals, and any combination thereof.

Pharmaceutical compositions can be formulated by combining the active compounds with pharmaceutically-acceptable carriers or excipients. Non-limiting examples of pharmaceutically-acceptable excipients suitable for use in the method disclosed herein include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, anti-microbial agents, plant cellulosic material and spheronization agents, and any combination thereof. Pharmaceutically acceptable excipients include, but are not limited to, liquids such as water, saline, glycerol and ethanol. Pharmaceutically acceptable salts such as inorganic acid salts such as hydrochloride, bromate, phosphate, sulfate, etc.; And salts of organic acids such as acetates, propionates, malonates, benzoates, etc. can be included in pharmaceutical compositions. Additionally, reinforcing materials such as wetting or emulsifying agents, pH buffering materials, and the like can be present in such vehicles. A wide variety of pharmaceutically acceptable excipients are known in the art and need not be discussed in detail herein. Pharmaceutically acceptable excipients are, for example, A. Gennaro (2000) “Remington: The Science and Practice of Pharmacy,” 20th edition, Lippincott, Williams, &Wilkins; Pharmaceutical Dosage Forms and Drug Delivery Systems (1999) H. C. Ansel et al., Eds., 7th ed., Lippincott, Williams, &Wilkins; and Handbook of Pharmaceutical Excipients (2000) A. H. Kibbe et al., Eds., 3rd ed. Amer.

Non-limiting examples of pharmaceutically-acceptable carriers include saline solution, Ringer's solution and dextrose solution. Further carriers include sustained release preparations such as semipermeable matrices of solid hydrophobic polymers containing the compound disclosed herein, where the matrices are in the form of shaped articles, such as films, liposomes, microparticles, and microcapsules.

Carbomers in an aqueous pharmaceutical composition serve as emulsifying agents and viscosity modifying agents. In certain embodiments, the pharmaceutically acceptable excipient comprises or consists of a carbomer. In certain embodiments, the carbomer comprises or consists of carbomer 910, carbomer 934, carbomer 934P, carbomer 940, carbomer 941, carbomer 1342, or combinations thereof. Cyclodextrins in an aqueous pharmaceutical composition serve as solubilizing and stabilizing agents. In certain embodiments, the pharmaceutically acceptable excipient comprises or consists of a cyclodextrin. In certain embodiments, the cyclodextrin comprises or consists of alpha cyclodextrin, beta cyclodextrin, gamma cyclodextrin, or combinations thereof. Lecithin in a pharmaceutical composition serve as a solubilizing agent. In certain embodiments, the solubilizing agent comprises or consists of lecithin. Poloxamers in a pharmaceutical composition serve as emulsifying agents, solubilizing agents, and dispersing agents. In certain embodiments, the pharmaceutically acceptable excipient comprises or consists of a poloxamer. In certain embodiments, the poloxamer comprises or consists of poloxamer 124, poloxamer 188, poloxamer 237, poloxamer 338, poloxamer 407, or combinations thereof. Polyoxyethylene sorbitan fatty acid esters in a pharmaceutical composition serve as emulsifying agents, solubilizing agents, surfactants, and dispersing agents. In certain embodiments, the pharmaceutically acceptable excipient comprises or consists of a polyoxyethylene sorbitan fatty acid ester. In certain embodiments, the polyoxyethylene sorbitan fatty acid ester comprises or consists of polysorbate 20, polysorbate 21, polysorbate 40, polysorbate 60, polysorbate 61, polysorbate 65, polysorbate 80, polysorbate 81, polysorbate 85, polysorbate 120, or combinations thereof. Polyoxyethylene stearates in a pharmaceutical composition serve as emulsifying agents, solubilizing agents, surfactants, and dispersing agents. In certain embodiments, the pharmaceutically acceptable excipient comprises or consists of a polyoxyethylene stearate. In certain embodiments, the polyoxyethylene stearate comprises or consists of polyoxyl 2 stearate, polyoxyl 4 stearate, polyoxyl 6 stearate, polyoxyl 8 stearate, polyoxyl 12 stearate, polyoxyl 20 stearate, polyoxyl 30 stearate, polyoxyl 40 stearate, polyoxyl 50 stearate, polyoxyl 100 stearate, polyoxyl 150 stearate, polyoxyl 4 distearate, polyoxyl 8 distearate, polyoxyl 12 distearate, polyoxyl 32 distearate, polyoxyl 150 distearate, or combinations thereof. Sorbitan esters in a pharmaceutical composition serve as emulsifying agents, solubilizing agents, and non-ionic surfactants, and dispersing agents. In certain embodiments, the pharmaceutically acceptable excipient comprises or consists of a sorbitan ester. In certain embodiments, the sorbitan ester comprises or consists of sorbitan laurate, sorbitan oleate, sorbitan palmitate, sorbitan stearate, sorbitan trioleate, sorbitan sesquioleate, or combinations thereof. In certain embodiments, solubility can be achieved with a protein carrier. In certain embodiments the protein carrier comprises albumin, human albumin.

In certain embodiments a polypeptide can be stabilized by polyuronides. In certain embodiments, the stabilizer comprises or consists of a polyuronide. In certain embodiments, the polyuronide comprises or consists of calcium alginate.

In certain embodiments, the rAAV virions of the present disclosure are administered suspended in a sterile solution. In certain embodiments, the solution comprises about 0.9% NaCl. In certain embodiments, the solution comprises about 5.0% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA.

In certain embodiments, the rAAV virions of the present disclosure are shipped/stored lyophilized and reconstituted before administration. In certain embodiments, lyophilized rAAV virion formulations comprise a bulking agent such as, mannitol, sorbitol, sucrose, trehalose, dextran 40, or combinations thereof. The lyophilized formulation can be contained in a vial comprised of glass or other suitable non-reactive material. The rAAV virions when formulated, whether reconstituted or not, can be buffered at a certain pH, generally less than 7.0. In certain embodiments, the pH can be between 4.5 and 6.5, 4.5 and 6.0, 4.5 and 5.5, 4.5 and 5.0, or 5.0 and 6.0.

A pharmaceutical composition can be administered in a local or systemic manner, for example, via injection of the compound directly into an organ, optionally in a depot or sustained release formulation or implant. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

In practicing the methods of treatment or use provided herein, therapeutically-effective amounts of the compounds described herein are administered in pharmaceutical compositions to a subject having a disease or condition to be treated. A therapeutically-effective amount can vary widely depending on the severity of the disease, the age and relative health of the subject, the potency of the compounds used, and other factors. The compounds can be used singly or in combination with one or more therapeutic agents as components of mixtures.

In some embodiments, the pharmaceutical administration is given to an animal including but not limited to a vertebrate, such as a mammal, avian or fish. An animal can be a human or a bovine, canine, caprine, cervine, cricetine, feline, galline, equine, lapine, murine, musteline and ovine. An animal can be a human or other mammalian animals including primates (e.g., monkeys), bovine (e.g., cattle or dairy cows), porcine (e.g., hogs or pigs), ovine (e.g., goats or sheep), equine (e.g., horses), canine (e.g., dogs), feline (e.g., house cats), antelopes, buffalos, camels, cervine (e.g., deer), donkeys, rabbits, and rodents (e.g., guinea pigs, squirrels, rats, mice, gerbils, and hamsters). In some embodiments, the pharmaceutical administration is administered to a human.

A pharmaceutically-acceptable excipient can be present in a pharmaceutical composition at a mass of between about 0.1% and about 99% by mass of the composition. For example, a pharmaceutically-acceptable excipient can be present in a pharmaceutical composition at a mass of between about 0.1% and about 95%, between about 0.1% and about 90%, between about 0.1% and about 85%, between about 0.1% and about 80%, between about 0.1% and about 75%, between about 0.1% and about 70%, between about 0.1% and about 65%, between about 0.1% and about 60%, between about 0.1% and about 55%, between about 0.1% and about 50%, between about 0.1% and about 45%, between about 0.1% and about 40%, between about 0.1% and about 35%, between about 0.1% and about 30%, between about 0.1% and about 25%, between about 0.1% and about 20%, between about 0.1% and about 15%, between about 0.1% and about 10%, between about 0.1% and about 5%, or between about 0.1% and about 1%, by mass of the formulation.

A pharmaceutically-acceptable excipient can be present at about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% by mass of the formulation.

### NUMBERED EMBODIMENTS

Disclosed herein are the following embodiments:
1. A retro adeno-associated virus (retro-AAV), wherein the retro-AAV comprises:
   a. an adeno-associated virus capsid polypeptide comprising one or more alterations that promote retrograde transport of the retro-AAV by a neuron, and
   b. a nucleic acid comprising a heterologous gene of interest operatively coupled to a GPR88 regulatory region or a R9P1 regulatory region.
2. The retro-AAV of embodiment 1, wherein the retro-AAV is a serotype selected from AAV2, AAV8 or a combination thereof.
3. The retro-AAV of embodiment 1 or 2, wherein the one or more alterations that promote retrograde transport of the retro-AAV by a neuron is selected from the list consisting of an insertion of SEQ ID NO: 31, an aspartic acid substitution at an amino acid residue corresponding to position 385 of SEQ ID NO: 1, an isoleucine and asparagine (IN) substitution at an amino acid residue corresponding to positions 721 and 722 of SEQ ID NO: 1, and combinations thereof.
4. The retro-AAV of any one of embodiments 1 to 3, comprising an alteration at one or more amino acid residues corresponding to V125, V183, N411, Y447, R490, T495, or F536 of SEQ ID NO: 1.
5. The retro-AAV of any one of embodiments 1 to 4, wherein the virus capsid polypeptide comprises an amino acid sequence that possesses at least 90%, 95%, 97%, 98%, 99% sequence identity or that is identical to the amino acid sequence set forth in SEQ ID NO: 1, wherein the virus capsid polypeptide comprises an alteration to SEQ ID NO: 1 at an amino acid selected from the list consisting of any one or more of V125, V183, N411, Y447, R490, T495, F536, and A606.
6. The retro-AAV of embodiment 5, wherein the virus capsid polypeptide comprises an alteration to SEQ ID NO: 1 corresponding to an amino acid selected from the list consisting of any two or more of V125, V183, N411, Y447, R490, T495, F536, and A606.
7. The retro-AAV of embodiment 5, wherein the virus capsid polypeptide comprises an alteration to SEQ ID NO: 1 corresponding to an amino acid selected from the list consisting of any three or more of V125, V183, N411, Y447, R490, T495, F536, and A606.
8. The retro-AAV of embodiment 5, wherein the virus capsid polypeptide comprises a substitution to SEQ ID NO: 1 corresponding to an amino acid selected from the list consisting of any one or more of V125I, V183E, N411S, Y447F, R490Q, T495A, F536Y, and A606S.
9. The retro-AAV of embodiment 5, wherein the virus capsid polypeptide comprises a substitution to SEQ ID NO: 1 corresponding to an amino acid selected from the list consisting of any two or more of V125I, V183E, N411S, Y447F, R490Q, T495A, F536Y, and A606S.
10. The retro-AAV of embodiment 5, wherein the virus capsid polypeptide comprises a substitution to SEQ ID NO: 1 corresponding to an amino acid selected from the list consisting of any three or more of V125I, V183E, N411S, Y447F, R490Q, T495A, F536Y, and A606S.
11. The retro-AAV of embodiment 5, wherein the virus capsid polypeptide comprises a substitution corresponding to V125I and N411 S of SEQ ID NO: 1.
12. The retro-AAV of embodiment 5, wherein the virus capsid polypeptide comprises a substitution corresponding to V125I, F536Y, T495A of SEQ ID NO: 1.
13. The retro-AAV of embodiment 5, wherein the virus capsid polypeptide comprises a substitution corresponding to V125I, A606S, and a T495A of SEQ ID NO: 1.
14. The retro-AAV of any one of embodiments 1 to 5, wherein the virus capsid polypeptide comprises the amino acid sequence set forth in any one of SEQ ID NOs 1 to 15.
15. The retro-AAV of any one of embodiments 1 to 5, wherein the virus capsid polypeptide comprises the amino acid sequence set forth in any one of SEQ ID NOs 2 to 15.
16. The retro-AAV of any one of embodiments 1 to 15, wherein the GPR88 regulatory region comprises a nucleotide sequence corresponding to a genomic sequence positioned 3′ to a translational start site of an endogenous GPR88 gene.
17. The retro-AAV of embodiment 16, wherein the genomic sequence positioned 3′ to a translational start site of the endogenous GPR88 gene is partially positioned in an intron.
18. The retro-AAV of embodiment 16, wherein the genomic sequence positioned 3′ to a translational start site of the endogenous GPR88 gene is positioned in an intron.
19. The retro-AAV of any one of embodiments 16 to 18, wherein the genomic sequence positioned 3′ to a translational start site of the endogenous GPR88 gene is positioned less than about 1,000 nucleotides 3′ to the translational start site of the endogenous GPR88 gene.
20. The retro-AAV of embodiment 16, wherein the GPR88 regulatory region comprises a nucleotide sequence that is at least 80%, 85%, 90%, 95%, 97%, 98%, 99% homologous to the nucleotide sequence set forth in SEQ ID NO: 39.
21. The retro-AAV of embodiment 16, wherein the GPR88 regulatory region comprises a nucleotide sequence that is identical to the nucleotide sequence set forth in SEQ ID NO: 39.
22. The retro-AAV of any one of embodiments 16 to 18, wherein the genomic sequence positioned 3′ to a translational start site of the endogenous GPR88 gene is positioned less than about 900 nucleotides 3′ to the translational start site of the endogenous GPR88 gene.
23. The retro-AAV of embodiment 22, wherein the GPR88 regulatory region comprises a nucleotide sequence that is at least 80%, 85%, 90%, 95%, 97%, 98%, 99% homologous to the nucleotide sequence set forth in SEQ ID NO: 40.
24. The retro-AAV of embodiment 22, wherein the GPR88 regulatory region comprises a nucleotide sequence that is identical to the nucleotide sequence set forth in SEQ ID NO: 40.
25. The retro-AAV of any one of embodiments 1 to 15, wherein the GPR88 regulatory region comprises a nucleotide sequence corresponding to a genomic sequence positioned 5′ to the translational start site of the endogenous GPR88 gene.
26. The retro-AAV of embodiment 25, wherein the genomic sequence positioned 5′ to a translational start site of the endogenous GPR88 gene is positioned less than about 100 nucleotides 5′ to the translational start site of the endogenous GPR88 gene.
27. The retro-AAV of embodiment 26, wherein the GPR88 regulatory region comprises a nucleotide sequence that is at least 80%, 85%, 90%, 95%, 97%, 98%, 99% homologous to the nucleotide sequence set forth in SEQ ID NO: 41.
28. The retro-AAV of embodiment 26, wherein the GPR88 regulatory region comprises a nucleotide sequence that is identical to the nucleotide sequence set forth in SEQ ID NO: 41.
29. The retro-AAV of embodiment 25, wherein the genomic sequence positioned 5′ to a translational start site of the endogenous GPR88 gene is positioned less than about 600 nucleotides 5′ to the translational start site of the endogenous GPR88 gene.
30. The retro-AAV of embodiment 29, wherein the GPR88 regulatory region comprises a nucleotide sequence that is at least 80%, 85%, 90%, 95%, 97%, 98%, 99% homologous to the nucleotide sequence set forth in SEQ ID NO: 42.
31. The retro-AAV of embodiment 29, wherein the regulatory element comprises a nucleotide sequence that is identical to the nucleotide sequence set forth in SEQ ID NO: 42.
32. The retro-AAV of any embodiment 25, wherein the genomic sequence positioned 5′ to a translational start site of the endogenous GPR88 gene is positioned less than about 1,500 nucleotides 5′ to the translational start site of the endogenous GPR88 gene.
33. The retro-AAV of embodiment 32, wherein the GPR88 regulatory region comprises a nucleotide sequence that is at least 80%, 85%, 90%, 95%, 97%, 98%, 99% homologous to the nucleotide sequence set forth in SEQ ID NO: 43.
34. The retro-AAV of embodiment 32, wherein the GPR88 regulatory region comprises a nucleotide sequence that is identical to the nucleotide sequence set forth in SEQ ID NO: 43.
35. The retro-AAV of any one of embodiments 1 to 15, wherein the GPR88 regulatory region comprises a nucleotide sequence that is at least 80%, 85%, 90%, 95%, 97%, 98%, 99% homologous to the nucleotide sequence set forth in any one of SEQ ID NOs: 44, 45, 46, or 48.
36. The retro-AAV of any one of embodiments 1 to 15, wherein the GPR88 regulatory region comprises a nucleotide sequence that is identical to the nucleotide sequence set forth in any one of SEQ ID NOs: 44, 45, 46, or 48.
37. The retro-AAV of any one of embodiments 1 to 15, wherein R9P1 regulatory region comprises a nucleotide sequence that is at least 80%, 85%, 90%, 95%, 97%, 98%, 99% homologous to the nucleotide sequence set forth in SEQ ID NO: 47.
38. The retro-AAV of any one of embodiments 10 to 37, wherein the heterologous gene of interest is 3′ to the GPR88 regulatory region or the R9P1 regulatory region.
39. The retro-AAV of any one of embodiments 1 to 38, wherein the heterologous gene of interest possesses therapeutic utility.
40. The retro-AAV of any one of embodiments 1 to 39, wherein the heterologous gene of interest comprises a neurotrophic factor, an RNA guided nuclease, an enzyme, or a DREADD.
41. The retro AAV of embodiment 40, wherein the heterologous gene of interest comprises a DREADD.
42. The retro-AAV of embodiment 40 or 41, wherein the DREADD is rM3Ds.
43. The retro-AAV of embodiment 40 or 41, wherein the DREADD comprises an amino acid sequence exhibiting at least about 90%, 95%, 97%, 98%, 99% identity to or is identical to SEQ ID NO: 38.
44. The retro-AAV of embodiment 40 or 41, wherein the DREADD is hM3Ds.
45. The retro-AAV of embodiment 40 or 41, wherein the DREADD comprises an amino acid sequence exhibiting at least about 90%, 95%, 97%, 98%, 99% identity to or is identical to SEQ ID NO: 49.
46. The retro-AAV of embodiment 40 or 41, wherein the DREADD is hM3Ds(A147S-F349Y). 47. The retro-AAV of embodiment 40 or 41, wherein the DREADD comprises an amino acid sequence exhibiting at least about 90%, 95%, 97%, 98%, 99% identity to or is identical to SEQ ID NO: 50.
48. The retro-AAV of any one of embodiments 1 to 47, wherein the heterologous gene of interest comprises one or more of hM3Dq, hM1Dq, hMD5q, hM4Di, hM2Di, or BDNF.
49. The retro-AAV of any one of embodiments 1 to 48, wherein the heterologous gene of interest exhibits increased expression of the heterologous gene of interest compared to the promoter of the hSYN1 gene in a neuron of the striatum.
50. The retro-AAV of any one of embodiments 1 to 48, wherein the heterologous gene of interest exhibits at least a 2-fold increase in expression of the heterologous gene of interest compared to the promoter of the hSYN1 gene in a neuron of the striatum.
51. The retro-AAV of any one of embodiments 1 to 48, wherein the heterologous gene of interest exhibits at least a 5-fold increase in expression of the heterologous gene of interest compared to the promoter of the hSYN1 gene in a neuron of the striatum.
52. The retro-AAV of any one of embodiments 1 to 48, wherein the heterologous gene of interest exhibits at least a 25-fold increase in expression of the heterologous gene of interest compared to the promoter of the hSYN1 gene in a neuron of the striatum.
53. A pharmaceutical composition comprising a pharmaceutically acceptable, carrier, excipient, or diluent and the retro-AAV of any one of embodiments 1 to 52.
54. The retro-AAV of any one of embodiments 1 to 52 or the pharmaceutical composition of embodiment 53, for use in a method to express a polypeptide in a neuron of the striatum.
55. The use of embodiment 54, wherein the neuron of the striatum is a D1 dopaminergic medium spiny neuron.
56. The retro-AAV of any one of embodiments 1 to 52 or the pharmaceutical composition of embodiment 53, for use in a method to genetically engineer a neuron of the striatum.
57. The use of embodiment 56, wherein the neuron of the striatum is a D1 dopaminergic medium spiny neuron.
58. The retro-AAV of any one of embodiments 1 to 52 or the pharmaceutical composition of embodiment 53, for use in a method to treat a neurodegenerative disease in an individual.
59. The use of embodiment 58, wherein the neurodegenerative disease comprises Parkinson's disease.
60. A method to express a polypeptide in a neuron of the striatum of an individual comprising administering the retro-AAV of any one of embodiments 1 to 52 or the pharmaceutical composition of embodiment 53, to the individual thereby expressing the polypeptide the neuron of the striatum.
61. The method of embodiment 60, wherein the neuron of the striatum is a D1 dopaminergic medium spiny neuron
62. A method to genetically engineer a neuron of the striatum of an individual comprising administering the retro-AAV of any one of embodiments 1 to 52 or the pharmaceutical composition of embodiment 53 to the individual thereby genetically engineering the neuron of the striatum.
63. The method of embodiment 62, wherein the neuron of the striatum is a D1 dopaminergic medium spiny neuron.
64. A method to treat an individual afflicted with a neurodegenerative disease comprising administering the retro-AAV of any one of embodiments 1 to 52 or the pharmaceutical composition of embodiment 53 to the individual afflicted with a neurodegenerative disease thereby treating the neurodegenerative disease.
65. The method of embodiment 64, wherein the neurodegenerative disease comprises Parkinson's disease.
66. The method of any one of embodiments 60 to 65, wherein the individual is a mammal.
67, The method of any one of embodiments 60 to 65, wherein the individual is a human.
68. A method to express and activate a DREADD in the central nervous system of an individual comprising administering to the individual the retro-AAV of any one of embodiments 1 to 52 and a ligand that activates the DREADD, thereby activating the DREADD in the central nervous system of the individual.
69. The method of embodiment 68, wherein the DREADD is expressed and activated int in a neuron of the striatum,
70. The method of embodiment 69, wherein the neuron of the striatum is a D1 dopaminergic medium spiny neuron.
71. The method of any one of embodiments 68 to 70 wherein the individual is a mammal.
72. The method of any one of embodiments 68 to 70, wherein the individual is a human.
73. The method ofany one of embodiments 68 to 72 wherein activating the DREADD in the central nervous system of the individual treats a neurodegenerative disorder.
74. The method of embodiment 73, wherein the neurodegenerative disorder comprises Parkinson's disease
75. The method of any one of embodiments 68 to 74, wherein the ligand that activates the DREADD comprises quetiapine or clozapine.
76. The method of any one of embodiments 68 to 74, wherein the ligand that activates the DREADD comprises quetiapine.
77. The method of any one of embodiments 68 to 74, wherein the ligand that activates the DREADD comprises clozapine.
78. The method of any one of embodiments 68 to 74, wherein the retro-AAV and the ligand that activates the DREADD are administered separately.

### EXAMPLES

The following illustrative examples are representative of embodiments of compositions and methods described herein and are not meant to be limiting in any way.

### Example 1—Development of Highly Efficient Retrograde AAV Capsids for D1 MSN

In this experiment, a highly efficient retrograde adeno-associated virus (AAV) capsid, AAV8R, was developed for use in D1-type medium spiny neurons (MSN).

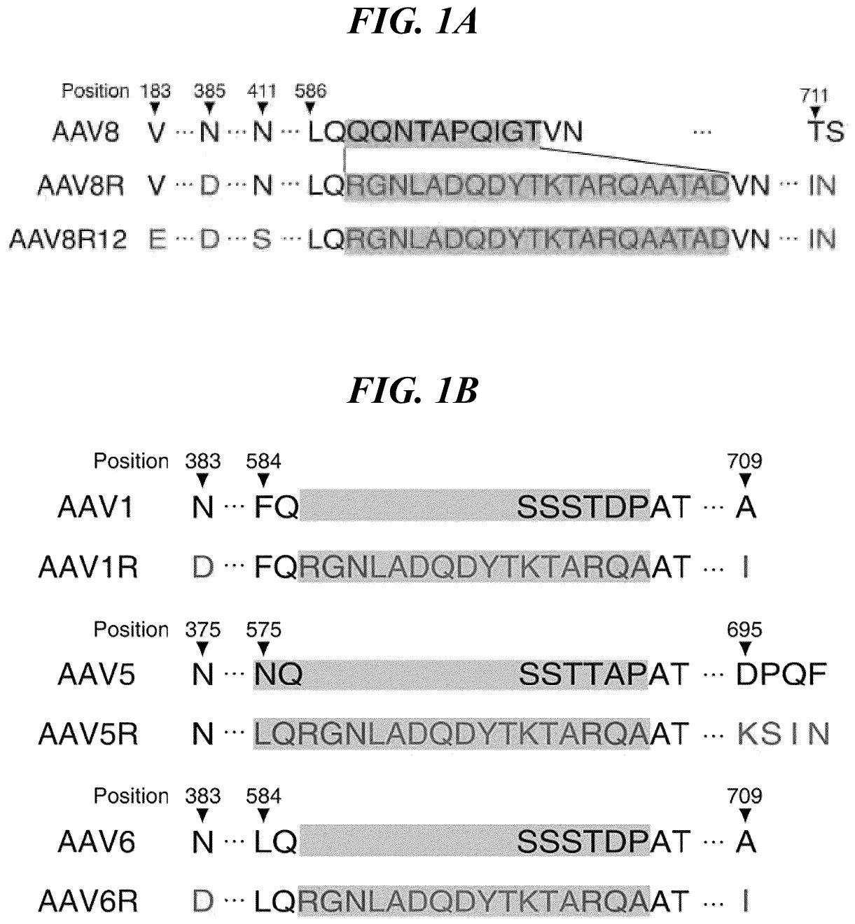

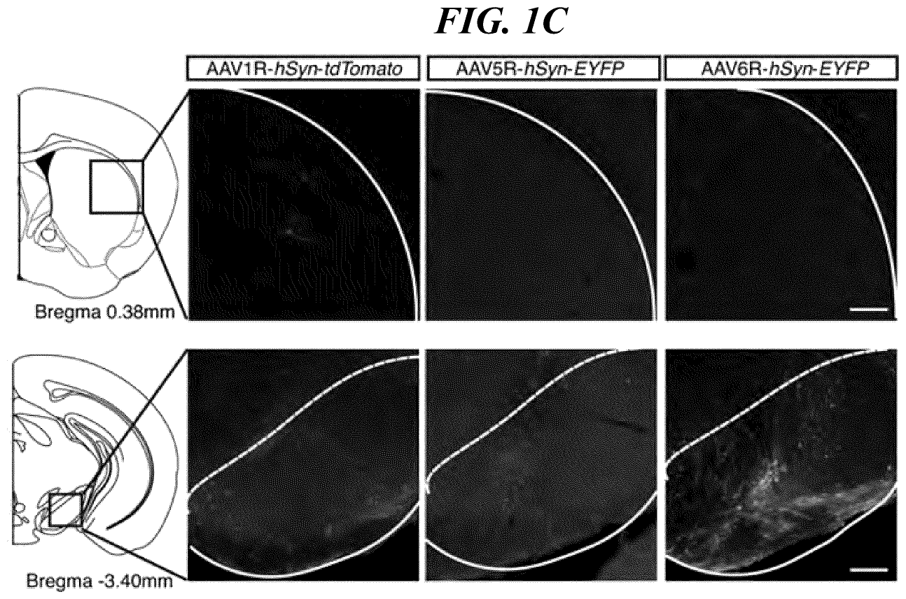

A standard retrograde AAV tracer, rAAV2-retro, is only moderately efficient at injecting D1 MSNs when injected into the substantia nigra pars reticulata (SNr), and striatal MSNs are only sparsely labeled by rAAV2-retro-hSyn-EYFP. To improve efficiency, multiple rounds of mutations of AAV capsids were performed on a series of different serotypes. Mutations were introduced at three sites in the AAV8 capsid protein to make AAV8R, including N385D, insertion of RGNLADQDYTKTARQAATAD (SEQ ID NO: 31) at position 588, and TS711-712IN. Two additional mutations of V183E and N411S were incorporated into the AAV8R12 capsid protein (FIG. 1A). The labeling pattern of the AAV8R12 capsid and G88 promoter combination was determined. Retrogradely labeled neurons in the SNr and its upstream brain regions after nigral injection of AAV8R12-G88P7-EYFP in mice showed 97.68±0.43% of labeled neurons in the striatum, while only 1.14±0.16% and 1.18±0.41% of labeled neurons were found in the SNr and its other upstream brain regions, respectively (FIGS. 1D and 1E; n=3 mice per group).

First, the polypeptide fragment

(SEQ ID NO.: 31)
RGNLADQDYTKTARQAATAD

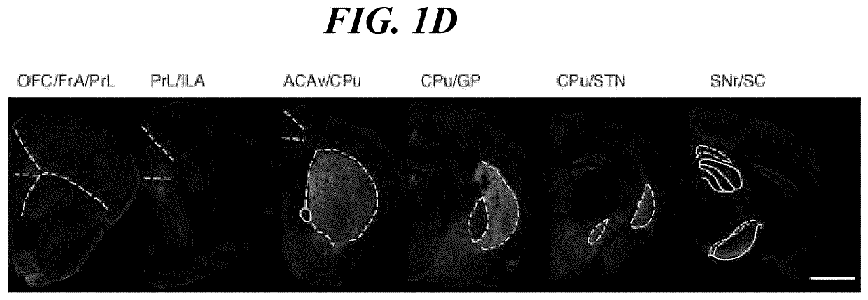

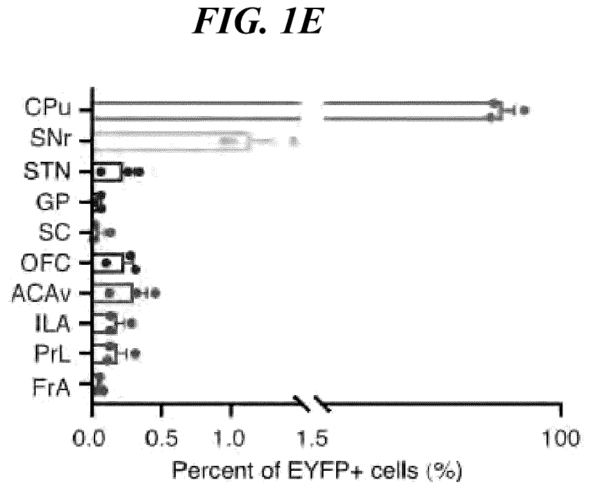

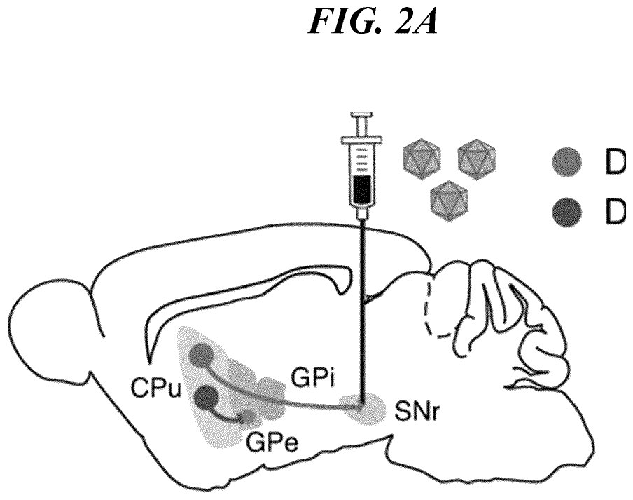

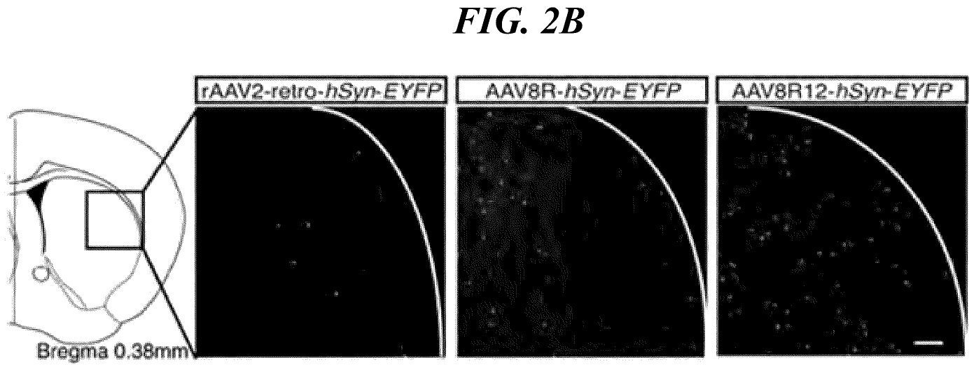

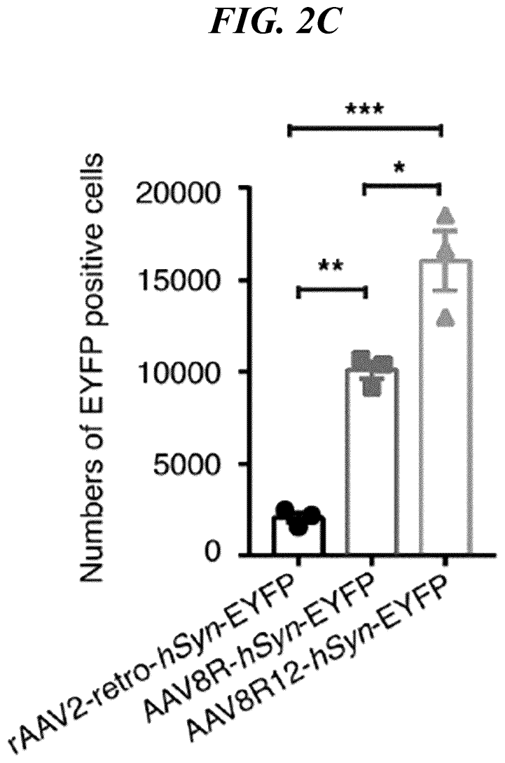

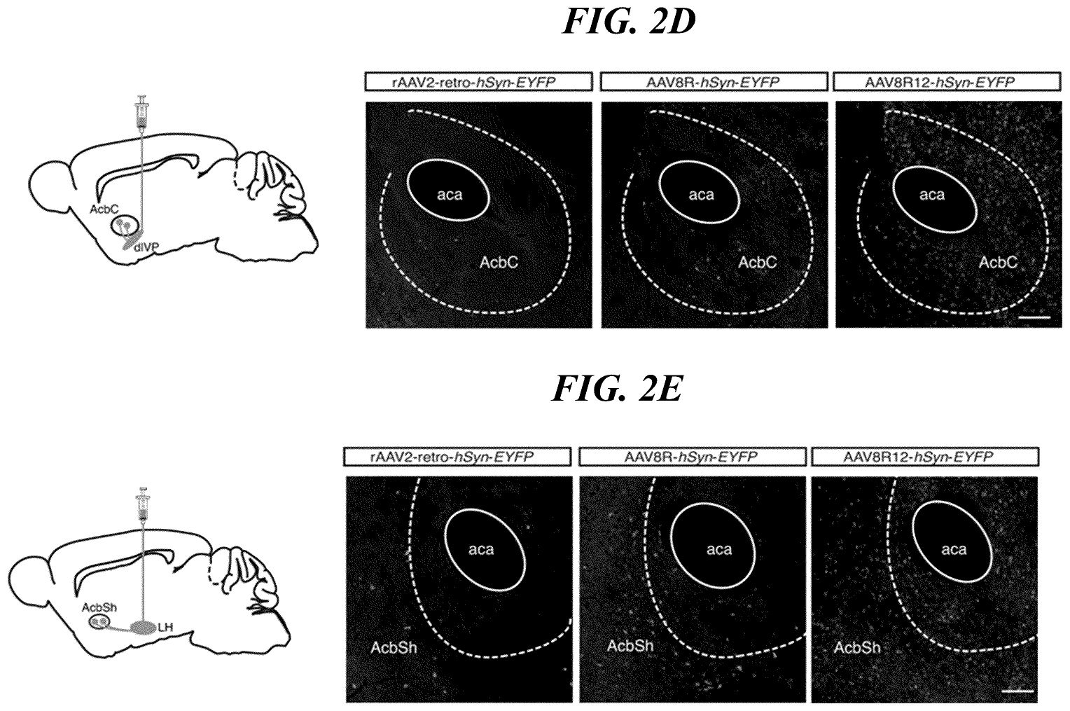

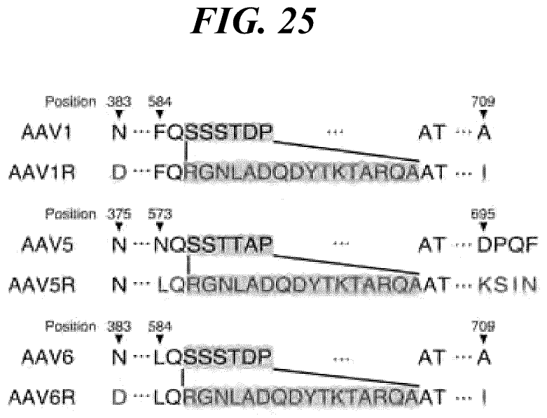

was inserted at the N587-R588 position; two other point mutations of the AAV2 Cap protein were made to develop rAAV2-retro. Similar mutations were then placed at equivalent positions for four other AAV serotypes (FIG. 1A-1C and 25, TABLE 1), AAV1/5/6/8 (FIG. 1A). All 4 modified AAVs maintained infectivity of the brain, but only the AAV8 mutant, AAV8R, acquired improved retrograde infectivity of D1 MSNs. When administered into the SNr, 4.86±0.22 times EYFP positive MSNs in mice infected by AAV8R-hSyn-EYFP were observed as compared to those infected by rAAV2-retro-hSyn-EYFP (FIG. 2A-2C).

Baseline infectivity of rAAV2-retro was approximately 0.2±0.03×10⁴ cells per striatal hemisphere (using a hSyn promoter) (TABLE 2). Among the 14 mutants tested, AAV8R12 displayed the mostly improved efficiency. Compared to the current standard retrograde AAV, rAAV2-retro, AAV8R12 labeled 7.72±0.78-times more MSNs after nigral delivery (FIG. 2B-2C, TABLE 1). Moreover, robust labeling of MSNs was observed in nucleus accumbens by AAV8R and AAV8R12 after stereotaxic delivery into ventral pallidum or lateral hypothalamus, indicating the unique ability of newly developed AAV capsids to infect axons of basal ganglia MSNs. The AAV8R12 was generated using the AAV8R plasmid as a template.

The sequence of the mutagenesis primer used to introduce the V183E mutation was 5′-TGGCGACTCAGAGTCAGAGCCAGACCCTCAACCTCT-3′ (SEQ ID NO.: 34). The sequence of the mutagenesis primer used to introduce the N411S mutation was

(SEQ ID NO.: 35)
5′- TGCTGAGAACCGGCAACAGCTTCCAGTTTACTTACACCT -3′.

TABLE 1
List of AAV8R mutants and their labeling
efficiency of striatal neurons

| | | Labeling Efficiency in |
|---|---|---|
| Name | Mutation | Striatonigral Pathway |
| AAV8R1 | V183E | + |
| AAV8R2 | N411S | + |
| AAV8R3 | Y447F | + |
| AAV8R4 | R490Q | + |
| AAV8R5 | V125I | + |
| AAV8R6 | F536Y | + |
| AAV8R7 | A606S | + |
| AAV8R8 | T495A | + |
| AAV8R9 | V125I + F536Y | + |
| AAV8R10 | V125I + A606S | + |
| AAV8R11 | V125I + T495A | + |
| AAV8R12 | V183E + N411S | +++ |
| AAV8R13 | V125I + F536Y + T495A | ++ |
| AAV8R14 | V125I + A606S + T495A | ++ |

This table shows the mutations in the 15 AAV8R capsid mutants. “+”, “++”, and “+++” indicate numbers of labeled neurons of <5k, 5k-10k, and 10k-25k per animal, respectively.

TABLE 2
Mean and Standard Deviation of AAVs with capsid and promoter

| AAV | Mean | SD | SEM |
|---|---|---|---|
| AAV2R-hsyn | 2078 | 454.5 | 262.4 |
| AAV8R-hsyn | 10092 | 803.5 | 463.9 |
| AAV8R12-hsyn | 16026 | 2865 | 1654 |
| AAV8R12-ef1a | 6954 | 1210 | 698.7 |
| AAV8R12-cag | 4610 | 1226 | 707.7 |
| AAV8R12-G88P2 | 36290 | 6417 | 3705 |
| AAV8R12-G88P3 | 37026 | 11511 | 6646 |
| AAV8R12-G88P7 | 39460 | 12611 | 7281 |

### Example 2—Development of Robust Promoters for D1 MSN

To find promoters that offer high levels of expression in MSNs, gene expression databases were scanned to identify a list of eight genes that have highly enriched striatal expression compared to other parts of the basal ganglia (BG). Genes highly expressed in the striatum, but not other parts of the basal ganglia, were first selected as candidates.

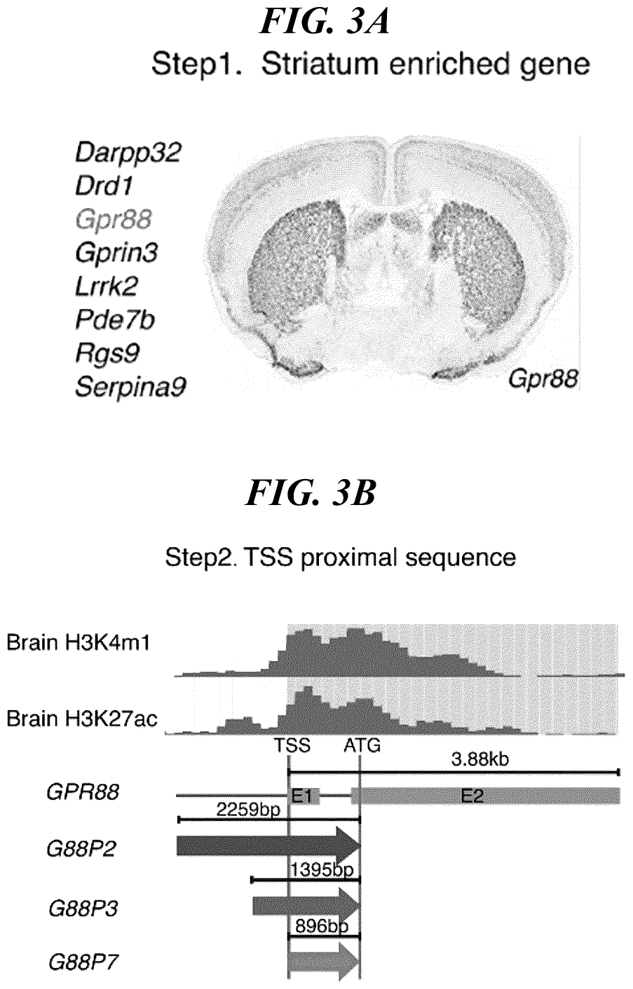

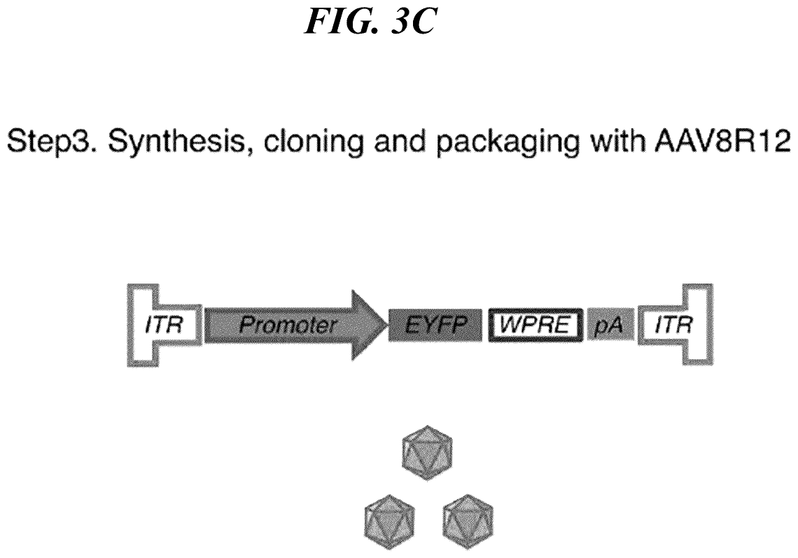

Brain profiles of two epigenetic marks for enhancers and promoters, monomethylation of histone H3 lysine 4 (H3K4me1) and acetylation of histone H3 lysine 27 (H3K27ac) were checked and approximately 2 kilobase-long sequences around the transcriptional start site (TSS) that have high levels of H3K4me1 and/or H3K27ac were identified in mouse brain. Homologous sequences at equivalent positions of the human genome were then cloned onto an AAV backbone, and the activity of the homologous sequences in directing reporter expression was tested in mice after injections of AAVs into the SNr (FIG. 3A-C, TABLE 3).

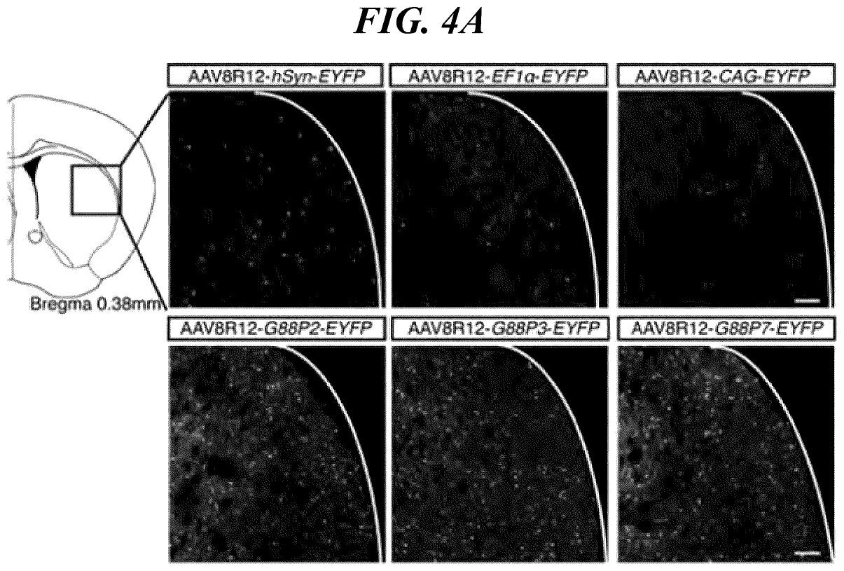

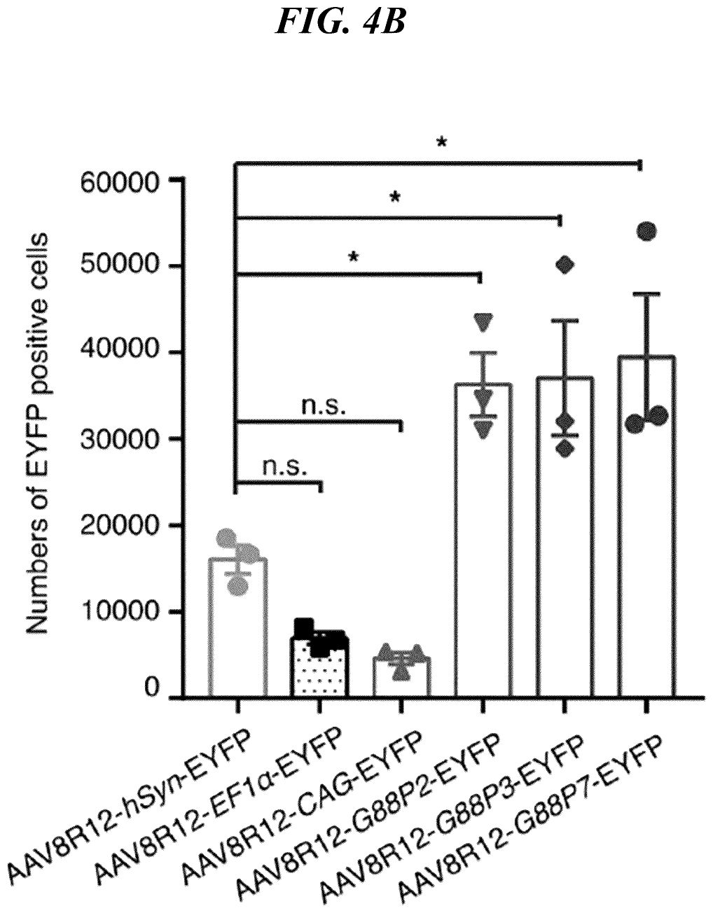

Among 11 promoters tested, A 2259-bp promoter, Neural Promoter 1 (G88P2) from the gene GPR88, showed the highest activity in driving gene expression in MSNs compared with commonly used promoters, CAG, EF1α, and hSyn (FIG. 4A-4B). G88P2 (2259 bp) was cloned with the following primers: 5′-CATCGCAAGGCTACATGATGG (SEQ ID NO.: 36) and 3-CTGGCCAACTCTTCACACCTC (SEQ ID NO.: 37). To increase the payload of AAV genome, the G88P2 promoter was shorted using restriction enzymes, and two derivatives, G88P3 and G88P7, were made which were 1395-bp and 896-bp long, respectively (FIG. 3A-3C). Comparative efficiency in labeling MSNs for the two shortened promoters was observed. For mice that received viral injections into the SNr, viruses expressing EYFP driven by G88P3 and G88P7 promoters labeled 3.7±0.66×104 and 3.95±0.73×104 MSNs per mouse, respectively, compared to 1.61±0.16×104 labeled MSNs by hSyn promoter (FIG. 4B). The shortest of these strong MSN promoters, G88P7, was composed of a short 67 bp sequence before the TSS, exon 1 (366 bp), intron 1 (391 bp), and a 72 bp fragment of exon 2 of the human GPR88 gene, suggesting that likely cis-regulatory elements in exon 1 and intron 1 of the GPR88 gene are sufficient in initiating strong striatal expressions.

TABLE 3
List of AAv8R mutants and their labeling
efficiency of striatal neurons

| | | | 5′ Distance | | | |
|---|---|---|---|---|---|---|
| | | Promoter | to TSS | Length | Activity Level | |
| # | Gene | Name | (bp) | (bp) | SN | STR |
| 1 | DARPP32 | D32P1 | 1646 | 2336 | + | − |
| 2 | DRD1 | D1P1 | 1073 | 2182 | + | + |
| 3 | GPR88 | G88P1 | 578 | 2299 | + | ++ |
| 4 | | G88P2 | 1430 | 2259 | + | ++++ |
| 5 | | G88P3 | 581 | 1395 | + | ++++ |
| 6 | | G88P7 | 67 | 896 | + | ++++ |
| 7 | GPRIN3 | G3P1 | 696 | 2001 | + | − |
| 8 | | G3P2 | 1810 | 2204 | + | − |
| 9 | LRRK2 | L2P1 | 950 | 2201 | + | + |
| 10 | PDE7B | P7P1 | 294 | 2309 | + | − |
| 11 | RGS9 | R9P1 | 591 | 2152 | + | +++ |
| 12 | | R9P2 | 2147 | 2219 | + | + |
| 13 | SERPINA9 | S9P1 | 2234 | 2309 | + | + |

This table shows the positions, sizes, and activity levels of tested promoters. “+”, “++”, “+++”, and “++++” indicate numbers of YFP-expressing neurons of<5k, 5k-10k, 10k-25k, and >25k per animal, respectively. SN, substantia nigra; STR, striatum.

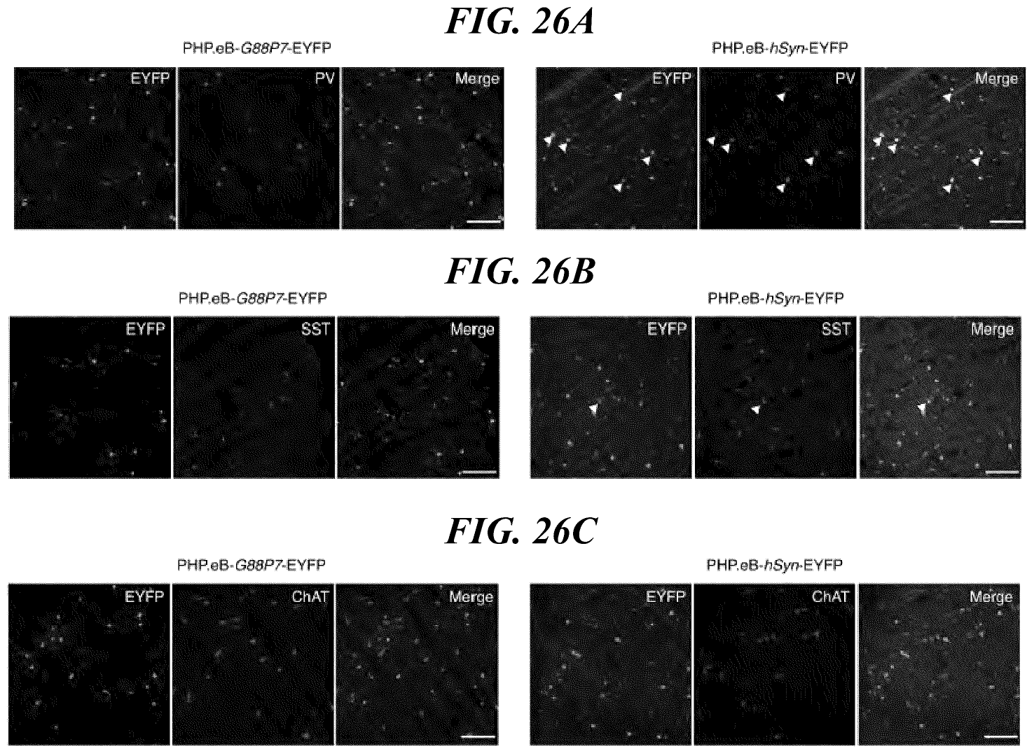

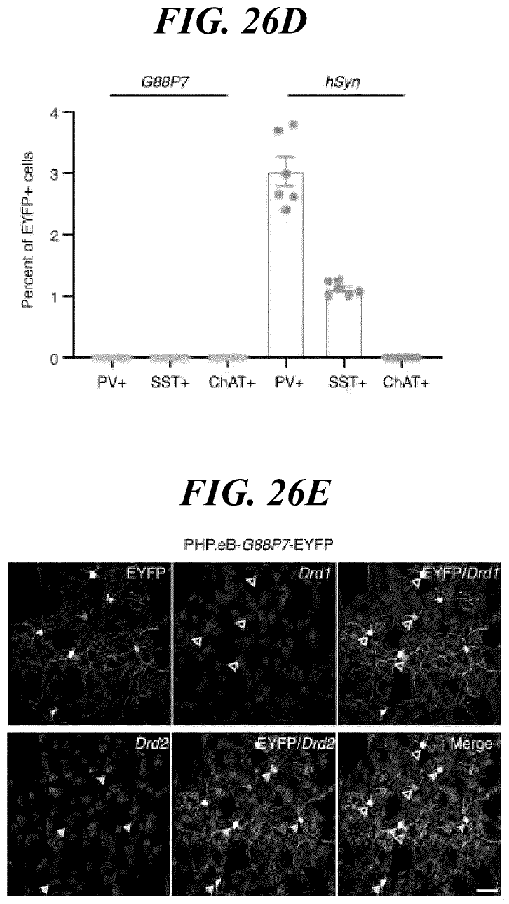

Consistent with exclusively high levels of gene expression of Gpr88 in MSNs but not other striatal cell types in the mouse striatum, co-staining of EYFP was observed with DRD1 and Drd2, but not ChAT, parvalbumin, or somatostatin, after intravenous delivery of AAV-PHP.eB-G88P7-EYFP in FIG. 26A-26E.

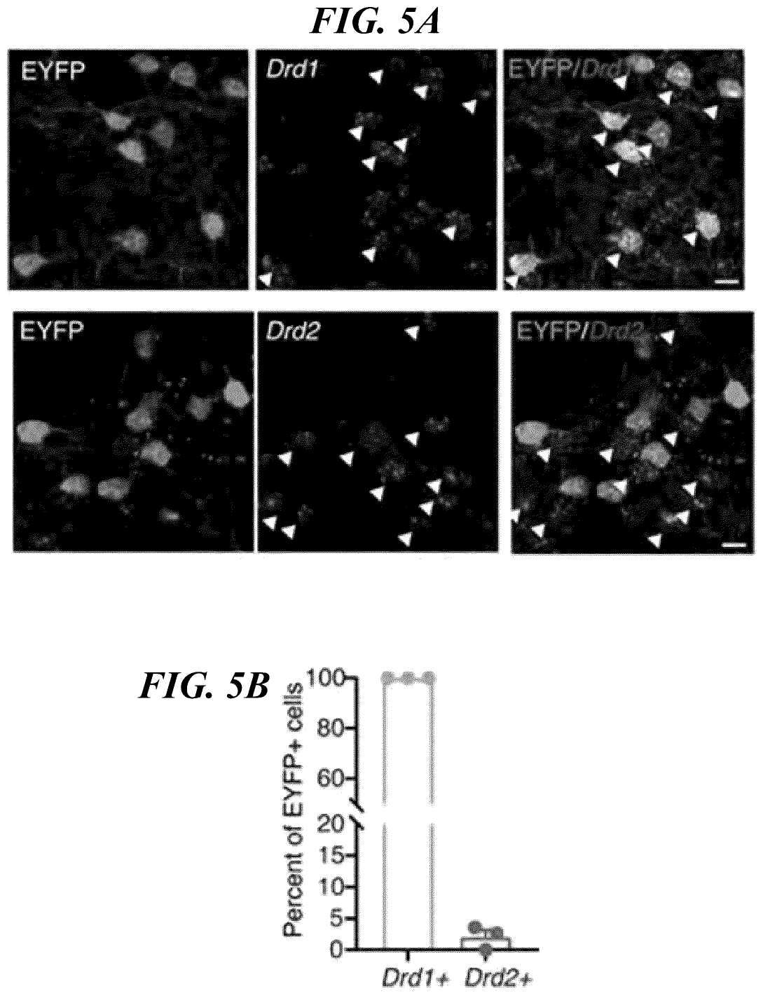

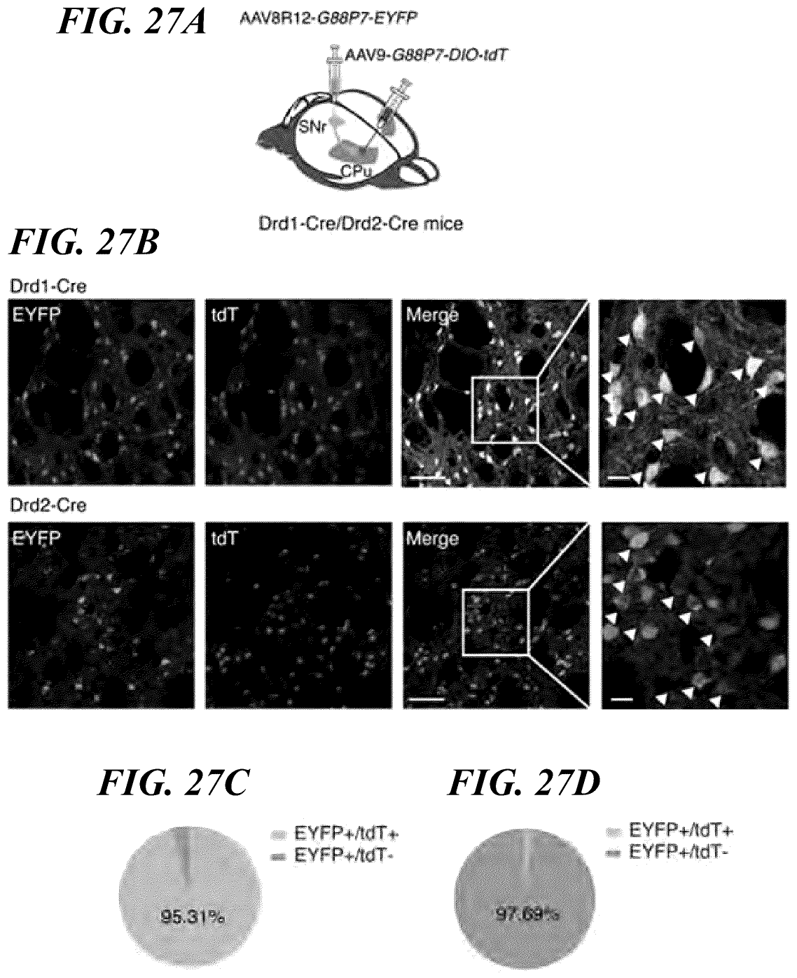

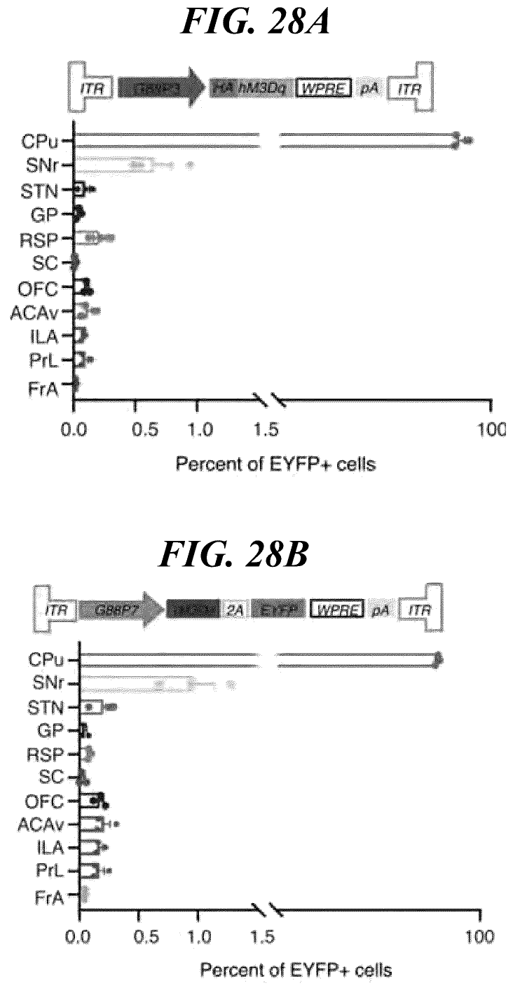

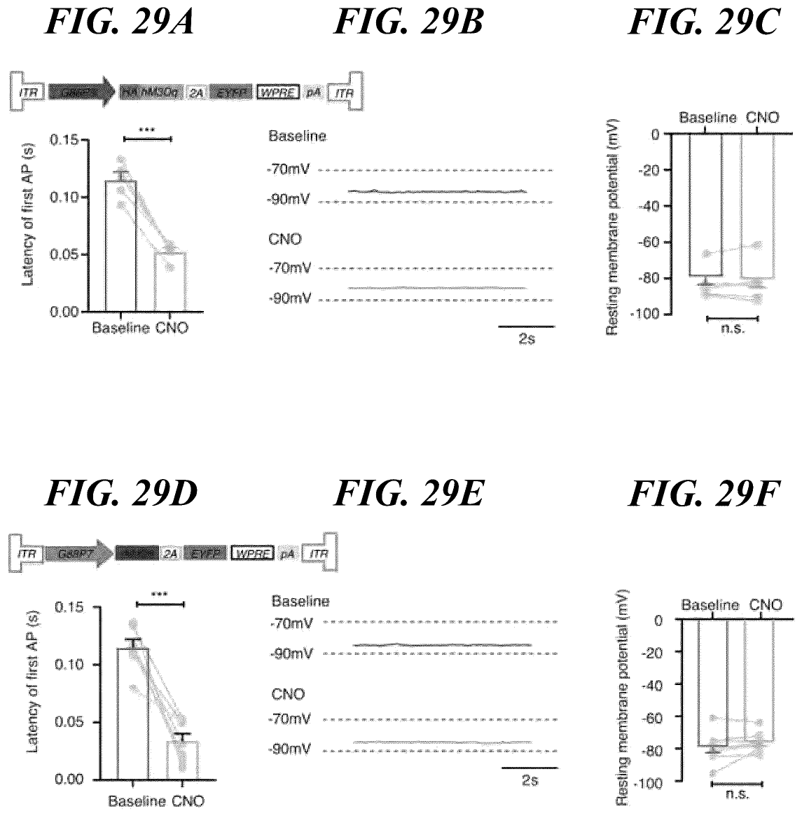

Labeling specificities of new retrograde AAV tracers were examined in mice. Dual labeling for EYFP and Drd1 or Drd2 after nigral injection of AAV8R12-G88P7-EYFP in mice indicated that retrogradely labeled neurons were Drd1+ with <3% showing positive Drd2 immunoreactivity (FIG. 5A-5B). In some instances, a very minor population of SNr-projecting MSNs express both Drd1 and Drd2 receptors. Consistently, further testing with simultaneous nigral injection of AAV8R12-G88P7-EYFP and striatal injection of AAV9-G88P7-DIO-tdTomato in Drd1-Cre and Drd2-Cre mice confirmed that a very small fraction (<3%) of SNr-targeting MSNs labeled by AAV8R12 were tdTomato+ in Drd2-Cre mice (FIG. 27A-27D). With this AAV-based retrograde labeling approach, it was found that 12.44±2.3% of D1-NMSNs were reporter-positive in mice.

### Example 3—Chemogenetic Manipulation of Basal Ganglia Direct Pathway in Mice

Mice were anesthetized with sodium pentobarbital (Nembutal; 80 mg/kg, i.p.), and then placed into a stereotaxic device (KOPF). Eye cream was applied on both corneas to avoid dehydration. The skull above the targeted areas was thinned with a dental drill and carefully removed. Injections were conducted with a 10 μL syringe connected to a 33-Gauge needle (Neuros; Hamilton), using a microsyringe pump (Legato 130, KD Scientific). A total volume of 200 nL virus was injected into the SNr at a speed of 20 nL/min. The coordinates for the SNr were 3.4 mm posterior, 1.3 mm lateral, 4.8 mm ventral to the bregma.

Mice that were used in the behavioral experiments were then implanted with guide cannulas (KOPF) unilaterally aiming at the dorsomedial striatum (0.5 mm anterior, 1.5 mm lateral, 3.5 mm ventral to the bregma). The cannulas were fixed to the skull with dental cement. Stainless steel obturators were inserted into the guide cannulas and were replaced every other day to maintain the patency until infusions were made. Mice were allowed to recover from the surgery for at least 3 weeks before further studies.

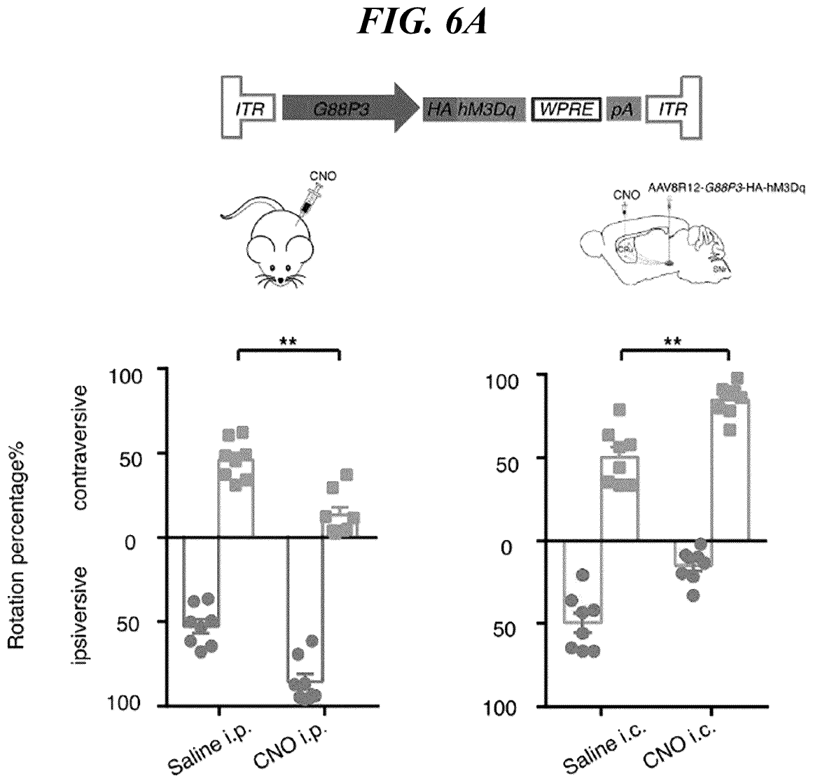

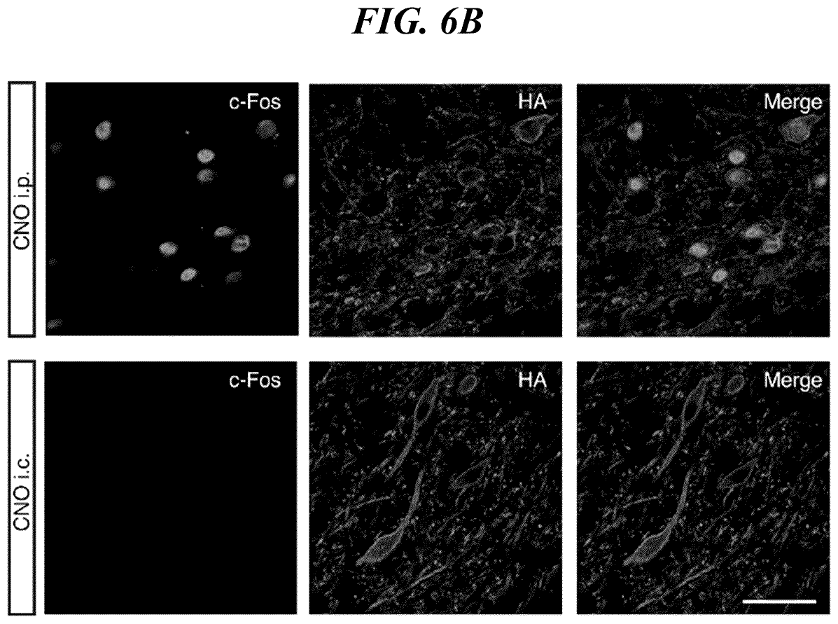

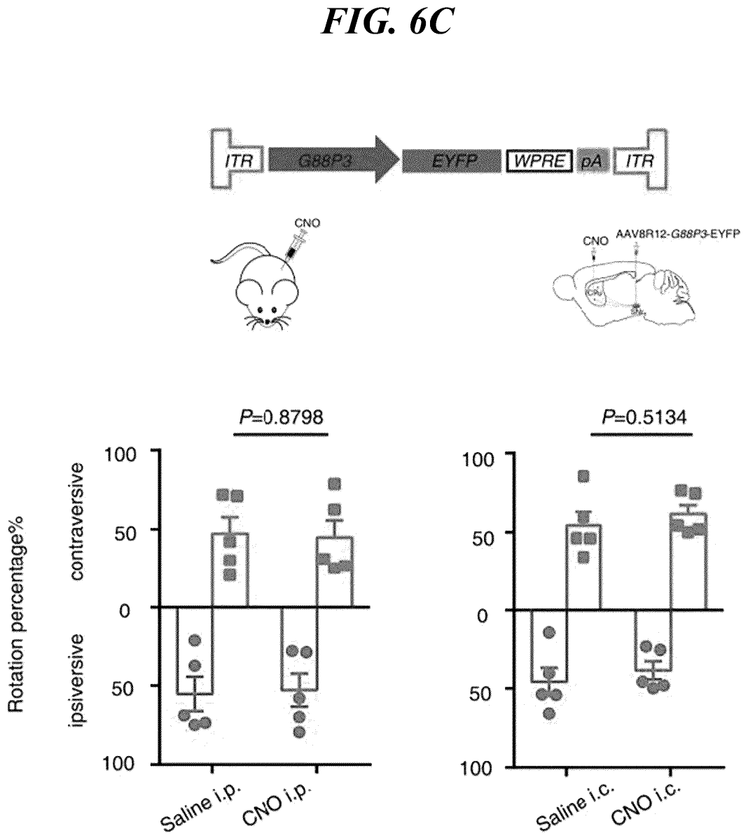

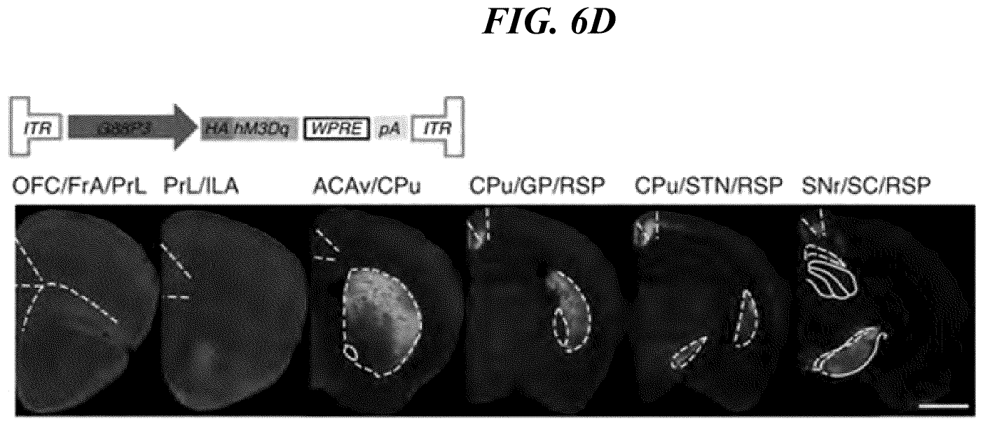

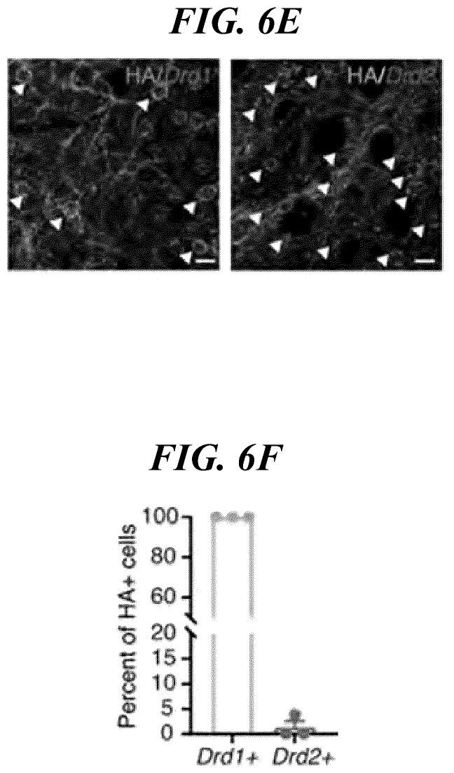

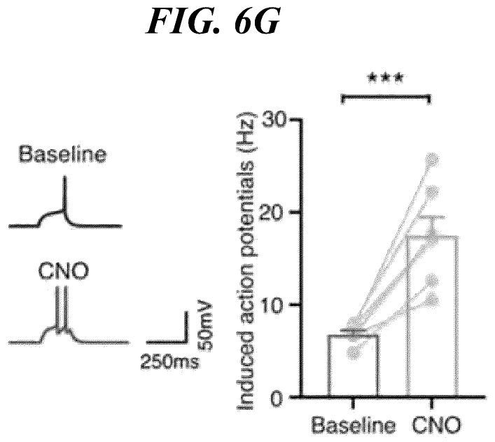

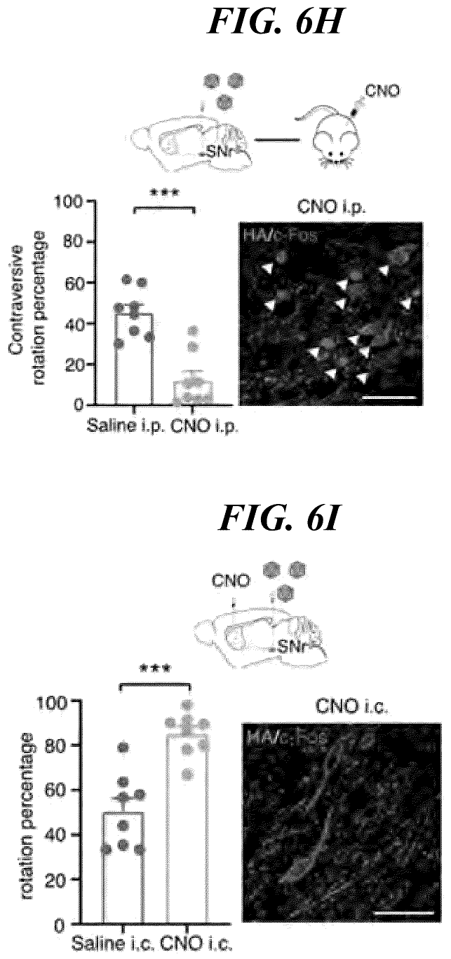

Labeling specificity of the retrograde AAV tracers was examined. Dual labeling for AAV8R12-G88P3-EYFP and Drd1 or Drd2 in mice indicated that the retrograde AAV marked exclusively D1 MSNs (FIG. 5A). To explore the functional features of retrograde AAV-labeled cells, AAV8R12-G88P3-HA-hM3Dq, which enables neuronal excitation upon clozapine N-oxide (CNO) administration, were injected into the right SNr of C57BL/6J mice. Three weeks after injection, CNO was delivered intraperitoneally, and elevated ipsiversive and reduced contraversive rotations were observed as compared to saline controls (FIG. 6A). This result indicated an inhibition of the BG movement control pathways, contrary to the predicted action of activated D1 MSNs in the right striatum. Based on the observed infection patterns of retrograde AAV, it was reasoned that the ipsiversive rotations were a consequence of the activation of the right SNr, which is a major inhibitory output center of the BG and was labeled by the locally injected AAV.

Immunohistochemistry analyses confirmed a significant increase in c-Fos+ cells in the right SNr after CNO but not saline administration (FIG. 6B). CNO or saline was then administered intracranially near the dorsomedial part of the right striatum after AAV delivery to the right SNr. Contraversive rotations were induced by CNO but not saline (FIG. 6A). No significant changes in the number of nigral c-Fos+ cells after intracranial CNO infusions was observed (FIG. 6B). Moreover, CNO didn't induce rotational behaviors in animals which received AAV8R12-G88P3-EYFP injections into the SNr (FIG. 6C). These results showed that pharmacogenetic activation of retrogradely labeled D1 MSNs by the proposed strategy is sufficient to drive behavioral changes in mice.

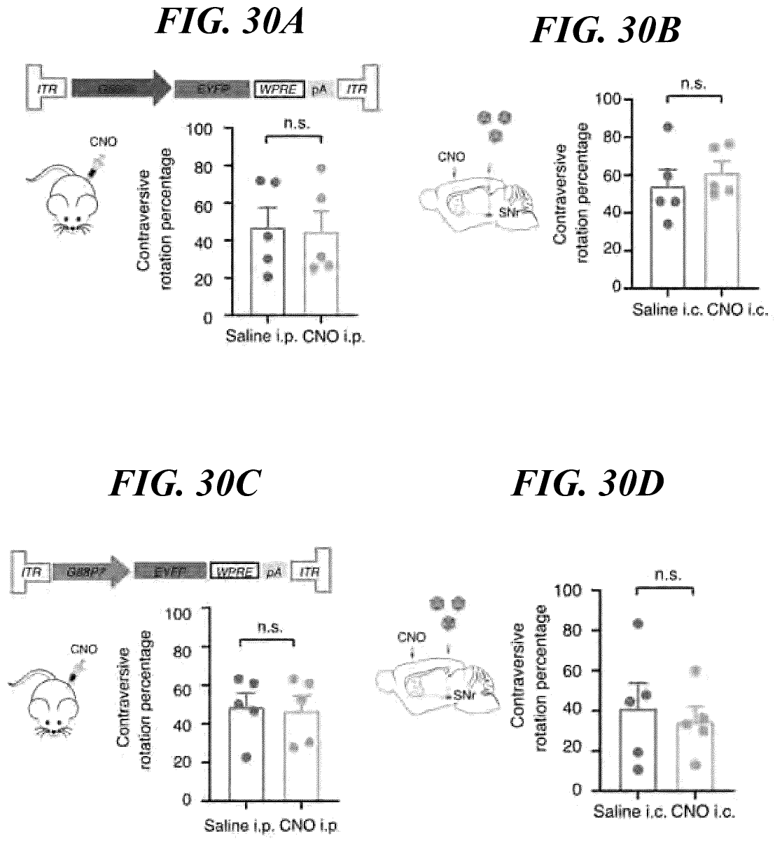

To explore functional features of retrograde AAV-labeled cells, AAV8R12-G88P3-HA-hM3Dq which expresses DREADD effector hM3Dq and may enable neuronal excitation upon Clozapine N-oxide (CNO) administration was unilaterally injected into the SNr of C57BL/6J mice. Three weeks after injection, brains were harvested and anatomical analyses revealed that the majority of labeled neurons were located in the striatum and that all labeled neurons were Drd1+ (FIG. 6D-6F, 28A). Using slice whole-cell voltage clamp recordings, an augmentation of excitability in AAV8R12-G88P3-HA-hM3Dq-2A-EYFP transduced-MSNs was found upon CNO administration without affecting the basal firing rate or resting membrane potential (FIG. 6G, 29A-29C). Surprisingly, reduced contraversive rotations after intraperitoneal CNO delivery were observed compared to saline injection (FIG. 6H). This result indicated an inhibition of the direct pathway or an excitation of the indirect pathway, contrary to the predicted outcome of unilateral activation of D1-MSNs in the striatum. Based on the observed infection patterns of the retrograde AAV, it was reasoned that the ipsiversive rotations may be a consequence of activating the ipsilateral SNr, which may be a major inhibitory output center of the BG and was mildly transduced by the locally injected AAV (FIG. 1D-1E, 5A-5B, 6D, 28A). Immunohistochemical analyses confirmed a significant increase in c-Fos+ cells in the injected SNr after CNO but not saline administration (FIG. 6H). To further explore this observation, CNO or saline was administered intracranially near the dorsomedial region of the ipsilateral striatum after unilateral nigral AAV delivery. With this approach, contraversive rotations were induced by CNO but not saline and significant changes in the number of nigral c-Fos+ cells were not observed (FIG. 6I). Moreover, CNO did not induce behavioral alterations in animals that received AAV8R12-G88P3-EYFP injections into the SNr (FIG. 30A-30B). These results demonstrate that chemogenetic activation of D1-MSNs labeled by newly developed retrograde AAVs may drive behavioral changes in mice.

To further optimize the system, an alternative chemogenetic effector that would potentially allow specific manipulation of the direct pathway compatible with systemic CNO infusions needed to be identified. The commonly used Gq-coupled effectors elicited a rise in the cellular concentration of Ca²⁺, which has been shown to be more effective in driving neuronal excitation in various neuronal subtypes than other second messengers. A different chemogenetic effector, rM3Ds, which uses cAMP as the second messenger and can effectively activate striatal MSNs, was used to avoid the activation of SNr neurons upon systemic CNO administration.

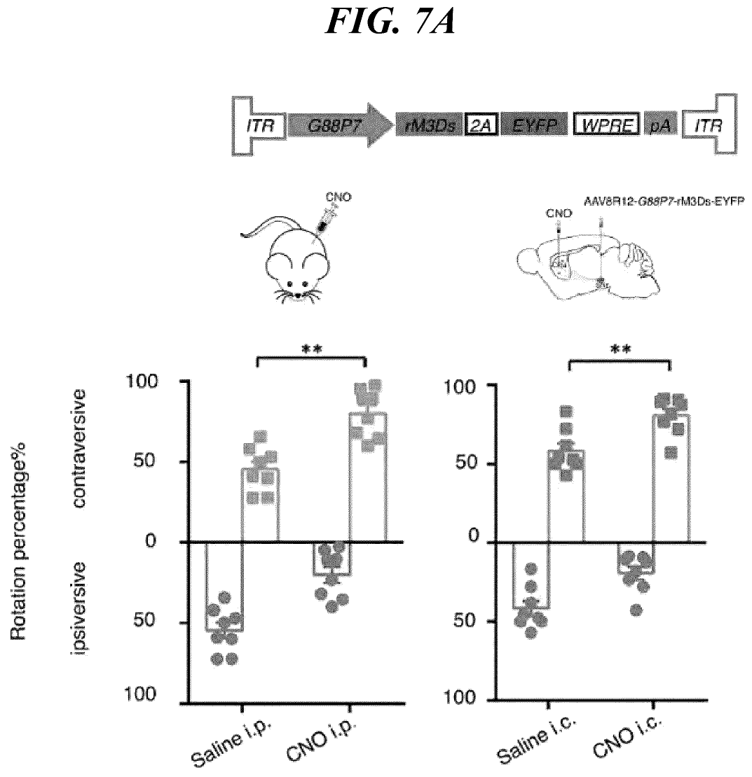

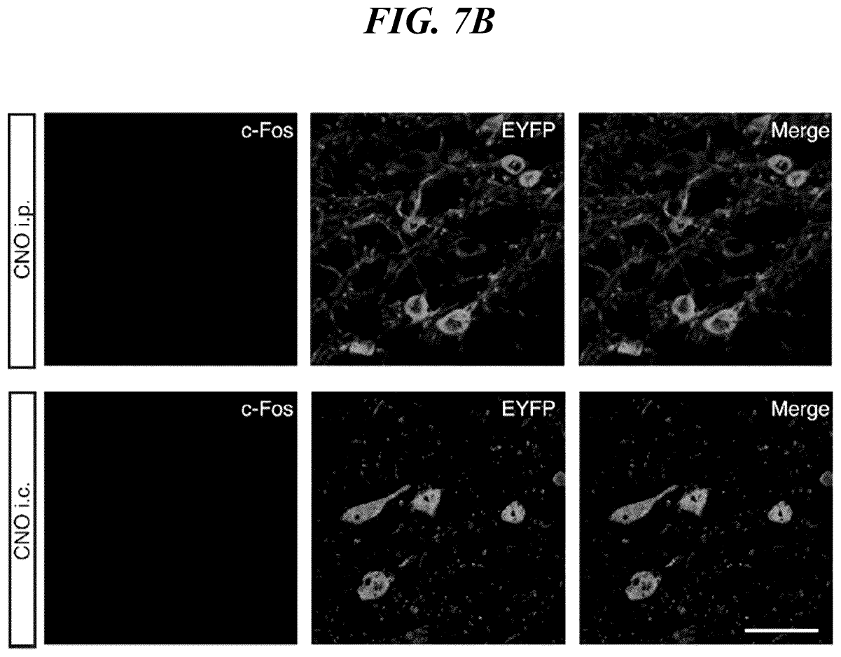

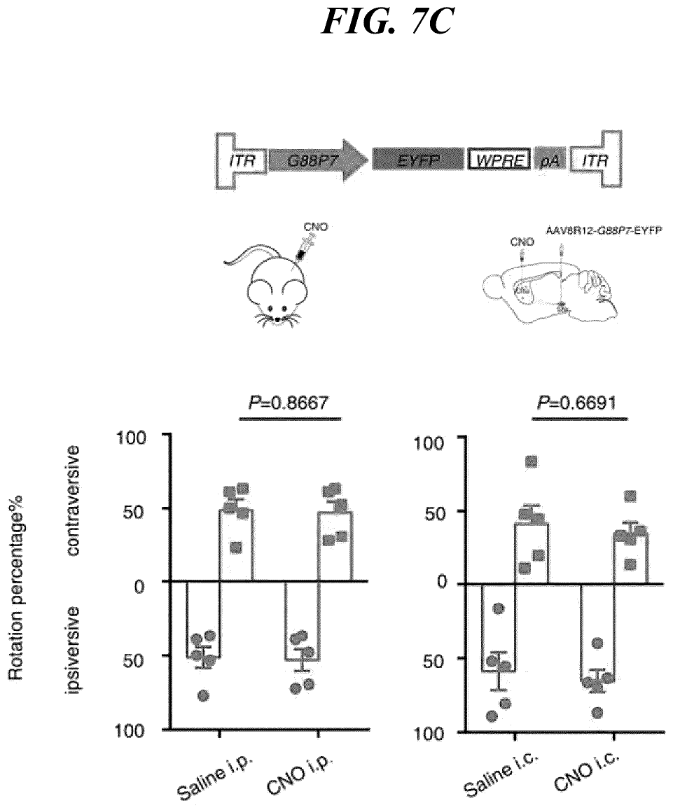

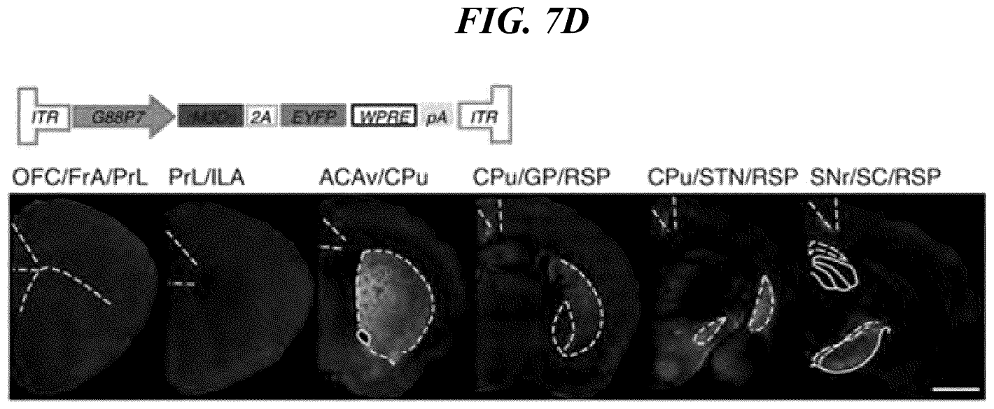

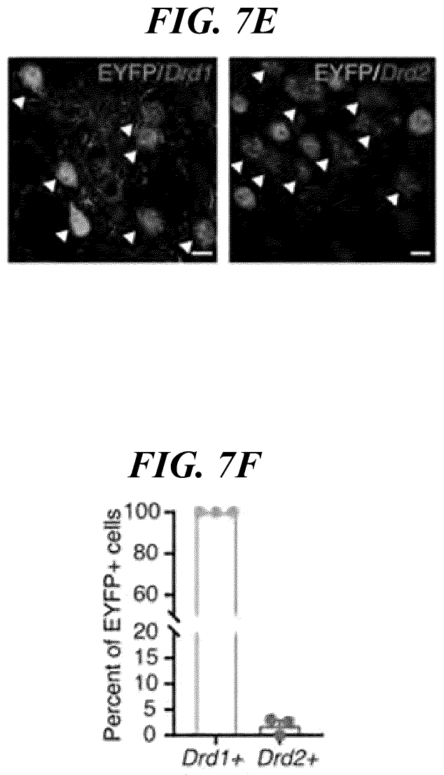

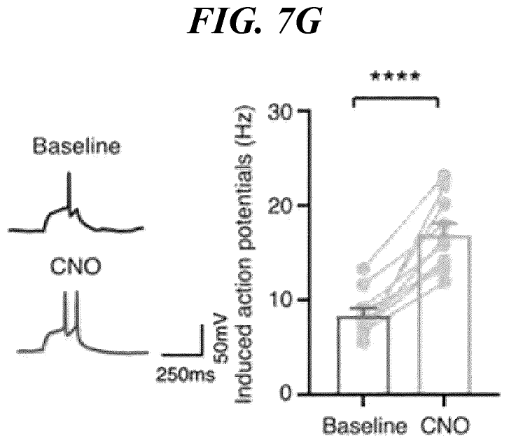

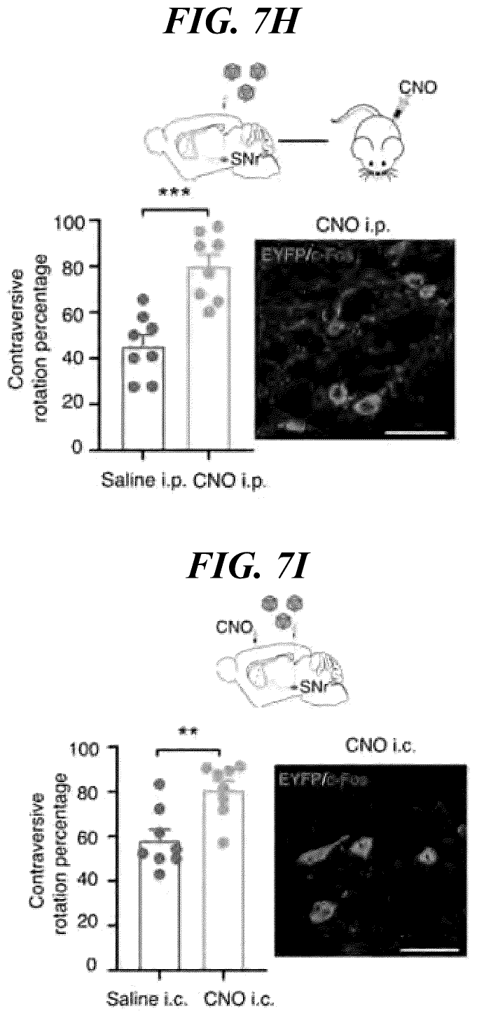

To test this, AAV8R12-G88P7-rM3Ds-2A-EYFP was injected unilaterally into the right SNr of adult mice, both intraperitoneal and intracranial infusions of CNO induced contraversive rotations (FIG. 7A). Immunohistochemistry and in situ hybridization analyses confirmed that most labeled neurons were located in the striatum and that retrogradely transduced neurons were Drd1+(FIG. 7D-7F, 28B). Slice electrophysiological recordings demonstrated an enhancement in the excitability of labeled striatal MSNs upon CNO administration, with no effect on basal firing rate or resting membrane potential (FIG. 7G, 29D-29F). CNO didn't increase the number of c-Fos+ cells in the SNr after intraperitoneal CNO administrations (FIG. 7B). Both intraperitoneal and intracranial infusions of CNO induced contraversive rotations and did not increase the number of c-Fos+ cells in the SNr (FIG. 7H-7I). Furthermore, CNO didn't induce rotational behaviors in animals received AAV8R12-G88P7-EYFP injections into the SNr (FIG. 7C, 30C-30D). These results demonstrated the specificity of rM3Ds in activating retrogradely labeled MSNs, but not nigral neurons at the injection site. To evaluate the durability of an approach, mice that received nigral AAV8R12-G88P7-rM3Ds-2A-EYFP infusion 12 months after the initial viral infection were tested, and consistently elevated contraversive rotations were found (FIG. 7J-7I, 9D-9E). Together, these findings confirm that the approach we developed is a durable solution for selective activity modulation of D1-MSNs and the BG direct pathway.

The results in this example confirmed that the constructed toolkit, including the highly efficient designer retrograde AAV tracer AAV8R12, strong striatal promoters G88P3/3, and chemogenetic effector rM3Ds or hM3Dq, comprise a recombinase-free system that selectively isolates neuronal subtypes for functional interrogations.

### Example 4—Chemogenetic Manipulation of Basal Ganglia Direct Pathway in Macaque Monkeys

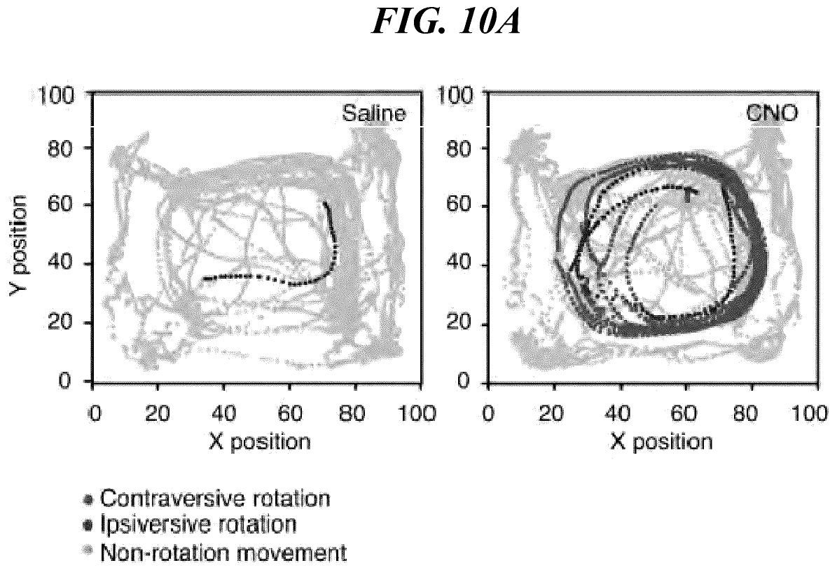

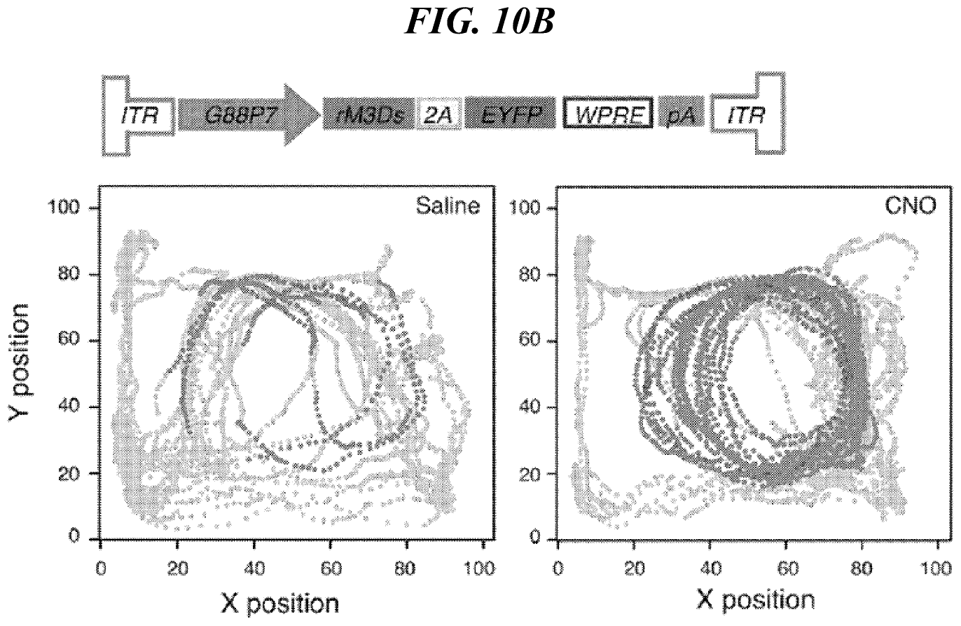

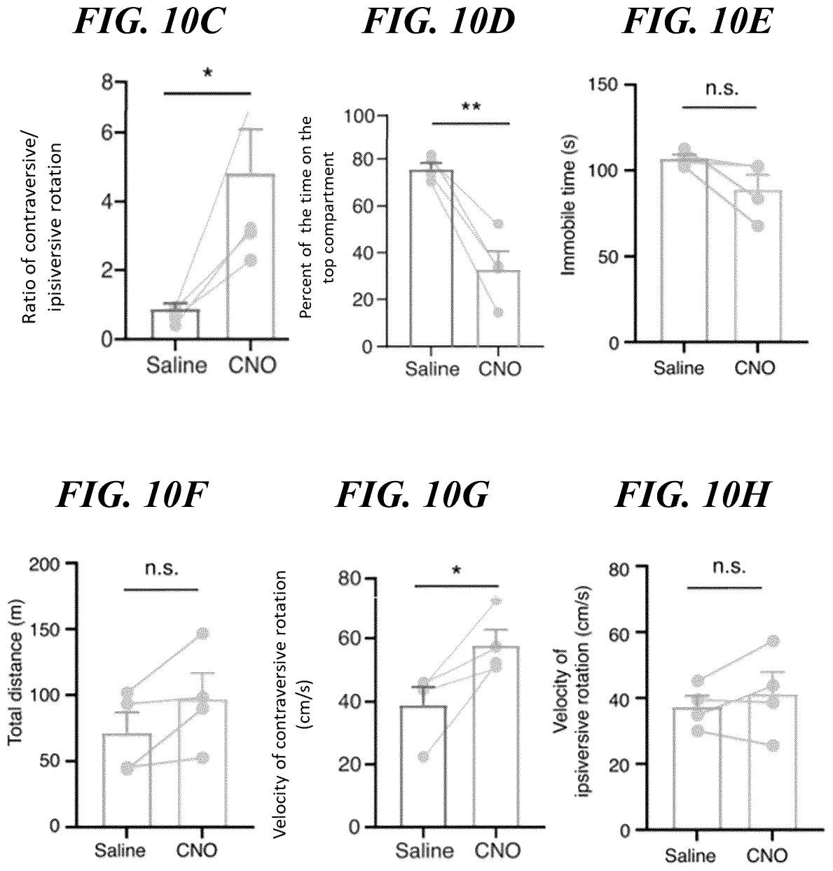

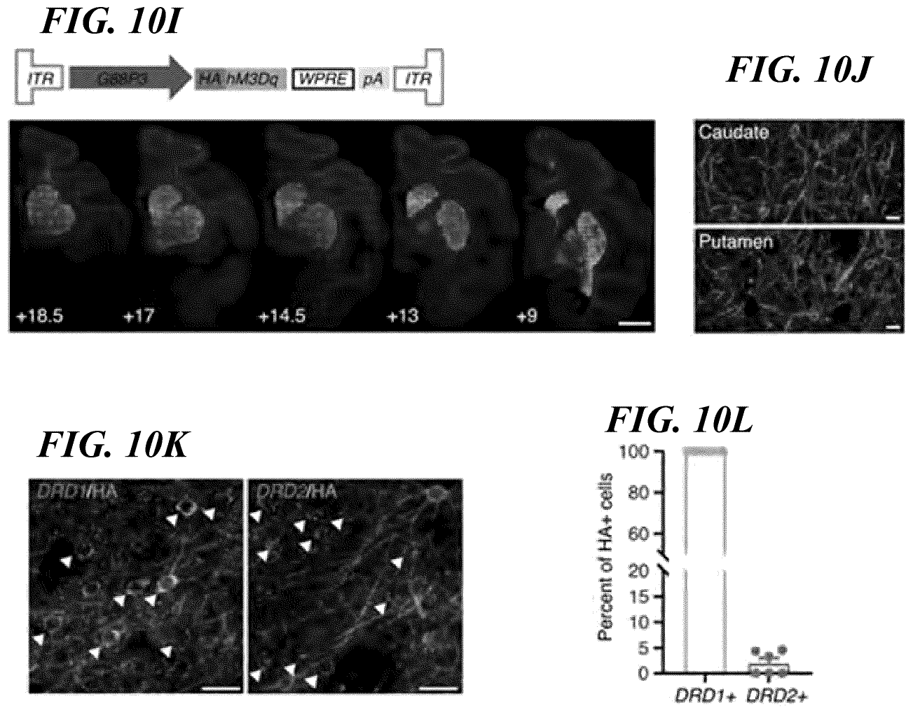

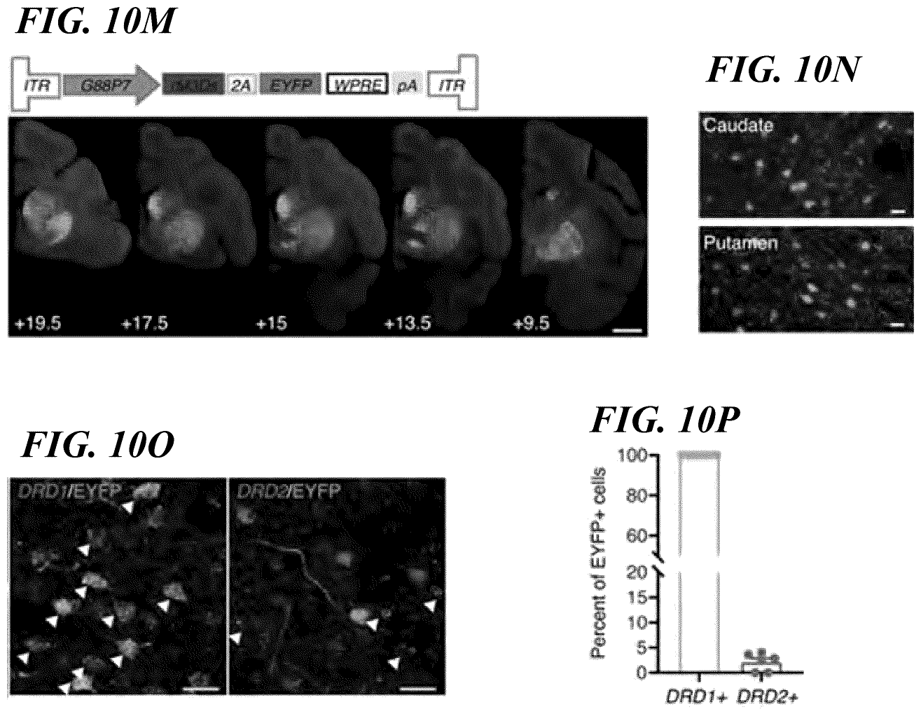

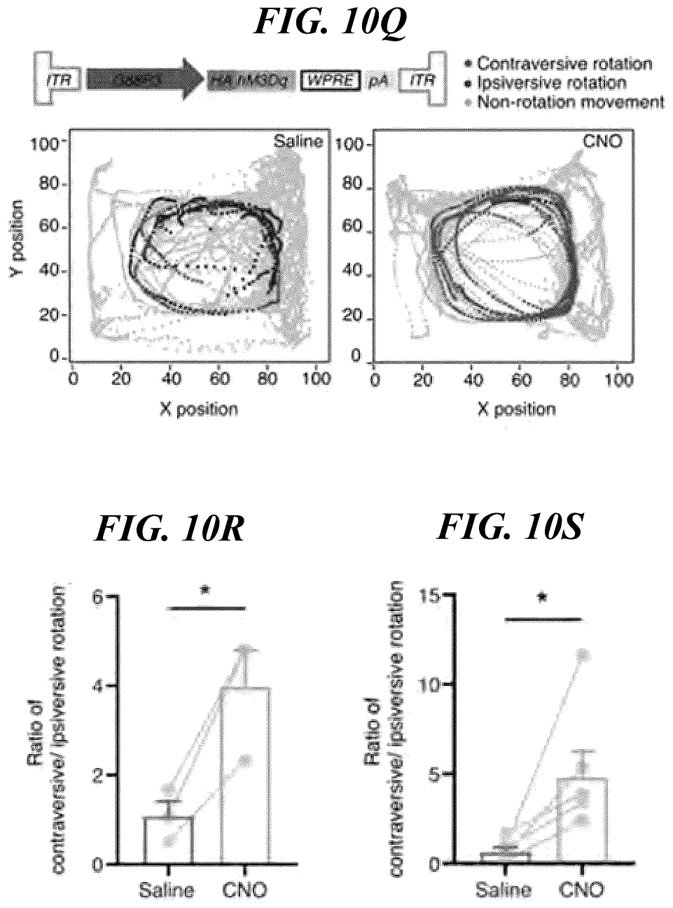

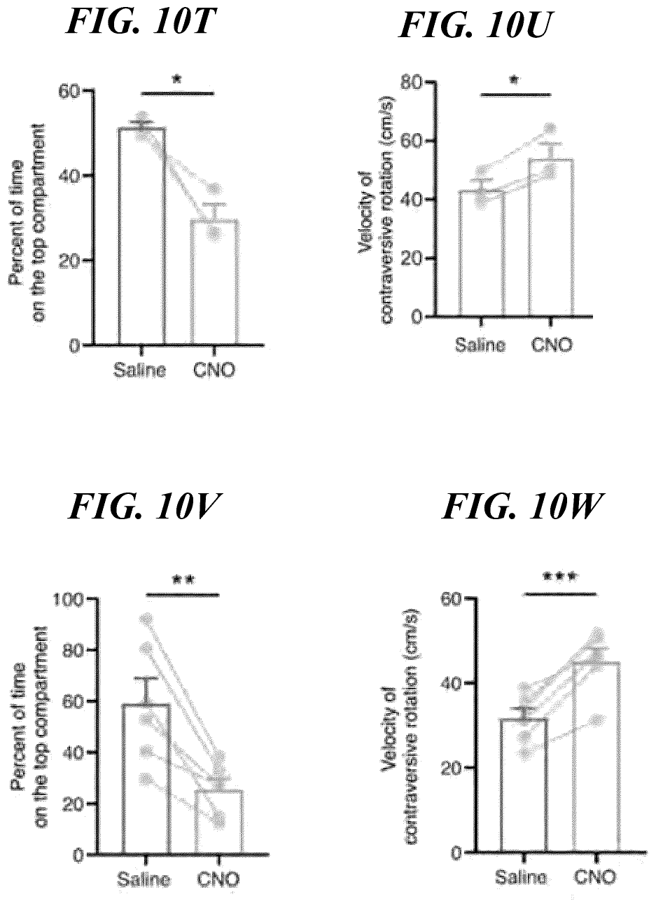

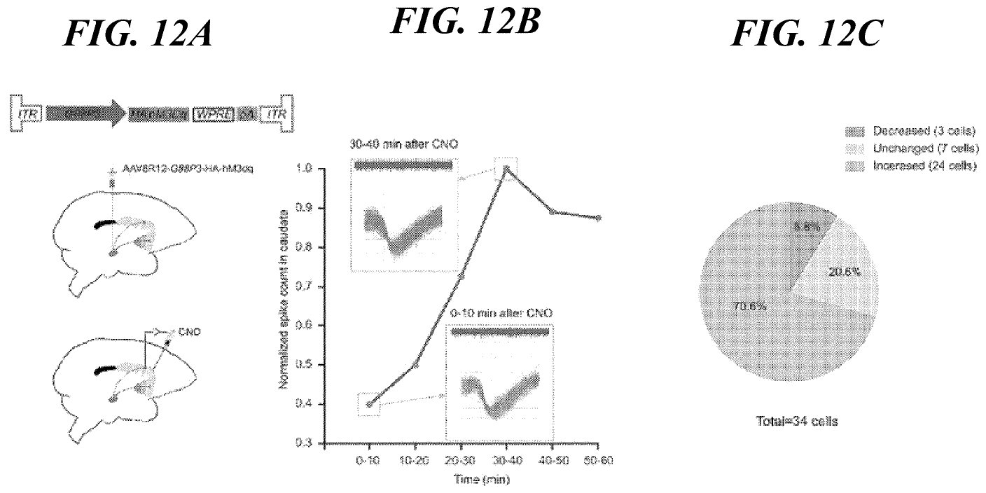

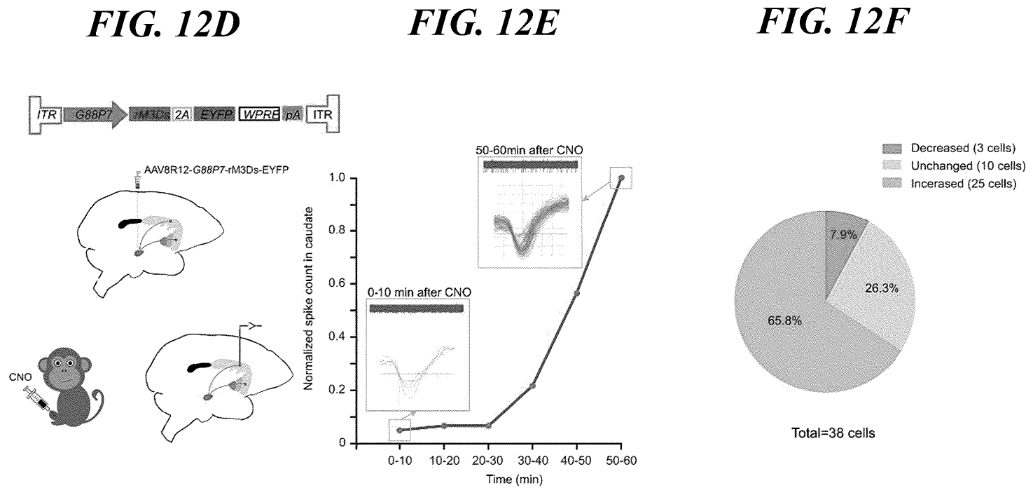

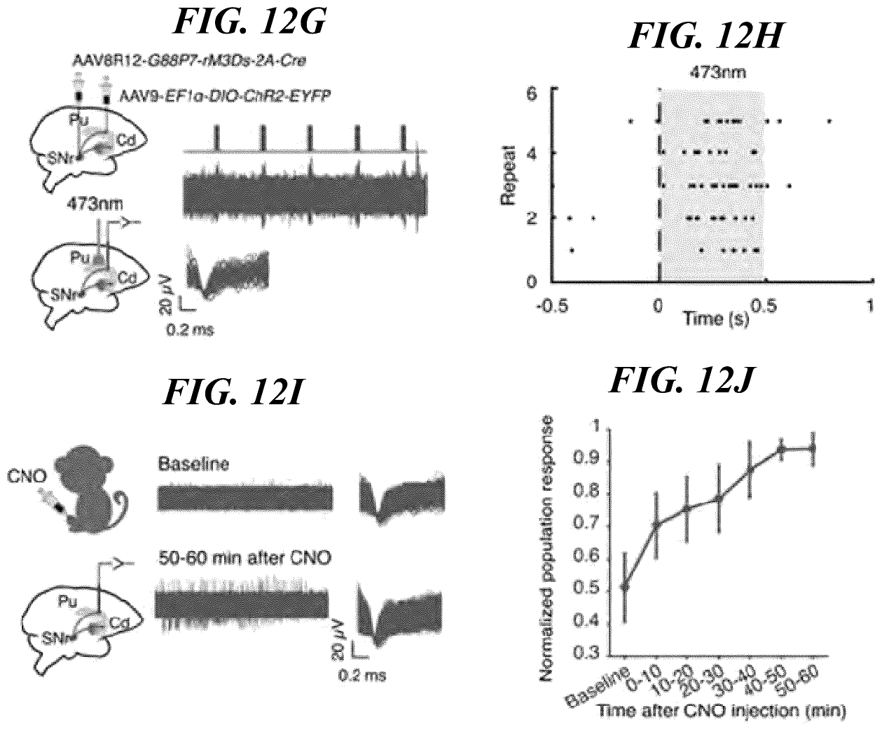

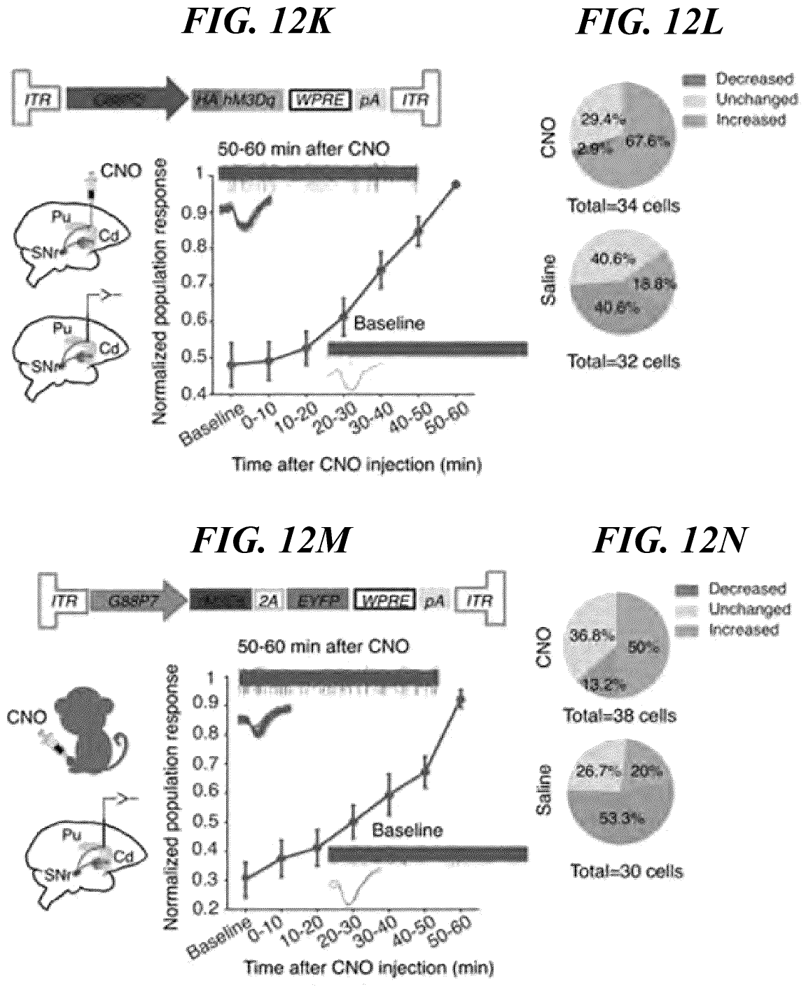

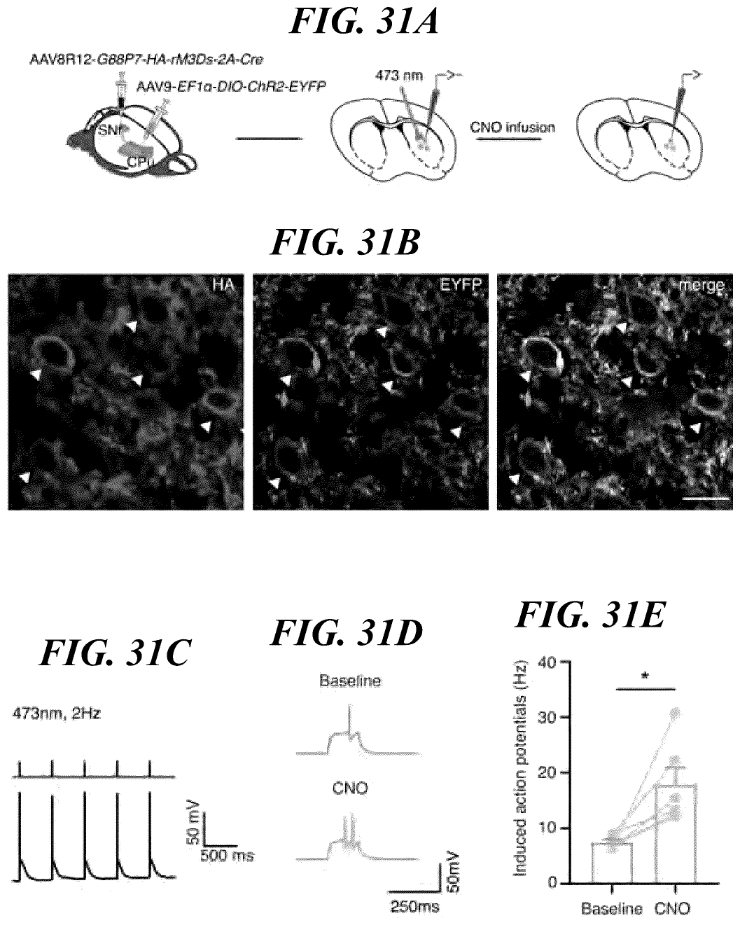

Chemogenetic manipulation of neuronal activity, although sometimes not targeted to specific neural circuitry, may be effective in a macaque brain. To test the effectiveness of a BG direct pathway circuit modulation approach in primate models, AAV8R12-G88P3-HA-hM3Dq or AAV8R12-G88P7-rM3Ds-2A-EYFP was unilaterally injected into the SNr in macaques. Anatomical analyses indicated that most labeled neurons were found in the caudate and putamen and that all striatal DREADD+ neurons were DRD1+ (FIG. 10I-10P). Electrophysiological recordings in anesthetized animals confirmed an increase in neuronal activity in the caudate/putamen following CNO, but not saline, infusions (FIG. 12K-12N). To directly assess how rM3Ds expression in D1-MSNs affected their activity, simultaneous nigral AAV8R12-G88P7-HA-rM3Ds-2A-Cre and striatal AAV9-EF1α-DIO-ChR2-EYFP injections were conducted in mice and macaques. Immunohistochemical analyses and slice recordings in mice confirmed co-expression of ChR2 and rM3Ds in striatonigral projection neurons and that labeled neurons were activated by both light (473 nm) and CNO (FIG. 31A-31E). In vivo opto-tagging recordings in anesthetized macaques revealed that CNO effectively induced an increase of neuronal activity in retrogradely labeled D1-MSNs (FIG. 12G-12J).

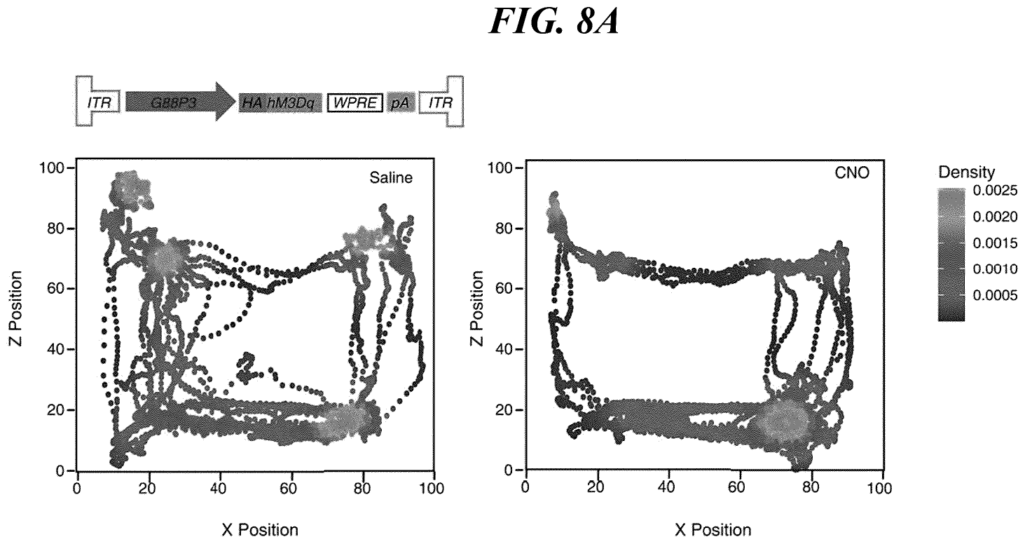

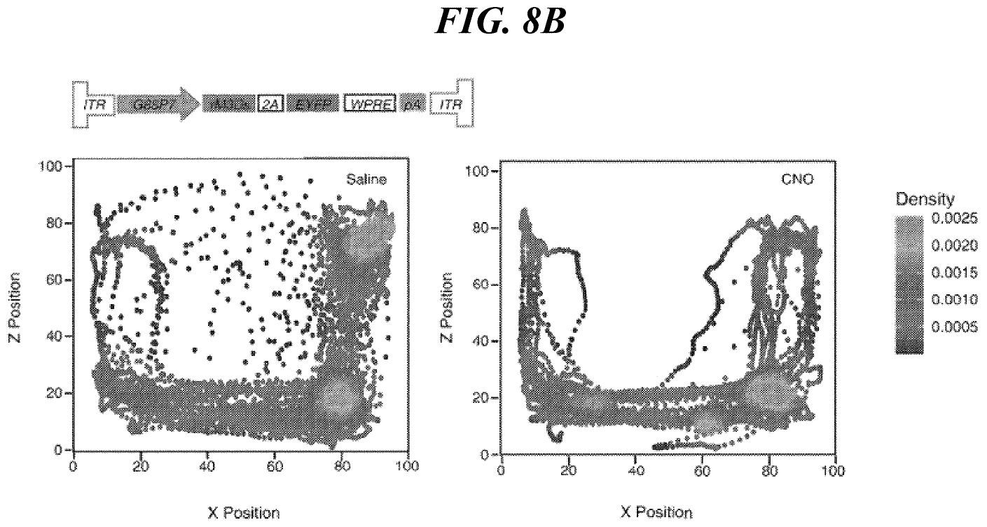

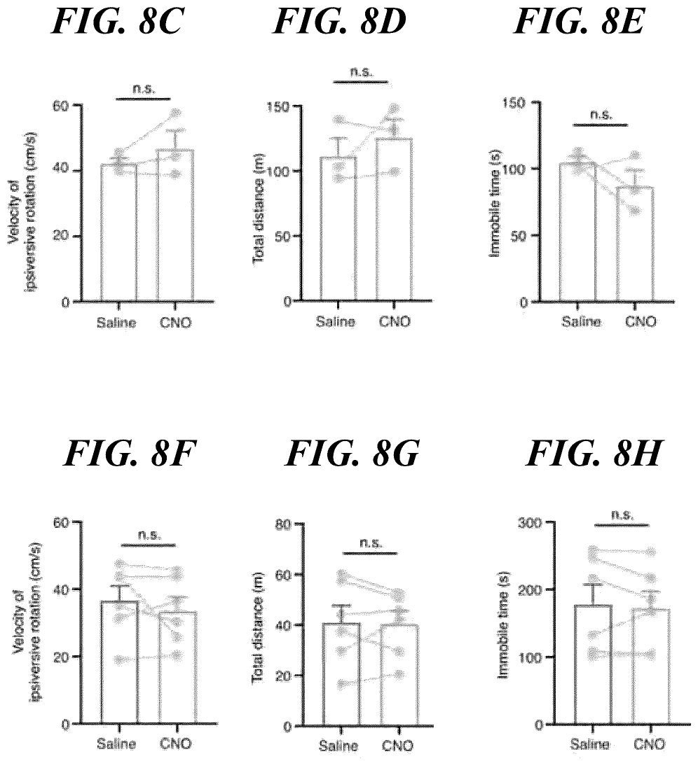

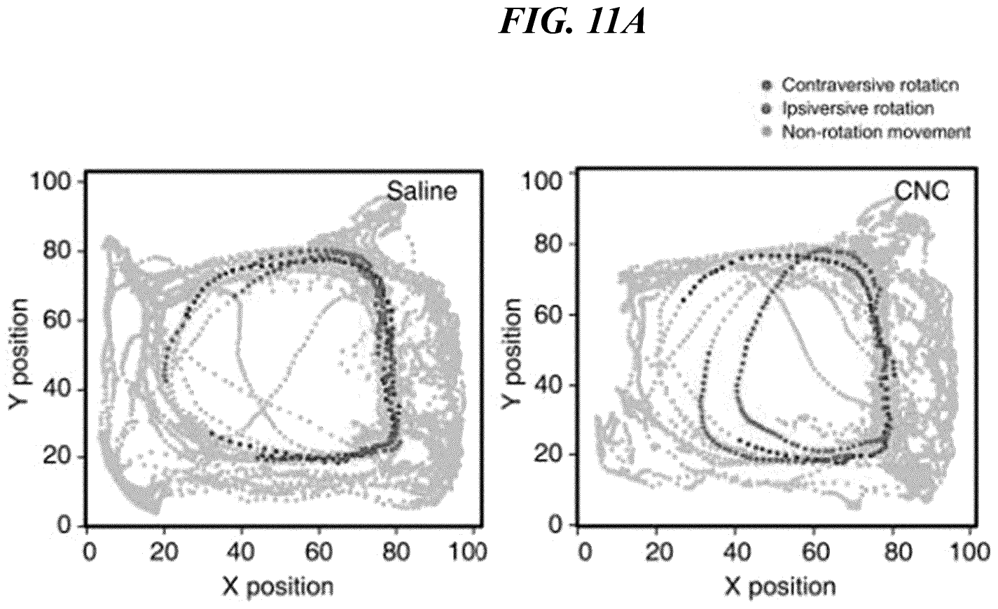

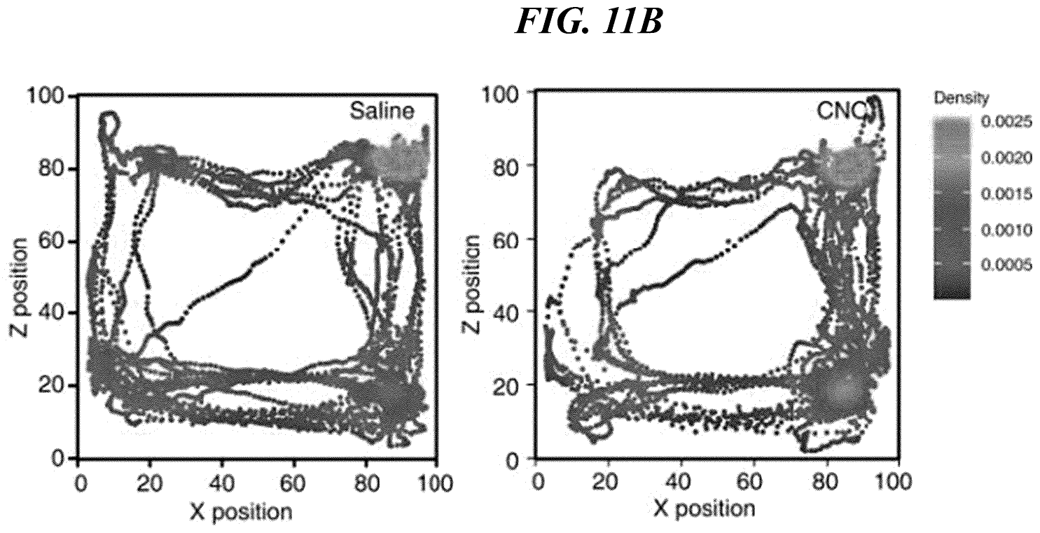

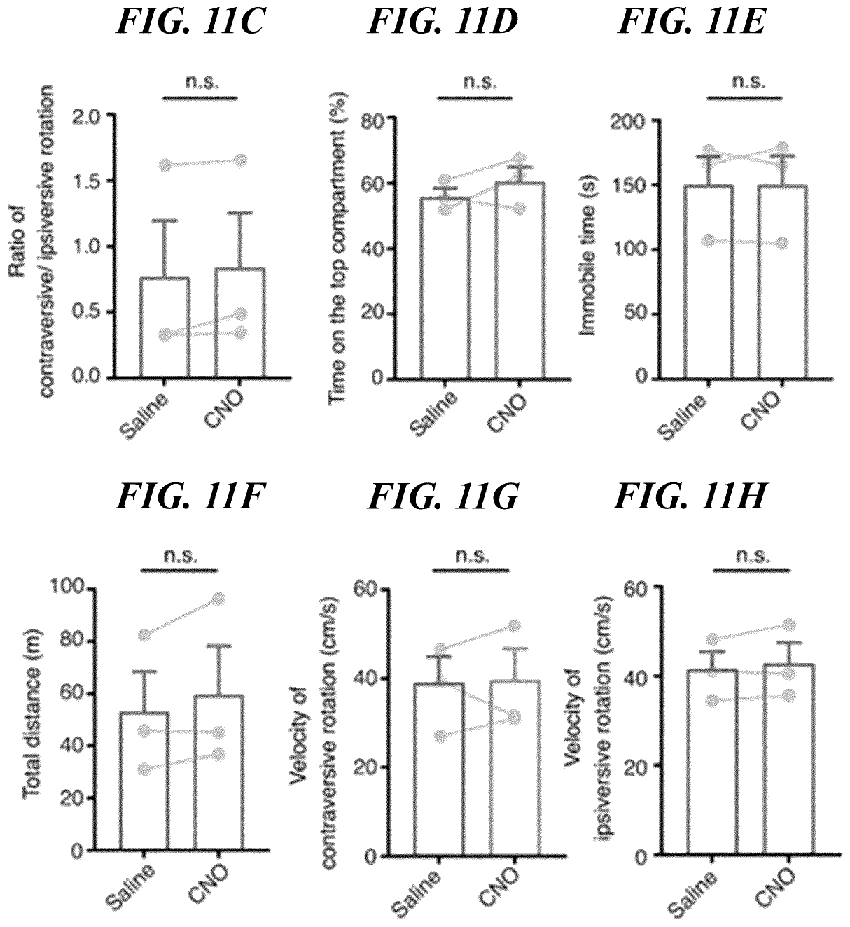

In behavioral tests, intracranial infusion into the dorsomedial caudate or systemic infusion of CNO in monkeys receiving hM3Dq or rM3Ds effectors, respectively, elicited drastic increases in contraversive rotations FIG. 10B, 10Q-10S,). After CNO treatment a significant reduction in the animals' residence time on the top compartment of the observation cage and an increase in the speed of contraversive rotations were observed (FIG. 8A-8B, 10T-10W). No significant differences were found for the speed of ipsiversive rotations, total distance traveled, or immobile time (FIG. 8C-8H) and CNO did not induce significant behavioral changes in naïve monkeys that did not receive viral injections (FIG. 11A-11H). Together, these results clearly show that the toolkit we developed can precisely isolate and efficiently activate direct pathway projection neurons in primates.

To test the effectiveness of the newly developed retrograde tool system in primate models, AAV8R12-G88P3-mCherry was injected into the SNr of a cynomolgus macaque (Macaca fascicularis). Since a cynomolgus macaque's brain is approximately 180 times larger than that of a mouse, the injections were delivered to a grid of 9 spots that cover the majority of the target structure.

To guide the virus injection in SNr, a guide grid of multiple holes spaced by 1 mm was installed on each subject vertically above the SN. By filling vitamin E in these holes, the accurate coordinates for injection could be obtained from the T1-weighted MRI images (3T Tim Trio scanner, Siemens). Virus injections were performed in nine sites covering the whole SNr, and a total volume of 27 μL virus was unilaterally injected in the right SNr at the speed of 300 nL/min.

To intracranially administrate drug and/or perform electrophysiological recording, a recording chamber covering from anterior caudate to posterior GPi was fixed on the skull with 6 titanium screws and dental cement. Each subject was allowed to recover from the surgery for at least 6 weeks prior to further studies.

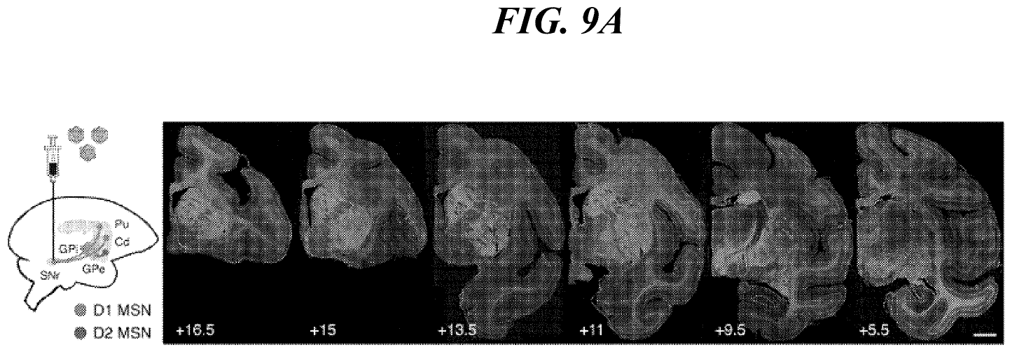

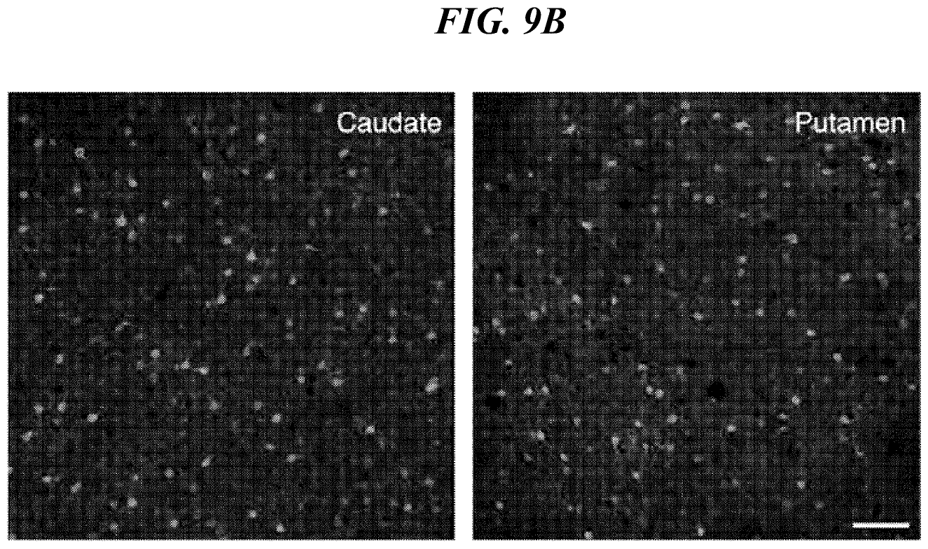

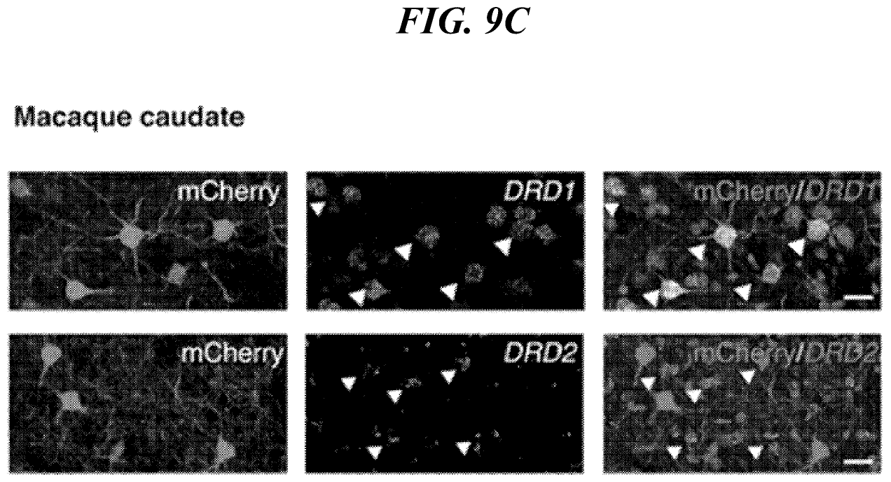

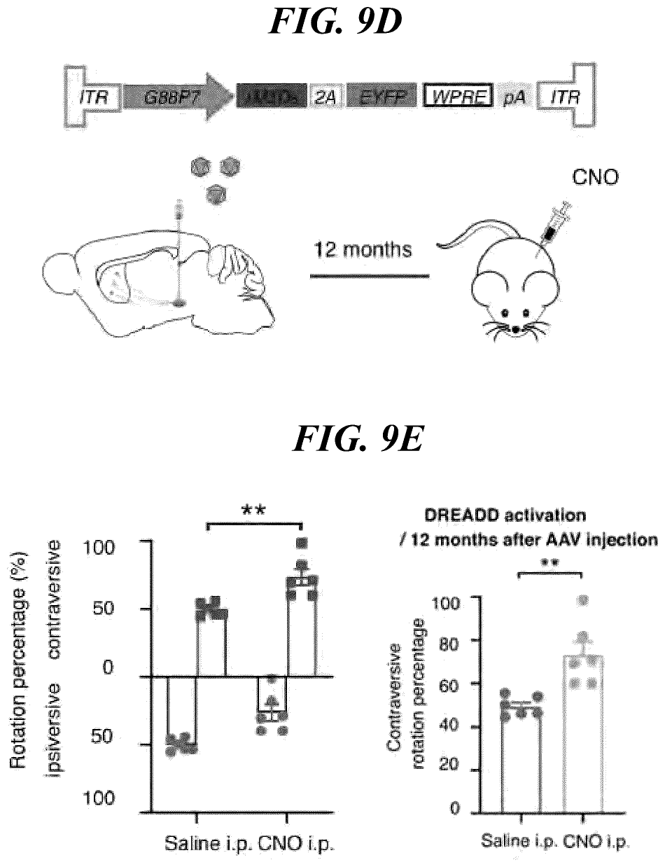

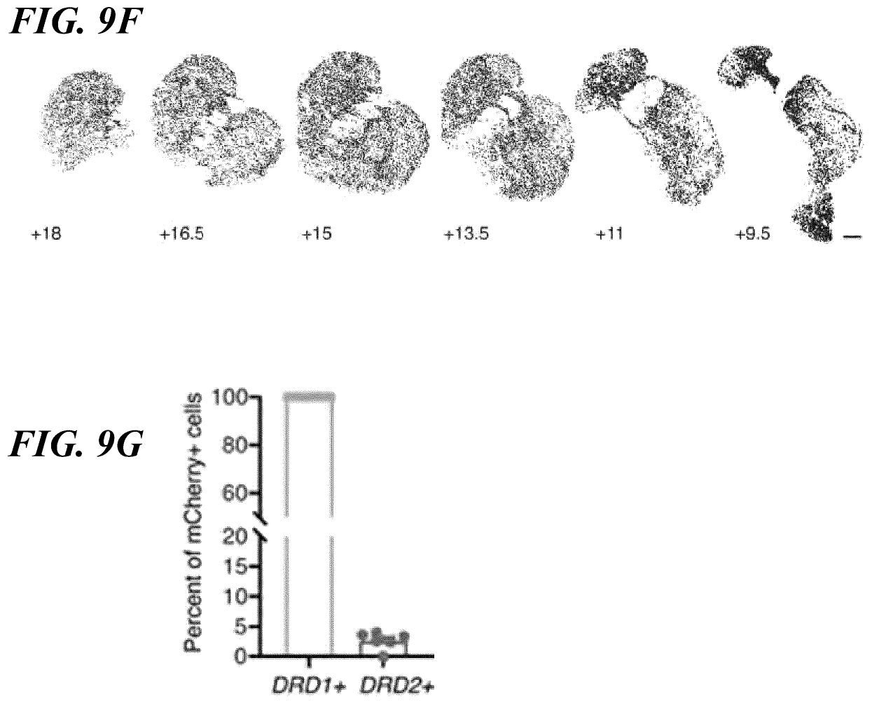

Macaques which received nigral injections of AAV8R12-G88P3-HA-hM3Dq or AAV8R12-G88P7-rM3Ds-2A-EYFP following CNO infusions spent less time in the higher portions of the observing cages than animals administered with saline infusions (FIG. 8A-8B). In situ hybridization analyses indicated that labeled neurons were exclusively D1 MSNs, confirming the labeling specificity of the retrograde AAV in macaques (FIG. 9C). Overall, about 20.55% of D1 MSNs were labeled (FIG. 9A-9G). Activation of BG direct pathway in mice 12 months after nigral delivery of AAV8R12-G88P7-rM3Ds-2A-EYFP and delivery of CNO via i.p. injection (FIG. 9D) and the Percentage of rotational behavior (ipsiversive rotations and contraversive rotations) was quantified (FIG. 9E), n=6 mice per group. The effectiveness of D1-MSN retrograde labeling system in macaque models, was assessed by unilaterally injecting AAV8R12-G88P3-mCherry into the SNr of a cynomolgus macaque (Macaca fascicularis). Robust labeling of projection neurons was detected in the caudate and putamen with minimal labeling elsewhere (FIG. 9K).

Next, AAV8R12-G88P3-HA-hM3Dq was injected into the SNr unilaterally. Intracranial infusion of CNO into the dorsomedial caudate elicited drastic increases in contraversive rotations. (FIGS. 10A and 10C). Intriguingly, increased contraversive rotations were also observed in macaques that received unilateral AAV8R12-G88P7-rM3Ds-2A-EYFP injections into the SNr 2.5 years after the nigral injections but not control animals after systemic CNO infusions (FIG. 10A-10C, FIG. 11A-11H), indicating the ability to effect a long-lasting expression of chemogenetic effectors by retrograde viral tracers and precision in isolation and efficiency in activating direct pathway projection neurons in primates. A significant increase in the speed of contraversive rotations and a reduction in the time animal residing on the top compartment of the observing cage after CNO treatment was also observed (FIGS. 10D and 10G, FIG. 11A-11H). Moreover, an increase in the muscle tone of the contralateral biceps brachii was observed with electromyogram recordings following CNO infusions. No significant differences were found for the immobile time, total traveling distance, or speed of ipsiversive rotations (FIG. 10E, 10F, and 10H). Electrophysiological recordings confirmed an increase in neuronal activity in the caudate following CNO infusions (FIG. 12A-12F). These results clearly show that the toolkit precisely isolates and activates selective projection neuron subtypes in primate models.

### Example 5—Chemogenetic Activation of the Direct Pathway Reversed Parkinsonian Symptoms in Rodent PD Models

Rodent Parkinson's disease mice models were created through bilateral injection of 6-OHDA using the same methods as described in virus injections (Example 3). A total volume of 1 ul 6-OHDA (5 mg/ml, dissolved in sterile saline containing 0.02% ascorbic acid, Sigma) was injected into the striatum at a speed of 100 nL/min. The coordinates for striatum were 0.5 mm anterior, 1.5 mm lateral, 3.2 mm ventral to the bregma. A premedication of desipramine (25 mg/kg, Sigma) was administered to animals prior to injections of 6-OHDA, in order to increase the selectivity and efficacy of 6-OHDA-induced lesions. Mice were supplemented with DietGel (ClearH2O) for one-week post-surgery. All staining and behavioral experiments were performed at least 14 days following surgery, when the amount of dopamine depletion was maximal and stable.

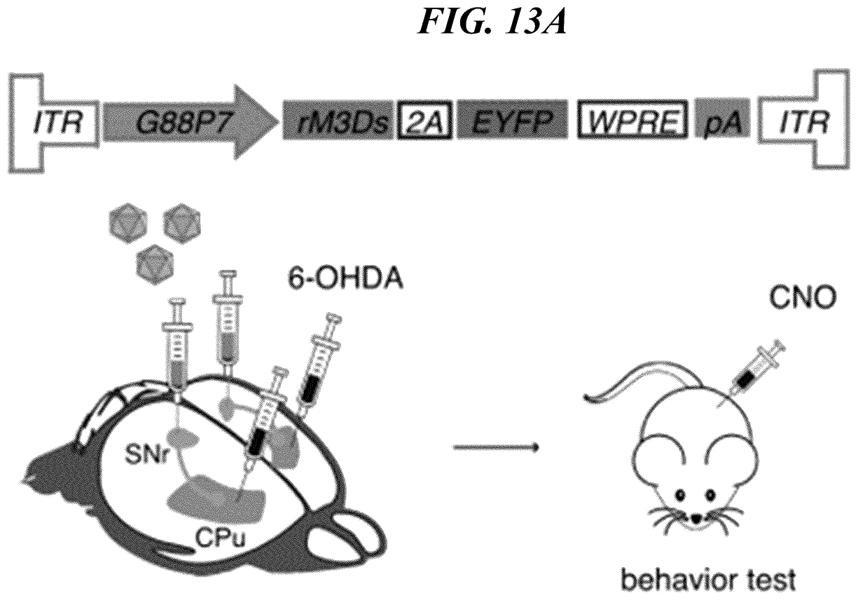

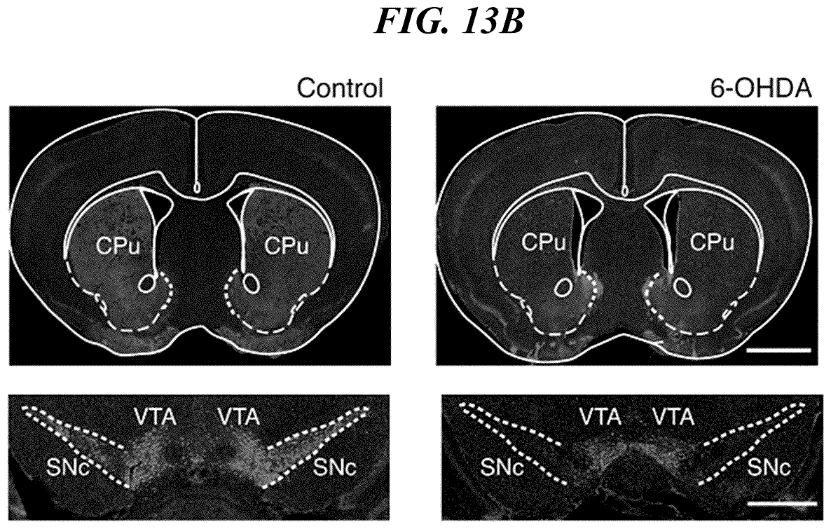

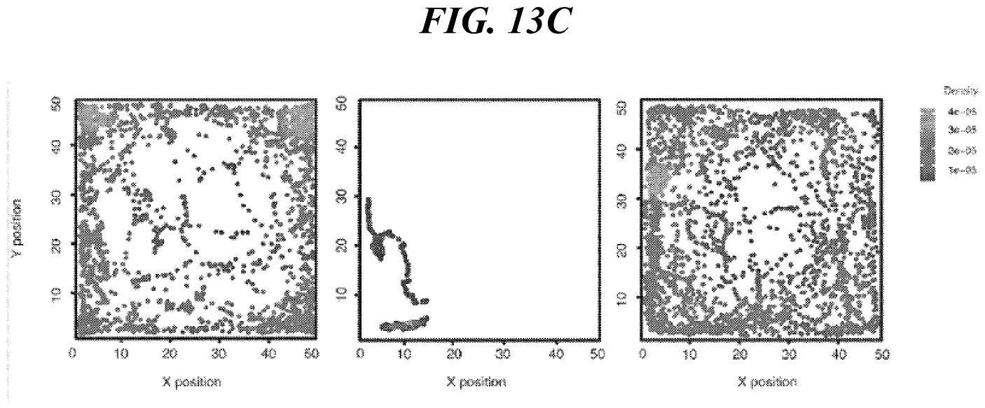

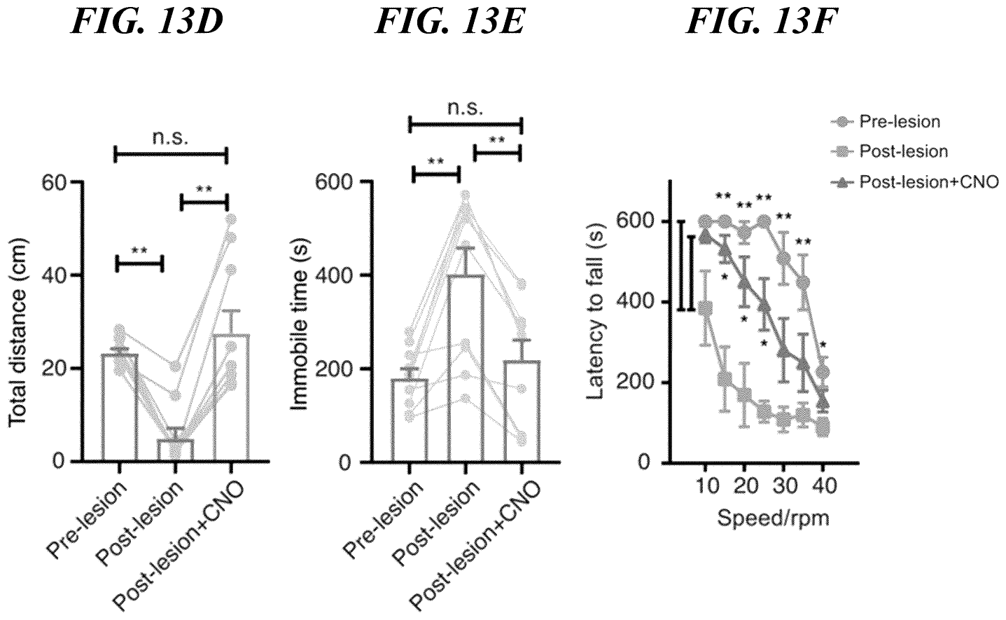

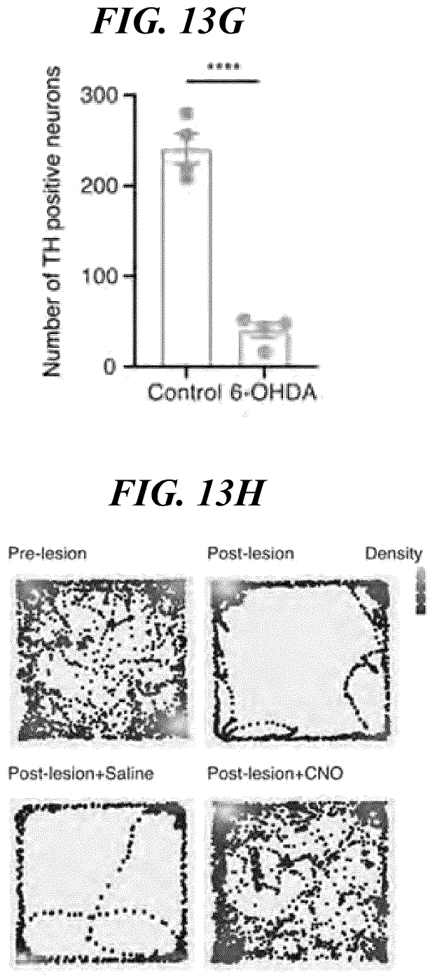

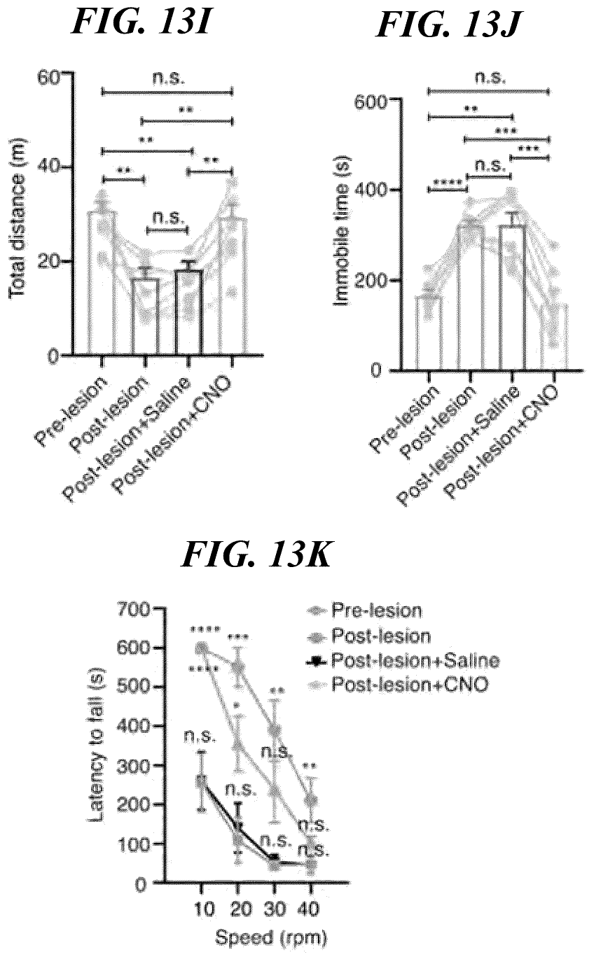

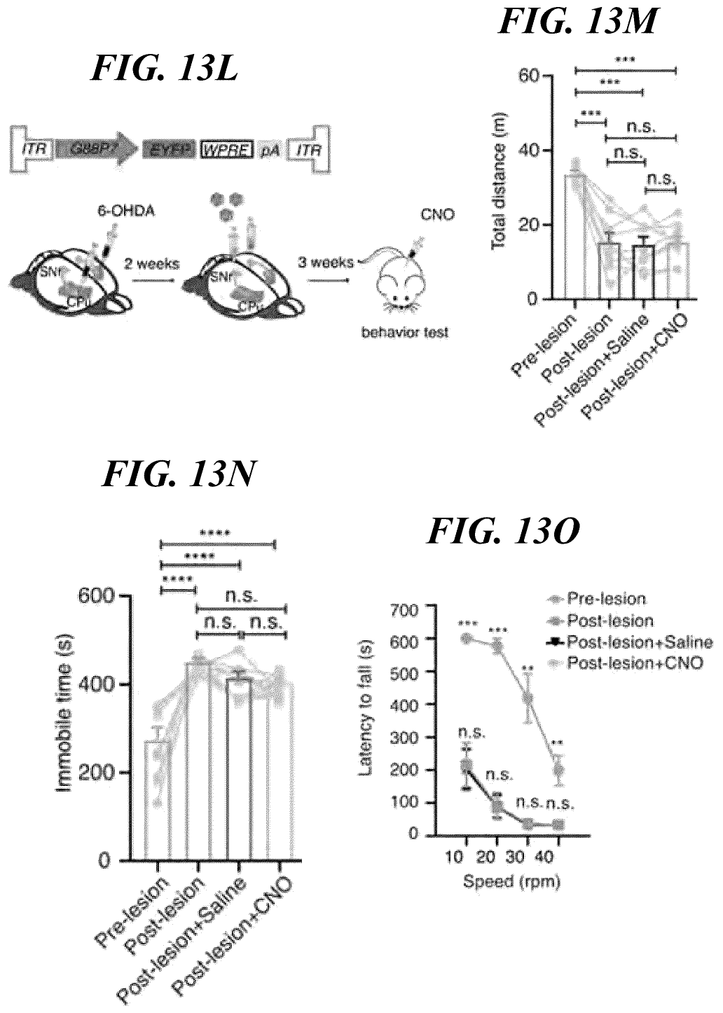

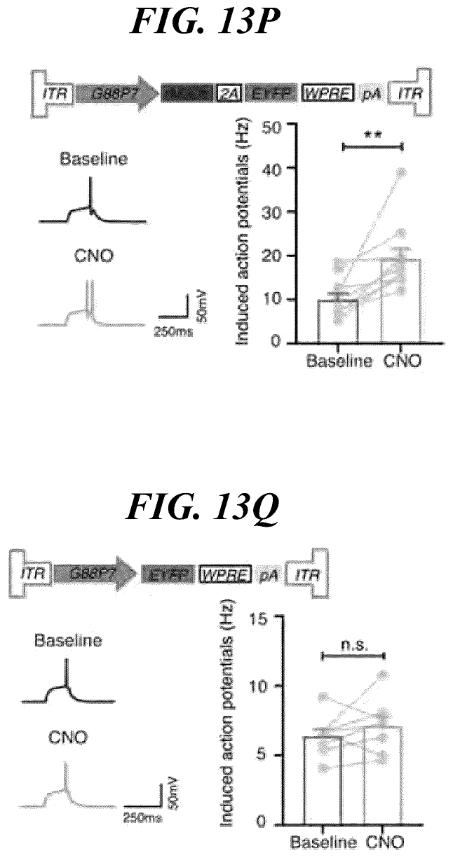

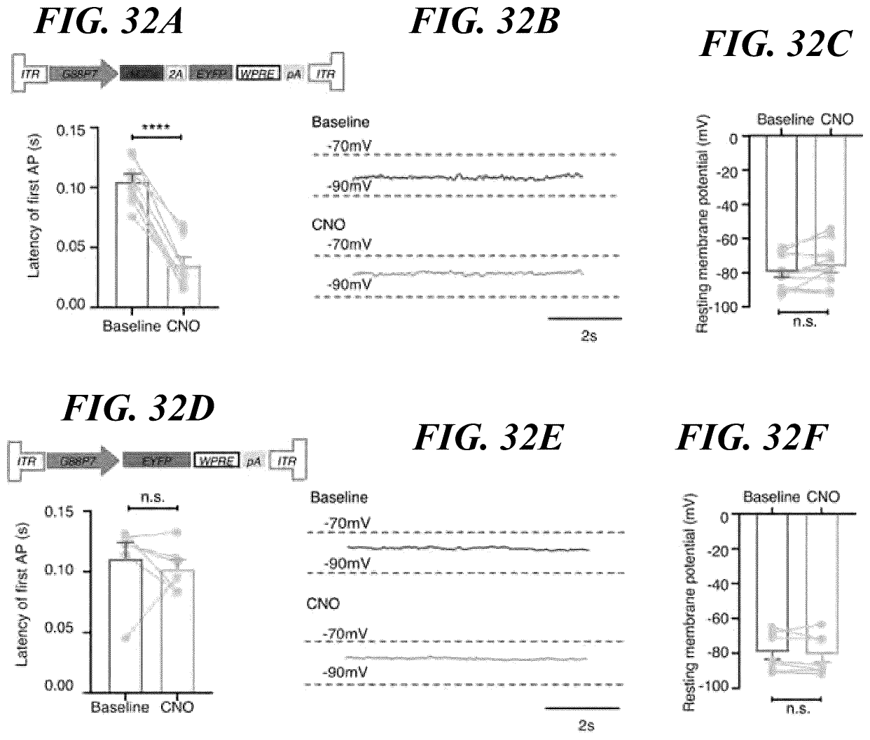

To investigate the efficacy and safety of D1-MSN specific neuromodulation strategy, AAV8R12-G88P7-rM3Ds-2A-EYFP was injected into the SNr of adult C57/BL6 mice. 6-OHDA was administered bilaterally into the striatum (FIG. 13A). TH immunohistochemistry revealed largely diminished dopamine innervation in the striatum and a great loss of dopamine neurons in the SNc (FIG. 13B, 13G). Analyses of spontaneous movement in an open field revealed that systemic CNO delivery, which selectively activates D1 MSNs, drastically reversed the dyskinesia-like phenotype of PD mice (FIG. 13C-13D, 13H-13I). Furthermore, CNO partially rescued the motor skill deficit of PD mice in a rotor-rod test (FIG. 13E, 13J). CNO partially rescued the motor skill deficit of 6-OHDA treated mice in a rotarod test (FIG. 13F, 13K). Nigral injection of AAV8R12-G88P7-EYFP failed to alleviate parkinsonian phenotypes (FIG. 13C-13O). Slice whole-cell patch-clamp recordings revealed increased excitability in D1-MSNs transduced by AAV8R12-G88P7-rM3Ds-2A-EYFP, but not AAV8R12-G88P7-EYFP (FIG. 13P-13Q, 32A, 32D), without affecting basal firing rate and resting membrane potential in either group (FIG. 32B-32C, 32E-32F).

These results demonstrated that targeted activation of the basal ganglia (BG) direct pathway with AAV-mediated retrograde scheme can effectively antagonize parkinsonian-like symptoms in rodent PD models.

### Example 6—Chemogenetic Activation of the Direct Pathway Reversed Parkinsonian Symptoms in Monkey PD Models

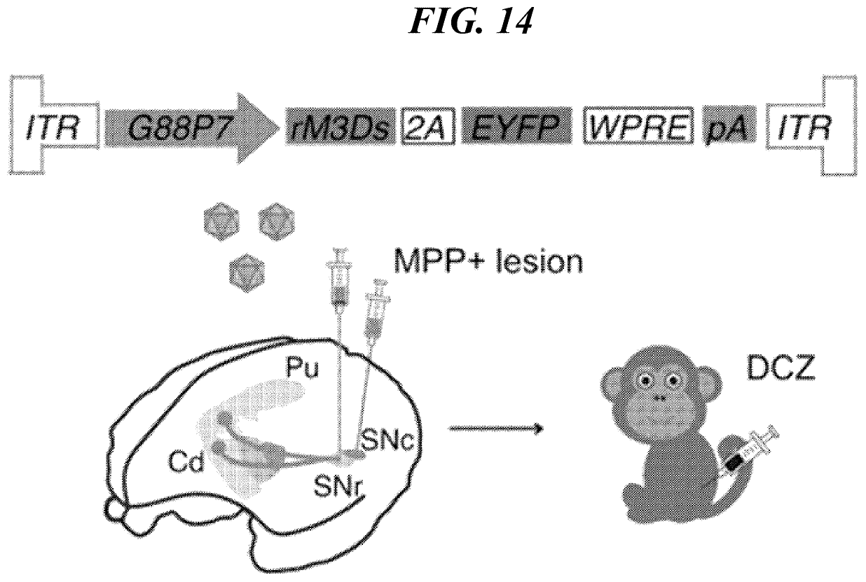

Parkinsonian conditions in macaque monkeys were established by unilaterally injecting 1-Methyl-4-phenylpyridinium (MPP+) into the SNc (FIG. 14). MPP+ was unilaterally injected using the same methods as described before in virus injections. Drug injections were performed in five sites covering the whole SNc. A total volume of 10 ul MPP+ was injected into SNc at a speed of 50 nL/min. Monkeys were continuously monitored by veterinarians after lesion. Parkinsonian symptoms, such as bradykinesia and impaired balance could be observed immediately after lesion surgery. Stable Parkinsonian symptoms were observed for more than 12 weeks before an animal was used for the experiments.

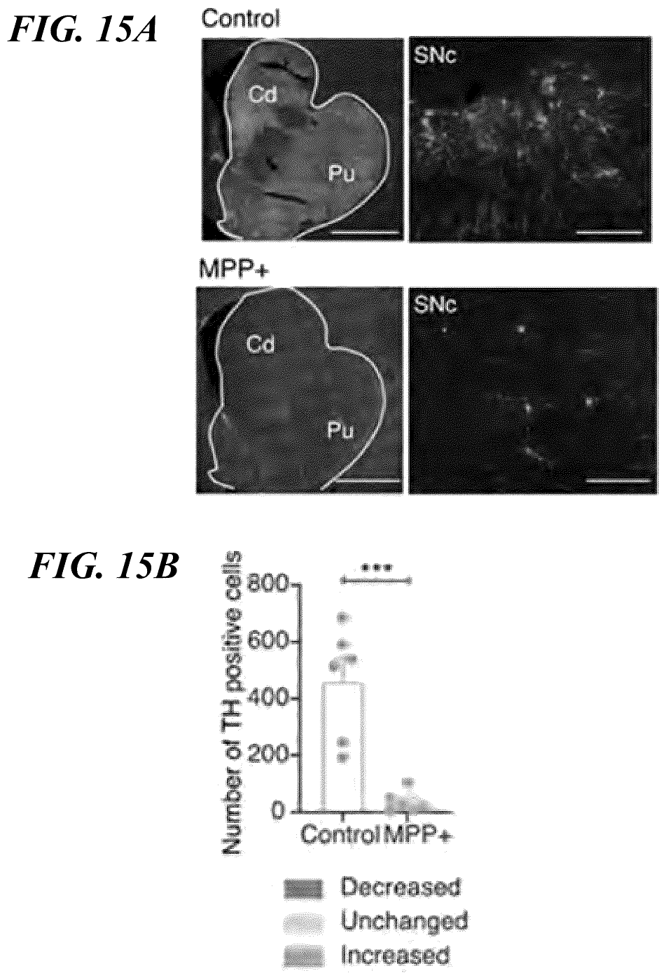

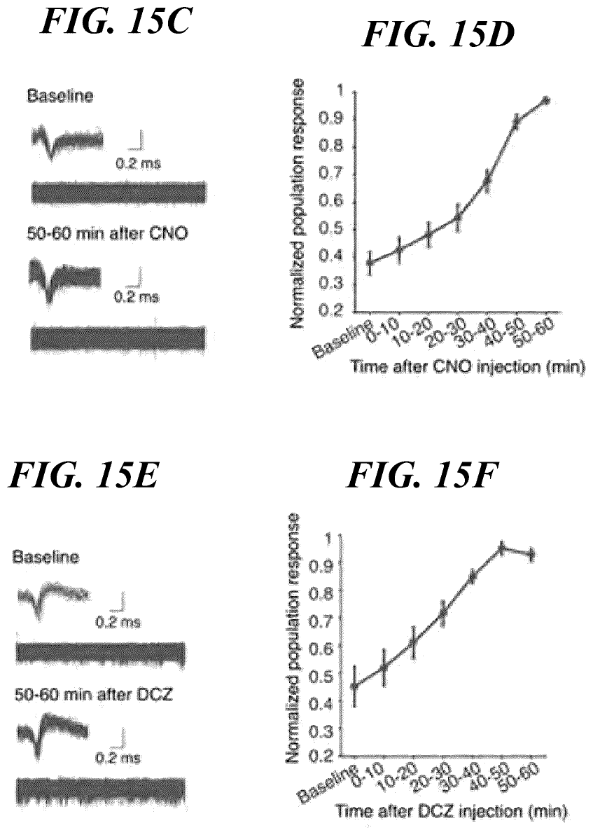

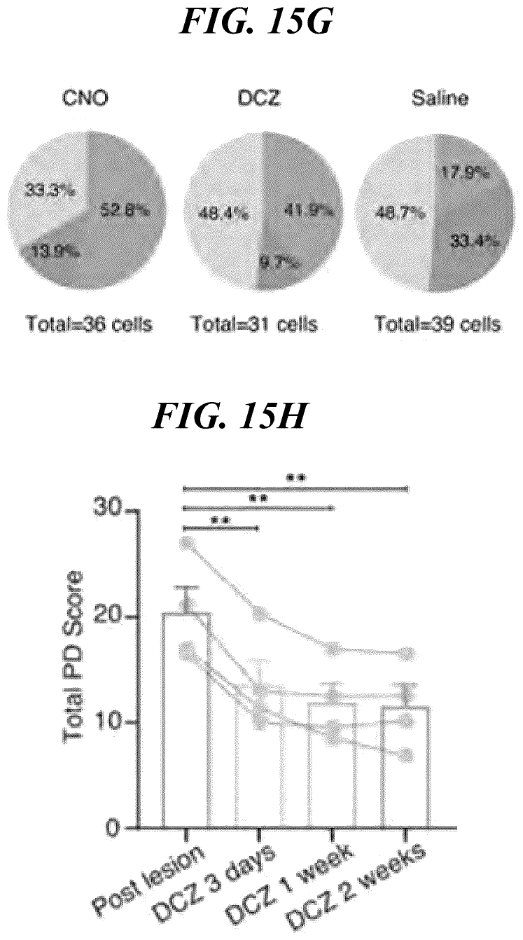

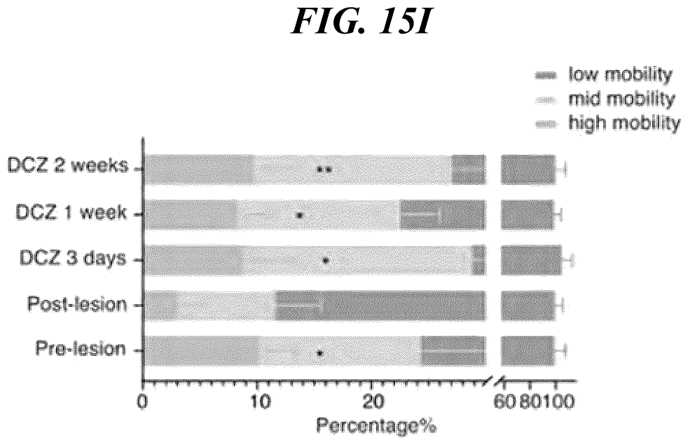

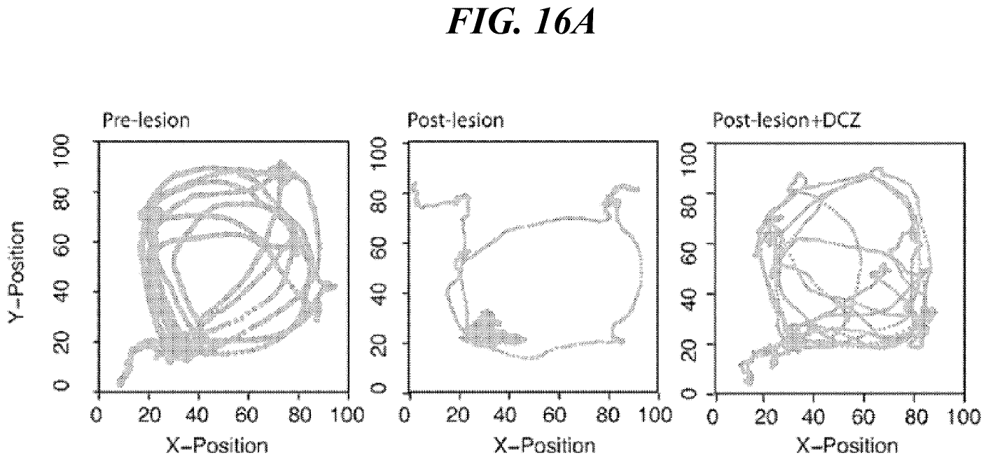

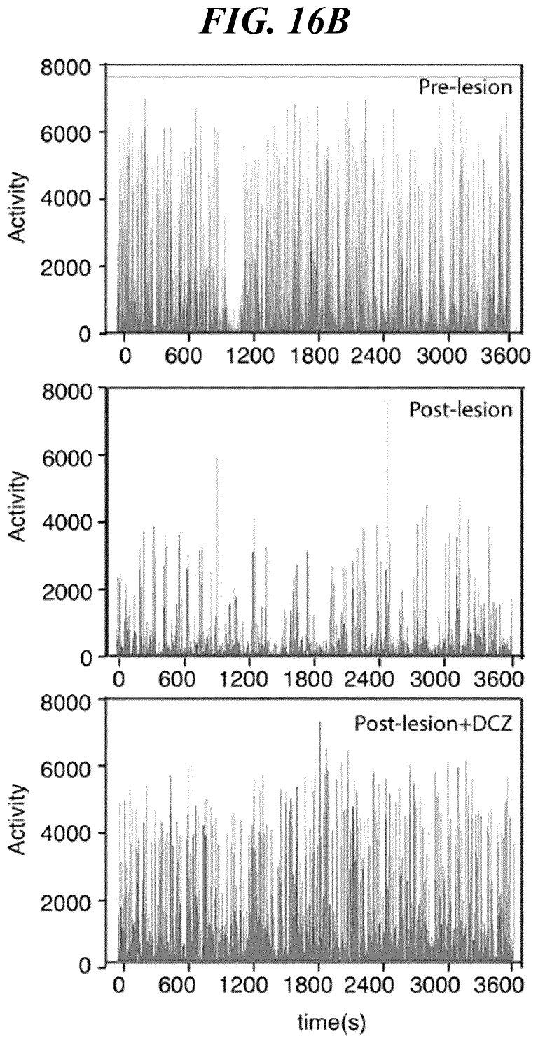

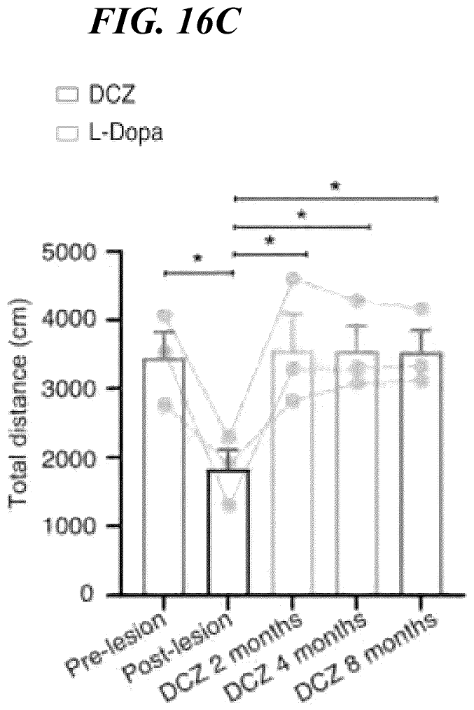

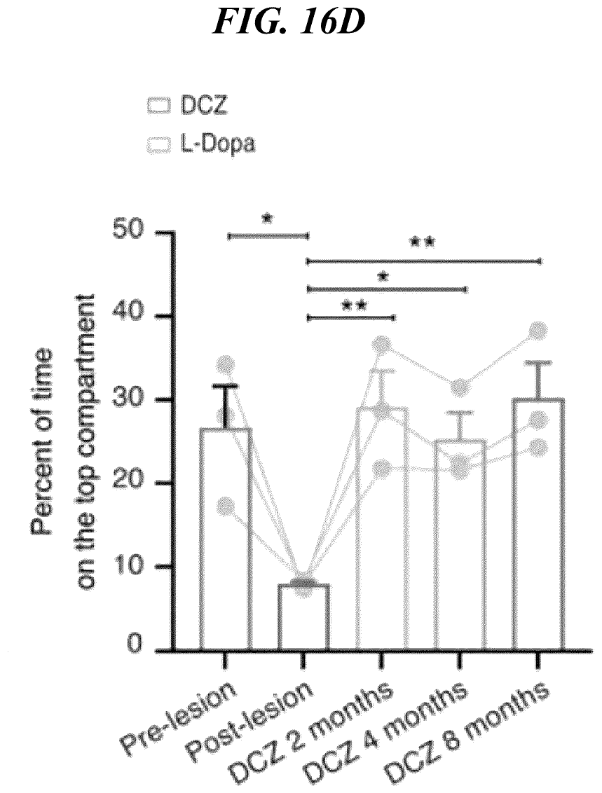

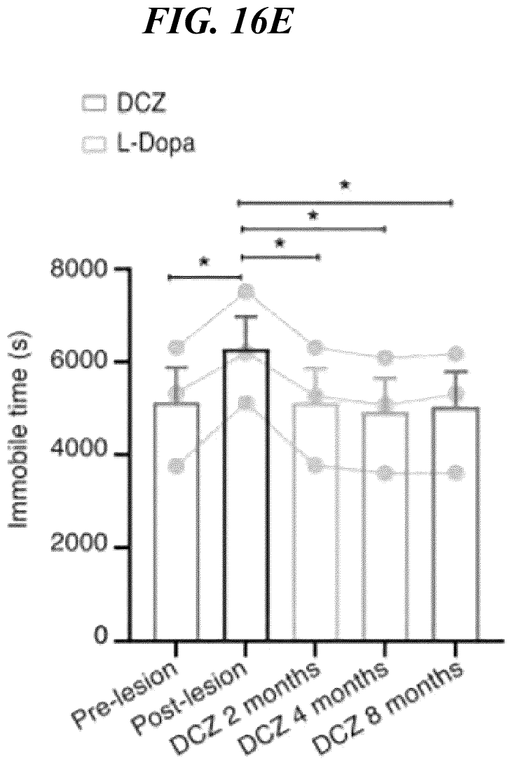

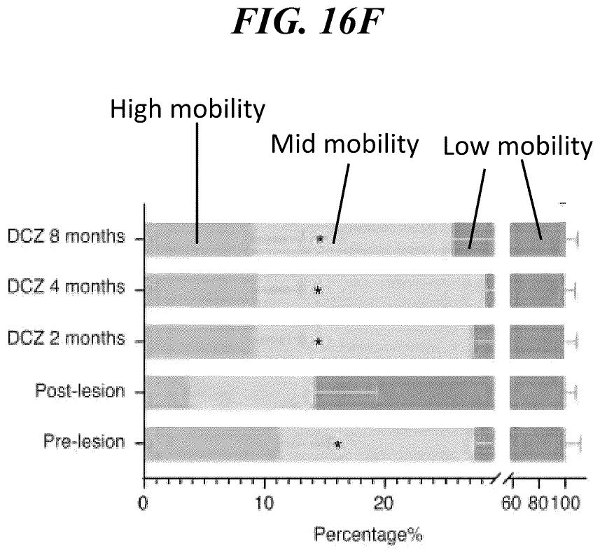

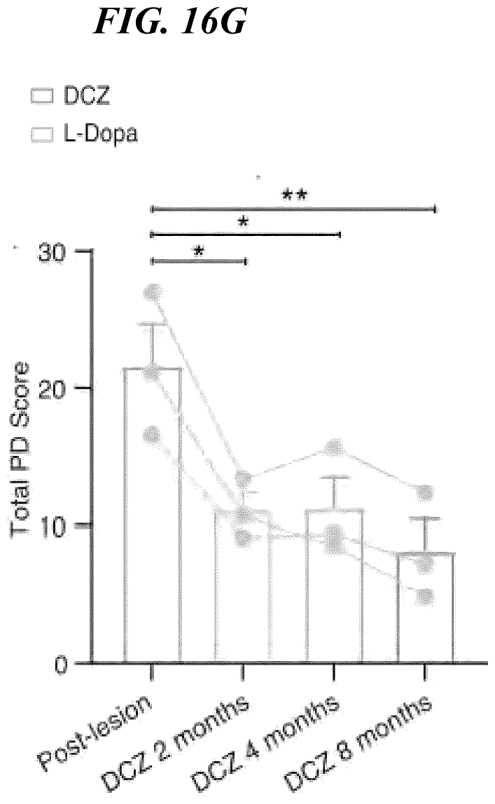

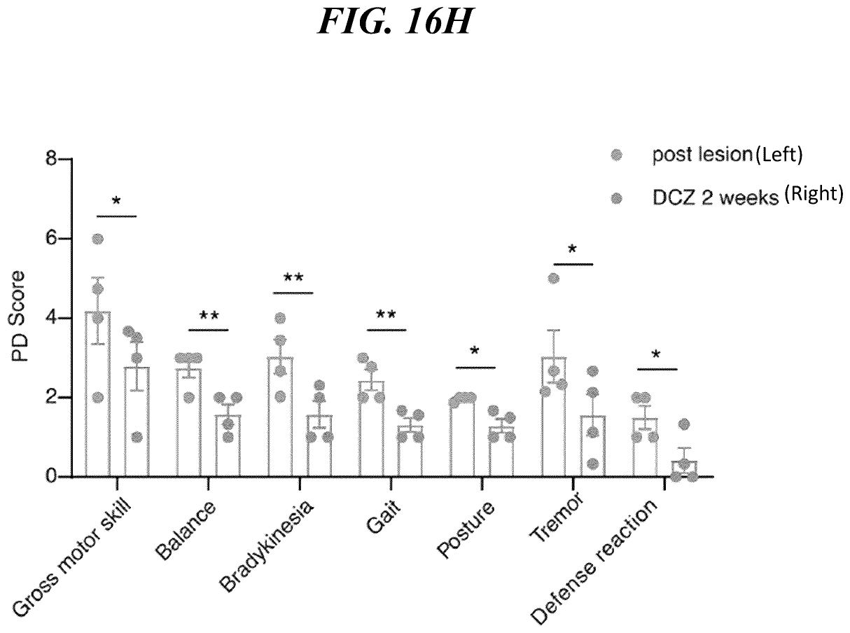

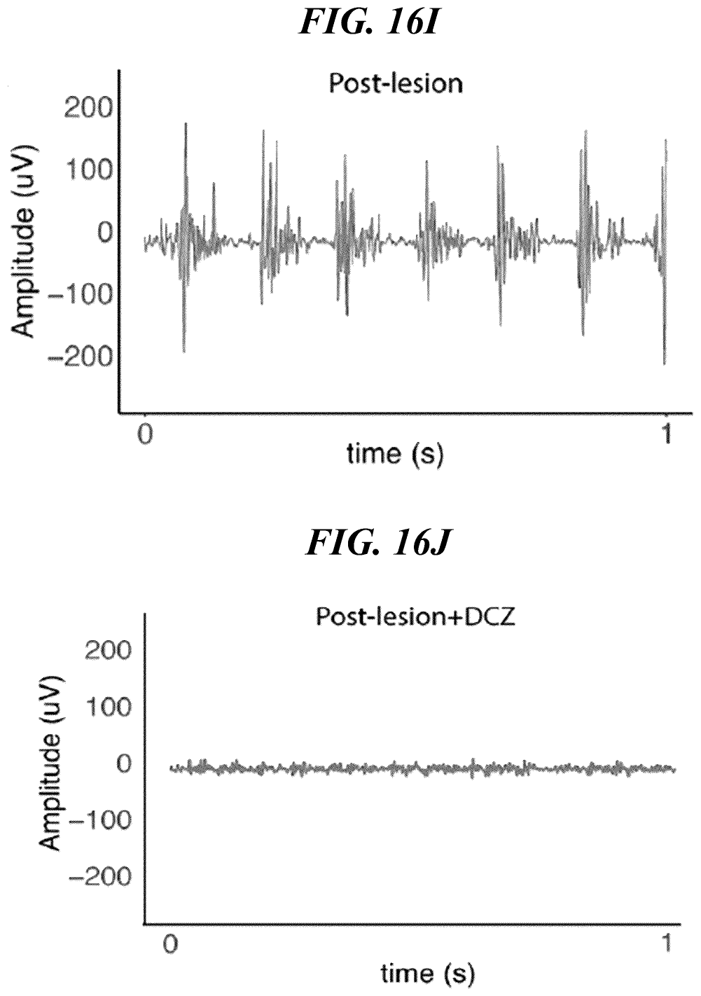

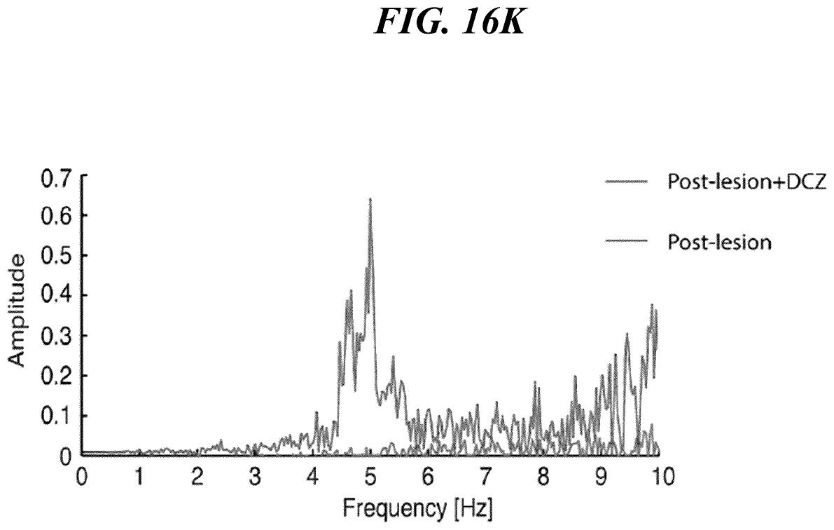

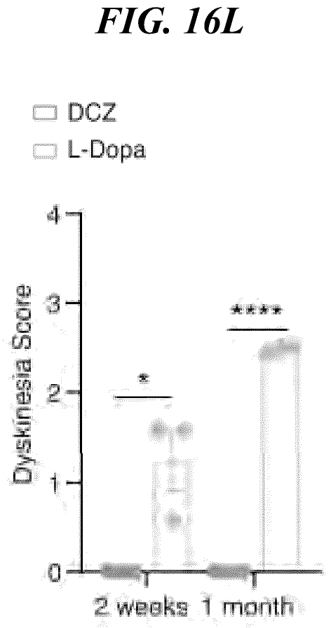

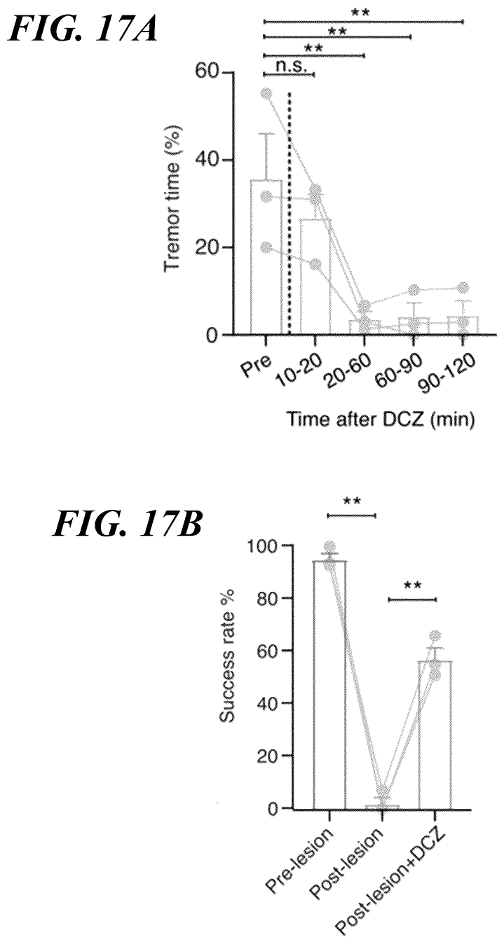

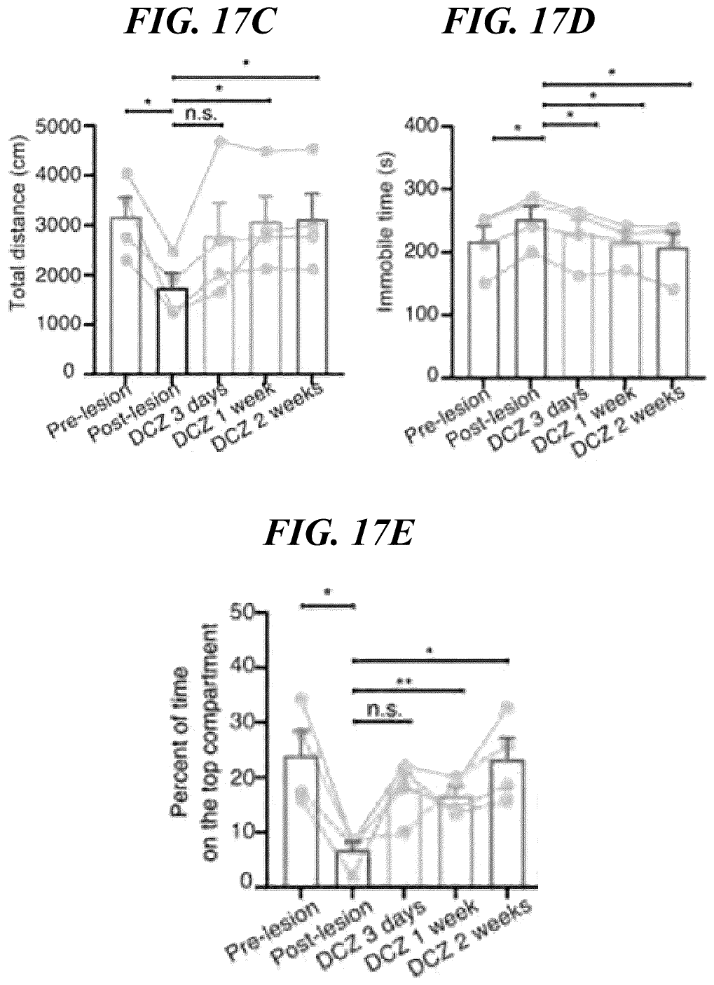

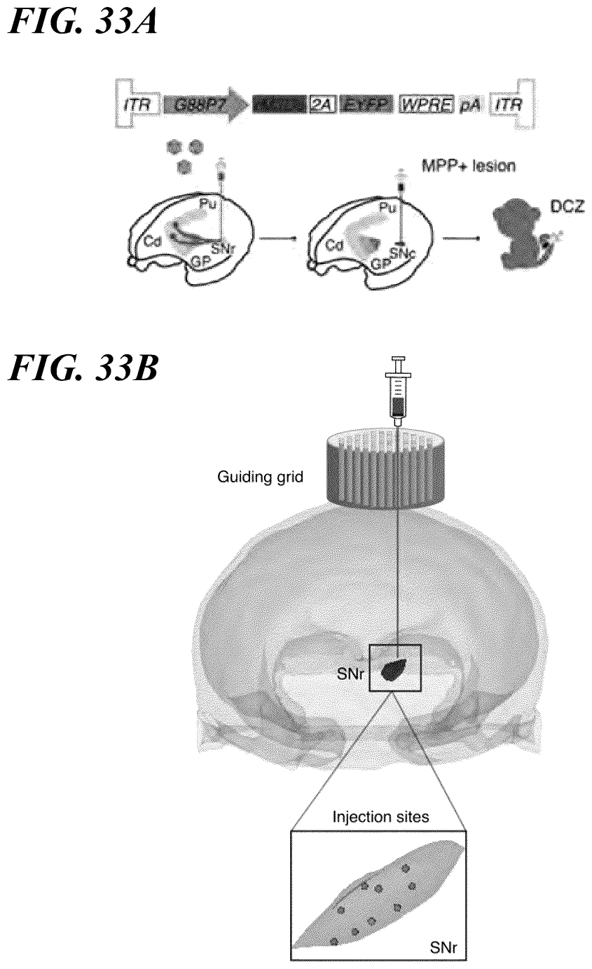

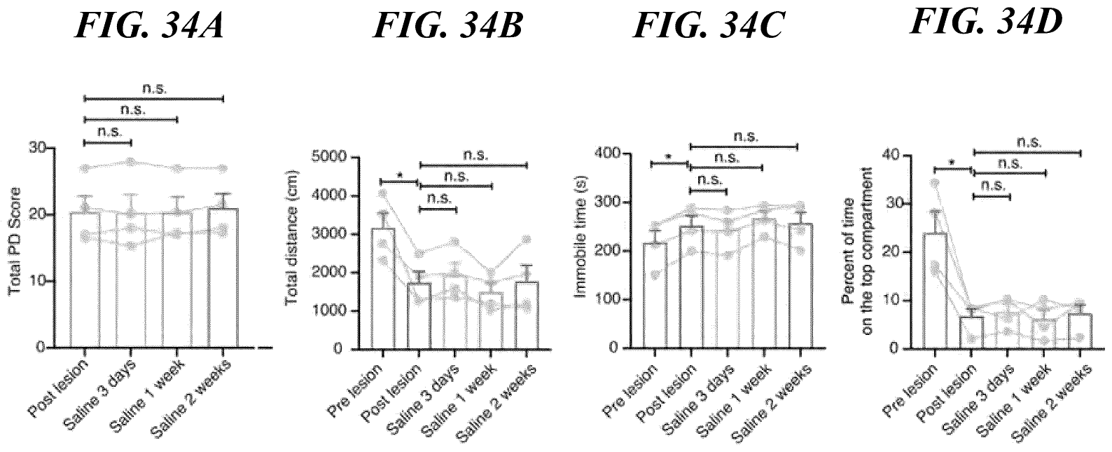

TH immunohistochemistry confirmed the loss of nigral dopamine neurons and their fibers in the caudate and putamen (FIG. 15A-15B). Stereotaxic injections of AAV8R12-G88P7-rM3Ds-2A-EYFP were performed into 6 sites of the SNr of adult cynomolgus macaques (FIG. 14). Monkeys with MPP+ injections displayed characteristic PD-like symptoms, including bradykinesia, tremor, rigidity, and postural abnormalities (FIG. 16A-16K). However, the D1-MSN targeted manipulation approach did not elicit dyskinesia-like behaviors (FIG. 16L). In some instances, stereotaxic injections of retrograde AAV8R12 were performed into 9 sites of the SNr of adult cynomolgus macaques (FIG. 33A-33B). MPP+ was then unilaterally injected into the SNc and the loss of nigral dopamine neurons in the SNc and their fibers in the caudate and putamen was observed via TH immunohistochemistry (FIGS. 6B and 6C).

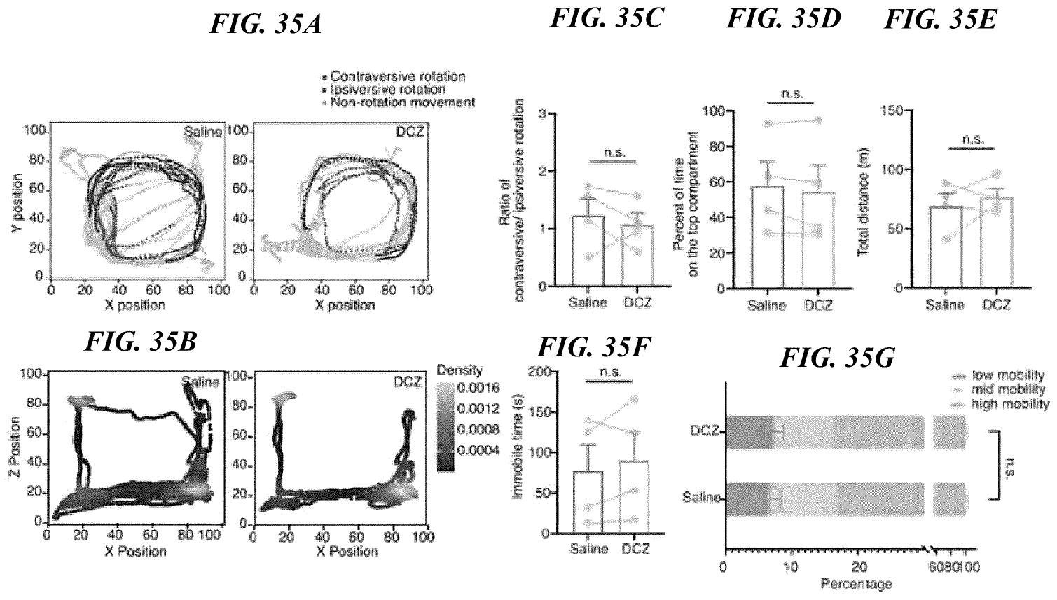

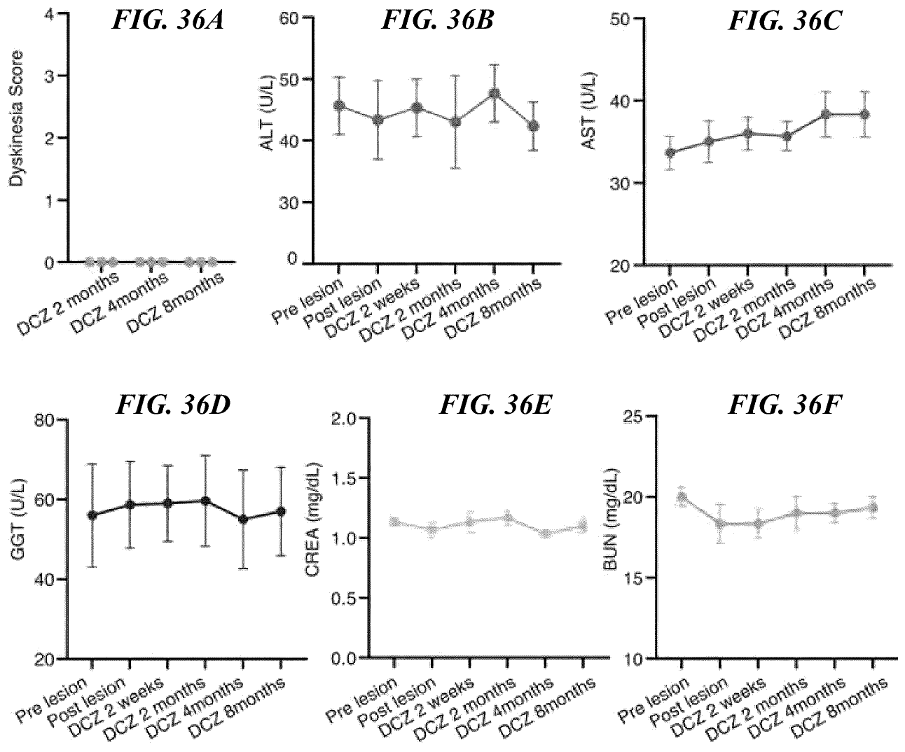

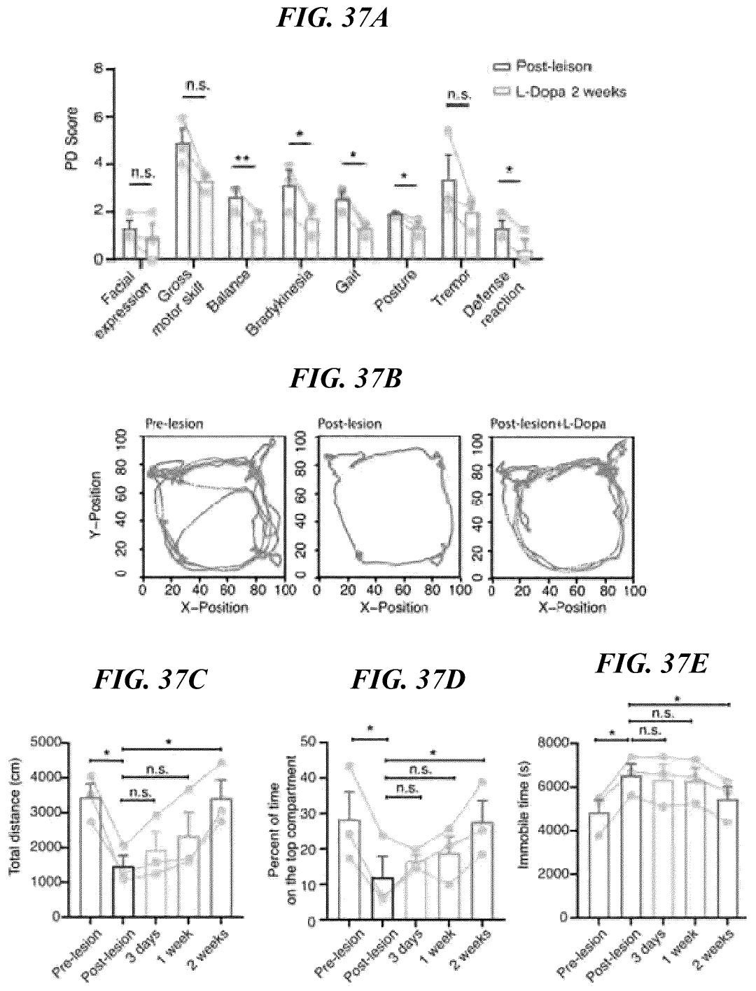

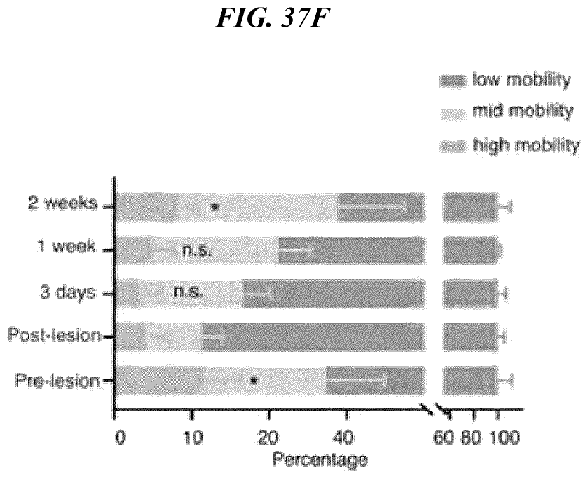

Intriguingly, many of the symptoms were greatly reversed after systemic deschloroclozapine (DCZ) administration. DCZ is a potent brain-penetrable agonist for rM3Ds with reduced off-targeting binding compared to CNO, and was administered to activate the DREADD system in the macaque brain. In vivo electrophysiological recordings in anesthetized animals indicated that DCZ or CNO, but not saline, induced an increase in neuronal activity in MPP+ lesioned macaques (FIG. 15C-15G).Monkeys given MPP+ injections displayed characteristic PD-like symptoms, including bradykinesia, tremor, rigidity, and postural abnormalities. With systemic DCZ treatment, but not saline, we observed a reversal of canonical parkinsonian symptoms in all tested monkeys (FIG. 15H-15I, 16A, 16H-16K, 17A-17D, 34A-34D). First, an increase in spontaneous movement in an observation cage, close to animals' level of activity before MPP+ injection was observed (FIG. 15I, 16A-16F, 17C-17D). Second, tremor is great reduced or even eliminated after DCZ treatment (FIG. 16H-16K, 17A). Third, a significant recovery of motor skills by chemogenetic manipulation (here, activation of D1-MSNs) was observed (FIG. 16H, 17A-17B). Furthermore, the effective dose of DCZ (0.3 mg/kg) did not alter motor-related behaviors in naïve monkeys (FIG. 35A-35G). Of note, the alleviation of parkinsonian symptoms appeared to be consistent during an 8-month continuous treatment with DCZ (FIG. 16C-16G). In addition, animals were dyskinesia-free and the blood levels of common liver- and kidney-related factors remained stable during the treatment (FIG. 36A-36F). These data strongly indicate that our targeted circuit manipulation approach can effectively and safely reverse core symptoms of parkinsonian primates.

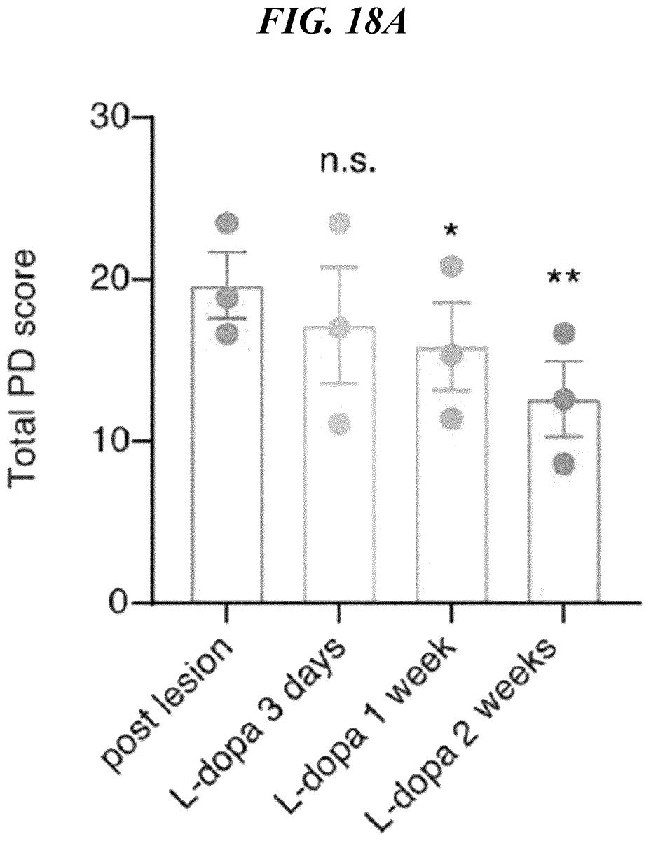

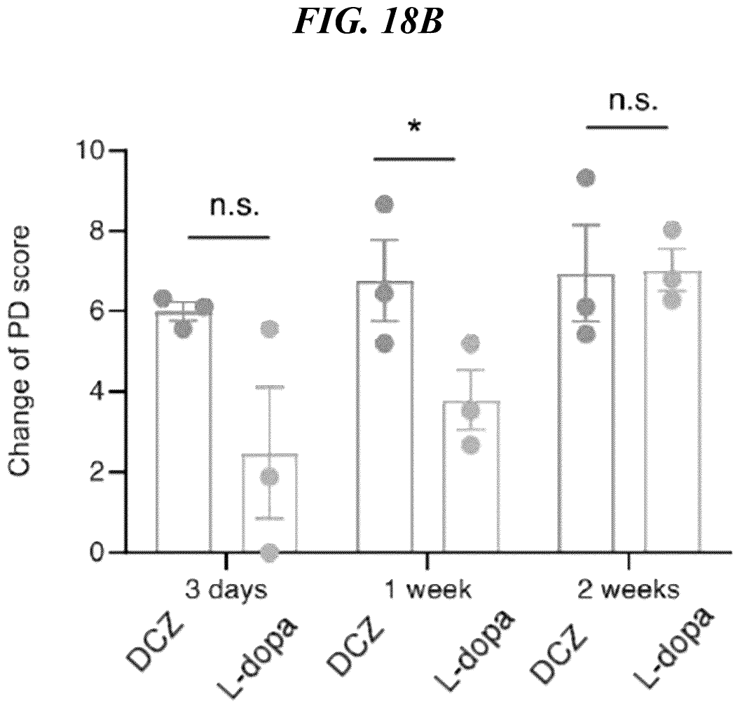

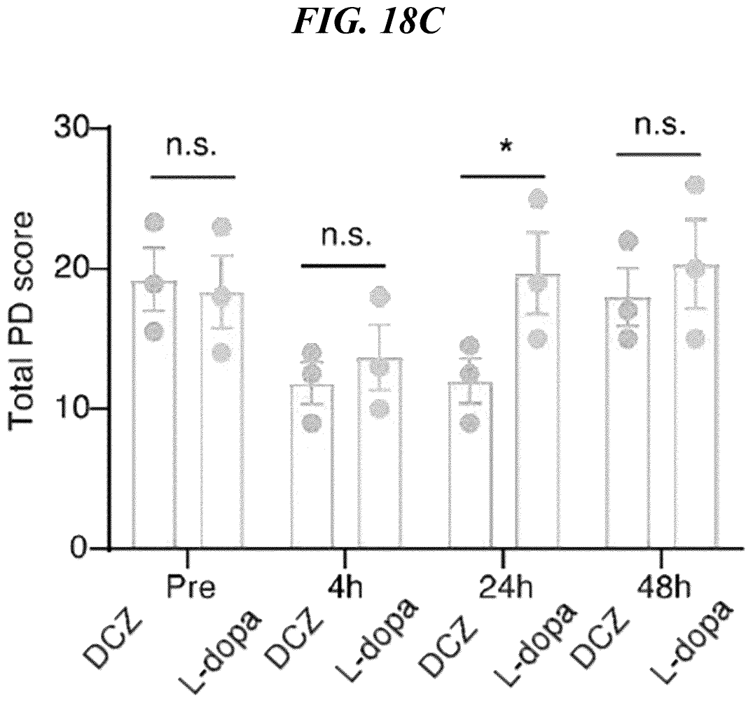

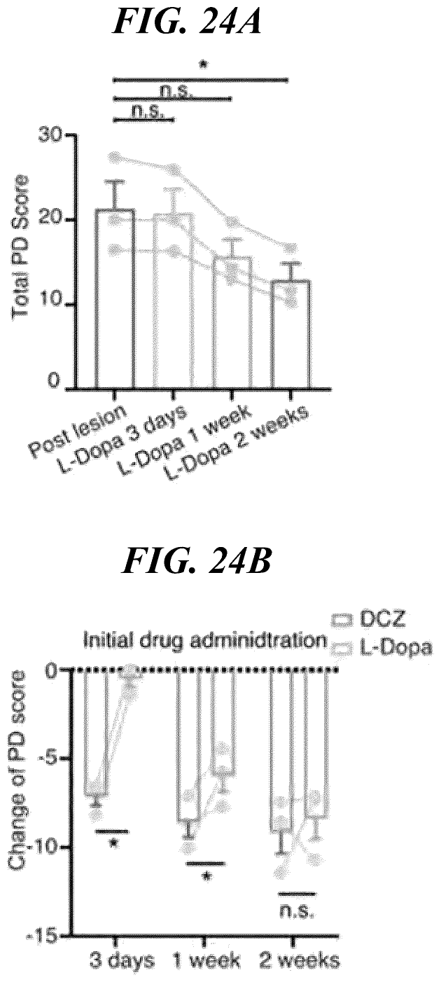

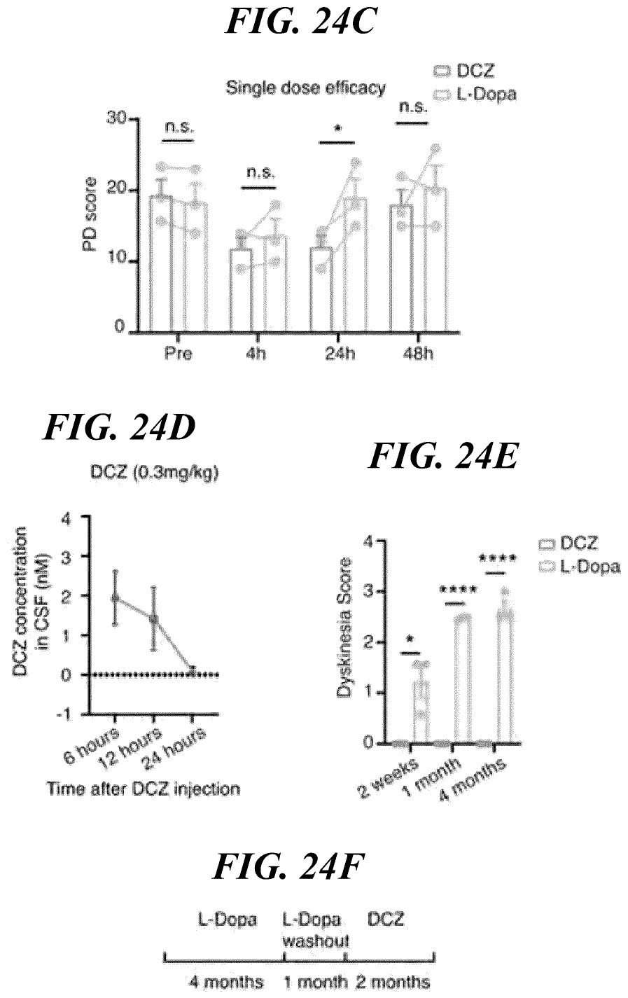

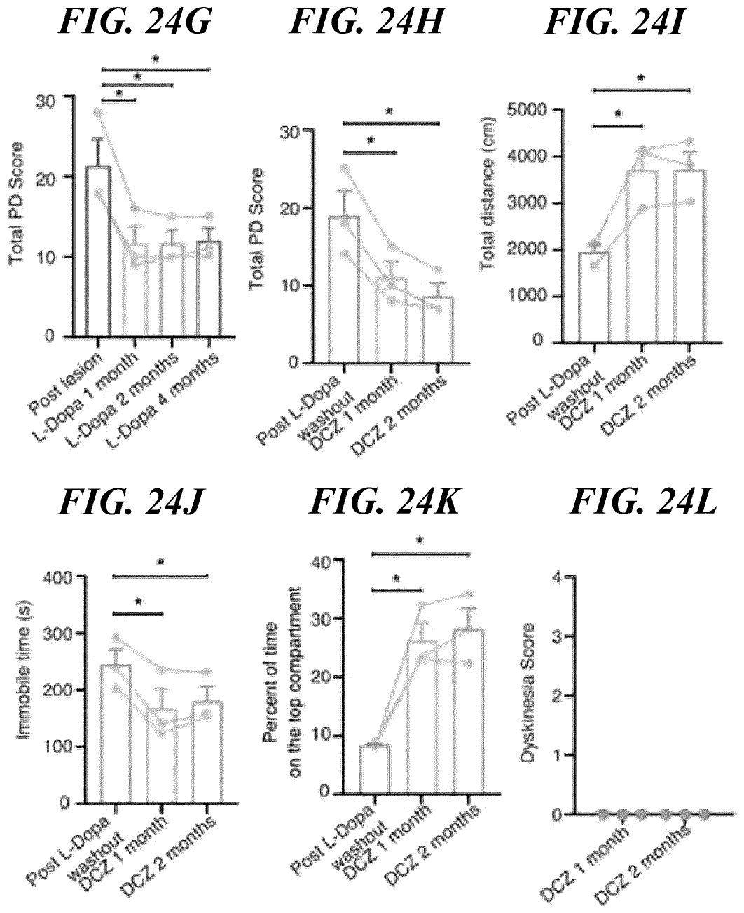

The efficacy of DCZ with levodopa, a first-line drug for treating PD patients was subsequently performed for comparison. Similar reversal of parkinsonian symptoms by the two chemicals was observed (FIG. 18A-18C, 24A, 37A-37F), but DCZ showed faster reversal of symptoms than levodopa. DCZ-mediated targeted chemogenetic circuit activation showed faster reversal of symptoms than L-Dopa during the initial phase of drug administration (FIG. 24B). Surprisingly, DCZ displayed greatly extended effective time compared with levodopa, exhibiting alleviation of symptoms 24 hr after drug administration (FIG. 18C). After the drug reached steady-state efficacy, DCZ extended the window of efficacy after each dose of treatment, alleviating symptoms for at least 24 hours after drug administration (FIG. 24C), much longer than the clinically observed therapeutic window of L-Dopa. Cerebrospinal fluid samples were collected 24 hours after drug infusion and no detectable levels of DCZ were found (FIG. 24D), implying a change in neural network dynamics or a significant effect by residual DREADD ligand in the brain. Furthermore, the D1-MSN targeted manipulation approach did not elicit dyskinesia-like behaviors, which was evident after long-term L-Dopa administration (FIG. 24E). An extended period (4 months) of prior L-Dopa administration did not affect the efficacy of DCZ treatment or the absence of dyskinesia (FIG. 24F-24L). Together, these results showcase the effectiveness of our approach in NHP PD models and strongly support its feasibility for treating PD in humans.

### Example 7—Seroquel of Novel DREADD Ligands for the Clinic

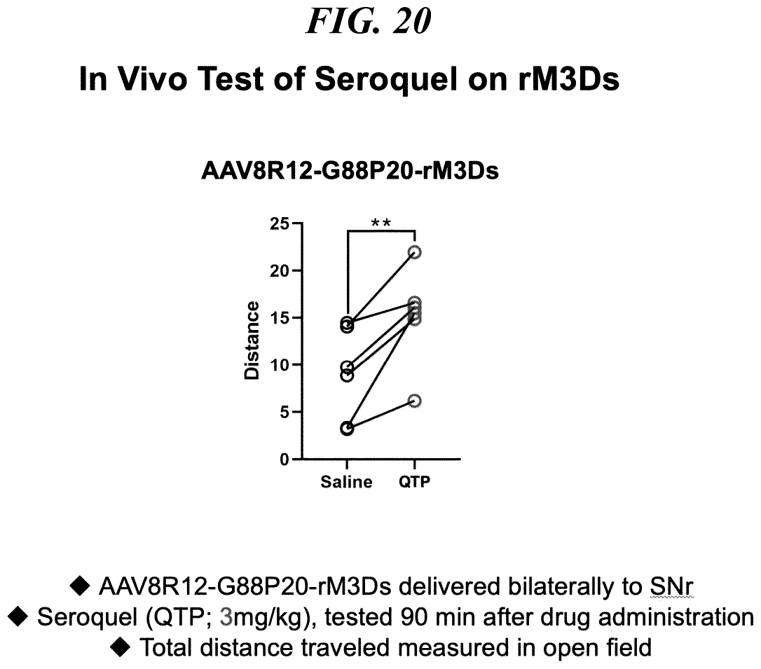

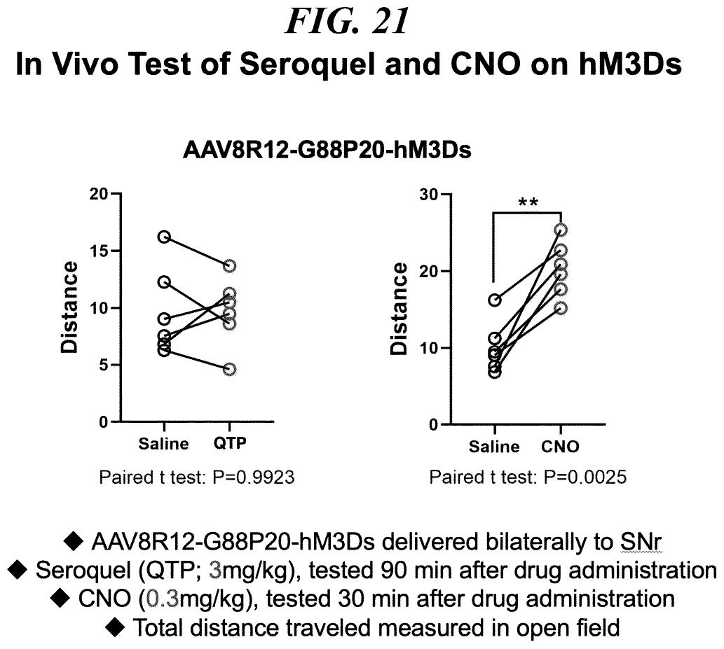

Parkinson's Disease patients receiving gene therapy would be well served by taking an FDA-approved ligand to activate the DREADD component the therapies described herein. Although clozapine (CNO), the activate metabolite of CNO, is a potential candidate for such a molecule, additional molecules with less clinical monitoring requirements and more favorable safety profiles were sought out. Using chemical structural analyses coupled with analyses of safety for use by PD patients, Seroquel (quetiapine; QTP) was identified as a potential candidate. The ability of Seroquel to activate rM3Ds was first tested in vivo. Following bilateral injection of AAV8R12-G88P7-rM3Ds-EYFP into the SNr of adult mice, either QTP or CNO was delivered via IP injection and total distance traveled was quantified in an open field. As shown in FIG. 20, QTP was capable of stimulating movement in an open field, indicating that it is a suitable in vivo ligand for rM3Ds. Next, we tested whether QTP could induce movement in animals injected with AAV8R12-G88P20-hM3Ds. Compared to CNO, however, we found that QTP administration did not increase distance traveled in these animals, shown in FIG. 21, suggesting that differences between the structures of hM3Ds and rM3Ds render QTP ineffective at activating hM3Ds.

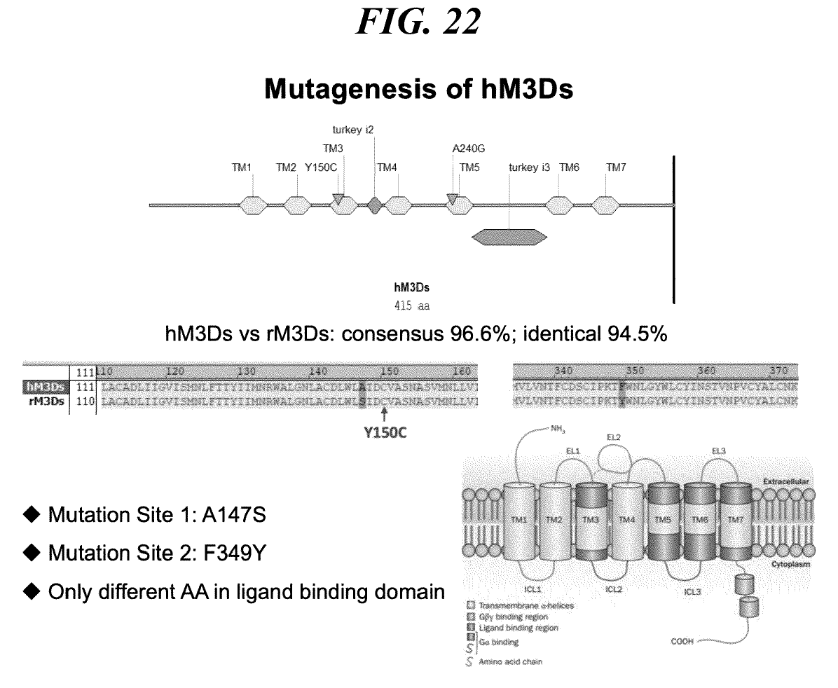

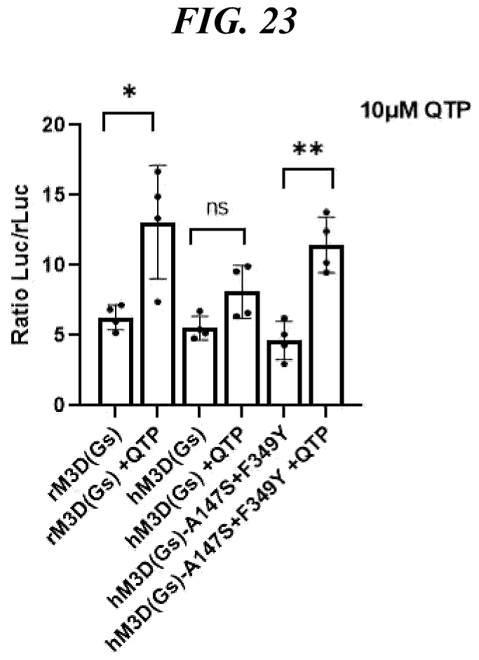

As a first step to designing an hM3Ds variant that can be activated by QTP, we aligned the sequences of rM3Ds and hM3Ds to identify differences. In general, rM3Ds and hM3Ds share 96.6% consensus and are 94.5% identical. However, only two mutations lie within the ligand binding domain FIG. 22, suggesting that these residues are responsible for the differences between rM3Ds and hM3Ds response to NQN. Next, we made two mutations, A147S and F349Y, at these sites in hM3Ds to revert them back to the rM3Ds sequence and tested the ability of QTP to activate this receptor in an in vitro luciferase activity assay in HEK293 cells. As seen in FIG. 23, treatment with 10 μM QTP significantly increased luciferase levels of hM3Ds-A147S-F349Y at the same levels observed for rM3Ds but did not increase luciferase levels for wild-type hM3Ds.

### Example 8—Additional Discussion of Study Results

One of the challenges for modern neuroscience has been to translate cutting-edge technological advances into effective therapeutic strategies for human brain disorders. In these examples, the relatively high-throughput nature of mouse studies was leveraged for discovery research and the further relevance of NHPs was further leveraged for developing and demonstrating a novel circuit-manipulating gene therapy to treat core symptoms of Parkinson's disease.

Some approaches developed here take advantage of distinct axonal targeting properties of different subtypes of projection neurons, which may be commonly seen in many brain regions and subregions. Some embodiments may therefore provide a feasible solution to access both the anatomical and functional characteristics of a variety of unique projection neuron types with many of them having direct therapeutic usefulness. Robust and specific targeting may rely upon AAV serotypes with enhanced labeling efficiencies for different cell types and appropriate cell type-selective promoters that allow targeting of subsets of neurons within mixed populations of cells. Moreover, it was here shown that the selection of chemogenetic effectors or other modulators of cellular activity can affect the specificity of functional manipulations. Continued advances in viral capsid evolution, identification and characterization of promoters and distal regulatory elements, and neural modulation technologies may further be developed for the development of a comprehensive toolkit that further enable rapid development of research strategies and therapeutic approaches based on circuit-specific activity modulation.

In parkinsonian macaque models, 0.3 mg/kg/day of DCZ was administered to achieve correction of some important motor symptoms. Given that the clinically-used standard dose of clozapine, a DREADD agonist with a core structure similar to that of DCZ, in treating schizophrenia is ˜4.5-9 mg/kg/day, the substantially lower dose of DCZ in treating primate PD animals may markedly reduce side effects observed in the clinical use of clozapine, such as neutropenia and weight gain. Nonetheless, the development and characterization of new and clinically safe DREADD ligands is a useful area for further translation of chemogenetic manipulation approaches in treating brain disorders. In primate PD models here, a therapeutic retrograde AAV was delivered to the SNr before lesioning the dopamine neurons in the SNc. This reversed experimental sequence was chosen because in the MPP+ SNc injection PD macaque models, massive destruction of dopamine neurons, although highly specific and efficient, may lead to strong local inflammation and immune cell activation and penetration in the SNc and neighboring SNr. This change in local environment may prevent efficient viral transduction in the SNr if the therapeutic AAV were to be delivered afterwards. To validate that the change in experimental sequence did not affect the evaluation of therapeutic effects, retrograde AAV injection was performed before striatal injection of 6-OHDA to remove SNc dopamine neurons in mice and a reversal of parkinsonian phenotypes was observed identical to those with pre-existing dopamine neuron elimination.

Systemic administration of L-Dopa is a treatment method for Parkinson's disease patients. L-Dopa's action on the central non-BG and peripheral dopamine systems may contribute to the occurrence of many side effects. Some approaches described herein can precisely modulate the basal ganglia direct pathway without affecting any other dopamine pathways in the body and can likely prevent the occurrence of most or all L-Dopa-induced side effects. Furthermore, L-Dopa may in some cases require survival of at least some nigral dopamine neurons to convert it to dopamine, which may serve as a cause for its efficacy fluctuation and decline after long-term use and progressive death of dopamine neurons in PD patients. Chemogenetic gene therapy methods devised herein, on the other hand, in some embodiments do not require the survival of nigral dopamine neurons and may provide a treatment option for late-stage PD patients who have lost most or all of their nigral dopaminergic neurons. The observation that approaches described here can reverse parkinsonian symptoms in PD primates that have received extended L-Dopa treatment indicate that it is a feasible candidate treatment for advanced PD. Moreover, pronounced L-Dopa-induced dyskinesia in parkinsonian primates was observed, but the same group of animals were dyskinesia free after an 8-month treatment with DCZ. Given that alteration of D1-MSN activity may be a major driver for the acute and chronic side effects observed with dopamine replacement therapy, lack of dyskinesia with some approaches herein may arise from the inability of DREADD to induce plasticity at corticostriatal synapses or its impact on the local striatal circuitry. Another feature of some approaches described herein is an extended efficacy window compared to a standard 6-hour window for L-Dopa. Some methods described herein are effective 24 hours after drug administration in parkinsonian monkeys and do not show signs of off time through the significantly extended therapeutic window. In addition, mixed results have been seen in trials applying dopamine agonists to treat depression, a common non-motor symptom of PD. Approaches described herein that specifically modulate one of the major dopamine-dependent circuits in the brain, may help differentiating roles of distinct dopamine systems in emotion modulation and provide an alternative strategy to alleviate parkinsonian mood symptoms. Overall, the precision gene therapy approaches here developed may be useful for treating neurological disorders such as PD.

### Example 9—Additional Method Details of Studies

### AAV Capsid Modification

Rep-Cap plasmids for AAV1/5/6/8R (retro) were chemically synthesized (GENEWIZ) by replacing the Cap sequence in the rAAV2-retro helper plasmid (Addgene, 81070) with sequences harboring intended sequence modifications. AAV8R1-14 Cap variants were generated by introducing 1-3 mutations to the AAV8R backbone. Mutagenesis was done using PCR with PrimeSTAR HS DNA polymerase (Takara, R010A) and a pair of primers for each site. For example, the sequences of the mutagenesis primers used to introduce the V183E mutation were 5′-TGGCGACTCAGAGTCAGAGCCAGACCCTCAACCTCT-3′ (SEQ ID NO: 34) and 5′-AGAGGTTGAGGGTCTGGCTCTGACTCTGAGTCGCCA-3′ (SEQ ID NO: 58). The PCR product was purified, digested with DpnI (NEB, R0176S) to remove template, and transformed into competent E. coli cells. DNA was then extracted from individual colonies with a Miniprep kit (Qiagen, 27106) and Sanger sequencing was performed to confirm the introduction of intended mutations.

AAV8RCap protein sequence
(SEQ ID NO: 1)
MAADGYLPDWLEDNLSEGIREWWALKPGAPKPKANQQKQDDGRGLVLPGY
KYLGPFNGLDKGEPVNAADAAALEHDKAYDQQLQAGDNPYLRYNHADAEF
QERLQEDTSFGGNLGRAVFQAKKRVLEPLGLVEEGAKTAPGKKRPVEPSP
QRSPDSSTGIGKKGQQPARKRLNFGQTGDSESVPDPQPLGEPPAAPSGVG
PNTMAAGGGAPMADNNEGADGVGSSSGNWHCDSTWLGDRVITTSTRTWAL
PTYNNHLYKQISNGTSGGATNDNTYFGYSTPWGYFDFNRFHCHFSPRDWQ
RLINNNWGFRPKRLSFKLFNIQVKEVTQNEGTKTIANNLTSTIQVFTDSE
YQLPYVLGSAHQGCLPPFPADVFMIPQYGYLTLNDGSQAVGRSSFYCLEY
FPSQMLRTGNNFQFTYTFEDVPFHSSYAHSQSLDRLMNPLIDQYLYYLSR
TQTTGGTANTQTLGFSQGGPNTMANQAKNWLPGPCYRQQRVSTTTGQNNN
SNFAWTAGTKYHLNGRNSLANPGIAMATHKDDEERFFPSNGILIFGKQNA
ARDNADYSDVMLTSEEEIKTTNPVATEEYGIVADNLQRGNLADQDYTKTA
RQAATADVNSQGALPGMVWQNRDVYLQGPIWAKIPHTDGNFHPSPLMGGF
GLKHPPPQILIKNTPVPADPPTTFNQSKLNSFITQYSTGQVSVEIEWELQ
KENSKRWNPEIQYTSNYYKSINVDFAVNTEGVYSEPRPIGTRYLTRNL*
AAV8R12 Cap protein sequence
(SEQ ID NO: 59)
MAADGYLPDWLEDNLSEGIREWWALKPGAPKPKANQQKQDDGRGLVLPGY
KYLGPFNGLDKGEPVNAADAAALEHDKAYDQQLQAGDNPYLRYNHADAEF
QERLQEDTSFGGNLGRAVFQAKKRVLEPLGLVEEGAKTAPGKKRPVEPSP
QRSPDSSTGIGKKGQQPARKRLNFGQTGDSESEPDPQPLGEPPAAPSGVG
PNTMAAGGGAPMADNNEGADGVGSSSGNWHCDSTWLGDRVITTSTRTWAL
PTYNNHLYKQISNGTSGGATNDNTYFGYSTPWGYFDFNRFHCHFSPRDWQ
RLINNNWGFRPKRLSFKLFNIQVKEVTQNEGTKTIANNLTSTIQVFTDSE
YQLPYVLGSAHQGCLPPFPADVFMIPQYGYLTLNDGSQAVGRSSFYCLEY
FPSQMLRTGNSFQFTYTFEDVPFHSSYAHSQSLDRLMNPLIDQYLYYLSR
TQTTGGTANTQTLGFSQGGPNTMANQAKNWLPGPCYRQQRVSTTTGQNNN
SNFAWTAGTKYHLNGRNSLANPGIAMATHKDDEERFFPSNGILIFGKQNA
ARDNADYSDVMLTSEEEIKTTNPVATEEYGIVADNLQRGNLADQDYTKTA
RQAATADVNSQGALPGMVWQNRDVYLQGPIWAKIPHTDGNFHPSPLMGGF
GLKHPPPQILIKNTPVPADPPTTFNQSKLNSFITQYSTGQVSVEIEWELQ
KENSKRWNPEIQYTSNYYKSINVDFAVNTEGVYSEPRPIGTRYLTRNL*

### Promoter Design and Screening

Region-selective promoter identification was based on the Allen Brain Atlas in situ hybridization (ISH) data (mouse.brain-map.org/). Genes highly expressed in the striatum but not in other parts of the basal ganglia were first selected as candidates. H3K4me1 and H3K27ac are known epigenetic marks for active promoters and enhancers. Thus, we identified regions with high levels of these two chromatin modification marks in mouse brain as candidate regions based on the ENCODE annotation data on the UCSC Genome Browser (genome.ucsc.edu/). To further extend the promoters' potential application in primates, the sequences of interest were PCR-amplified from human genomic DNA. Based on the above strategies, GPR88 was selected as a candidate gene. The nucleotide sequence upstream of the start codon with high H3K4me1 and H3K27ac levels was selected as the candidate promoter sequence. G88P2 (2259 bp) was cloned with the following primers: 5′-CATCGCAAGGCTACATGATGG-3′ (SEQ ID NO: 36), 5′-CTGGCCAACTCTTCACACCTC-3′ (SEQ ID NO: 60). G88P3 (1395 bp) and G88P7 (896 bp) were further shortened by subcloning.

Sequences of G88P2/3/7 promoters
G88P2 promoter
(SEQ ID NO: 44)
CATCGCAAGGCTACATGATGGATGCTCGGCTTAGTATCTGGGTGTGCTGCAACCCCACCTAAC
ATTTTTTTCTATTATGCCATGTTACCATATTCCCATATTGAGTTACATTTTCAAATACATGACTTTTCCAGTG
AATAATTGAGAGAACTCCTTTTCTGAAAAAATTCCCACATGGAATGGATAAATGAACACTTGCACCAAAG
AAGATGCCAACATCAGACTGCAAATCTCCAAGACAGACAAGACAAAAAGTTCTGAAGTGAAAAGTCATCT
TTGTTCACACTGTGGATTATTCAGGTTTCTTGCTTTCCCCCTGACACACATTTTTTTAAAATTGACTTTATTT
TTTTAGAGCAATTTTAGATTCATGGCAAATTTGAGTGAAAGATACAGAGATTTCCCATGTACTCCCTGCCC
CTCGCATACATAGCCTCTCCCATTATCAAAATCCCCCACCAGAGTGGTTCGTCTGTTACAATTGAATGTACG
TTGACACATCATTCCCCAAAGTCCATGGCTTACACTGATGTTTCACATTCTGCCACTTTGGGCAAATTTATA
AGTACACAGATCCACTATTATGGTATCATACAGAGTAGTTCCACTGTCCTAAGAATCTTCCGTGATCATCT
ATTCATTCCTCCTTTCCCCCTAACTCGGGCAACCACTGGTCTTTTTAACTGTCTCCATAGTTTTGCCTTCTCA
AGAATGTCATAGTTGGTAGCATACAGTATGTAGTCCTTTCCAGTTGGCTTCTGTCACTTAGTGATACACACT
TAAGGTTCCTTCATGTCTTTTCATGGCTGGACAGCTCATTTCTTTTTAGCACTGAATAATATTCCATTGTCTA
GATGTACCACTGACATCTGCTTTAAATCTTTTGCCCATTTCATTTTTACCCTCTAACCATTTCATTACTAAAA
CACAAAACAAACTCCGGAAAGGAGTTTGTTGATGAGAAAACACTGAAAGTACGATTTAAATTGAGGAATG
GAGATTTTCTGTGGATAATTGTCTATGTTCCACTTTACCAAAGAAAAATGAATTTCAAGTAGTGCATAAAG
TAATTCTCGTTAAAAAATGTGGGAATAAAGAGATCAGTCCTGGGTACTGGGAATATATTTATCTCACAGAA
AAATCTTGCCCACAAAATGCATCAGACTGAGATGAAATACTCTAGGGTATATTAAATTTTATTCTAATATT
GAAGGGAGGGAAAAAAAGGACGGAAAGAAAAATGAAACAGGATGAAAAATAAGGTGATTGGGAGAGAG
AAAAGACAGAGAAACTTGTTAACGACAGGACTTTCTGAGCAAGATATTTTTCTTCCTTTTACAAGTTGGAA
GCTATTGAATAATTCATGCCAAGCGTGGAAAGGCTGTCTTTTCCCTGGATGATGTATAACTAAGCAGGAGA
GAGCTTCGAGTGGGTTCACCACATCAGCCACCACTCCTGCTTCTGAGCACAGGGTGCTCTCCTCTTGAGCT
CAGCTTCTGCTTTTGCAGCCAAGCATTCTTGCTGCTGCTGCCTGCCTGCCCACCCGCCTGGGCTTGCAGCCC
GCCACTTTACTTTCTCCAGCCCTGATACCAGCTGAGAAGTCTCCCTGCAGCTGCTAGTTCCTGCCCAGGACC
ATGTGTGTGGATGCTGCTTGGGAGAAGCGGGCACTTGCTCCTGGCACTGATCCCAGCTGAGTTTCTCCTGT
TGATTTCTGGACCACTGATGCTGTTGCTGAGGAGGTATTTCCCGGCATCCCTCCCCCTGAGACACCGGCTA
AGGACCAGCCTAAACGCAAGGTAACCTTCACCTCTTTCCCATGGGATTTTCCCCAGCTGGGATATCTCTGC
TTTCTCCTGGAACTAGGTCCTCCAGCTGGCAACATATCTCTCTCTCTCTCTCTCTCTCTCTCTCTCTCTCTCTC
TCTGTGTGTGTGTGTGTGTGTTGGGGGAGAAGAGGAGACTGTTTTAGGTATTGCTCTGTCCTAGGGCTTCCC
CGATACCTGCTGAATCCAATGCAAAGTGGCCTTTCCCCTGGCTCTGGAGACATTTCCGAGGCAGGCACCTG
TCATCTGCCACCCCCGTCCTCCTCCTCGAGCTCCCTTTCTCCCCCTCCCCCAGCCCATTATTCTGCTTCAGCC
TTTTGTGTCAGTGGCAGAGGGCTGAAGGGATGTCTTTGCCCTTCTGGCAGGCAGGACAGTGTCAGGATGGA
CCGCGCTGCCAGAAGCCGACGCTAGCGAGGGAGGTGTGAAGAGTTGGCCAG
G88P3 promoter
(SEQ ID NO: 45)
TCTAGATGTACCACTGACATCTGCTTTAAATCTTTTGCCCATTTCATTTTTACCCTCTAACCATT
TCATTACTAAAACACAAAACAAACTCCGGAAAGGAGTTTGTTGATGAGAAAACACTGAAAGTACGATTTA
AATTGAGGAATGGAGATTTTCTGTGGATAATTGTCTATGTTCCACTTTACCAAAGAAAAATGAATTTCAAG
TAGTGCATAAAGTAATTCTCGTTAAAAAATGTGGGAATAAAGAGATCAGTCCTGGGTACTGGGAATATATT
TATCTCACAGAAAAATCTTGCCCACAAAATGCATCAGACTGAGATGAAATACTCTAGGGTATATTAAATTT
TATTCTAATATTGAAGGGAGGGAAAAAAAGGACGGAAAGAAAAATGAAACAGGATGAAAAATAAGGTGA
TTGGGAGAGAGAAAAGACAGAGAAACTTGTTAACGACAGGACTTTCTGAGCAAGATATTTTTCTTCCTTTT
ACAAGTTGGAAGCTATTGAATAATTCATGCCAAGCGTGGAAAGGCTGTCTTTTCCCTGGATGATGTATAAC
TAAGCAGGAGAGAGCTTCGAGTGGGTTCACCACATCAGCCACCACTCCTGCTTCTGAGCACAGGGTGCTCT
CCTCTTGAGCTCAGCTTCTGCTTTTGCAGCCAAGCATTCTTGCTGCTGCTGCCTGCCTGCCCACCCGCCTGG
GCTTGCAGCCCGCCACTTTACTTTCTCCAGCCCTGATACCAGCTGAGAAGTCTCCCTGCAGCTGCTAGTTCC
TGCCCAGGACCATGTGTGTGGATGCTGCTTGGGAGAAGCGGGCACTTGCTCCTGGCACTGATCCCAGCTGA
GTTTCTCCTGTTGATTTCTGGACCACTGATGCTGTTGCTGAGGAGGTATTTCCCGGCATCCCTCCCCCTGAG
ACACCGGCTAAGGACCAGCCTAAACGCAAGGTAACCTTCACCTCTTTCCCATGGGATTTTCCCCAGCTGGG
ATATCTCTGCTTTCTCCTGGAACTAGGTCCTCCAGCTGGCAACATATCTCTCTCTCTCTCTCTCTCTCTCTCT
CTCTCTCTCTGTGTGTGTGTGTGTGTGTTGGGGGAGAAGAGGAGACTGTTTTAGGTATTGCTCTGTCCTAGG
GCTTCCCCGATACCTGCTGAATCCAATGCAAAGTGGCCTTTCCCCTGGCTCTGGAGACATTTCCGAGGCAG
GCACCTGTCATTTGCCACCCCCGTCCTCCTCCTCGAGCTCCCTTTCTCCCCCTCCCCCAGCCCATTATTCTGC
TTCAGCCTTTTGTGTCAGTGGCAGAGGGCTGAAGGGATGTCTTTGCCCTTCTGGCAGGCAGGACAGTGTCA
GGATGGACCGCGCTGCCAGAAGCCGACGCTAGCGAGGGAGGTGTGAA
G88P7 promoter
(SEQ ID NO: 46)
ATGCCAAGCGTGGAAAGGCTGTCTTTTCCCTGGATGATGTATAACTAAGCAGGAGAGAGCTTC
GAGTGGGTTCACCACATCAGCCACCACTCCTGCTTCTGAGCACAGGGTGCTCTCCTCTTGAGCTCAGCTTCT
GCTTTTGCAGCCAAGCATTCTTGCTGCTGCTGCCTGCCTGCCCACCCGCCTGGGCTTGCAGCCCGCCACTTT
ACTTTCTCCAGCCCTGATACCAGCTGAGAAGTCTCCCTGCAGCTGCTAGTTCCTGCCCAGGACCATGTGTGT
GGATGCTGCTTGGGAGAAGCGGGCACTTGCTCCTGGCACTGATCCCAGCTGAGTTTCTCCTGTTGATTTCTG
GACCACTGATGCTGTTGCTGAGGAGGTATTTCCCGGCATCCCTCCCCCTGAGACACCGGCTAAGGACCAGC
CTAAACGCAAGGTAACCTTCACCTCTTTCCCATGGGATTTTCCCCAGCTGGGATATCTCTGCTTTCTCCTGG
AACTAGGTCCTCCAGCTGGCAACATATCTCTCTCTCTCTCTCTCTCTCTCTCTCTCTCTCTCTCTCTGTGTGT
GTGTGTGTGTGTTGGGGGAGAAGAGGAGACTGTTTTAGGTATTGCTCTGTCCTAGGGCTTCCCCGATACCT
GCTGAATCCAATGCAAAGTGGCCTTTCCCCTGGCTCTGGAGACATTTCCGAGGCAGGCACCTGTCATCTGC
CACCCCCGTCCTCCTCCTCGAGCTCCCTTTCTCCCCCTCCCCCAGCCCATTATTCTGCTTCAGCCTTTTGTGT
CAGTGGCAGAGGGCTGAAGGGATGTCTTTGCCCTTCTGGCAGGCAGGACAGTGTCAGGATGGACCGCGCT
GCCAGAAGCCGACGCTAGCGAGGGAGGTGTGAAGAGTTGGCCAG

### AAV Vector Construction

Promoters were subcloned into a pAAV-hSyn-EYFP vector derived from pAAV-hSyn-EGFP (Addgene, 50465) to replace the hSyn promoter by using appropriate restriction enzyme combinations. To generate pAAV-G88P7-DIO-tdTomato, a DIO-tdTomato cassette was subcloned into pAAV-G88P7-EYFP to replace EYFP by restriction enzyme digestion. To generate pAAV-G88P3-HA-hM3Dq, promoter G88P3 was subcloned into pAAV-hSyn-HA-hM3Dq-IRES-mCitrine (Addgene, 50463) via the EcoRI/BamHI restriction sites to replace the hSyn promoter and IRES-mCitrine was removed afterwards by restriction enzyme digestion. To generate pAAV-G88P3-HA-hM3Dq-2A-EYFP, a 2A-EYFP fragment was subcloned into pAAV-G88P3-HA-hM3Dq after hM3Dq. To generate pAAV-G88P7-rM3Ds-2A-EYFP, promoter G88P7 was subcloned into pAAV-hSyn-DIO-rM3Ds-mCherry (Addgene, 50458) to replace the hSyn promoter and mCherry was subsequently replaced by 2A-EYFP followed by the removal of the DIO structure through restriction enzyme digestions. To generate pAAV-G88P7-HA-rM3Ds-2A-Cre, EYFP was replaced by Cre in pAAV-G88P7-HA-rM3Ds-2A-EYFP through restriction enzyme digestions.

### AAV Production and Titration

HEK293T cells (ATCC) were co-transfected by calcium phosphate with an AAV vector plasmid, an AAV Rep-Cap plasmid (rAAV2-retro, AAV1R, AAV5R, AAV6R, AAV8R, and AAV8R1-14), and the pAdDeltaF6 helper plasmid (Addgene, 112867) harboring the adenoviral genes required for the AAV life cycle. HEK293T cells grown in 15 cm cell culture dishes were co-transfected with the mixture of three plasmids (1:1:1) at 80% confluency. 48-72 hours post-transfection, cells were collected and resuspended in a buffer containing 150 mM NaCl and 100 mM Tris-HCl (pH 8.0). Cells were lysed by repeated freeze-thaw cycles in liquid nitrogen and a 37° C. water bath. AAV particles were purified and concentrated using Millipore Amicon 100K columns (Merck Millipore, UFC910008). Encapsidated viral DNA was quantitated by qPCR (Thermo Fisher) with primers recognizing the viral WPRE and/or ITR sequences following denaturation of the AAV particles using Proteinase K. Titers were calculated as genome copies per milliliter.

### Surgeries and Virus Injections

### For Mice:

For retrograde labeling in Drd1-Cre or Drd2-Cre mice, a total volume of 200 nL of AAV8R12-G88P7-EYFP was unilaterally injected into the SNr. Simultaneously, a total volume of 300 nL of AAV9-G88P7-DIO-tdTomato was ipsilaterally injected into the striatum (0.5 mm anterior, 1.5 mm lateral, 3.5 mm ventral to bregma). For opto-tagging in mice, a total volume of 200 nL of AAV8R12-G88P7-HA-rM3Ds-2A-Cre was unilaterally injected into SNr. AAV9-EF1α-DIO-ChR2-EYFP was ipsilaterally injected into two sites to maximally cover the striatum (250 nL/site, site 1: 1.2 mm anterior, 1.5 mm lateral, 3.2 mm ventral to bregma, site 2: 0.4 mm anterior, 1.6 mm lateral, 3.3 mm ventral to bregma).

### For Monkeys:

All neurosurgical procedures were performed using sterile methods while the subject was anesthetized. For general anesthesia, monkeys were given atropine (0.05 mg/kg, intramuscular) to decrease bronchial secretions before ketamine administration (15 mg/kg, intramuscular). Propofol (6 mg/kg, i.v.) was used to maintain anesthesia. The anesthetic level was adjusted to eliminate movement as assessed by toe pinches. Corneal reflexes were consistently absent. The subject was placed on a standard operating table with constant heating and the head of the subject was rigidly fixed on a stereotaxic frame (David Kopf Instruments). Electrocardiography, heart rate, oxygen saturation (SpO2) (range 95-100%) and rectal temperature (37.5-38.5° C.) were continuously monitored by a physiological monitor (Mindray, uMEC7).

Virus injections in the striatum were conducted in both the caudate (12 uL virus, 4 sites) and the putamen (18 uL virus, 6 sites) at a speed of 300 nL/min.

To intracranially administer drug and/or perform electrophysiological recording, a recording chamber covering from anterior caudate to posterior GPi was fixed on the skull with 6 titanium screws and dental cement. Each subject was allowed to recover from the surgery for at least 6 weeks prior to further studies.

### Model Generation for Parkinson's Disease for Mice

6-OHDA was bilaterally injected using the same methods described for virus injections. A total volume of 1 μL 6-OHDA (5 mg/mL, dissolved in sterile saline containing 0.02% ascorbic acid, Sigma) was injected into the striatum at a speed of 100 nL/min. The coordinates for the striatum were 0.5 mm anterior, 1.5 mm lateral, and 3.2 mm ventral to bregma. A premedication of desipramine (25 mg/kg, Sigma) was administered to animals prior to injections of 6-OHDA to increase the selectivity and efficacy of 6-OHDA-induced lesions. Mice were supplemented with DietGela (ClearH2O) for one week after surgery. All staining and behavioral experiments were performed at least 14 days after surgery, when dopamine depletion was maximal and stable.

### PD Score

Two experienced observers blindly evaluated parkinsonian symptoms of the monkeys three days per week throughout the observation periods. Parkinsonian symptoms were quantified according to the well-established Kurlan scale (Part I. Parkinsonian features), a widely used scale to quantify PD symptoms in old world monkeys. A score of zero indicates a normal monkey, whereas a maximum score of 29 indicates an animal with severe PD symptoms. For separate behavioral categories, scoring of the upper limb and the lower limb were added together. Additionally, action or intention tremors and resting tremors were added together.

### Dyskinesia Score

Two experienced observers blindly evaluated dyskinesia symptoms of the monkeys three days per week throughout the observation periods. Dyskinesia score criteria were: 0: Absent. 1: Mild, fleeting, present <30% of the observation period. 2: Moderate, not interfering with normal activity, present >30% of the observation period. 3: Marked, at times interfering with normal activity; present <70% of the observation period.4: Severe, continuous, replacing normal activity, present >70% of the observation.

### Immunofluorescence

Animals were deeply anesthetized with sodium pentobarbital (Nembutal; 80 mg/kg, i.p.) and perfused with PBS (0.1M) and 4% paraformaldehyde in PBS (4% PFA/PBS, 4° C., 30 mL for mice and 500 mL for monkeys). The dissected brains were post-fixed at 4° C. in 4% PFA/PBS and cryo-protected at 4° C. in 30% sucrose/PBS. Coronal sections (40 μm for mice and 50 μm for monkeys) were prepared using a cryostat (Leica, CM1950). All sections were post-fixed for 20 minutes at 4° C. in 4% PFA/PBS. The sections were blocked and permeabilized for 1 hour at room temperature in a PBS solution containing 5% bovine serum albumin (BSA) and 0.3% Triton X-100. The primary antibody application was performed by incubating the sections overnight at 4° C. in a PBS solution containing 5% BSA and polyclonal anti-GFP (Rockland, 600-101-215M), anti-RFP (Rockland, 600-401-379), anti c-Fos (Cell Signaling Technology, 2250), anti-HA (Biolegend, 923501), and/or anti-TH (Abcam, ab76442) antibodies. The secondary antibody incubation was performed for 1 hour using Alexa Fluor 488 donkey anti-goat IgG, Alexa Fluor 594 donkey anti-rabbit IgG, Alexa Fluor 488 goat anti-chicken IgG, and/or Alexa Fluor 488 streptavidin (Thermo Fisher, A32814, A32754, A11039 and S11223, respectively.). Nuclei were stained with DAPI (Sigma, D9542). The brain sections were mounted onto slides using Fluoromount-G mounting medium (SouthernBiotech, 0100-01).

### Cell Counting

Images were acquired using a confocal microscope (Carl Zeiss, LSM880) and an Axiolmager.Z1 microscope with apotome (Carl Zeiss). Brain structures were identified microscopically and in digital photos using a mouse brain atlas. To analyze the number of EYFP+ cells in the striatum, slices from rostral to caudal striatum were used. Every sixth section was analyzed, and numbers of EYFP+ cells were multiplied by six to acquire the approximate total number and mean number of cells per animal. Images were processed using ImageJ (NIH, USA), and final quantifications were independently performed manually by two blinded experimenters.

### In Situ Hybridization

Coding region fragments of mouse Drd1/Drd2 or macaque DRD1/DRD2 were isolated from brain cDNA using PCR amplification. The amplified fragments were cloned into the pCR4 TOPO vector (Thermo Fisher). In situ hybridization was performed as previously described with minor modifications. Briefly, Digoxigenin (DIG)-labeled cRNA probes (riboprobes) were prepared using the DIG RNA Labeling Mix (Roche). Brains were frozen in OCT (Tissue-Tek), and coronal cryostat sections of 40-50 μm thickness were hybridized with DIG-labeled cRNA probes at 56° C. for 15-18 hours. After hybridization, sections were washed twice in 0.2×SSC at 62° C. for 30 minutes, incubated with peroxidase (POD)-conjugated anti-DIG antibodies (Roche, 1207733910) at 37° C. for 2 hours, and then treated with the TSA-plus kit (Perkin Elmer). Sections were then incubated with anti-RFP antibody (Rockland, 600-401-379) or anti-GFP antibody (Rockland, 600-101-215M) at 4° C. overnight and finally with Alexa Fluor 594 donkey anti-rabbit IgG (Thermo Fisher, A32754) or Alexa Fluor 488 donkey anti-goat IgG (Thermo Fisher, A32814) at room temperature for 2 hours before mounting with Fluoromount-G (SouthernBiotech, 0100-01).

### Behavioral Assays for Mice

### Open Field Test

Mice were individually handled for 10-20 seconds per day for at least 5 days to habituate them to the experimenter. The open field test was conducted in a test apparatus (50 cm×50 cm×50 cm) with an HD digital camera (Sony) positioned above the arena. On day one, mice were habituated to the apparatus for 10 minutes. On day 2, mice were administrated saline (0.1 mL i.p. or 200 nL intracranially [i.e.] through the guide cannulas). On day 3, mice were administrated with Clozapine-N-oxide (CNO, Hello Bio, HB1807; 0.3 mg/kg i.p. or 200 nL at 100 μM i.e.). All behavioral tests were conducted 30 minutes after injection. During the test, mice were allowed to freely explore the apparatus for 10 minutes. The behavioral data were analyzed with ANY-maze software (Stoelting Co.).

### Rotarod Test

Mice were individually handled as described in the open field test. Mice were transferred to the testing room and acclimated for 15 minutes before the test session. Mice were placed on the rod (Shanghai Xinruan) with the apparatus set to mode (10-40 r.p.m.). Latency to fall for each trial was automatically recorded by the apparatus. Each mouse was tested for three trials in a single day for two consecutive days, with a minimum of 15 minutes between trials. Fall latency was averaged across the three trials within each day.

### Behavioral Assays for Monkeys

### Locomotion Test:

The locomotion tests for monkeys were conducted in a custom-made observation cage (100 cm×100 cm×100 cm). The top and front of the cage were made of toughened glass in order to gain a clear view for behavioral recording. Monkeys were habituated to the observation cage by placing them inside for 30 minutes on three separate days.

For monkeys injected with AAV8R12-G88P3-HA-hM3Dq, CNO was intracranially infused into the dorsomedial caudate through the recording chamber. To infuse while the animal was awake, the monkey was trained to sit in a primate chair specially designed with its head fixed to the primate chair by a mask made of thermoplastic materials. Injections were conducted through a 33-Gauge needle connected to a 250 μL syringe via a polyethylene pipe (Hamilton, Neuros). A total volume of 3 μL CNO (100 μM) was infused at a rate of 0.5 μL/min using a microsyringe pump (KD Scientific, Legato 130). The needle was held for 5 minutes for drug diffusion before retraction. For monkeys injected with AAV8R12-G88P7-rM3Ds-2A-EYFP, CNO was infused via intramuscular injection (10 mg/kg). After CNO was successfully infused, monkeys were immediately transferred to the observation cage for video recording. Videos capturing the subjects' behavior were recorded for at least 90 minutes. Animal behaviors from 30-90 minutes after saline/CNO infusion were further analyzed and quantified.

For parkinsonian monkeys, DCZ (Deschloroclozapine, 0.3 mg/kg, MCE) was administered via intramuscular injection. After DCZ was successfully infused, monkeys were immediately transferred to the observation cage for video recording. For levodopa treatment, L-Dopa/benserazide (20/5 mg/kg/day, 4:1, L-Dopa/benserazide ratio as Madopar®) was administered orally. For initial test, DCZ treatment trials and Levodopa treatment trials were separated by 2 weeks to allow for drug washout. For long-term DCZ treatment, parkinsonian monkeys were given DCZ at a dose of 0.3 mg/kg every other day via intramuscular injection. For extended L-Dopa treatment, animals were administered with L-Dopa once daily for 4 months. One month of washout was allowed before administration of DCZ for a 2 months treatment.

### Motor Skill Test

A grasp-to-eat/hand-to-mouth movement test was conducted as an indication of motor skills in monkeys. Monkeys were first trained to sit in a primate chair with the healthy hand restricted. Monkeys were then trained to grasp food (a piece of apple, ˜1 cm3) using the MPP+-affected hand from the experimenter. The task contained three blocks, with 20-minute intervals between different blocks. Each block included ten continuous trials. Trials were defined as successful when monkeys were able to bring food to their mouth within a minute. Before MPP+ lesion, monkeys were trained to reach an 85% success rate.

### Monkey Behavioral Analysis

During video recording, a top (x/y) and a side (x/z) camera were used to record the observation cage. The VigiePrimate system (Viewpoint) was used to analyze the subject's activity and extract movement data from the recorded videos. The tracking data were synchronously sampled at 25 Hz for both top view and side view, and then combined to generate one dataset representing the 3D space. The tracking data were normalized to 0-100 cm for all x-, y-, and z-axes, of which x represents right to left, y represents front to back, and z represents bottom to top.

For rotation analysis, only movements that occurred lower than 30 cm on the z-axis were counted. To determine the direction of the rotation, we first computed the center for the rotation data in the x-y plane as (x0, y0) by averaging all data points, and then we computed the angle relative to (x0, y0) for each time point by the following formula:

$${θ}_{i} = {tan}^{- 1} \frac{{y}_{i} - {y}_{0}}{{x}_{i} - {x}_{0}} \times \frac{180}{π}$$

where θᵢ represents the rotating angle at time i, and (xᵢ, yᵢ) represents the x-y coordinate at time i. We then calculated the change of rotating angle as Δθᵢ=θᵢ−θᵢ₋₁. For right nigral injections, when Δθ<0, the direction of the rotation was anticlockwise and was labeled as a contraversive rotation. When Δθ>0, the direction of the rotation was clockwise and was labeled as an ipsiversive rotation. The raw data were analyzed and plotted through a custom R package. Code is available on Github (github.com/chenyef/PD_ana/blob/main/Behavior_ana.txt).

### Electrophysiological Recording

### For Mice:

Mice from different groups were anesthetized with 0.04% isoflurane, and then decapitated. The brain was quickly extracted and placed in ice-cold N-methyl-D-glucamine (NMDG) cutting solution containing (in mM): 92 NMDG, 2.5 KCl, 25 NaHCO3, 1.25 NaH2PO4, 4.5 D-Glucose, 20 HEPES, 5 L-ascorbic acid, 3 Na-pyruvate, 2 Thiourea, 10 MgSO4, 0.5 CaCl2). The pH was adjusted to 7.2±0.1 using HCl and the osmolarity was adjusted to 305±5 mosmol/L using NMDG. The solution was bubbled with 95% O2/5% CO2 prior to use. Coronal striatum slices were cut in 250-□m thickness with a vibratome slicer (Leica, VT1200 S), and then incubated in artificial cerebrospinal fluid (aCSF) saturated with 95% O2/5% CO2 for at least 45 min at 37° C. before recording. The aCSF contains (in mM) 125 NaCl, 1.25 KCl, 25 NaHCO3, 1.25 KH2PO4, 25 D-Glucose, 2 CaCl2) and 1 MgCl2, supplemented with 2 mM Na-pyruvate, 3 mM Myo-inositol and 0.4 mM L-ascorbic acid.

The acute striatum slice was transferred to the recording chamber with a constant perfusion of oxygenized aCSF at the rate of 3 ml/min. Neurons were visualized using an upright microscope (Olympus, BX-51) with a 40×water immersion objective at room temperature (˜25° C.). In the striatum, EYFP positive neurons were selected for whole-cell recording. Neurons were quickly visualized under fluorescence microscope and then patch clamp were achieved under infrared light illumination and CCD camera. Data were collected using Axopatch 700B patch clamp amplifier, Digidate-1444A data acquisition system, and pCLAMP 10.6 software (Axon CNS). The patch pipette electrodes (OD=1.5 mm, Sutter Instrument) were pulled with Model P-1000 puller (Sutter Instrument) to a final tip resistance of 6-7 MΩ. The patched cells were stimulated with a current step injection (150-200 pA, 150 ms long) once per minute. The stimulation intensity was adjusted to evoke 1 action potential with 50% probability. Baseline recording was performed with 18 times current injection, then the perfusion solution was switched to CNO (10 μM)-containing aCSF and was allowed to stabilize for 3 minutes. Current clamp recordings were made with same current stimulation protocol in the presence of CNO for 20 minutes. The recording electrodes were filled with K methane sulfonate (KMeSO3)-based internal solution containing (in mM): 135 KMeSO3, 10 KCl, 10 HEPES, 5 MgATP, 0.5 NaGTP, 1 EGTA, pH was adjusted to 7.2±0.1 using KOH and the osmolarity was adjusted to 305±5 mosmol/L using KMeSO3.

### For Monkeys:

Electrophysiological recording was performed through a recording chamber while the monkey was anesthetized. Neural responses were recorded by a 16-channel linear probe (Plexon Inc, Uprobe) driven by a mechanical microdrive (Alpha Omega, FlexMT). Signals were passed through a head stage (Plexon Inc, HST/16V-G20 LN) and then split and filtered between 300 Hz and 5 kHz to identify spiking activity with an amplifier system (Plexon Inc, OmniPlex). Detected spikes were then sorted by commercially available software (Plexon Inc, Offline Sorter) for further analysis. To quantify the effect of CNO/DCZ injection, neural activity was recorded for 60 minutes following CNO/DCZ injection. The response time-course for each channel was obtained by counting the spikes within every 10-minute window and then normalizing to the maximal.

To determine the effect of CNO/DCZ injection on the direction of signal change, we performed Pearson correlation between neural responses and time using the ‘corr’ function in Matlab, which generates the correlation coefficient ‘r’ and significant level ‘p’. A neuron was considered to have “Increased” activity when r>0 and p<0.05; and considered to have “Decreased” activity when r<0 and p<0.05. Otherwise, neurons were considered to be “Unchanged” in their neural activities. The responses of all increased neurons were then averaged after being normalized by the maximal response (Crowe et al., 2014; Dai and Wang, 2018; Hirokawa et al., 2019) following CNO/DCZ injection, and then plotted as a function of time to generate the normalized population response.

### Opto-Tagging

### For Mice:

Brain slices were prepared under low illumination conditions. Blue LED light at 473 nm was given through the imaging objective (40×/0.8 Water Immersion Objective, Olympus, Japan). Recordings were carried out under current-clamp mode. In recordings to verify the ChR2 function, 200 μM CdCl2 were included in the bath to prevent back propagated calcium current from dendrites. 2-ms-long light pulses at 2 Hz with 86.89 mW/mm2 (sanwa-LP10, Japan) intensity was given to activate ChR2. Cells faithfully stimulated by light pulses were further recorded with CNO treatment.

### For Monkeys:

To enable in-vivo verification of the strategy via an opto-tagging approach, AAV virus encoding opsin ChR2 (AAV9-EF1α-DIO-ChR2-EYFP) was injected into the striatum with simultaneous nigral infusion of a retrograde AAV encoding Cre and rM3Ds (AAV8R12-G88P7-HA-rM3Ds-2A-Cre). A total volume of 27 μl (3 μl×9 sites) and 30 μl (3 μl×10 sites) were injected in the SNr and the striatum, respectively. A recording chamber was implanted above the striatum to allow optical stimulation and electrophysiological recording.

Optogenetic verification was conducted six weeks after virus injections. A 16-channel linear probe with an embedded optic fiber (Plexon Inc., Uprobe) was used to deliver light stimulation and record neural responses. A mechanical Microdrive (FlexMT, Alpha Omega, Nazareth, Israel) was used to mount and drive the probe. A blue laser (473 nm, Changchun New Industries Tech, MBL-III-473) was used to generate light stimulation, which was controlled by a DAQ board (National Instruments, PCIe-6321) through the MonkeyLogic toolbox (NIMH version).

Light pulses of 40 Hz that last for 500 ms were used to probe neurons' responses to optical stimulation. Once a neuron showed repeatable responses to light stimulation, a further test was conducted to verify its responses to CNO from 10 minutes before to 60 minutes after ligand administration. Responses from these optically identified cells were grouped to generate the normalized population response as a function of time.

### Electromyography (EMG) Recording and Data Analysis

Customized surface EMG electrodes were used for EMG recordings. The target skin area was shaved and cleaned thoroughly with alcohol wipes. Two electrodes were placed approximately halfway between the two tendinous insertions along the longitudinal axes of the biceps. Monkeys were trained to sit in a primate chair in the awake state with their upper limbs moving freely. The recording procedure was conducted immediately after DCZ infusions. EMG signals from biceps brachii were collected for at least 120 minutes. EMG signals were amplified by a signal acquisition and processing system (TECHMAN, BL-420N) collected at a sampling rate of 1000 Hz. The raw EMG data were then plotted and analyzed using custom Matlab (MathWorks) code (https://github.com/chenyef/PD_ana/blob/main/EMG_FFT_ana.txt).

### Quantification and Statistical Analysis

All statistics were performed in GraphPad Prism 9.0. Paired t-test, unpaired t-test, one-way ANOVA, Tukey's test, and Dunnett's test were used when appropriate. All t-tests were performed as two-tailed. In all statistical tests, a P-value <0.05 was considered statistically significant. Sample sizes were chosen based on previous publications or experience to yield sufficient power to detect specific effects. All statistical tests used are indicated in the figure legends.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Sequence listings provided herein

| SEQ | | |
|---|---|---|
| ID | | |
| NO: | Sequence | Origin |
| 1 | MAADGYLPDWLEDNLSEGIREWWALKPGAPKPKANQQKQDDGRGLVLPGY | AAV8R |
| | KYLGPFNGLDKGEPVNAADAAALEHDKAYDQQLQAGDNPYLRYNHADAEF | |
| | QERLQEDTSFGGNLGRAVFQAKKRVLEPLGLVEEGAKTAPGKKRPVEPSPQRS | |
| | PDSSTGIGKKGQQPARKRLNFGQTGDSESVPDPQPLGEPPAAPSGVGPNTMAA | |
| | GGGAPMADNNEGADGVGSSSGNWHCDSTWLGDRVITTSTRTWALPTYNNHL | |
| | YKQISNGTSGGATNDNTYFGYSTPWGYFDFNRFHCHFSPRDWQRLINNNWGF | |
| | RPKRLSFKLFNIQVKEVTQNEGTKTIANNLTSTIQVFTDSEYQLPYVLGSAHQG | |
| | CLPPFPADVFMIPQYGYLTLNDGSQAVGRSSFYCLEYFPSQMLRTGNNFQFTY | |
| | TFEDVPFHSSYAHSQSLDRLMNPLIDQYLYYLSRTQTTGGTANTQTLGFSQGG | |
| | PNTMANQAKNWLPGPCYRQQRVSTTTGQNNNSNFAWTAGTKYHLNGRNSL | |
| | ANPGIAMATHKDDEERFFPSNGILIFGKQNAARDNADYSDVMLTSEEEIKTTN | |
| | PVATEEYGIVADNLQRGNLADQDYTKTARQAATADVNSQGALPGMVWQNR | |
| | DVYLQGPIWAKIPHTDGNFHPSPLMGGFGLKHPPPQILIKNTPVPADPPTTFNQ | |
| | SKLNSFITQYSTGQVSVEIEWELQKENSKRWNPEIQYTSNYYKSINVDFAVNTE | |
| | GVYSEPRPIGTRYLTRNL | |
| 2 | MAADGYLPDWLEDNLSEGIREWWALKPGAPKPKANQQKQDDGRGLVLPGY | AAV8R1 |
| | KYLGPFNGLDKGEPVNAADAAALEHDKAYDQQLQAGDNPYLRYNHADAEF | (V183E) |
| | QERLQEDTSFGGNLGRAVFQAKKRVLEPLGLVEEGAKTAPGKKRPVEPSPQRS | |
| | PDSSTGIGKKGQQPARKRLNFGQTGDSESEPDPQPLGEPPAAPSGVGPNTMAA | |
| | GGGAPMADNNEGADGVGSSSGNWHCDSTWLGDRVITTSTRTWALPTYNNHL | |
| | YKQISNGTSGGATNDNTYFGYSTPWGYFDFNRFHCHFSPRDWQRLINNNWGF | |
| | RPKRLSFKLFNIQVKEVTQNEGTKTIANNLTSTIQVFTDSEYQLPYVLGSAHQG | |
| | CLPPFPADVFMIPQYGYLTLNDGSQAVGRSSFYCLEYFPSQMLRTGNNFQFTY | |
| | TFEDVPFHSSYAHSQSLDRLMNPLIDQYLYYLSRTQTTGGTANTQTLGFSQGG | |
| | PNTMANQAKNWLPGPCYRQQRVSTTTGQNNNSNFAWTAGTKYHLNGRNSL | |
| | ANPGIAMATHKDDEERFFPSNGILIFGKQNAARDNADYSDVMLTSEEEIKTTN | |
| | PVATEEYGIVADNLQRGNLADQDYTKTARQAATADVNSQGALPGMVWQNR | |
| | DVYLQGPIWAKIPHTDGNFHPSPLMGGFGLKHPPPQILIKNTPVPADPPTTFNQ | |
| | SKLNSFITQYSTGQVSVEIEWELQKENSKRWNPEIQYTSNYYKSINVDFAVNTE | |
| | GVYSEPRPIGTRYLTRNL | |
| 3 | MAADGYLPDWLEDNLSEGIREWWALKPGAPKPKANQQKQDDGRGLVLPGY | AAV8R2 |
| | KYLGPFNGLDKGEPVNAADAAALEHDKAYDQQLQAGDNPYLRYNHADAEF | (N411S) |
| | QERLQEDTSFGGNLGRAVFQAKKRVLEPLGLVEEGAKTAPGKKRPVEPSPQRS | |
| | PDSSTGIGKKGQQPARKRLNFGQTGDSESVPDPQPLGEPPAAPSGVGPNTMAA | |
| | GGGAPMADNNEGADGVGSSSGNWHCDSTWLGDRVITTSTRTWALPTYNNHL | |
| | YKQISNGTSGGATNDNTYFGYSTPWGYFDFNRFHCHFSPRDWQRLINNNWGF | |
| | RPKRLSFKLFNIQVKEVTQNEGTKTIANNLTSTIQVFTDSEYQLPYVLGSAHQG | |
| | CLPPFPADVFMIPQYGYLTLNDGSQAVGRSSFYCLEYFPSQMLRTGNSFQFTY | |
| | TFEDVPFHSSYAHSQSLDRLMNPLIDQYLYYLSRTQTTGGTANTQTLGFSQGG | |
| | PNTMANQAKNWLPGPCYRQQRVSTTTGQNNNSNFAWTAGTKYHLNGRNSL | |
| | ANPGIAMATHKDDEERFFPSNGILIFGKQNAARDNADYSDVMLTSEEEIKTTN | |
| | PVATEEYGIVADNLQRGNLADQDYTKTARQAATADVNSQGALPGMVWQNR | |
| | DVYLQGPIWAKIPHTDGNFHPSPLMGGFGLKHPPPQILIKNTPVPADPPTTFNQ | |
| | SKLNSFITQYSTGQVSVEIEWELQKENSKRWNPEIQYTSNYYKSINVDFAVNTE | |
| | GVYSEPRPIGTRYLTRNL | |
| 4 | MAADGYLPDWLEDNLSEGIREWWALKPGAPKPKANQQKQDDGRGLVLPGY | AAV8R3 |
| | KYLGPFNGLDKGEPVNAADAAALEHDKAYDQQLQAGDNPYLRYNHADAEF | (Y447F) |
| | QERLQEDTSFGGNLGRAVFQAKKRVLEPLGLVEEGAKTAPGKKRPVEPSPQRS | |
| | PDSSTGIGKKGQQPARKRLNFGQTGDSESVPDPQPLGEPPAAPSGVGPNTMAA | |
| | GGGAPMADNNEGADGVGSSSGNWHCDSTWLGDRVITTSTRTWALPTYNNHL | |
| | YKQISNGTSGGATNDNTYFGYSTPWGYFDFNRFHCHFSPRDWQRLINNNWGF | |
| | RPKRLSFKLFNIQVKEVTQNEGTKTIANNLTSTIQVFTDSEYQLPYVLGSAHQG | |
| | CLPPFPADVFMIPQYGYLTLNDGSQAVGRSSFYCLEYFPSQMLRTGNNFQFTY | |
| | TFEDVPFHSSYAHSQSLDRLMNPLIDQYLYFLSRTQTTGGTANTQTLGFSQGG | |
| | PNTMANQAKNWLPGPCYRQQRVSTTTGQNNNSNFAWTAGTKYHLNGRNSL | |
| | ANPGIAMATHKDDEERFFPSNGILIFGKQNAARDNADYSDVMLTSEEEIKTTN | |
| | PVATEEYGIVADNLQRGNLADQDYTKTARQAATADVNSQGALPGMVWQNR | |
| | DVYLQGPIWAKIPHTDGNFHPSPLMGGFGLKHPPPQILIKNTPVPADPPTTFNQ | |
| | SKLNSFITQYSTGQVSVEIEWELQKENSKRWNPEIQYTSNYYKSINVDFAVNTE | |
| | GVYSEPRPIGTRYLTRNL | |
| 5 | MAADGYLPDWLEDNLSEGIREWWALKPGAPKPKANQQKQDDGRGLVLPGY | AAV8R4 |
| | KYLGPFNGLDKGEPVNAADAAALEHDKAYDQQLQAGDNPYLRYNHADAEF | (R490Q) |
| | QERLQEDTSFGGNLGRAVFQAKKRVLEPLGLVEEGAKTAPGKKRPVEPSPQRS | |
| | PDSSTGIGKKGQQPARKRLNFGQTGDSESVPDPQPLGEPPAAPSGVGPNTMAA | |
| | GGGAPMADNNEGADGVGSSSGNWHCDSTWLGDRVITTSTRTWALPTYNNHL | |
| | YKQISNGTSGGATNDNTYFGYSTPWGYFDFNRFHCHFSPRDWQRLINNNWGF | |
| | RPKRLSFKLFNIQVKEVTQNEGTKTIANNLTSTIQVFTDSEYQLPYVLGSAHQG | |
| | CLPPFPADVFMIPQYGYLTLNDGSQAVGRSSFYCLEYFPSQMLRTGNNFQFTY | |
| | TFEDVPFHSSYAHSQSLDRLMNPLIDQYLYYLSRTQTTGGTANTQTLGFSQGG | |
| | PNTMANQAKNWLPGPCYRQQQVSTTTGQNNNSNFAWTAGTKYHLNGRNSL | |
| | ANPGIAMATHKDDEERFFPSNGILIFGKQNAARDNADYSDVMLTSEEEIKTTN | |
| | PVATEEYGIVADNLQRGNLADQDYTKTARQAATADVNSQGALPGMVWQNR | |
| | DVYLQGPIWAKIPHTDGNFHPSPLMGGFGLKHPPPQILIKNTPVPADPPTTFNQ | |
| | SKLNSFITQYSTGQVSVEIEWELQKENSKRWNPEIQYTSNYYKSINVDFAVNTE | |
| | GVYSEPRPIGTRYLTRNL | |
| 6 | MAADGYLPDWLEDNLSEGIREWWALKPGAPKPKANQQKQDDGRGLVLPGY | AAV8R5 |
| | KYLGPFNGLDKGEPVNAADAAALEHDKAYDQQLQAGDNPYLRYNHADAEF | (V125I) |
| | QERLQEDTSFGGNLGRAVFQAKKRILEPLGLVEEGAKTAPGKKRPVEPSPQRS | |
| | PDSSTGIGKKGQQPARKRLNFGQTGDSESVPDPQPLGEPPAAPSGVGPNTMAA | |
| | GGGAPMADNNEGADGVGSSSGNWHCDSTWLGDRVITTSTRTWALPTYNNHL | |
| | YKQISNGTSGGATNDNTYFGYSTPWGYFDFNRFHCHFSPRDWQRLINNNWGF | |
| | RPKRLSFKLFNIQVKEVTQNEGTKTIANNLTSTIQVFTDSEYQLPYVLGSAHQG | |
| | CLPPFPADVFMIPQYGYLTLNDGSQAVGRSSFYCLEYFPSQMLRTGNNFQFTY | |
| | TFEDVPFHSSYAHSQSLDRLMNPLIDQYLYYLSRTQTTGGTANTQTLGFSQGG | |
| | PNTMANQAKNWLPGPCYRQQRVSTTTGQNNNSNFAWTAGTKYHLNGRNSL | |
| | ANPGIAMATHKDDEERFFPSNGILIFGKQNAARDNADYSDVMLTSEEEIKTTN | |
| | PVATEEYGIVADNLQRGNLADQDYTKTARQAATADVNSQGALPGMVWQNR | |
| | DVYLQGPIWAKIPHTDGNFHPSPLMGGFGLKHPPPQILIKNTPVPADPPTTFNQ | |
| | SKLNSFITQYSTGQVSVEIEWELQKENSKRWNPEIQYTSNYYKSINVDFAVNTE | |
| | GVYSEPRPIGTRYLTRNL | |
| 7 | MAADGYLPDWLEDNLSEGIREWWALKPGAPKPKANQQKQDDGRGLVLPGY | AAV8R6 |
| | KYLGPFNGLDKGEPVNAADAAALEHDKAYDQQLQAGDNPYLRYNHADAEF | (F536Y) |
| | QERLQEDTSFGGNLGRAVFQAKKRVLEPLGLVEEGAKTAPGKKRPVEPSPQRS | |
| | PDSSTGIGKKGQQPARKRLNFGQTGDSESVPDPQPLGEPPAAPSGVGPNTMAA | |
| | GGGAPMADNNEGADGVGSSSGNWHCDSTWLGDRVITTSTRTWALPTYNNHL | |
| | YKQISNGTSGGATNDNTYFGYSTPWGYFDFNRFHCHFSPRDWQRLINNNWGF | |
| | RPKRLSFKLFNIQVKEVTQNEGTKTIANNLTSTIQVFTDSEYQLPYVLGSAHQG | |
| | CLPPFPADVFMIPQYGYLTLNDGSQAVGRSSFYCLEYFPSQMLRTGNNFQFTY | |
| | TFEDVPFHSSYAHSQSLDRLMNPLIDQYLYYLSRTQTTGGTANTQTLGFSQGG | |
| | PNTMANQAKNWLPGPCYRQQRVSTTTGQNNNSNFAWTAGTKYHLNGRNSL | |
| | ANPGIAMATHKDDEERYFPSNGILIFGKQNAARDNADYSDVMLTSEEEIKTTN | |
| | PVATEEYGIVADNLQRGNLADQDYTKTARQAATADVNSQGALPGMVWQNR | |
| | DVYLQGPIWAKIPHTDGNFHPSPLMGGFGLKHPPPQILIKNTPVPADPPTTFNQ | |
| | SKLNSFITQYSTGQVSVEIEWELQKENSKRWNPEIQYTSNYYKSINVDFAVNTE | |
| | GVYSEPRPIGTRYLTRNL | |
| 8 | MAADGYLPDWLEDNLSEGIREWWALKPGAPKPKANQQKQDDGRGLVLPGY | AAV8R7 |
| | KYLGPFNGLDKGEPVNAADAAALEHDKAYDQQLQAGDNPYLRYNHADAEF | (A606S) |
| | QERLQEDTSFGGNLGRAVFQAKKRVLEPLGLVEEGAKTAPGKKRPVEPSPQRS | |
| | PDSSTGIGKKGQQPARKRLNFGQTGDSESVPDPQPLGEPPAAPSGVGPNTMAA | |
| | GGGAPMADNNEGADGVGSSSGNWHCDSTWLGDRVITTSTRTWALPTYNNHL | |
| | YKQISNGTSGGATNDNTYFGYSTPWGYFDFNRFHCHFSPRDWQRLINNNWGF | |
| | RPKRLSFKLFNIQVKEVTQNEGTKTIANNLTSTIQVFTDSEYQLPYVLGSAHQG | |
| | CLPPFPADVFMIPQYGYLTLNDGSQAVGRSSFYCLEYFPSQMLRTGNNFQFTY | |
| | TFEDVPFHSSYAHSQSLDRLMNPLIDQYLYYLSRTQTTGGTANTQTLGFSQGG | |
| | PNTMANQAKNWLPGPCYRQQRVSTTTGQNNNSNFAWTAGTKYHLNGRNSL | |
| | ANPGIAMATHKDDEERFFPSNGILIFGKQNAARDNADYSDVMLTSEEEIKTTN | |
| | PVATEEYGIVADNLQRGNLADQDYTKTARQAATSDVNSQGALPGMVWQNR | |
| | DVYLQGPIWAKIPHTDGNFHPSPLMGGFGLKHPPPQILIKNTPVPADPPTTFNQ | |
| | SKLNSFITQYSTGQVSVEIEWELQKENSKRWNPEIQYTSNYYKSINVDFAVNTE | |
| | GVYSEPRPIGTRYLTRNL | |
| 9 | MAADGYLPDWLEDNLSEGIREWWALKPGAPKPKANQQKQDDGRGLVLPGY | AAV8R8 |
| | KYLGPFNGLDKGEPVNAADAAALEHDKAYDQQLQAGDNPYLRYNHADAEF | (T495A) |
| | QERLQEDTSFGGNLGRAVFQAKKRVLEPLGLVEEGAKTAPGKKRPVEPSPQRS | |
| | PDSSTGIGKKGQQPARKRLNFGQTGDSESVPDPQPLGEPPAAPSGVGPNTMAA | |
| | GGGAPMADNNEGADGVGSSSGNWHCDSTWLGDRVITTSTRTWALPTYNNHL | |
| | YKQISNGTSGGATNDNTYFGYSTPWGYFDFNRFHCHFSPRDWQRLINNNWGF | |
| | RPKRLSFKLFNIQVKEVTQNEGTKTIANNLTSTIQVFTDSEYQLPYVLGSAHQG | |
| | CLPPFPADVFMIPQYGYLTLNDGSQAVGRSSFYCLEYFPSQMLRTGNNFQFTY | |
| | TFEDVPFHSSYAHSQSLDRLMNPLIDQYLYYLSRTQTTGGTANTQTLGFSQGG | |
| | PNTMANQAKNWLPGPCYRQQRVSTTAGQNNNSNFAWTAGTKYHLNGRNSL | |
| | ANPGIAMATHKDDEERFFPSNGILIFGKQNAARDNADYSDVMLTSEEEIKTTN | |
| | PVATEEYGIVADNLQRGNLADQDYTKTARQAATADVNSQGALPGMVWQNR | |
| | DVYLQGPIWAKIPHTDGNFHPSPLMGGFGLKHPPPQILIKNTPVPADPPTTFNQ | |
| | SKLNSFITQYSTGQVSVEIEWELQKENSKRWNPEIQYTSNYYKSINVDFAVNTE | |
| | GVYSEPRPIGTRYLTRNL | |
| 10 | MAADGYLPDWLEDNLSEGIREWWALKPGAPKPKANQQKQDDGRGLVLPGY | AAV8R9 |
| | KYLGPFNGLDKGEPVNAADAAALEHDKAYDQQLQAGDNPYLRYNHADAEF | (V125I + |
| | QERLQEDTSFGGNLGRAVFQAKKRILEPLGLVEEGAKTAPGKKRPVEPSPQRS | F536Y) |
| | PDSSTGIGKKGQQPARKRLNFGQTGDSESVPDPQPLGEPPAAPSGVGPNTMAA | |
| | GGGAPMADNNEGADGVGSSSGNWHCDSTWLGDRVITTSTRTWALPTYNNHL | |
| | YKQISNGTSGGATNDNTYFGYSTPWGYFDFNRFHCHFSPRDWQRLINNNWGF | |
| | RPKRLSFKLFNIQVKEVTQNEGTKTIANNLTSTIQVFTDSEYQLPYVLGSAHQG | |
| | CLPPFPADVFMIPQYGYLTLNDGSQAVGRSSFYCLEYFPSQMLRTGNNFQFTY | |
| | TFEDVPFHSSYAHSQSLDRLMNPLIDQYLYYLSRTQTTGGTANTQTLGFSQGG | |
| | PNTMANQAKNWLPGPCYRQQRVSTTTGQNNNSNFAWTAGTKYHLNGRNSL | |
| | ANPGIAMATHKDDEERYFPSNGILIFGKQNAARDNADYSDVMLTSEEEIKTTN | |
| | PVATEEYGIVADNLQRGNLADQDYTKTARQAATADVNSQGALPGMVWQNR | |
| | DVYLQGPIWAKIPHTDGNFHPSPLMGGFGLKHPPPQILIKNTPVPADPPTTFNQ | |
| | SKLNSFITQYSTGQVSVEIEWELQKENSKRWNPEIQYTSNYYKSINVDFAVNTE | |
| | GVYSEPRPIGTRYLTRNL | |
| 11 | MAADGYLPDWLEDNLSEGIREWWALKPGAPKPKANQQKQDDGRGLVLPGY | AAV8R10 |
| | KYLGPFNGLDKGEPVNAADAAALEHDKAYDQQLQAGDNPYLRYNHADAEF | (V125I + |
| | QERLQEDTSFGGNLGRAVFQAKKRILEPLGLVEEGAKTAPGKKRPVEPSPQRS | A606S) |
| | PDSSTGIGKKGQQPARKRLNFGQTGDSESVPDPQPLGEPPAAPSGVGPNTMAA | |
| | GGGAPMADNNEGADGVGSSSGNWHCDSTWLGDRVITTSTRTWALPTYNNHL | |
| | YKQISNGTSGGATNDNTYFGYSTPWGYFDFNRFHCHFSPRDWQRLINNNWGF | |
| | RPKRLSFKLFNIQVKEVTQNEGTKTIANNLTSTIQVFTDSEYQLPYVLGSAHQG | |
| | CLPPFPADVFMIPQYGYLTLNDGSQAVGRSSFYCLEYFPSQMLRTGNNFQFTY | |
| | TFEDVPFHSSYAHSQSLDRLMNPLIDQYLYYLSRTQTTGGTANTQTLGFSQGG | |
| | PNTMANQAKNWLPGPCYRQQRVSTTTGQNNNSNFAWTAGTKYHLNGRNSL | |
| | ANPGIAMATHKDDEERFFPSNGILIFGKQNAARDNADYSDVMLTSEEEIKTTN | |
| | PVATEEYGIVADNLQRGNLADQDYTKTARQAATSDVNSQGALPGMVWQNR | |
| | DVYLQGPIWAKIPHTDGNFHPSPLMGGFGLKHPPPQILIKNTPVPADPPTTFNQ | |
| | SKLNSFITQYSTGQVSVEIEWELQKENSKRWNPEIQYTSNYYKSINVDFAVNTE | |
| | GVYSEPRPIGTRYLTRNL | |
| 12 | MAADGYLPDWLEDNLSEGIREWWALKPGAPKPKANQQKQDDGRGLVLPGY | AAV8R11 |
| | KYLGPFNGLDKGEPVNAADAAALEHDKAYDQQLQAGDNPYLRYNHADAEF | (V125I + |
| | QERLQEDTSFGGNLGRAVFQAKKRILEPLGLVEEGAKTAPGKKRPVEPSPQRS | T495A) |
| | PDSSTGIGKKGQQPARKRLNFGQTGDSESVPDPQPLGEPPAAPSGVGPNTMAA | |
| | GGGAPMADNNEGADGVGSSSGNWHCDSTWLGDRVITTSTRTWALPTYNNHL | |
| | YKQISNGTSGGATNDNTYFGYSTPWGYFDFNRFHCHFSPRDWQRLINNNWGF | |
| | RPKRLSFKLFNIQVKEVTQNEGTKTIANNLTSTIQVFTDSEYQLPYVLGSAHQG | |
| | CLPPFPADVFMIPQYGYLTLNDGSQAVGRSSFYCLEYFPSQMLRTGNNFQFTY | |
| | TFEDVPFHSSYAHSQSLDRLMNPLIDQYLYYLSRTQTTGGTANTQTLGFSQGG | |
| | PNTMANQAKNWLPGPCYRQQRVSTTAGQNNNSNFAWTAGTKYHLNGRNSL | |
| | ANPGIAMATHKDDEERFFPSNGILIFGKQNAARDNADYSDVMLTSEEEIKTTN | |
| | PVATEEYGIVADNLQRGNLADQDYTKTARQAATADVNSQGALPGMVWQNR | |
| | DVYLQGPIWAKIPHTDGNFHPSPLMGGFGLKHPPPQILIKNTPVPADPPTTFNQ | |
| | SKLNSFITQYSTGQVSVEIEWELQKENSKRWNPEIQYTSNYYKSINVDFAVNTE | |
| | GVYSEPRPIGTRYLTRNL | |
| | MAADGYLPDWLEDNLSEGIREWWALKPGAPKPKANQQKQDDGRGLVLPGY | |
| 13 | KYLGPFNGLDKGEPVNAADAAALEHDKAYDQQLQAGDNPYLRYNHADAEF | AAV8R12 |
| | QERLQEDTSFGGNLGRAVFQAKKRVLEPLGLVEEGAKTAPGKKRPVEPSPQRS | (V183E + |
| | PDSSTGIGKKGQQPARKRLNFGQTGDSESEPDPQPLGEPPAAPSGVGPNTMAA | N411S) |
| | GGGAPMADNNEGADGVGSSSGNWHCDSTWLGDRVITTSTRTWALPTYNNHL | |
| | YKQISNGTSGGATNDNTYFGYSTPWGYFDFNRFHCHFSPRDWQRLINNNWGF | |
| | RPKRLSFKLFNIQVKEVTQNEGTKTIANNLTSTIQVFTDSEYQLPYVLGSAHQG | |
| | CLPPFPADVFMIPQYGYLTLNDGSQAVGRSSFYCLEYFPSQMLRTGNSFQFTY | |
| | TFEDVPFHSSYAHSQSLDRLMNPLIDQYLYYLSRTQTTGGTANTQTLGFSQGG | |
| | PNTMANQAKNWLPGPCYRQQRVSTTTGQNNNSNFAWTAGTKYHLNGRNSL | |
| | ANPGIAMATHKDDEERFFPSNGILIFGKQNAARDNADYSDVMLTSEEEIKTTN | |
| | PVATEEYGIVADNLQRGNLADQDYTKTARQAATADVNSQGALPGMVWQNR | |
| | DVYLQGPIWAKIPHTDGNFHPSPLMGGFGLKHPPPQILIKNTPVPADPPTTFNQ | |
| | SKLNSFITQYSTGQVSVEIEWELQKENSKRWNPEIQYTSNYYKSINVDFAVNTE | |
| | GVYSEPRPIGTRYLTRN | |
| 14 | MAADGYLPDWLEDNLSEGIREWWALKPGAPKPKANQQKQDDGRGLVLPGY | AAV8R13 |
| | KYLGPFNGLDKGEPVNAADAAALEHDKAYDQQLQAGDNPYLRYNHADAEF | (V125I + |
| | QERLQEDTSFGGNLGRAVFQAKKRILEPLGLVEEGAKTAPGKKRPVEPSPQRS | F536Y + |
| | PDSSTGIGKKGQQPARKRLNFGQTGDSESVPDPQPLGEPPAAPSGVGPNTMAA | T495A): |
| | GGGAPMADNNEGADGVGSSSGNWHCDSTWLGDRVITTSTRTWALPTYNNHL | |
| | YKQISNGTSGGATNDNTYFGYSTPWGYFDFNRFHCHFSPRDWQRLINNNWGF | |
| | RPKRLSFKLFNIQVKEVTQNEGTKTIANNLTSTIQVFTDSEYQLPYVLGSAHQG | |
| | CLPPFPADVFMIPQYGYLTLNDGSQAVGRSSFYCLEYFPSQMLRTGNNFQFTY | |
| | TFEDVPFHSSYAHSQSLDRLMNPLIDQYLYYLSRTQTTGGTANTQTLGFSQGG | |
| | PNTMANQAKNWLPGPCYRQQRVSTTAGQNNNSNFAWTAGTKYHLNGRNSL | |
| | ANPGIAMATHKDDEERYFPSNGILIFGKQNAARDNADYSDVMLTSEEEIKTTN | |
| | PVATEEYGIVADNLQRGNLADQDYTKTARQAATADVNSQGALPGMVWQNR | |
| | DVYLQGPIWAKIPHTDGNFHPSPLMGGFGLKHPPPQILIKNTPVPADPPTTFNQ | |
| | SKLNSFITQYSTGQVSVEIEWELQKENSKRWNPEIQYTSNYYKSINVDFAVNTE | |
| | GVYSEPRPIGTRYLTRNL | |
| 15 | MAADGYLPDWLEDNLSEGIREWWALKPGAPKPKANQQKQDDGRGLVLPGY | AAV8R14 |
| | KYLGPFNGLDKGEPVNAADAAALEHDKAYDQQLQAGDNPYLRYNHADAEF | (V125I + |
| | QERLQEDTSFGGNLGRAVFQAKKRILEPLGLVEEGAKTAPGKKRPVEPSPQRS | A606S + |
| | PDSSTGIGKKGQQPARKRLNFGQTGDSESVPDPQPLGEPPAAPSGVGPNTMAA | T495A) |
| | GGGAPMADNNEGADGVGSSSGNWHCDSTWLGDRVITTSTRTWALPTYNNHL | |
| | YKQISNGTSGGATNDNTYFGYSTPWGYFDFNRFHCHFSPRDWQRLINNNWGF | |
| | RPKRLSFKLFNIQVKEVTQNEGTKTIANNLTSTIQVFTDSEYQLPYVLGSAHQG | |
| | CLPPFPADVFMIPQYGYLTLNDGSQAVGRSSFYCLEYFPSQMLRTGNNFQFTY | |
| | TFEDVPFHSSYAHSQSLDRLMNPLIDQYLYYLSRTQTTGGTANTQTLGFSQGG | |
| | PNTMANQAKNWLPGPCYRQQRVSTTAGQNNNSNFAWTAGTKYHLNGRNSL | |
| | ANPGIAMATHKDDEERFFPSNGILIFGKQNAARDNADYSDVMLTSEEEIKTTN | |
| | PVATEEYGIVADNLQRGNLADQDYTKTARQAATSDVNSQGALPGMVWQNR | |
| | DVYLQGPIWAKIPHTDGNFHPSPLMGGFGLKHPPPQILIKNTPVPADPPTTFNQ | |
| | SKLNSFITQYSTGQVSVEIEWELQKENSKRWNPEIQYTSNYYKSINVDFAVNTE | |
| | GVYSEPRPIGTRYLTRNL | |
| 20 | MAADGYLPDWLEDTLSEGIRQWWKLKPGPPPPKPAERHKDDSRGLVLPGYK | AAV2 |
| | YLGPFNGLDKGEPVNEADAAALEHDKAYDRQLDSGDNPYLKYNHADAEFQE | |
| | RLKEDTSFGGNLGRAVFQAKKRVLEPLGLVEEPVKTAPGKKRPVEHSPVEPDS | |
| | SSGTGKAGQQPARKRLNFGQTGDADSVPDPQPLGQPPAAPSGLGTNTMATGS | |
| | GAPMADNNEGADGVGNSSGNWHCDSTWMGDRVITTSTRTWALPTYNNHLY | |
| | KQISSQSGASNDNHYFGYSTPWGYFDFNRFHCHFSPRDWQRLINNNWGFRPK | |
| | RLNFKLFNIQVKEVTQNDGTTTIANNLTSTVQVFTDSEYQLPYVLGSAHQGCL | |
| | PPFPADVFMVPQYGYLTLNNGSQAVGRSSFYCLEYFPSQMLRTGNNFTFSYTF | |
| | EDVPFHSSYAHSQSLDRLMNPLIDQYLYYLSRTNTPSGTTTQSRLQFSQAGAS | |
| | DIRDQSRNWLPGPCYRQQRVSKTSADNNNSEYSWTGATKYHLNGRDSLVNP | |
| | GPAMASHKDDEEKFFPQSGVLIFGKQGSEKTNVDIEKVMITDEEEIRTTNPVAT | |
| | EQYGSVSTNLQRGNRQAATADVNTQGVLPGMVWQDRDVYLQGPIWAKIPHT | |
| | DGHFHPSPLMGGFGLKHPPPQILIKNTPVPANPSTTFSAAKFASFITQYSTGQVS | |
| | VEIEWELQKENSKRWNPEIQYTSNYNKSVNVDFTVDTNGVYSEPRPIGTRYLT | |
| | RNL* | |
| 21 | MAADGYLPDWLEDTLSEGIRQWWKLKPGPPPPKPAERHKDDSRGLVLPGYK | rAAV2- |
| | YLGPFNGLDKGEPVNEADAAALEHDKAYDRQLDSGDNPYLKYNHADAEFQE | retro |
| | RLKEDTSFGGNLGRAVFQAKKRVLEPLGLVEEPVKTAPGKKRPVEHSPVEPDS | |
| | SSGTGKAGQQPARKRLNFGQTGDADSVPDPQPLGQPPAAPSGLGTNTMATGS | |
| | GAPMADNNEGADGVGNSSGNWHCDSTWMGDRVITTSTRTWALPTYNNHLY | |
| | KQISSQSGASNDNHYFGYSTPWGYFDFNRFHCHFSPRDWQRLINNNWGFRPK | |
| | RLNFKLFNIQVKEVTQNDGTTTIANNLTSTVQVFTDSEYQLPYVLGSAHQGCL | |
| | PPFPADVFMVPQYGYLTLNDGSQAVGRSSFYCLEYFPSQMLRTGNNFTFSYTF | |
| | EDVPFHSSYAHSQSLDRLMNPLIDQYLYYLSRTNTPSGTTTQSRLQFSQAGAS | |
| | DIRDQSRNWLPGPCYRQQRVSKTSADNNNSEYSWTGATKYHLNGRDSLVNP | |
| | GPAMASHKDDEEKFFPQSGVLIFGKQGSEKTNVDIEKVMITDEEEIRTTNPVAT | |
| | EQYGSVSTNLQRGNLADQDYTKTARQAATADVNTQGVLPGMVWQDRDVY | |
| | LQGPIWAKIPHTDGHFHPSPLMGGFGLKHPPPQILIKNTPVPANPSTTFSAAKFA | |
| | SFITQYSTGQVSVEIEWELQKENSKRWNPEIQYTSNYNKSINVDFTVDTNGVY | |
| | SEPRPIGTRYLTRNL* | |
| 30 | MAADGYLPDWLEDNLSEGIREWWALKPGAPKPKANQQKQDDGRGLVLPGY | AAV8 |
| | KYLGPFNGLDKGEPVNAADAAALEHDKAYDQQLQAGDNPYLRYNHADAEF | |
| | QERLQEDTSFGGNLGRAVFQAKKRVLEPLGLVEEGAKTAPGKKRPVEPSPQRS | |
| | PDSSTGIGKKGQQPARKRLNFGQTGDSESVPDPQPLGEPPAAPSGVGPNTMAA | |
| | GGGAPMADNNEGADGVGSSSGNWHCDSTWLGDRVITTSTRTWALPTYNNHL | |
| | YKQISNGTSGGATNDNTYFGYSTPWGYFDFNRFHCHFSPRDWQRLINNNWGF | |
| | RPKRLSFKLFNIQVKEVTQNEGTKTIANNLTSTIQVFTDSEYQLPYVLGSAHQG | |
| | CLPPFPADVFMIPQYGYLTLNNGSQAVGRSSFYCLEYFPSQMLRTGNNFQFTY | |
| | TFEDVPFHSSYAHSQSLDRLMNPLIDQYLYYLSRTQTTGGTANTQTLGFSQGG | |
| | PNTMANQAKNWLPGPCYRQQRVSTTTGQNNNSNFAWTAGTKYHLNGRNSL | |
| | ANPGIAMATHKDDEERFFPSNGILIFGKQNAARDNADYSDVMLTSEEEIKTTN | |
| | PVATEEYGIVADNLQGQRGNEARVREAQAAQIGTVNSQGALPGMVWQNRDV | |
| | YLQGPIWAKIPHTDGNFHPSPLMGGFGLKHPPPQILIKNTPVPADPPTTFNQSKL | |
| | NSFITQYSTGQVSVEIEWELQKENSKRWNPEIQYTSNYYKSTSVDFAVNTEGV | |
| | YSEPRPIGTRYLTRNL* | |
| 31 | RGNLADQDYTKTARQAATAD | AAV |
| | | retro |
| | | inser- |
| | | tion |
| 32 | RGNLADQDYTKTARQAATSD | AAV |
| | | retro |
| | | inser- |
| | | tion |
| 33 | LQQONTAPQIGTVN | |
| 34 | TGGCGACTCAGAGTCAGAGCCAGACCCTCAACCTCT | |
| 35 | TGCTGAGAACCGGCAACAGCTTCCAGTTTACTTACACCT | |
| 36 | CATCGCAAGGCTACATGATGG | |
| 52 | GAGGTGTGAAGAGTTGGCCAG | |
| 38 | MTLHSNSTTSPLFPNISSSWVHSPSEAGLPLGTVTQLGSYNISQETGNFSSNDTS | rM3Ds |
| | SDPLGGHTIWQVVFIAFLTGFLALVTIIGNILVIVAFKVNKQLKTVNNYFLLSLA | |
| | CADLIIGVISMNLFTTYIIMNRWALGNLACDLWLSIDCVASNASVMNLLVISFD | |
| | RYFSITSPFRYQSLMTRARAGVMIGLAWVISFVLWAPAILFWQYFVGKRTVPP | |
| | GECFIQFLSEPTITFGTAIAGFYMPVTIMTILYWRVYREAKEQIRKIDRCEGRFY | |
| | GSQEQPQPPPLPQHQPILGNGRASKRKTSRVMAMREHKALQTLSAILLAFIITW | |
| | TPYNIMVLVNTFCDSCIPKTYWNLGYWLCYINSTVNPVCYALCNKTFRTTFKT | |
| | LLLCQCDKRKRRKQQYQQRQSVIFHKRVPEQALADPPV | |
| 39 | GGGTTCACCACATCAGCCACCACTCCTGCTTCTGAGCACAGGGTGCTCTCC | |
| | TCTTGAGCTCAGCTTCTGCTTTTGCAGCCAAGCATTCTTGCTGCTGCTGCCT | |
| | GCCTGCCCACCCGCCTGGGCTTGCAGCCCGCCACTTTACTTTCTCCAGCCCT | |
| | GATACCAGCTGAGAAGTCTCCCTGCAGCTGCTAGTTCCTGCCCAGGACCAT | |
| | GTGTGTGGATGCTGCTTGGGAGAAGCGGGCACTTGCTCCTGGCACTGATCC | |
| | CAGCTGAGTTTCTCCTGTTGATTTCTGGACCACTGATGCTGTTGCTGAGGA | |
| | GGTATTTCCCGGCATCCCTCCCCCTGAGACACCGGCTAAGGACCAGCCTAA | |
| | ACGCAAGGTAACCTTCACCTCTTTCCCATGGGATTTTCCCCAGCTGGGATA | |
| | TCTCTGCTTTCTCCTGGAACTAGGTCCTCCAGCTGGCAACATATCTCTCTCT | |
| | CTCTCTCTCTCTCTCTCTCTCTCTCTCTCTCTGTGTGTGTGTGTGTGTGTTGG | |
| | GGGAGAAGAGGAGACTGTTTTAGGTATTGCTCTGTCCTAGGGCTTCCCCGA | |
| | TACCTGCTGAATCCAATGCAAAGTGGCCTTTCCCCTGGCTCTGGAGACATT | |
| | TCCGAGGCAGGCACCTGTCATCTGCCACCCCCGTCCTCCTCCTCGAGCTCC | |
| | CTTTCTCCCCCTCCCCCAGCCCATTATTCTGCTTCAGCCTTTTGTGTCAGTG | |
| | GCAGAGGGCTGAAGGGATGTCTTTGCCCTTCTGGCAGGCAGGACAGTGTC | |
| | AGGATGGACCGCGCTGCCAGAAGCCGACGCTAGCGAGGGAGGTGTGAAG | |
| | AGTTGGCCAGAATGACCAACTCCTCCTCCACATCCACCTCCTCCACCACCG | |
| | GTGGCTCGCTGCTGCTGCTCTGCGAGGAAGAGGAGTCGTGGGGGGCCGG | |
| | CGCATCCCGGTGTCACTCCTGTATTCGGGCCTGGCCATCGGGGGCACGCTG | |
| | GCCAACGGCATGGTCATCTATCTCGTGTC | |
| 40 | GGGTTCACCACATCAGCCACCACTCCTGCTTCTGAGCACAGGGTGCTCTCC | |
| | TCTTGAGCTCAGCTTCTGCTTTTGCAGCCAAGCATTCTTGCTGCTGCTGCCT | |
| | GCCTGCCCACCCGCCTGGGCTTGCAGCCCGCCACTTTACTTTCTCCAGCCCT | |
| | GATACCAGCTGAGAAGTCTCCCTGCAGCTGCTAGTTCCTGCCCAGGACCAT | |
| | GTGTGTGGATGCTGCTTGGGAGAAGCGGGCACTTGCTCCTGGCACTGATCC | |
| | CAGCTGAGTTTCTCCTGTTGATTTCTGGACCACTGATGCTGTTGCTGAGGA | |
| | GGTATTTCCCGGCATCCCTCCCCCTGAGACACCGGCTAAGGACCAGCCTAA | |
| | ACGCAAGGTAACCTTCACCTCTTTCCCATGGGATTTTCCCCAGCTGGGATA | |
| | TCTCTGCTTTCTCCTGGAACTAGGTCCTCCAGCTGGCAACATATCTCTCTCT | |
| | CTCTCTCTCTCTCTCTCTCTCTCTCTCTCTCTGTGTGTGTGTGTGTGTGTTGG | |
| | GGGAGAAGAGGAGACTGTTTTAGGTATTGCTCTGTCCTAGGGCTTCCCCGA | |
| | TACCTGCTGAATCCAATGCAAAGTGGCCTTTCCCCTGGCTCTGGAGACATT | |
| | TCCGAGGCAGGCACCTGTCATCTGCCACCCCCGTCCTCCTCCTCGAGCTCC | |
| | CTTTCTCCCCCTCCCCCAGCCCATTATTCTGCTTCAGCCTTTTGTGTCAGTG | |
| | GCAGAGGGCTGAAGGGATGTCTTTGCCCTTCTGGCAGGCAGGACAGTGTC | |
| | AGGATGGACCGCGCTGCCAGAAGCCGACGCTAGCGAGGGAGGTGTGAAG | |
| | AGTTGGCCAGAATGACCAACTCCTCCTCCACATCCACCTCCTCCACCACCG | |
| | GTGGCTCGCTGCTGCTGCTCTGCGAGGAAG | |
| 41 | TCCTTTTACAAGTTGGAAGCTATTGAATAATTCATGCCAAGCGTGGAAAGG | |
| | CTGTCTTTTCCCTGGATGATGTATAACTAAGCAGGAGAGAGCTTCGAGT | |
| 42 | ACTGAATAATATTCCATTGTCTAGATGTACCACTGACATCTGCTTTAAATCT | |
| | TTTGCCCATTTCATTTTTACCCTCTAACCATTTCATTACTAAAACACAAAAC | |
| | AAACTCCGGAAAGGAGTTTGTTGATGAGAAAACACTGAAAGTACGATTTA | |
| | AATTGAGGAATGGAGATTTTCTGTGGATAATTGTCTATGTTCCACTTTACC | |
| | AAAGAAAAATGAATTTCAAGTAGTGCATAAAGTAATTCTCGTTAAAAAAT | |
| | GTGGGAATAAAGAGATCAGTCCTGGGTACTGGGAATATATTTATCTCACAG | |
| | AAAAATCTTGCCCACAAAATGCATCAGACTGAGATGAAATACTCTAGGGT | |
| | ATATTAAATTTTATTCTAATATTGAAGGGAGGGAAAAAAAGGACGGAAAG | |
| | AAAAATGAAACAGGATGAAAAATAAGGTGATTGGGAGAGAGAAAAGACA | |
| | GAGAAACTTGTTAACGACAGGACTTTCTGAGCAAGATATTTTTCTTCCTTTT | |
| | ACAAGTTGGAAGCTATTGAATAATTCATGCCAAGCGTGGAAAGGCTGTCTT | |
| | TTCCCTGGATGATGTATAACTAAGCAGGAGAGAGCTTCGAGT | |
| 43 | CAGAAAGTTTTAACAGAAAGGGCCTTTGAAGTGACTGACTGTGAACTTATT | |
| | TGACAAGATGAAAATAATCCATCGCAAGGCTACATGATGGATGCTCGGCT | |
| | TAGTATCTGGGTGTGCTGCAACCCCACCTAACATTTTTTTCTATTATGCCAT | |
| | GTTACCATATTCCCATATTGAGTTACATTTTCAAATACATGACTTTTCCAGT | |
| | GAATAATTGAGAGAACTCCTTTTCTGAAAAAATTCCCACATGGAATGGATA | |
| | AATGAACACTTGCACCAAAGAAGATGCCAACATCAGACTGCAAATCTCCA | |
| | AGACAGACAAGACAAAAAGTTCTGAAGTGAAAAGTCATCTTTGTTCACAC | |
| | TGTGGATTATTCAGGTTTCTTGCTTTCCCCCTGACACACATTTTTTTAAAAT | |
| | TGACTTTATTTTTTTAGAGCAATTTTAGATTCATGGCAAATTTGAGTGAAAG | |
| | ATACAGAGATTTCCCATGTACTCCCTGCCCCTCGCATACATAGCCTCTCCC | |
| | ATTATCAAAATCCCCCACCAGAGTGGTTCGTCTGTTACAATTGAATGTACG | |
| | TTGACACATCATTCCCCAAAGTCCATGGCTTACACTGATGTTTCACATTCTG | |
| | CCACTTTGGGCAAATTTATAAGTACACAGATCCACTATTATGGTATCATAC | |
| | AGAGTAGTTCCACTGTCCTAAGAATCTTCCGTGATCATCTATTCATTCCTCC | |
| | TTTCCCCCTAACTCGGGCAACCACTGGTCTTTTTAACTGTCTCCATAGTTTT | |
| | GCCTTCTCAAGAATGTCATAGTTGGTAGCATACAGTATGTAGTCCTTTCCA | |
| | GTTGGCTTCTGTCACTTAGTGATACACACTTAAGGTTCCTTCATGTCTTTTC | |
| | ATGGCTGGACAGCTCATTTCTTTTTAGCACTGAATAATATTCCATTGTCTAG | |
| | ATGTACCACTGACATCTGCTTTAAATCTTTTGCCCATTTCATTTTTACCCTCT | |
| | AACCATTTCATTACTAAAACACAAAACAAACTCCGGAAAGGAGTTTGTTG | |
| | ATGAGAAAACACTGAAAGTACGATTTAAATTGAGGAATGGAGATTTTCTG | |
| | TGGATAATTGTCTATGTTCCACTTTACCAAAGAAAAATGAATTTCAAGTAG | |
| | TGCATAAAGTAATTCTCGTTAAAAAATGTGGGAATAAAGAGATCAGTCCT | |
| | GGGTACTGGGAATATATTTATCTCACAGAAAAATCTTGCCCACAAAATGCA | |
| | TCAGACTGAGATGAAATACTCTAGGGTATATTAAATTTTATTCTAATATTG | |
| | AAGGGAGGGAAAAAAAGGACGGAAAGAAAAATGAAACAGGATGAAAAA | |
| | TAAGGTGATTGGGAGAGAGAAAAGACAGAGAAACTTGTTAACGACAGGA | |
| | CTTTCTGAGCAAGATATTTTTCTTCCTTTTACAAGTTGGAAGCTATTGAATA | |
| | ATTCATGCCAAGCGTGGAAAGGCTGTCTTTTCCCTGGATGATGTATAACTA | |
| | AGCAGGAGAGAGCTTCGAGT | |
| 44 | CATCGCAAGGCTACATGATGGATGCTCGGCTTAGTATCTGGGTGTGCTGCA | |
| | ACCCCACCTAACATTTTTTTCTATTATGCCATGTTACCATATTCCCATATTG | G88P2 |
| | AGTTACATTTTCAAATACATGACTTTTCCAGTGAATAATTGAGAGAACTCC | |
| | TTTTCTGAAAAAATTCCCACATGGAATGGATAAATGAACACTTGCACCAAA | |
| | GAAGATGCCAACATCAGACTGCAAATCTCCAAGACAGACAAGACAAAAAG | |
| | TTCTGAAGTGAAAAGTCATCTTTGTTCACACTGTGGATTATTCAGGTTTCTT | |
| | GCTTTCCCCCTGACACACATTTTTTTAAAATTGACTTTATTTTTTTAGAGCA | |
| | ATTTTAGATTCATGGCAAATTTGAGTGAAAGATACAGAGATTTCCCATGTA | |
| | CTCCCTGCCCCTCGCATACATAGCCTCTCCCATTATCAAAATCCCCCACCA | |
| | GAGTGGTTCGTCTGTTACAATTGAATGTACGTTGACACATCATTCCCCAAA | |
| | GTCCATGGCTTACACTGATGTTTCACATTCTGCCACTTTGGGCAAATTTATA | |
| | AGTACACAGATCCACTATTATGGTATCATACAGAGTAGTTCCACTGTCCTA | |
| | AGAATCTTCCGTGATCATCTATTCATTCCTCCTTTCCCCCTAACTCGGGCAA | |
| | CCACTGGTCTTTTTAACTGTCTCCATAGTTTTGCCTTCTCAAGAATGTCATA | |
| | GTTGGTAGCATACAGTATGTAGTCCTTTCCAGTTGGCTTCTGTCACTTAGTG | |
| | ATACACACTTAAGGTTCCTTCATGTCTTTTCATGGCTGGACAGCTCATTTCT | |
| | TTTTAGCACTGAATAATATTCCATTGTCTAGATGTACCACTGACATCTGCTT | |
| | TAAATCTTTTGCCCATTTCATTTTTACCCTCTAACCATTTCATTACTAAAAC | |
| | ACAAAACAAACTCCGGAAAGGAGTTTGTTGATGAGAAAACACTGAAAGTA | |
| | CGATTTAAATTGAGGAATGGAGATTTTCTGTGGATAATTGTCTATGTTCCA | |
| | CTTTACCAAAGAAAAATGAATTTCAAGTAGTGCATAAAGTAATTCTCGTTA | |
| | AAAAATGTGGGAATAAAGAGATCAGTCCTGGGTACTGGGAATATATTTAT | |
| | CTCACAGAAAAATCTTGCCCACAAAATGCATCAGACTGAGATGAAATACT | |
| | CTAGGGTATATTAAATTTTATTCTAATATTGAAGGGAGGGAAAAAAAGGA | |
| | CGGAAAGAAAAATGAAACAGGATGAAAAATAAGGTGATTGGGAGAGAGA | |
| | AAAGACAGAGAAACTTGTTAACGACAGGACTTTCTGAGCAAGATATTTTTC | |
| | TTCCTTTTACAAGTTGGAAGCTATTGAATAATTCATGCCAAGCGTGGAAAG | |
| | GCTGTCTTTTCCCTGGATGATGTATAACTAAGCAGGAGAGAGCTTCGAGTG | |
| | GGTTCACCACATCAGCCACCACTCCTGCTTCTGAGCACAGGGTGCTCTCCT | |
| | CTTGAGCTCAGCTTCTGCTTTTGCAGCCAAGCATTCTTGCTGCTGCTGCCTG | |
| | CCTGCCCACCCGCCTGGGCTTGCAGCCCGCCACTTTACTTTCTCCAGCCCTG | |
| | ATACCAGCTGAGAAGTCTCCCTGCAGCTGCTAGTTCCTGCCCAGGACCATG | |
| | TGTGTGGATGCTGCTTGGGAGAAGCGGGCACTTGCTCCTGGCACTGATCCC | |
| | AGCTGAGTTTCTCCTGTTGATTTCTGGACCACTGATGCTGTTGCTGAGGAG | |
| | GTATTTCCCGGCATCCCTCCCCCTGAGACACCGGCTAAGGACCAGCCTAAA | |
| | CGCAAGGTAACCTTCACCTCTTTCCCATGGGATTTTCCCCAGCTGGGATAT | |
| | CTCTGCTTTCTCCTGGAACTAGGTCCTCCAGCTGGCAACATATCTCTCTCTC | |
| | TCTCTCTCTCTCTCTCTCTCTCTCTCTCTCTGTGTGTGTGTGTGTGTGTTGGG | |
| | GGAGAAGAGGAGACTGTTTTAGGTATTGCTCTGTCCTAGGGCTTCCCCGAT | |
| | ACCTGCTGAATCCAATGCAAAGTGGCCTTTCCCCTGGCTCTGGAGACATTT | |
| | CCGAGGCAGGCACCTGTCATCTGCCACCCCCGTCCTCCTCCTCGAGCTCCC | |
| | TTTCTCCCCCTCCCCCAGCCCATTATTCTGCTTCAGCCTTTTGTGTCAGTGG | |
| | CAGAGGGCTGAAGGGATGTCTTTGCCCTTCTGGCAGGCAGGACAGTGTCA | |
| | GGATGGACCGCGCTGCCAGAAGCCGACGCTAGCGAGGGAGGTGTGAAGA | |
| | GTTGGCCAG | |
| 45 | TCTAGATGTACCACTGACATCTGCTTTAAATCTTTTGCCCATTTCATTTTTA | G88P3 |
| | CCCTCTAACCATTTCATTACTAAAACACAAAACAAACTCCGGAAAGGAGTT | |
| | TGTTGATGAGAAAACACTGAAAGTACGATTTAAATTGAGGAATGGAGATT | |
| | TTCTGTGGATAATTGTCTATGTTCCACTTTACCAAAGAAAAATGAATTTCA | |
| | AGTAGTGCATAAAGTAATTCTCGTTAAAAAATGTGGGAATAAAGAGATCA | |
| | GTCCTGGGTACTGGGAATATATTTATCTCACAGAAAAATCTTGCCCACAAA | |
| | ATGCATCAGACTGAGATGAAATACTCTAGGGTATATTAAATTTTATTCTAA | |
| | TATTGAAGGGAGGGAAAAAAAGGACGGAAAGAAAAATGAAACAGGATGA | |
| | AAAATAAGGTGATTGGGAGAGAGAAAAGACAGAGAAACTTGTTAACGAC | |
| | AGGACTTTCTGAGCAAGATATTTTTCTTCCTTTTACAAGTTGGAAGCTATTG | |
| | AATAATTCATGCCAAGCGTGGAAAGGCTGTCTTTTCCCTGGATGATGTATA | |
| | ACTAAGCAGGAGAGAGCTTCGAGTGGGTTCACCACATCAGCCACCACTCC | |
| | TGCTTCTGAGCACAGGGTGCTCTCCTCTTGAGCTCAGCTTCTGCTTTTGCAG | |
| | CCAAGCATTCTTGCTGCTGCTGCCTGCCTGCCCACCCGCCTGGGCTTGCAG | |
| | CCCGCCACTTTACTTTCTCCAGCCCTGATACCAGCTGAGAAGTCTCCCTGC | |
| | AGCTGCTAGTTCCTGCCCAGGACCATGTGTGTGGATGCTGCTTGGGAGAAG | |
| | CGGGCACTTGCTCCTGGCACTGATCCCAGCTGAGTTTCTCCTGTTGATTTCT | |
| | GGACCACTGATGCTGTTGCTGAGGAGGTATTTCCCGGCATCCCTCCCCCTG | |
| | AGACACCGGCTAAGGACCAGCCTAAACGCAAGGTAACCTTCACCTCTTTCC | |
| | CATGGGATTTTCCCCAGCTGGGATATCTCTGCTTTCTCCTGGAACTAGGTCC | |
| | TCCAGCTGGCAACATATCTCTCTCTCTCTCTCTCTCTCTCTCTCTCTCTCTCT | |
| | GTGTGTGTGTGTGTGTGTTGGGGGAGAAGAGGAGACTGTTTTAGGTATTGC | |
| | TCTGTCCTAGGGCTTCCCCGATACCTGCTGAATCCAATGCAAAGTGGCCTT | |
| | TCCCCTGGCTCTGGAGACATTTCCGAGGCAGGCACCTGTCATTTGCCACCC | |
| | CCGTCCTCCTCCTCGAGCTCCCTTTCTCCCCCTCCCCCAGCCCATTATTCTG | |
| | CTTCAGCCTTTTGTGTCAGTGGCAGAGGGCTGAAGGGATGTCTTTGCCCTT | |
| | CTGGCAGGCAGGACAGTGTCAGGATGGACCGCGCTGCCAGAAGCCGACGC | |
| | TAGCGAGGGAGGTGTGAA | |
| 46 | ATGCCAAGCGTGGAAAGGCTGTCTTTTCCCTGGATGATGTATAACTAAGCA | G88P7 |
| | GGAGAGAGCTTCGAGTGGGTTCACCACATCAGCCACCACTCCTGCTTCTGA | |
| | GCACAGGGTGCTCTCCTCTTGAGCTCAGCTTCTGCTTTTGCAGCCAAGCATT | |
| | CTTGCTGCTGCTGCCTGCCTGCCCACCCGCCTGGGCTTGCAGCCCGCCACTT | |
| | TACTTTCTCCAGCCCTGATACCAGCTGAGAAGTCTCCCTGCAGCTGCTAGT | |
| | TCCTGCCCAGGACCATGTGTGTGGATGCTGCTTGGGAGAAGCGGGCACTTG | |
| | CTCCTGGCACTGATCCCAGCTGAGTTTCTCCTGTTGATTTCTGGACCACTGA | |
| | TGCTGTTGCTGAGGAGGTATTTCCCGGCATCCCTCCCCCTGAGACACCGGC | |
| | TAAGGACCAGCCTAAACGCAAGGTAACCTTCACCTCTTTCCCATGGGATTT | |
| | TCCCCAGCTGGGATATCTCTGCTTTCTCCTGGAACTAGGTCCTCCAGCTGGC | |
| | AACATATCTCTCTCTCTCTCTCTCTCTCTCTCTCTCTCTCTCTCTCTGTGTGT | |
| | GTGTGTGTGTGTTGGGGGAGAAGAGGAGACTGTTTTAGGTATTGCTCTGTC | |
| | CTAGGGCTTCCCCGATACCTGCTGAATCCAATGCAAAGTGGCCTTTCCCCT | |
| | GGCTCTGGAGACATTTCCGAGGCAGGCACCTGTCATCTGCCACCCCCGTCC | |
| | TCCTCCTCGAGCTCCCTTTCTCCCCCTCCCCCAGCCCATTATTCTGCTTCAG | |
| | CCTTTTGTGTCAGTGGCAGAGGGCTGAAGGGATGTCTTTGCCCTTCTGGCA | |
| | GGCAGGACAGTGTCAGGATGGACCGCGCTGCCAGAAGCCGACGCTAGCGA | |
| | GGGAGGTGTGAAGAGTTGGCCAG | |
| 47 | CCAAGCACTGTGTTACACATCTCCTCATTAAAATGGTATAGCAACCTTTTCT | R9P1 |
| | GGTCGATATTATGATCCCATTTTTCAATTTTTACTTTTGAGTGAAGGACATT | |
| | GATTCTTAGAGATTGTAAGGAACTTGCCCAAGGCTGCCGGCTGGTTAGTGG | |
| | CAGAGCTGGGATGGGAAAGTCCGTATTTTCCACCGCGCTGTATTAGTGAGG | |
| | GCTCTTTGTGTCACTTCTGTGCATAACTCCGCCCCAAGTTAAAAGGCTCGCT | |
| | GCTCTCGACAGGTCCTCCTCCTCCTCCCGCTAGGTCCTCCTCCTCCTCCCGC | |
| | CAGGTCCTCCTCCTCCTTCTCCTCCCGGGGCCCTGCCCTGCGGGTCTCACCT | |
| | GCTGCTCCGCGGGGTCCCGGCGGGGGCCGGATAGCGCCCCCTGGCGGCAG | |
| | GTGGGCGCCCGCGCAGCGCATGCTAATGAGCCCGCGGCGCCGCTCGCGGA | |
| | TTGGCTGGCGCGGATTCCAGCTGCTTTCCAAGTCAGCGGCGCCTAGTGAGA | |
| | GTCAGGGGGGCCCGGCCCGCGCCCTCCCCGCCCAGCCGCCTCCCCGTCGAC | |
| | GCCCAGGGCTGGGGCGAGCCAGGCTGCCTTTCGAACTTGGGGGGCTTCTCC | |
| | TCTTGTCTCCCACTGGTGCTCTGGCTGTGAATCCATCCAGGGGCCAGGATG | |
| | ACAATCCGACACCAAGGCCAGCAGTACAGGCCGAGGATGGCATTTCTCCA | |
| | AAAGGTAACCCTGGCCCCTCACCTGGACCCTGGGAGGAGGGCGGGGGACC | |
| | GCATCTGCGAAGCGTCCGAGGACAAGAGGGGCAGGAGTGGAGAGCACCC | |
| | CTTTGGGTCGATTTGTGCTGCTCGATTTTATTAAGTGACTTCTTTGCTGTGT | |
| | GTGTCGATATTGATGGCTATAATAATTAGGTTTTCGTGTGCCCTGCCTGTAG | |
| | TGGAGAATCTGAAGCCAGCGCTGATGGTCTGCGGGAGGTGAAAACAACCT | |
| | GTTACTCGGGAGAGGCAGGTTCAGTTCAGGCTGTGGCAGATTTGAGACAG | |
| | CCTGCTGATATTGTTGTAAGACGACCAGATGTGACATTGCCATTTTTGCCTT | |
| | TTTTTTCTGATCCCAAACTGCACTATTTTAAAGTGTGTGTGTGTGTACACAT | |
| | AGACCATGAAAACGGGGTGATTTGTAGGATAAAGGGCCACCCCATTTACA | |
| | ATGCCCTGAGAGCCCTCATTTGAGTGTGTCTATCTGTGCATCTCCTGCTGTC | |
| | TGCGCTGGCGGCAGAAGTCCCAACCTTTTCGCTTCCCTTGCGAATGCTTTCC | |
| | CAGACAAAGTGACCGGGCACAGGGTGTGCACACAGGATGGACATGTGGGA | |
| | GGTGGAATGCACCTGTGGGGTGATGTGTTTGGGGACAAAGTGGTTCATTTA | |
| | GTTCTGAGCTTCTGGTATAATCACTGTCCGATGAGATCTACAGAGGATTAG | |
| | GTCCCGGGTCACATCTTTGTCCTTCCTCCCACCCGTAGCCACTTTTCACAGC | |
| | GTAGTGTCCATCTTCTTAGGCTTATCTTGTTGAGTAGAATTAGTTGAATGTT | |
| | TTGTGACAAGCTGAGCTCTCCCTGAGCCCCTGGAGCTCAGCAAATTTGTAT | |
| | AGTCAGTGACCGCTGTCCTGATTTGCCCAGGACTTTCAATGCTAAAACCTG | |
| | GACAGTCCTGGGGACCCCGAGACGGTCGCATTTGGTCCACCTGTTTCAAGA | |
| | TGCTCGTGCTCATCTTCCGGGTAGCAGAGGTGTGGGTATCATCTGTAAAGC | |
| | AGACAGGCTGAATGTTGTCTCACTCAGAAAGAACCCTGAGAGCCTGTATTG | |
| | CAGATACCAGTTACTAAAAGCAGAAGAAACTGCCTAAGTCGGCTATCTCA | |
| | AAGCCACACATCATCTTCCAGGATTTATCCTAGGACTGTCTCCTCCCAGGC | |
| | AGGACAGCTCTGGCTTGGGGGCTCAGAGGGTAATTGTCAAGAGATAAATT | |
| | CAGTTTGTTTATTGAATCTCTGGCATTCTTTTCCATCATGACTCTTCTCTGGT | |
| | AGATTCTCACCCCTGTGTCCACCTTTGCATTTTGAGGTCCCTCTCAGTGCAG | |
| | TGGAAATTGCCCTGCACAACTTTCAAAATTGTTCTCGTCAGTCGGCTTTTTC | |
| | CTGTTCTGTCTTGCAGATTGAAGCGCTCGTGAAGGACATGCAGAACCCAGA | |
| | GACAG | |
| 48 | tacagagtagttccactgtcctaagaatcttccgtgatcatctattcattcctcctttccccctaactcgggcaaccactggtctttt | G88P21 |
| | taactgtctccatagttttgccttctcaagaatgtcatagttggtagcatacagtatgtagtcctttccagttggcttctgtcacttag | |
| | tgatacacacttaaggttccttcatgtcttttcatggctggacagctcatttctttttagcactgaataatattccattgtctagatgta | |
| | ccactgacatctgctttaaatcttttgcccatttcatttttaccctctaaccatttcattactaaaacacaaaacaaactccggaaag | |
| | gagtttgttgatgagaaaacactgaaagtacgatttaaattgaggaatggagattttctgtggataattgtctatgttccactttac | |
| | caaagaaaaatgaatttcaagtagtgcataaagtaattctcgttaaaaaatgtgggaataaagagatcagtcctgggtactggg | |
| | aatatatttatctcacagaaaaatcttgcccacaaaatgcatcagactgagatgaaatactctagggtatattaaattttattctaat | |
| | attgaagggagggaaaaaaaggacggaaagaaaaatgaaacaggatgaaaaataaggtgattgggagagagaaaagac | |
| | agagaaacttgttaacgacaggactttctgagcaagatatttttcttccttttacaagttggaagctattgaataattcatgccaag | |
| | cgtggaaaggctgtcttttccctggGtgatgtataactaagcaggagagagcttcgagtgggttcaccacatcagccaccac | |
| | tcctgcttctgagcacagggtgctctcctcttgagctcagcttctgcttttgcagccaagcattcttgctgctgctgcctgcctgc | |
| | ccacccgcctgggcttgcagcccgccactttactttctccagccctgataccagctgagaagtctccctgcagctgctagttcc | |
| | tgcccaggaccaggtgtgtggacgctgcttgggagaagcgggcacttgctcctggcactgatcccagctgagtttctcctgtt | |
| | gatttctggaccactgacgctgttgctgaggaggtatttcctggcatccctccccctgagacaccggctaaggaccagcctaa | |
| | acgcaaggtaaccttcacctctttcccatgggattttccccagctgggatatctctgctttctcctggaactaggtcctccagctg | |
| | gcaacatctctctctctctctctctctctctctctctctGtGtGtGtgtgtgtgtgtgtgtgtgttgggggagaagaggagactg | |
| | ttttaggtattgctctgtcctagggcttccccgatacctgctgaatccaatgcaaagtggcctttcccctggctctggagacattt | |
| | ccgaggcaggcacctgtcatctgccacccccgtcctcctcctcgagctccctttctccccctcccccagcccattattctgcttc | |
| | agccttttgtgtcagtggcagagggctgaagggatgtctttgcccttctggcaggcaggacagtgtcaggatggaccgcgct | |
| | gccagaagccgacgctagcgagggaggtgtgaagagttggccag | |
| 49 | MGTLHNNSTTSPLFPNISSSWIHSPSDAGLPPGTVTHFGSYNVSRAAGNFSSPD | hM3Ds |
| | GTTDDPLGGHTVWQVVFIAFLTGILALVTIIGNILVIVSFKVNKQLKTVNNYFL | |
| | LSLACADLIIGVISMNLFTTYIIMNRWALGNLACDLWLAIDCVASNASVMNLL | |
| | VISFDRYFSITSPFRYQSLMTRARAGVMIGLAWVISFVLWAPAILFWQYFVGK | |
| | RTVPPGECFIQFLSEPTITFGTAIAGFYMPVTIMTILYWRVYREAKEQIRKIDRC | |
| | EGRFYGSQEQPQPPPLPQHQPILGNGRASKRKTSRVMAMREHKALQTLSAILL | |
| | AFIITWTPYNIMVLVNTFCDSCIPKTFWNLGYWLCYINSTVNPVCYALCNKTF | |
| | RTTFKMLLLCQCDKKKRRKQQYQQRQSVIFHKRAPEQAL | |
| 50 | MGTLHNNSTTSPLFPNISSSWIHSPSDAGLPPGTVTHFGSYNVSRAAGNFSSPD | hM3Ds |
| | GTTDDPLGGHTVWQVVFIAFLTGILALVTIIGNILVIVSFKVNKQLKTVNNYFL | (A147S- |
| | LSLACADLIIGVISMNLFTTYIIMNRWALGNLACDLWLSIDCVASNASVMNLL | F349Y) |
| | VISFDRYFSITSPFRYQSLMTRARAGVMIGLAWVISFVLWAPAILFWQYFVGK | |
| | RTVPPGECFIQFLSEPTITFGTAIAGFYMPVTIMTILYWRVYREAKEQIRKIDRC | |
| | EGRFYGSQEQPQPPPLPQHQPILGNGRASKRKTSRVMAMREHKALQTLSAILL | |
| | AFIITWTPYNIMVLVNTFCDSCIPKTYWNLGYWLCYINSTVNPVCYALCNKTF | |
| | RTTFKMLLLCQCDKKKRRKQQYQQRQSVIFHKRAPEQAL* | |
| 51 | tacagagtagttccactgtcctaagaatcttccgtgatcatctattcattcctcctttccccctaactcgggcaaccactggtctttt | G88P20 |
| | taactgtctccatagttttgccttctcaagaatgtcatagttggtagcatacagtatgtagtcctttccagttggcttctgtcacttag | |
| | tgatacacacttaaggttccttcatgtcttttcatggctggacagctcatttctttttagcactgaataatattccattgtctagatgta | |
| | ccactgacatctgctttaaatcttttgcccatttcatttttaccctctaaccatttcattactaaaacacaaaacaaactccggaaag | |
| | gagtttgttgatgagaaaacactgaaagtacgatttaaattgaggaatggagattttctgtggataattgtctatgttccactttac | |
| | caaagaaaaatgaatttcaagtagtgcataaagtaattctcgttaaaaaatgtgggaataaagagatcagtcctgggtactggg | |
| | aatatatttatctcacagaaaaatcttgcccacaaaatgcatcagactgagatgaaatactctagggtatattaaattttattctaat | |
| | attgaagggagggaaaaaaaggacggaaagaaaaatgaaacaggatgaaaaataaggtgattgggagagagaaaagac | |
| | agagaaacttgttaacgacaggactttctgagcaagatatttttcttccttttacaagttggaagctattgaataattcatgccaag | |
| | cgtggaaaggctgtcttttccctggatgatgtataactaagcaggagagagcttcgagtgggttcaccacatcagccaccact | |
| | cctgcttctgagcacagggtgctctcctcttgagctcagcttctgcttttgcagccaagcattcttgctgctgctgcctgcctgcc | |
| | cacccgcctgggcttgcagcccgccactttactttctccagccctgataccagctgagaagtctccctgcagctgctagttcct | |
| | gcccaggaccatgtgtgtggatgctgcttgggagaagcgggcacttgctcctggcactgatcccagctgagtttctcctgttg | |
| | atttctggaccactgatgctgttgctgaggaggtatttcccggcatccctccccctgagacaccggctaaggaccagcctaaa | |
| | cgcaaggtaaccttcacctctttcccatgggattttccccagctgggatatctctgctttctcctggaactaggtcctccagctgg | |
| | caacatatctctctctctctctctctctctctctctctctctctctctgtgtgtgtgtgtgtgtgttgggggagaagaggagactgttt | |
| | taggtattgctctgtcctagggcttccccgatacctgctgaatccaatgcaaagtggcctttcccctggctctggagacatttcc | |
| | gaggcaggcacctgtcatctgccacccccgtcctcctcctegagctccctttctccccctcccccagcccattattctgcttca | |
| | gccttttgtgtcagtggcagagggctgaagggatgtctttgcccttctggcaggcaggacagtgtcaggatggaccgcgctg | |
| | ccagaagccgacgctagcgagggaggtgtgaagagttggccag | |

What is claimed is:

1. A retro adeno-associated virus (retro-AAV), wherein the retro-AAV comprises:
a. an adeno-associated virus capsid polypeptide comprising one or more alterations that promote retrograde transport of the retro-AAV by a neuron, and
b. a nucleic acid comprising a heterologous gene of interest operatively coupled to a GPR88 regulatory region or a R9P1 regulatory region.

2. The retro-AAV of claim 1, wherein the retro-AAV is a serotype selected from AAV2, AAV8 or a combination thereof.

3. The retro-AAV of claim 1, wherein the one or more alterations that promote retrograde transport of the retro-AAV by a neuron is selected from the list consisting of an insertion of SEQ ID NO: 31, an aspartic acid substitution at an amino acid residue corresponding to position 385 of SEQ ID NO: 1, an isoleucine and asparagine (IN) substitution at an amino acid residue corresponding to positions 721 and 722 of SEQ ID NO: 1, and combinations thereof.

4. The retro-AAV of claim 1, comprising an alteration at one or more amino acid residues corresponding to V125, V183, N411, Y447, R490, T495, or F536 of SEQ ID NO: 1.

5. The retro-AAV of claim 1, wherein the virus capsid polypeptide comprises an amino acid sequence that possesses at least 90%, 95%, 97%, 98%, 99% sequence identity or that is identical to the amino acid sequence set forth in SEQ ID NO: 1, wherein the virus capsid polypeptide comprises an alteration to SEQ ID NO: 1 at an amino acid selected from the list consisting of any one or more of V125, V183, N411, Y447, R490, T495, F536, and A606.

6. The retro-AAV of claim 5, wherein the virus capsid polypeptide comprises an alteration to SEQ ID NO: 1 corresponding to an amino acid selected from the list consisting of any two or more of V125, V183, N411, Y447, R490, T495, F536, and A606.

7. The retro-AAV of claim 5, wherein the virus capsid polypeptide comprises an alteration to SEQ ID NO: 1 corresponding to an amino acid selected from the list consisting of any three or more of V125, V183, N411, Y447, R490, T495, F536, and A606.

8. The retro-AAV of claim 5, wherein the virus capsid polypeptide comprises a substitution to SEQ ID NO: 1 corresponding to an amino acid selected from the list consisting of any one or more of V125I, V183E, N411S, Y447F, R490Q, T495A, F536Y, and A606S.

9. The retro-AAV of claim 5, wherein the virus capsid polypeptide comprises a substitution to SEQ ID NO: 1 corresponding to an amino acid selected from the list consisting of any two or more of V125I, V183E, N411S, Y447F, R490Q, T495A, F536Y, and A606S.

10. The retro-AAV of claim 5, wherein the virus capsid polypeptide comprises a substitution to SEQ ID NO: 1 corresponding to an amino acid selected from the list consisting of any three or more of V125I, V183E, N411S, Y447F, R490Q, T495A, F536Y, and A606S.

11. The retro-AAV of claim 5, wherein the virus capsid polypeptide comprises a substitution corresponding to V125I and N411S of SEQ ID NO: 1.

12. The retro-AAV of claim 5, wherein the virus capsid polypeptide comprises a substitution corresponding to V125I, F536Y, T495A of SEQ ID NO: 1.

13. The retro-AAV of claim 5, wherein the virus capsid polypeptide comprises a substitution corresponding to V125I, A606S, and a T495A of SEQ ID NO: 1.

14. The retro-AAV of claim 1, wherein the virus capsid polypeptide comprises the amino acid sequence set forth in any one of SEQ ID NOs 1 to 15.

15. The retro-AAV of claim 1, wherein the virus capsid polypeptide comprises the amino acid sequence set forth in any one of SEQ ID NOs 2 to 15.

16. The retro-AAV of claim 1, wherein the GPR88 regulatory region comprises a nucleotide sequence corresponding to a genomic sequence positioned 3′ to a translational start site of an endogenous GPR88 gene.

17. The retro-AAV of claim 16, wherein the genomic sequence positioned 3′ to a translational start site of the endogenous GPR88 gene is partially positioned in an intron.

18. The retro-AAV of claim 16, wherein the genomic sequence positioned 3′ to a translational start site of the endogenous GPR88 gene is positioned in an intron.

19. The retro-AAV of claim 16, wherein the genomic sequence positioned 3′ to a translational start site of the endogenous GPR88 gene is positioned less than about 1,000 nucleotides 3′ to the translational start site of the endogenous GPR88 gene.

20. The retro-AAV of claim 16, wherein the GPR88 regulatory region comprises a nucleotide sequence that is at least 80%, 85%, 90%, 95%, 97%, 98%, 99% homologous to the nucleotide sequence set forth in SEQ ID NO: 39.

21. The retro-AAV of claim 16, wherein the GPR88 regulatory region comprises a nucleotide sequence that is identical to the nucleotide sequence set forth in SEQ ID NO: 39.

22. The retro-AAV of claim 16, wherein the genomic sequence positioned 3′ to a translational start site of the endogenous GPR88 gene is positioned less than about 900 nucleotides 3′ to the translational start site of the endogenous GPR88 gene.

23. The retro-AAV of claim 22, wherein the GPR88 regulatory region comprises a nucleotide sequence that is at least 80%, 85%, 90%, 95%, 97%, 98%, 99% homologous to the nucleotide sequence set forth in SEQ ID NO: 40.

24. The retro-AAV of claim 22, wherein the GPR88 regulatory region comprises a nucleotide sequence that is identical to the nucleotide sequence set forth in SEQ ID NO: 40.

25. The retro-AAV of claim 1, wherein the GPR88 regulatory region comprises a nucleotide sequence corresponding to a genomic sequence positioned 5′ to the translational start site of the endogenous GPR88 gene.

26. The retro-AAV of claim 25, wherein the genomic sequence positioned 5′ to a translational start site of the endogenous GPR88 gene is positioned less than about 100 nucleotides 5′ to the translational start site of the endogenous GPR88 gene.

27. The retro-AAV of claim 26, wherein the GPR88 regulatory region comprises a nucleotide sequence that is at least 80%, 85%, 90%, 95%, 97%, 98%, 99% homologous to the nucleotide sequence set forth in SEQ ID NO: 41.

28. The retro-AAV of claim 26, wherein the GPR88 regulatory region comprises a nucleotide sequence that is identical to the nucleotide sequence set forth in SEQ ID NO: 41.

29. The retro-AAV of claim 25, wherein the genomic sequence positioned 5′ to a translational start site of the endogenous GPR88 gene is positioned less than about 600 nucleotides 5′ to the translational start site of the endogenous GPR88 gene.

30. The retro-AAV of claim 29, wherein the GPR88 regulatory region comprises a nucleotide sequence that is at least 80%, 85%, 90%, 95%, 97%, 98%, 99% homologous to the nucleotide sequence set forth in SEQ ID NO: 42.

31. The retro-AAV of claim 29, wherein the regulatory element comprises a nucleotide sequence that is identical to the nucleotide sequence set forth in SEQ ID NO: 42.

32. The retro-AAV of claim 25, wherein the genomic sequence positioned 5′ to a translational start site of the endogenous GPR88 gene is positioned less than about 1,500 nucleotides 5′ to the translational start site of the endogenous GPR88 gene.

33. The retro-AAV of claim 32, wherein the GPR88 regulatory region comprises a nucleotide sequence that is at least 80%, 85%, 90%, 95%, 97%, 98%, 99% homologous to the nucleotide sequence set forth in SEQ ID NO: 43.

34. The retro-AAV of claim 32, wherein the GPR88 regulatory region comprises a nucleotide sequence that is identical to the nucleotide sequence set forth in SEQ ID NO: 43.

35. The retro-AAV of claim 1, wherein the GPR88 regulatory region comprises a nucleotide sequence that is at least 80%, 85%, 90%, 95%, 97%, 98%, 99% homologous to the nucleotide sequence set forth in any one of SEQ ID NOs: 44, 45, 46, or 48.

36. The retro-AAV of claim 1, wherein the GPR88 regulatory region comprises a nucleotide sequence that is identical to the nucleotide sequence set forth in any one of SEQ ID NOs: 44, 45, 46, or 48.

37. The retro-AAV of claim 1, wherein R9P1 regulatory region comprises a nucleotide sequence that is at least 80%, 85%, 90%, 95%, 97%, 98%, 99% homologous to the nucleotide sequence set forth in SEQ ID NO:
47.

38. The retro-AAV of claim 10, wherein the heterologous gene of interest is 3′ to the GPR88 regulatory region or the R9P1 regulatory region.

39. The retro-AAV of claim 1, wherein the heterologous gene of interest possesses therapeutic utility.

40. The retro-AAV of claim 1, wherein the heterologous gene of interest comprises a neurotrophic factor, an RNA guided nuclease, an enzyme, or a DREADD.

41. The retro AAV of claim 40, wherein the heterologous gene of interest comprises a DREADD.

42. The retro-AAV of claim 40, wherein the DREADD is rM3Ds.

43. The retro-AAV of claim 40, wherein the DREADD comprises an amino acid sequence exhibiting at least about 90%, 95%, 97%, 98%, 99% identity to or is identical to SEQ ID NO: 38.

44. The retro-AAV of claim 40, wherein the DREADD is hM3Ds.

45. The retro-AAV of claim 40, wherein the DREADD comprises an amino acid sequence exhibiting at least about 90%, 95%, 97%, 98%, 99% identity to or is identical to SEQ ID NO: 49.

46. The retro-AAV of claim 40, wherein the DREADD is hM3Ds(A147S-F349Y).

47. The retro-AAV of claim 40, wherein the DREADD comprises an amino acid sequence exhibiting at least about 90%, 95%, 97%, 98%, 99% identity to or is identical to SEQ ID NO: 50.

48. The retro-AAV of claim 1, wherein the heterologous gene of interest comprises one or more of hM3Dq, hM1Dq, hMD5q, hM4Di, hM2Di, or BDNF.

49. The retro-AAV of claim 1, wherein the heterologous gene of interest exhibits increased expression of the heterologous gene of interest compared to the promoter of the hSYN1 gene in a neuron of the striatum.

50. The retro-AAV of claim 1, wherein the heterologous gene of interest exhibits at least a 2-fold increase in expression of the heterologous gene of interest compared to the promoter of the hSYN1 gene in a neuron of the striatum.

51. The retro-AAV of claim 1, wherein the heterologous gene of interest exhibits at least a 5-fold increase in expression of the heterologous gene of interest compared to the promoter of the hSYN1 gene in a neuron of the striatum.

52. The retro-AAV of claim 1, wherein the heterologous gene of interest exhibits at least a 25-fold increase in expression of the heterologous gene of interest compared to the promoter of the hSYN1 gene in a neuron of the striatum.

53. A pharmaceutical composition comprising a pharmaceutically acceptable, carrier, excipient, or diluent and the retro-AAV of claim 1.

54. The pharmaceutical composition of claim 53, for use in a method to express a polypeptide in a neuron of the striatum.

55. The use of claim 54, wherein the neuron of the striatum is a D1 dopaminergic medium spiny neuron.

56. The pharmaceutical composition of claim 53, for use in a method to genetically engineer a neuron of the striatum.

57. The use of claim 56, wherein the neuron of the striatum is a D1 dopaminergic medium spiny neuron.

58. The pharmaceutical composition of claim 53, for use in a method to treat a neurodegenerative disease in an individual.

59. The use of claim 58, wherein the neurodegenerative disease comprises Parkinson's disease.

60. A method to express a polypeptide in a neuron of the striatum of an individual comprising administering the pharmaceutical composition of claim 53, to the individual thereby expressing the polypeptide the neuron of the striatum.

61. The method of claim 60, wherein the neuron of the striatum is a D1 dopaminergic medium spiny neuron.

62. A method to genetically engineer a neuron of the striatum of an individual comprising administering the pharmaceutical composition of claim 53 to the individual thereby genetically engineering the neuron of the striatum.

63. The method of claim 62, wherein the neuron of the striatum is a D1 dopaminergic medium spiny neuron.

64. A method to treat an individual afflicted with a neurodegenerative disease comprising administering the pharmaceutical composition of claim 53 to the individual afflicted with a neurodegenerative disease thereby treating the neurodegenerative disease.

65. The method of claim 64, wherein the neurodegenerative disease comprises Parkinson's disease.

66. The method of claim 60, wherein the individual is a mammal.

67. The method of claim 60, wherein the individual is a human.

68. A method to express and activate a DREADD in the central nervous system of an individual comprising administering to the individual the retro-AAV of claim 1 and a ligand that activates the DREADD, thereby activating the DREADD in the central nervous system of the individual.

69. The method of claim 68, wherein the DREADD is expressed and activated int in a neuron of the striatum.

70. The method of claim 69, wherein the neuron of the striatum is a D1 dopaminergic medium spiny neuron.

71. The method of claim 68, wherein the individual is a mammal.

72. The method of claim 68, wherein the individual is a human.

73. The method of claim 68, wherein activating the DREADD in the central nervous system of the individual treats a neurodegenerative disorder.

74. The method of claim 73, wherein the neurodegenerative disorder comprises Parkinson's disease.

75. The method of claim 68, wherein the ligand that activates the DREADD comprises quetiapine or clozapine.

76. The method of claim 68, wherein the ligand that activates the DREADD comprises quetiapine.

77. The method of claim 68, wherein the ligand that activates the DREADD comprises clozapine.

78. The method of claim 68, wherein the retro-AAV and the ligand that activates the DREADD are administered separately.
